(19) United States
(12) Patent Application Publication
(10) Pub. No.: US 2009/0196912 A1
(43) Pub. Date: Aug. 6, 2009

(54) Pyridinylamines

(75) Inventors: Jan Elke Eickhoff, Munich (DE); Doris Hafenbradl, Pullach (DE); Wilfried Schwab, Neuried (DE); Matthew Cotton, Fajara Banjul (GM); Bert Matthias Kiebl, Gunzlhofen (DE); Birgit Zech, Munich (DE); Stefan Muller, Munich (DE); John Harris, Kent (GB); Vladimir Savic, Belgrade (SA); Jackie MacRitchie, Essex (GB)

Correspondence Address: ROPES & GRAY LLP, PATENT DOCKETING 39/41, ONE INTERNATIONAL PLACE, BOSTON, MA, 02110-2624, US

(73) Assignee: GPC Botech AG, Martinsried/Munich (DE)

(21) Appl. No.: 11/659,013

(22) PCT Filed: Aug. 1, 2005
(86) PCT No.: PCT/EP2005/008321

§ 371 Date: Jul. 22, 2007

Related U.S. Application Data
(60) Provisional application No. 60/599,307, filed on Aug. 6, 2004.

(30) Foreign Application Priority Data
Jul. 30, 2004 (EP) 04018131.5

(51) Int. Cl. A61K 9/127; C07D 213/72; A61K 31/44; C07D 401/04; A61K 31/506; A61K 31/443; C07D 405/12; C07D 413/02; A61K 31/5377; A61K 9/28; A61P 35/00; A61P 3/10

(52) U.S. Cl. 424/450; 546/312; 514/352; 544/310; 514/274; 514/338; 546/283.7; 544/124; 514/235.5; 424/474

(57) ABSTRACT

Described are pyridinylamines and pharmaceutically acceptable salts thereof, the use of these pyridinylamines for the prophylaxis and/or treatment of various diseases such as infectious diseases, including opportunistic infections, prion diseases immunological diseases, autoimmune diseases, bipolar and clinical disorders, cardiovascular diseases, cell proliferative diseases, diabetes, inflammation, transplant rejections, erectile dysfunction, neurodegenerative diseases and stroke, as well as pharmaceutical compositions containing at least one pyridinylamine and/or pharmaceutically acceptable salts thereof. Furthermore, reaction procedures for the synthesis of the pyridinylamines are disclosed.

The present invention relates to pyridinylamines and pharmaceutically acceptable salts thereof, the use of these compounds for the prophylaxis and/or treatment of various diseases such as infectious diseases, including infectious diseases and opportunistic infections, prion diseases, immunological diseases, autoimmune diseases, bipolar and clinical disorders, cardiovascular diseases, cell proliferative diseases, diabetes, inflammation, transplant rejections, erectile dysfunction, neurodegenerative diseases and stroke, as well as compositions containing at least one pyridinylamine and/or pharmaceutically acceptable salts thereof. Furthermore, reaction procedures for the synthesis of said pyridinylamines are disclosed.

Object of the present invention is to provide pharmaceutically active compounds for prophylaxis and treatment of various diseases such as infections, inflammations, immunological diseases, cardiovascular diseases, cell proliferative diseases, transplant rejections, or neurodegenerative diseases, methods for the synthesis of said compounds and pharmaceutical compositions containing at least one pharmaceutically active compound.

This object is solved by the pyridinylamines as described herein below, and/or pharmaceutically acceptable salts of said compounds, the use of at least one of those compounds and/or the pharmaceutically acceptable salts thereof as pharmaceutically active agents as described herein below, the use of the compounds as an inhibitor for a protein kinase as described herein below, the use of the compounds for prophylaxis and/or treatment of various diseases as described herein below, and the pharmaceutical composition as described herein below. Further advantageous features, aspects and details of the invention are evident from the claims, the description, the examples and the drawings.

One aspect of the present invention is related to compounds of the general formula (I):

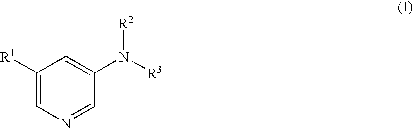

wherein:
R¹ represents —CR²³(R²⁴)R²⁵, —CR²⁸(R²⁹)—CR²⁶(R²⁷)—CR²³(R²⁴)R²⁵, —CCRR²²⁶⁶((RR²²⁷⁷))—CC—RR²²³³((RR²²⁴⁴))RR²²⁵⁵. —((CCHH₂)), n-CR²⁸(R²⁹)—CR²⁶(R²⁷)—CR²³(R²⁴) R²⁵, —(CH₂)ₙ—CH═CH—(CH₂)r-CR²³(R²⁴)R²⁵,

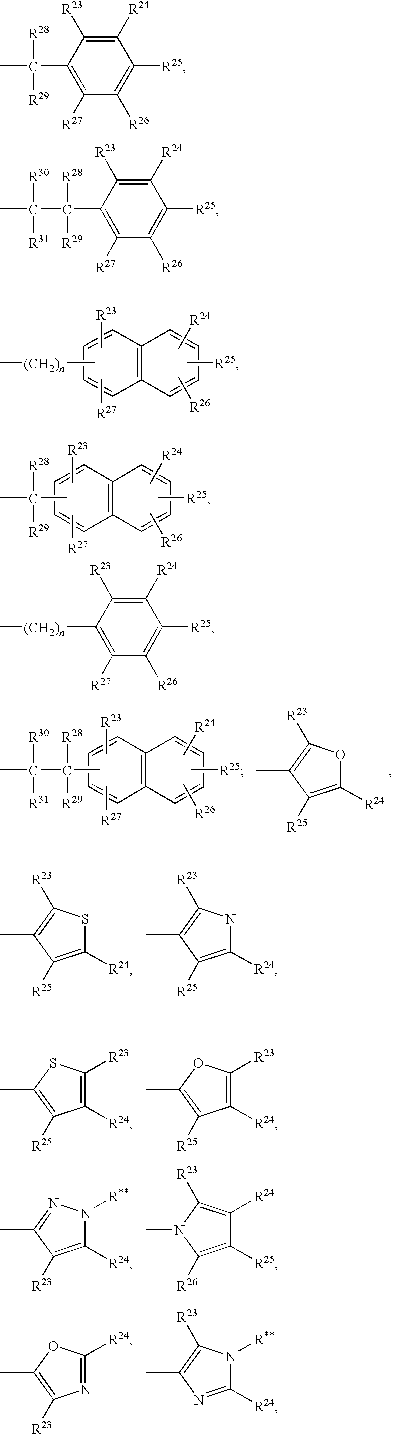

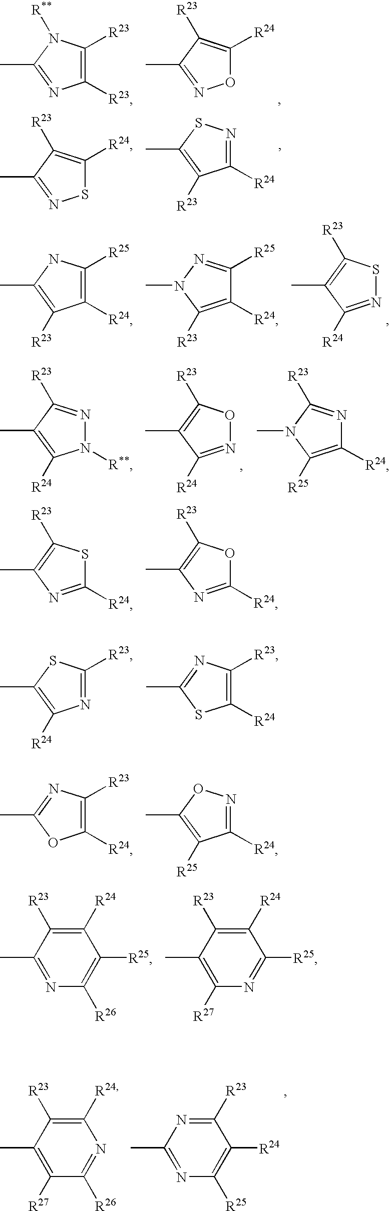

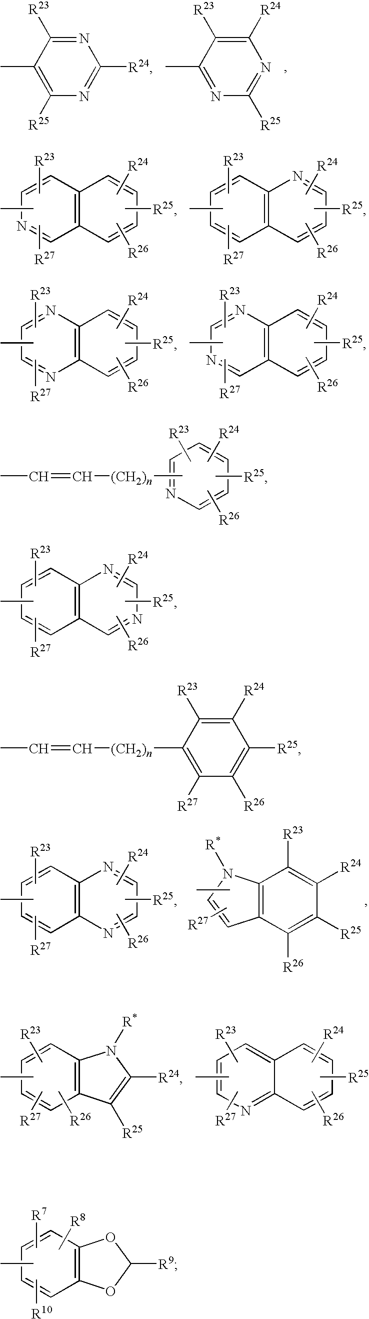

R², R* and R** represent independently of each other —H, —CH₃, —C₂H₅, —CH═CH₂, —C═CH, —C₃H₇, -cyclo-C₃H₅, —CH(CHs)₂, —CH₂—CH═CH₂, —C(CHs)═CH₂, —CH═CH—CH₃, —C≡C—CH₃, —CH₂—C═CH, —C₄H₉, -cyclo-C₄H₇, —CH₂—CH(CHs)₂, —CH(CHs)—C₂H₅, —C(CHs)₃, —C₅H₁₁, —R¹, —R′, —R¹′, -CyClO—C₅H₉, —C₆H₁₃, -CyClo-C₆H₁₁, -Ph, —CH₂Ph, —C₆H₄—CH₃, —CW)R″¹, —C₂(ROs, —CH₂—CRXR″)̂¹, —CHR^{l}—CH(R″)R^{m}, —C(R^{l})R″—CH₂—R^{l}″, —C₃(R′)₇, —C₂H₄—C(R′)₃, —CHO, —COCH₃, —COC₂H₅, —COC₃H₇, —COC₄H₉, —CO—CyClo-C₃H₅, —COCH(CHs)₂, —COC(CHs)₃, —COPh, —CO—CH₂Ph, —CO—C₆H₄—CH₃, —COOCH₃, —COOC₂H₅, —COOC₃H₇, COOC₄H₉, COO—CyClO—C₃H₅, —COOCH(CHs)₂, —COOC(CH₃)₃, —COOPh, —COO—CH₂Ph, —COO—C₆H₄—CH₃;
R′, R″ and R¹″ represent independently of each other —H, —F, —Cl, —Br, —I, —CN, —SO₃H, —CONH₂, —OH, —SH, —OCH₃, —OC₂H₅, —SCH₃, —SC₂H₅, —NH₂, —NO₂, —NH(CH₃), —N(CHg)₂, —NH(C₂H₅), —N(C₂Hg)₂, —OCF₃, —CH₂F —CHF₂, —CF₃, —CH₂Cl, —CH₂Br, —CH₂l, —CH₂—CH₂F, —CH₂—CF₃, —CH₂—CH₂Cl, —CH₂—CH₂Br, —CH₂—CH₂l, —CH₃, —C₂H₅, —C₃H₇, —CH(CH₃)₂, —C₄H₉, —CH₂—CH(CHs)₂, —CH(CHs)—C₂H₅, —C(CHs)₃, C₅Hn, —CH(CH₃)—C₃H₇, —CH₂—CH(CH₃)—C₂H₅, —CH(CHs)—CH(CHs)₂, —C(CHs)₂-C₂H₅, —CH₂—C(CHs)₃, —CH(C₂Hg)₂, —C₂H₄—CH(CHs)₂, —C₆H₁₃—, —C₃He—CH(CHs)₂, —C₂H₄—CH(CHs)—C₂H₅, —CH(CHs)—C₄Hg, —CH₂—CH(CHs)—CsH₇, —CH(CH₃)—CH₂—CH(CH₃)₂, —CH(CHs)—CH(CHs)—C₂H₅, —CH₂—CH(CHs)—CH(CHs)₂, —CH₂—C(CHs)₂-C₂H₅, —C(CH₃)₂—C₃H₇, —C(CH₃)₂—CH(CHs)₂, —C₂H₄—C(CHs)₃, —CH(CH₃)—C(CH₃)₃, -Ph, —CH₂-Ph, —C₆H₄—CH₃, —CPh₃, —CH═CH₂, —CH₂—CH═CH₂, —C(CHs)═CH₂, —CH═CH—CH₃, —C₂H₄—CH═CH₂, —CH═C(CHs)₂, —CH₂—CH═CH—CH₃, —C≡CH, —C═C—CH₃, —CH₂—C═CH, —CHO, —COCH₃, —COC₂H₅, —COC₃H₇, —CO—CyClo-C₃H₅, —COCH(CH₃)₂, —COC(CH₃)₃, —OOC—CH₃, —OOC—C₂H₅, —COOH, —COOCH₃, —COOC₂H₅, —COOC₃H₇, —COO—CyClO—C₃H₅, —COOCH(CH₃)₂, —COOC(CHs)₃;
R³ represents —R⁴, —CO—R⁴, —CO—CH(R⁵)—R⁴. —CH(R⁵)—R⁴, —CH(R⁵)—CH(R⁶)—F>⁴. —CH(R⁵)—CO—R⁴, —CH(R⁵)—CH(R⁶)—CO—R⁴, —CH(R⁵)—O—CO—R⁴, —CH(R⁵)—CH(R⁶)—O—CO—R⁴, —CO—NH—R⁴, —CO—O—R⁴, —SO₂—R⁴, —CH(R⁵)—SO₂—R⁴, —CH(R⁵)—CH(R⁶)—SO2-R⁴;
R⁴ represents —CR¹⁶(R¹⁷)R¹⁸, —CR²¹(R²²)—CR¹⁹(R²⁰)—CR¹⁶(R¹⁷)R¹⁸, —CR¹⁹(R²VCR¹⁶(R¹⁷)R¹⁸, —(CH₂)ₙ—CR²¹(R [symbol] ²²)—CR [symbol] ¹⁹( [symbol] R [symbol] )—CR [symbol] ( [symbol] R [symbol] ) R¹⁸,

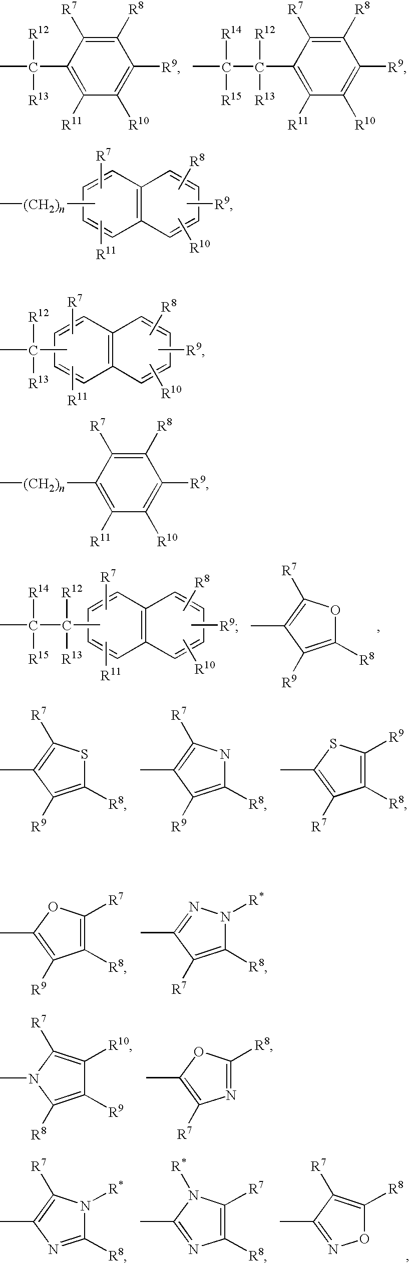

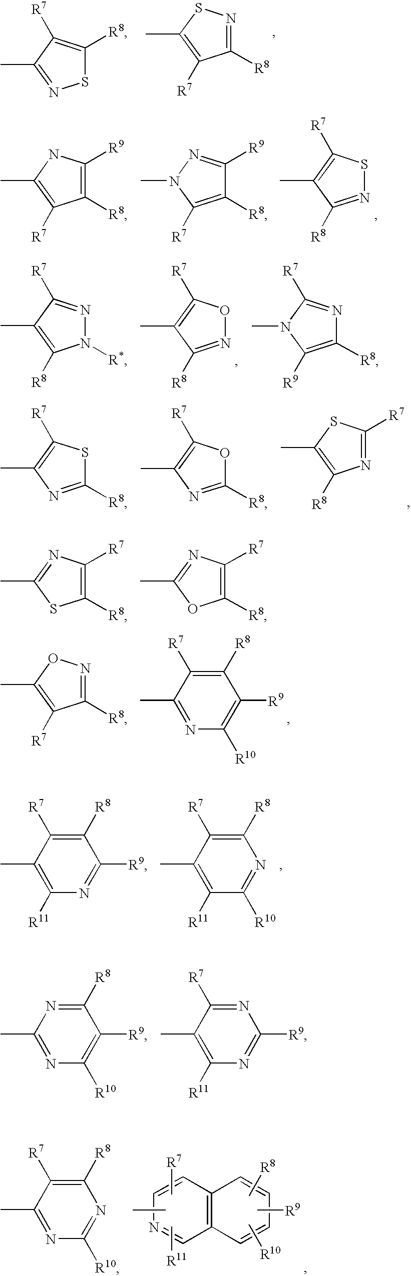

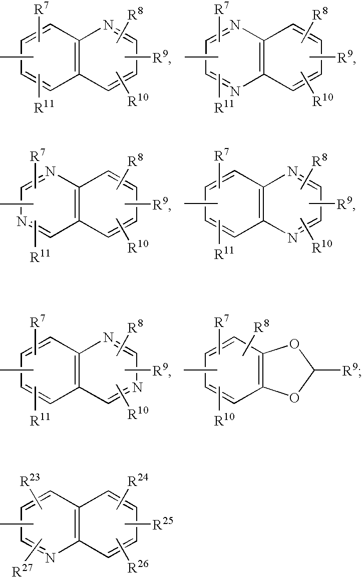

R² and R³ can form together a heterocyclic ring wherein the residue

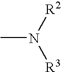

represents one of the following moieties:

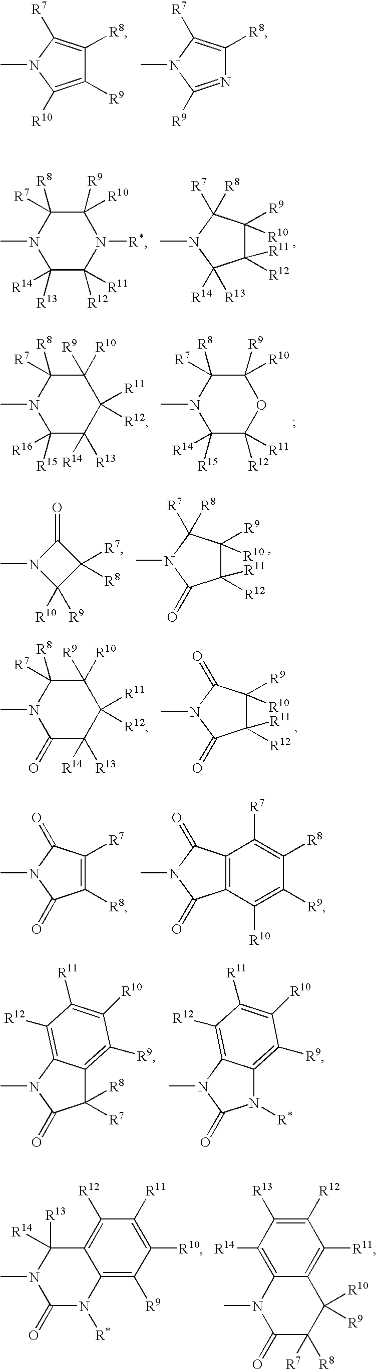

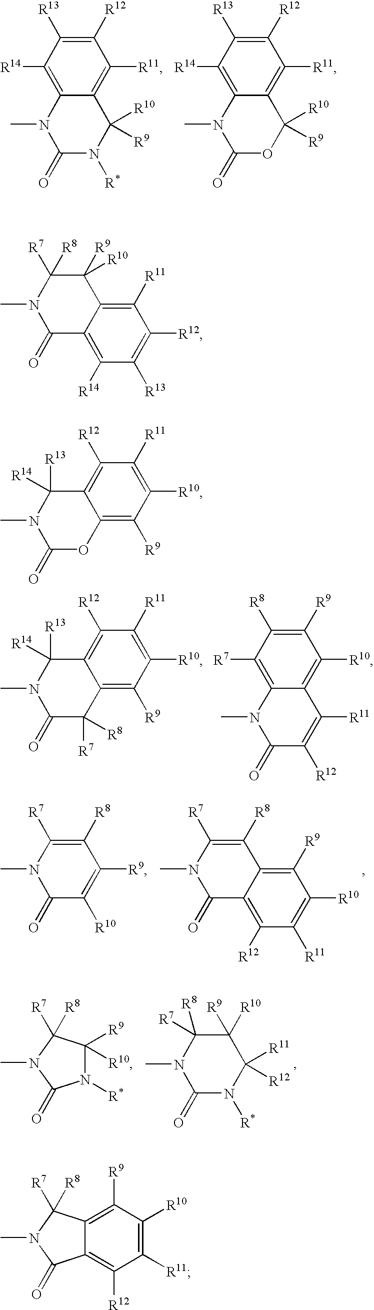

R⁵-R³¹ represent independently of each other
—(CH₂)ₘ—CR³²R³³R³⁴, —CR³⁵R³⁶R³⁷, —CR⁴³R⁴⁴—CR⁴⁵R⁴⁶—CR⁴⁷R⁴⁸R⁴⁹, —CR³⁸R³⁹—CR⁴⁰R⁴¹R⁴², —X—(CH₂)ₘ—CR³²R³³R³⁴, —X—CR³⁵ [symbol] R³⁶ [symbol] R³⁷, —X—CR³⁸R³⁹—CR⁴⁰R⁴¹R⁴², —X—CR⁴³R⁴⁴—CR⁴⁵R⁴⁶—CR⁴⁷R⁴⁸R⁴⁹, —CH₂R⁵⁰, —X—CH₂R⁵¹, —(CH₂)ₚ—R⁵³, —X—CH₂)_{q}—R⁵⁴;
X represents —CO—, —O—, —S—, —NR—*, —NH—CO—, —CO—NH—, —O—CO—, —CO—O—, —SO₂—, —SO—, —SO₂—O—, —NH—SO₂—, —O—SO₂—, —O—CO—O—, —O—CO—NH—, —NH—CO—O—, —NH—CO—NH—, —NH—CS—NH—, —NH—C(═NH)—NH—, —CF₂—, —C₂F₄—, —C₃F₆—;
R⁵-R⁵⁴ represent independently of each other
—H, —OH, —OCH₃, —OC₂H₅, —OC₃H₇, —O-cyclo-C₃H₅, —OCH(CH₃)₂, —OC(CH₃)₃, —OPh, —OCH₂-Ph, —SH, —SCH₃, —SC₂H₅, —SC₃H₇, —S-cyclo-C₃H₅, —SCH(CHs)₂, —SC(CHs)₃, —NO₂, —F, —Cl, —Br, —I, —N₃, —CN, —CHO, —COCH₃, —COC₂H₅, —COC₃H₇, —CO-cyclo-C₃H₅, —COCH(CH₃)₂, —COC(CHs)₃, —COOH, —COOCH₃, —COOC₂H₅, —COOC₃H₇, —COO-cyclo-C₃H₅, —COOCH(CH₃)₂, —COOC(CH₃)₃, —OOC—CH₃, —OOC—C₂H₅, —OOC—C₃H₇, —OOC-cyclo-C₃H₅, —OOC—CH(CH₃)₂, —OOC—C(CH₃)₃, —CONH₂, —CONHCH₃, —CONHC₂H₅, —CONHC₃H₇, —CONH-cyclo-C₃H₅, —CONH[CH(CHs)₂], —CONH[C(CHs)₃], —CON(CHs)₂, —CON(C₂Hs)₂, —CON(C₃H₇)₂, —CON[CH(CHs)₂]₂, —NH₂, —NHCH₃, —NHC₂H₅, —NHC₃H₇, —NH-cyclo-C₃H₅, —NHCH(CHs)₂, —NHC(CHs)₃, —N(CH₃)₂, —N(C₂Hg)₂, —N(C₃H₇)₂, —N(cyclo-C₃H)₂, —N[CH(CH₃)₂]₂, —N[C(CH₃)₃]₂, —SOCH₃, —SOC₂H₅, —SOC₃H₇, —SO—CyClO—C₃H₅, —SOCH(CH₃)₂, —SOC(CH₃)₃, —SO₂CH₃, —SO₂C₂H₅, —SO₂C₃H₇, —SO₂—CyClo-C₃H₅, —SO₂CH(CH₃)₂, —SO₂C(CHs)₃, —SO₃H, —SO₃CH₃, —SO₃C₂H₅, —SO₃C₃H₇, —SO₃—CyClo-C₃H₅, —SO₃CH(CHs)₂, —SO₃C(CH₃)_{S}, —NH—SO₂CH₃, —NH—SO₂C₂H₅, —NH—SO₂Ph, —NH—SO₂C₄H₆—CH₃, SO₂NH₂, —OCF₃, —OC₂F₅, —O—COOCH₃,
—O—COOC₂H₅, —O—COOC₃H₇, —O—COO-cyclo-CsHs, —O—COOCH(CH₃)₂, —O—COOC(CH₃)s, —NH—CO—NH₂, —NH—CO—NHCHs, —NH—CO—NHC₂H₅, —NH—CO—NHC₃H₇, —NH—CO—NH-CyClO-C₃H₅, —NH—CO—NH[CH(CHs)₂], —NH—CO—NH[C(CH₃)₃], —NH—CO—N(CH₃)₂, —NH—CO—N(C₂Hs)₂, —NH—CO—N(C₃H₇)₂, —NH—CO—N(cyclo-C₃H₅)₂, —NH—CO—N[CH(CH₃)₂]₂, —NH—CO—N[C(CH₃)s]₂, —NH—CS—NH₂, —NH—CS—NHCH₃, —NH—CS—NHC₂H₅, —NH—CS—NHC₃H₇, —NH—CS—NH-CyClo-C₃H₅, —NH—CS—NH[CH(CHs)₂], —NH—CS—NH[C(CH₃)₃], —NH—CS—N(CH₃)₂, —NH—CS—N(C₂Hs)₂, —NH—CS—N(C₃H₇)₂, —NH—CS—N(cyclo-C₃H₅)₂, —NH—CS—N[CH(CHs)₂₋₂, —NH—CS—N[C(CH₃)₃]₂, —NH—C(═NH)—NH₂ₗ—NH—CC═NH)—NHCH₃₁—NH—CC═NH)—NHC₂H₅, —NH—CC═NH)—NHC₃H₇, —NH—CC═NH)—NH-CyClo-C₃H₅₁—NH—ĈNHJ-NHtCHCCH-Oa], —NH—C(═NH)—NH[C(CH₃)₃], —O—CO—NHCH₃, —NH—CC═NH)—N(CHs)₂, NH—C(═NH)—N(C₂H₅)₂, —O—CO—NHC₃H₇, —NH—C(═NH)—N(C₃H7)₂, —NH—C(═NH)—N(cyclo-C₃H₅)2, —O—CO—NH₂, —NH—C(═NH)—N[CH(CH₃)₂]₂, —NH—C(═NH)—N[C(CH₃)₃]₂, —O—CO—NHC₂H₅, —O—CO—NH-cyclo-CsHs, —O—CO—NH[CH(CH₃)₂], —O—CO—NH[C(CH₃)₃], —O—CO—N(CH₃)₂, —O—CO—N(C₂H₅)₂, —O—CO—N(C₃Hr)₂, —O—CO—N(cyclo-C₃H₅)₂, —O—CO—N[CH(CH₃)₂]2, —O—CO—N[C(CH₃)₃]₂, —O—CO—OCH₃, —O—CO—OC₂H₅, —O—CO—OC₃H₇, —O—CO—O—CyClo-C₃H₅, —O—CO—OCH(CHs)₂, —O—CO—OC(CH₃)s, —CH₂F—CHF₂, —CF₃, —CH₂Cl, —CHCl₂, —CCl₃, —CH₂Br—CHBr₂, —Cl₃, —CH₂—CH₂F—CH₂—CHF₂, —CH₂—CF₃, —CH₂—CH₂Cl, —CH₂—CHCl₂, —CH₂—CCl₃, —CH₂—CH₂Br—CH₂—CHBr₂, —CH₂—CBr₃, —CH₂—Cl₃, —CH₃, —C₂H₅, —C₃H₇, —CH(CHs)₂, —C₄H₉, —CH₂—CH(CHs)₂, —CH(CHs)—C₂H₅, —C(CHs)₃, —C₅Hi₁, —CH(CHs)—C₃H₇, —CH₂—CH(CHs)—C₂H₅, —CH(CH₃)—CH(CH₃)₂, —C(CHs)₂-C₂H₅, —CH₂—C(CHs)₃, —CH(C₂Hg)₂, —C₂H₄—CH(CHs)₂, —C₆H₁₃, —C₃H₆—CH(CHs)₂, —C₂H₄—CH(CH₃)—C₂H₅, —CH(CHs)—C₄H₉, —CH₂—CH(CHs)—C₃H₇, —C(CH₃)₂—CH(CH₃)₂, —CH(CHs)—CH₂—CH(CHs)₂, —CH(CHs)—CH(CHs)—C₂H₅, —C(CHs)₂-C₃H₇, —CH₂—CH(CH₃)—CH(CH₃)₂, —CH₂—C(CHs)₂-C₂H₅, —C₂H₄—C(CHs)₃, —CH(CH₃)—C(CH₃)₃, -Ph, —CH₂-Ph, —C₆H₄—CH₃, —CPh₃, —CH═CH₂, —CH₂—CH═CH₂, —C(CHs)═CH₂, —CH═CH—CH₃, —C₂H₄—CH═CH₂, —CH═C(CH₃)₂, —CH₂—CH═CH—CH₃, —C≡CH, —C≡C—CHs, —CH₂—C═CH;

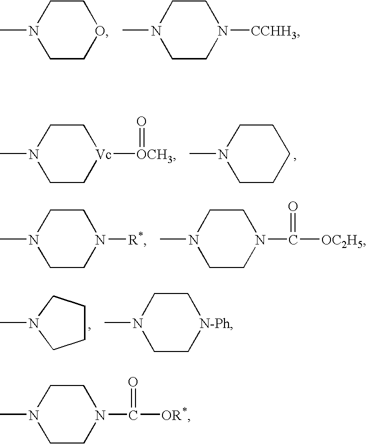

n, r are independently of each other integers from 0-8,
m, p, q are independently of each other integers from 1-8,
and stereoisomeric and regioisomeric forms and pharmaceutically acceptable salts of the compounds of general formula (I).

In another aspect, the present invention is related to compounds of the general formula (I):

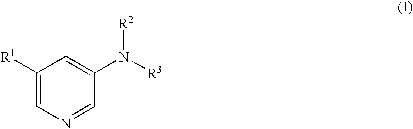

wherein:
R¹ represents —CR²³(R²⁴)R²⁵, —CR²⁸(R²VCR²⁶(R²VCR²³(R²⁴)R²⁵, —CR²⁶(R²⁷)—CR²³(R²⁴)R²⁵, —(CH₂)n-CR²⁸(R²⁹)—CR²⁶(R²⁷)—CR²³(R²⁴)R²⁵, —(CH₂)n-CH═CH—(CH₂)r-CR²³(R²⁴)R²⁵,

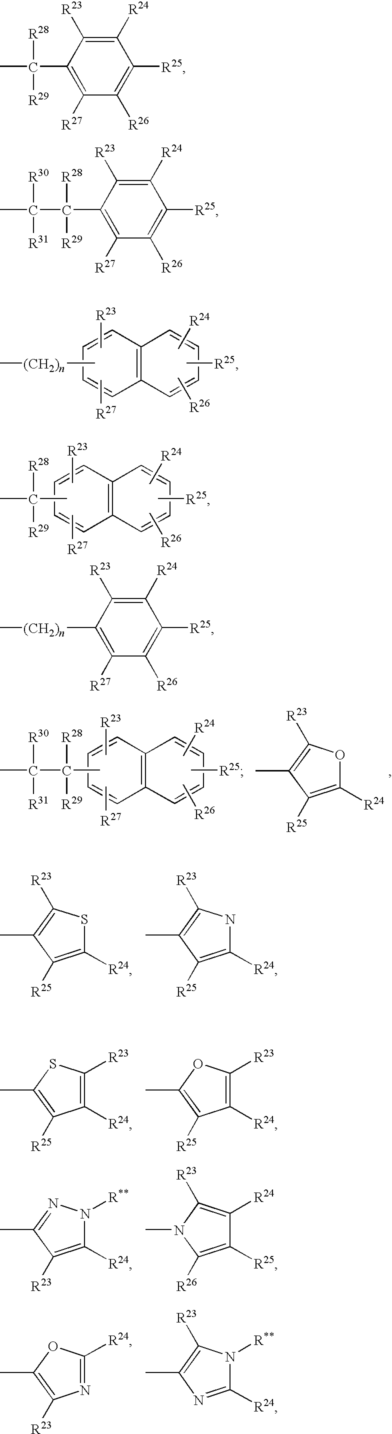

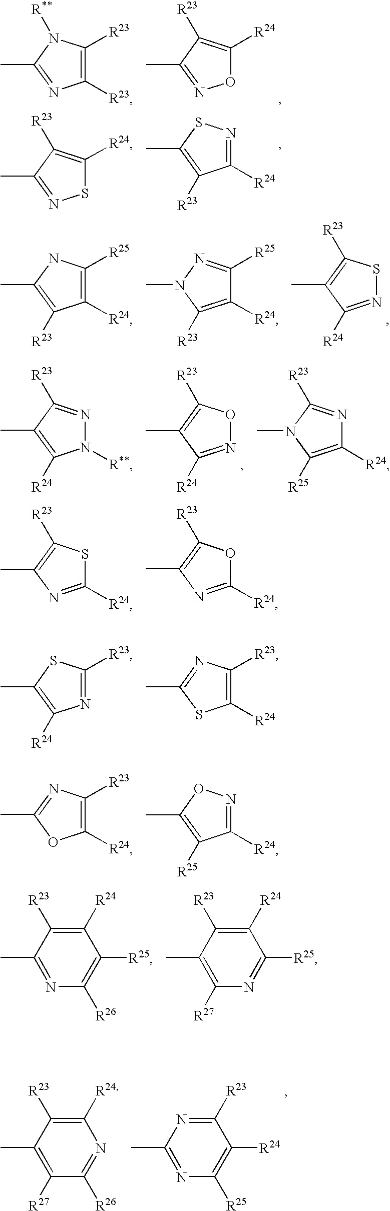

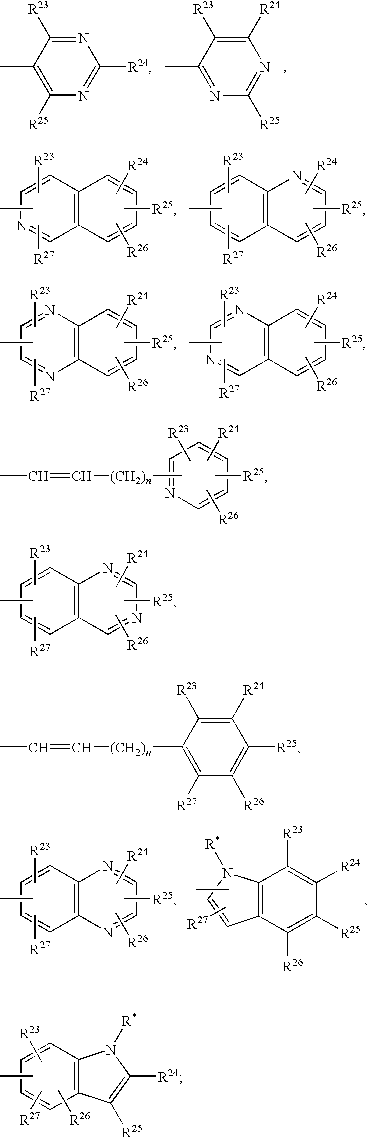

R², R* and R** represent independently of each other —H, —CH₃, —C₂H₅, —CH═CH₂, —C═CH, —C₃H₇, -cyclo-C₃H₅, —CH(CH₃)₂, —CH₂—CH═CH₂, —C(CHs)═CH₂, —CH═CH—CH₃, —C═C—CH₃, —CH₂—C═CH, —C₄H₉, -cyclo-C₄H₇, —CH₂—CH(CH₃)₂, —CH(CH₃)—C₂H₅, —C(CH₃)₃, —C₅Hn, —R¹, —R′, —R″¹, -cyclo-CsHg, —C₆Hi₃, -CyClo-C₆H₁₁, -Ph, —CH₂Ph, —C₆H₄—CH₃, —CR′(R″)R^{m}, —C₂(R¹)₅, —CH₂—CR—(R″)̂″, —CHR′—CH(R″)R^{m}, —C(R^{l})R″—CH₂—R¹′, —C₃(R′)₇, —C₂H₄—C(R¹)₃, —CHO, —COCH₃, —COC₂H₅, —COC₃H₇, —COC₄H₉, —CO—CyClo-C₃H₅, —COCH(CH₃)₂, —COC(CH₃)₃, —COPh, —CO—CH₂Ph, —CO—C₆H₄—CH₃, —COOCH₃, —COOC₂H₅, —COOC₃H₇, —COOC₄H₉, —COO—CyClo-C₃H₅, —COOCH(CH₃)₂, —COOC(CH₃)₃, —COOPh, —COO—CH₂Ph, —COO—C₆H₄—CH₃;
R′, R″ and R¹′ represent independently of each other —H, —F, —Cl, —Br, —I, —CN, —SO₃H, —CONH₂, —OH, —SH, —OCH₃, —OC₂H₅, —SCH₃, —SC₂H₅, —NH₂, —NO₂, —NH(CH₃), —N(CH₃)₂, —NH(C₂H₅), —N(C₂H₅)₂, —OCF₃, —CH₂F —CHF₂, —CF₃, —CH₂Cl, —CH₂Br, —CH₂l, —CH₂—CH₂F, —CH₂—CF₃, —CH₂—CH₂Cl, —CH₂—CH₂Br, —CH₂—CH₂l, —CH₃, —C₂H₅, —C₃H₇, —CH(CH₃)₂, —C₄Hg, —CH₂—CH(CH₃)₂, —CH(CH₃)—C₂H₅, —C(CH₃)₃, —C₅Hn, —CH(CH₃)—C₃H₇, —CH₂—CH(CHs)—C₂H₅, —CH(CH₃)—CH(CH₃)₂, —C(CHs)₂-C₂H₅, —CH₂—C(CHs)₃, —CH(C₂H₅)₂, —C₂H₄—CH(CH₃)₂, —CeHi₃, —C₃Hg—CH(CHs)₂, —C₂H4-CH(CHs)—C₂H₅, —CH(CHs)—C₄H₉, —CH₂—CH(CHs)—C₃H₇, —CH(CH₃)—CH₂—CH(CH3)₂, —CH(CHs)—CH(CHs)—C₂H₅, —CH₂—CH(CH₃)—CH(CH₃)₂, —CH₂—C(CHs)₂-C₂H₅, —C(CHs)₂-C₃H₇, —C(CHs)₂-CH(CHs)₂, —C₂H₄—C(CH₃)s, —CH(CHs)—C(CH₃)s, -Ph, —CH₂-Ph, —C₆H₄—CH₃, —CPh₃, —CH═CH₂, —CH₂—CH═CH₂, —C(CHs)═CH₂, —CH═CH—CH₃, —C₂H₄—CH═CH₂, —CH═C(CH₃)₂, —CH₂—CH═CH—CH₃, —C═CH, —C═C—CH₃, —CH₂—C═CH, —CHO, —COCH₃, —COC₂H₅, —COC₃H₇, —CO—CyClO—C₃Hs, —COCH(CH₃)₂, —COC(CHs)₃, —OOC—CH₃, —OOC—C₂H₅, —COOH, —COOCH₃, —COOC₂H₅, —COOC₃H₇, —COO—CyClO—C₃H₅, —COOCH(CH₃)₂, —COOC(CHs)₃;
R³ represents —R⁴, —CO—R⁴, —CO—CH(R⁵)—R⁴, —CH(R^{δ})—R⁴, —CH(R⁵)—CH(R⁶)—R⁴, —CH(R⁵)—CO—R⁴, —CH(R>CH(R⁶)—CO—R⁴, —CH(R⁵)—O—CO—R⁴, —CH(R⁵)—CH(R⁶)—O—CO—R⁴, —CO—NH—R⁴, —CO—O—R⁴, —SO₂—R⁴, —CH(R⁵)—SO₂—R⁴, —CH(R⁵)—CH(R⁶)—SO₂—R⁴
R⁴ represents —CR¹⁶(R¹⁷)R¹⁸, —CR²¹(R²²)—CR¹⁹(R²⁰)—CR¹⁶(R¹⁷)R¹⁸, —CR¹⁹(R²VCR¹⁶(R¹⁷)R¹⁸, —(CH₂)n-CR²¹(R²²)—CR¹⁹(R²⁰)—CR¹¹⁶⁶((RR¹¹⁷⁷))RR¹¹⁸⁸.

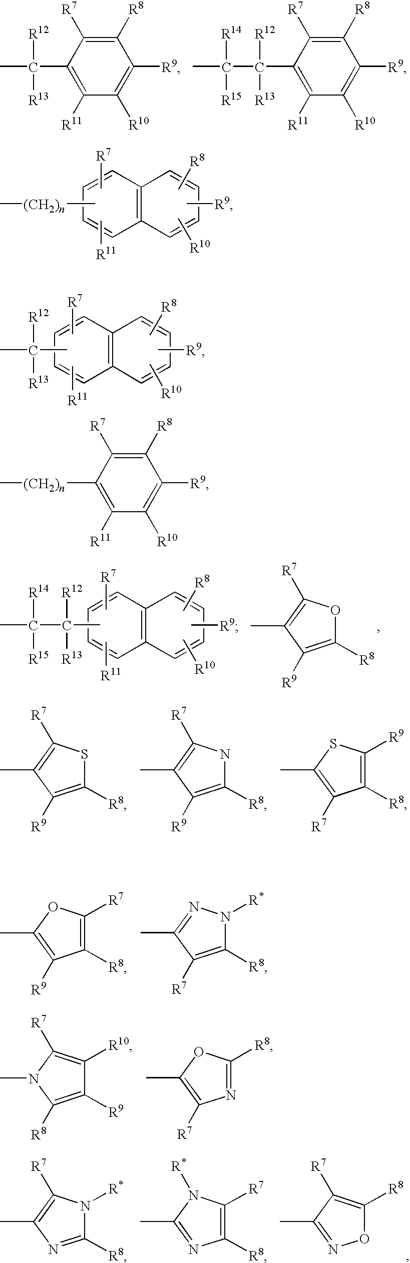

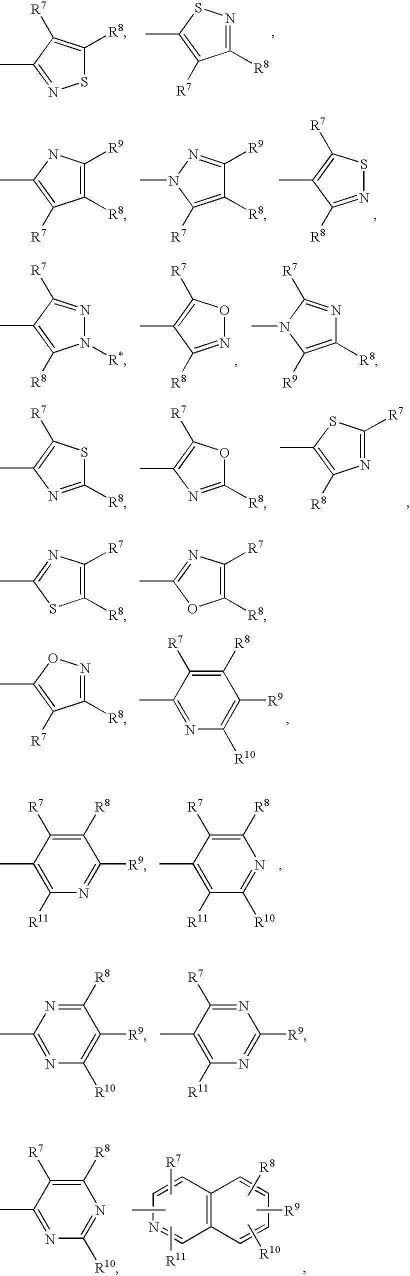

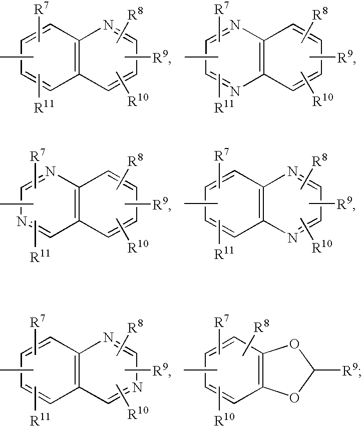

R² and R³ can form together a heterocyclic ring wherein the residue

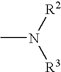

represents one of the following moieties:

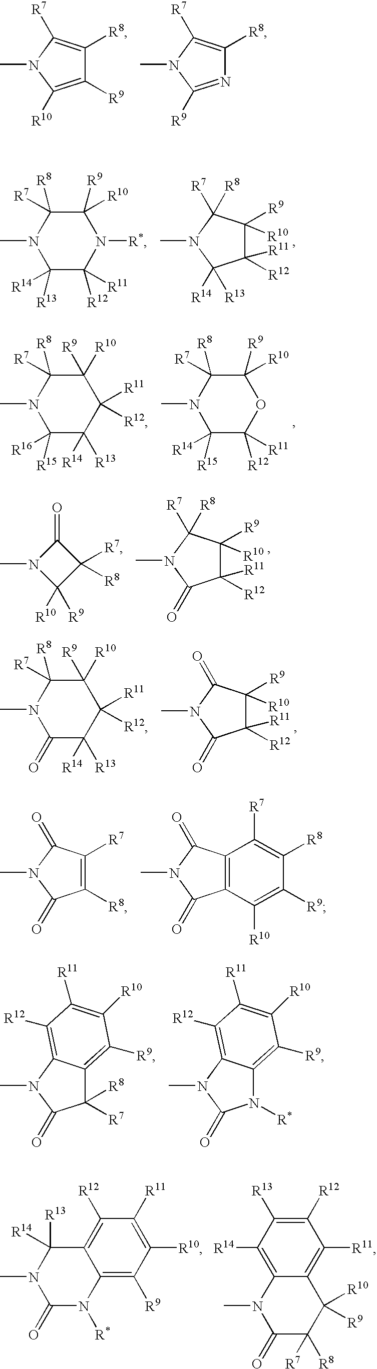

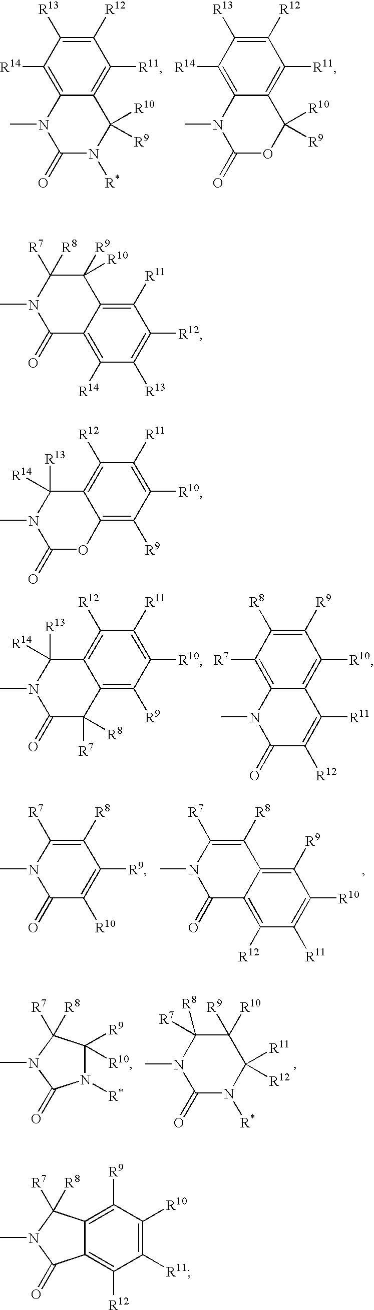

R⁵-R³¹ represent independently of each other
—(CH₂)m-CR³²R³³R³⁴, —CR³⁵R³⁶R³⁷, —CR³⁸R³⁹—CR⁴⁰R⁴¹R⁴², —CR⁴³R⁴⁴—CR⁴⁵R⁴⁶—CR⁴⁷R⁴⁸R⁴⁹, —X—(CH₂)ₘ—CR³²R³³R³⁴, —X—CR³⁵R³⁶R³⁷, —X—CR³⁸R³⁹—CR⁴⁰R⁴¹R⁴², —X—CR⁴³R⁴⁴—CR⁴⁵R⁴⁶—CR⁴⁷R⁴⁸R⁴⁹, —CH₂R⁵⁰, —X—CH₂R⁵¹, —(CH₂)_{P}—R⁵³, —X—(CH₂)_{q}—R⁵⁴;
X represents —CO—, —O—, —S—, —NR—*, —NH—CO—, —CO—NH—, —O—CO—, —CO—O—, —SO₂—, —SO—, —SO₂—O—, —NH—SO₂—, —O—SO₂—, —O—CO—O—, —O—CO—NH—, —NH—CO—O—, —NH—CO—NH—, —NH—CS—NH—, —NH—C(═NH)—NH—, —CF₂—, —C₂F₄—, —C₃F₆—;
R⁵-R⁵⁴ represent independently of each other
—H, —OH, —OCH₃, —OC₂H₅, —OC₃H₇, —O-cyclo-C₃H₅, —OCH(CH₃)₂> —OC(CH₃)₃, —OPh, —OCH₂-Ph, —SH, —SCH₃, —SC₂H₅, —SC₃H₇, —S-CyClO—C₃H₅, —SCH(CHs)₂, —SC(CH₃)₃, —NO₂, —F, —Cl, —Br, —I, —N₃, —CN, —CHO, —COCH₃, —COC₂H₅, —COC₃H₇, —CO—CyClo-C₃H₅, —COCH(CH₃)₂, —COC(CH₃)₃, —COOH, —COOCH₃, —COOC₂H₅, —COOC₃H₇, —COO—CyClO—C₃H₅, —COOCH(CHs)₂, —COOC(CH₃)₃, —O—OC—CH₃, —O—OC—C₂H₅, —OOC—C₃H₇, —OOC-CyClo-C₃H₅, —OOC—CH(CH₃)₂, —OOC—C(CHs)₃, —CONH₂, —CONHCH₃, —CONHC₂H₅, —CONHC₃H₇, —CONH-CyClO-C₃H₅, —CONH[CH(CH₃)₂], —CONH[C(CHs)₃], —CON(CH₃)₂, —CON(C₂Hs)₂, —CON(C₃H₇)₂, —CON[CH(CH₃)₂]₂, —NH₂, —NHCH₃, —NHC₂H₅, —NHC₃H₇, —NH-cyclo-C₃H₅, —NHCH(CHs)₂, —NHC(CH₃)₃, —N(CHs)₂, —N(C₂Hs)₂, —N(C₃H₇)₂, —N(cyclo-C₃H₅)₂, —N[CH(CH₃)₂]₂, —N[C(CH₃)s]₂, —SOCH₃, —SOC₂H₅, —SOC₃H₇, —SO—CyClo-C₃H₅, —SOCH(CHs)₂, —SOC(CH₃)₃, —SO₂CH₃, —SO₂C₂H₅, —SO₂C₃H₇, —SO₂-cyclo-C₃H₅, —SO₂CH(CHs)₂, —SO₂C(CH₃)_{S}, —SO₃H, —SO₃CH₃, —SO₃C₂H₅, —SO₃C₃H₇, —SOs-cyclo-CsHs, —SO₃CH(CHs)₂, —SO₃C(CH₃)₃, —NH—SO₂CH₃, —NH—SO₂C₂H₅, —NH—SO₂Ph, —NH—SO₂C₄H₆—CH₃, —OCF₃, —OC₂F₅, —O—COOCH₃, —O—COOC₂Hs, —O—COOC₃H₇, —O—COO-cyclo-CsHs, —O—COOCH(CH₃)₂, —O—COOC(CH₃)₃> —NH—CO—NH₂, —NH—CO—NHCH₃, —NH—CO—NHC₂H₅, —NH—CO—NHC₃H₇, —NH—CO—NH-CyClO—C₃H₅, —NH—CO—NH[CH(CHs)₂], —NH—CO—NH[C(CHs)₃], —NH—CO—N(CH₃)₂, —NH—CO—N(C₂Hs)₂, —NH—CO—N(C₃HZ)₂, —NH—CO—N(cyclo-C₃H₅)₂, —NH—CO—N[CH(CH₃)₂]₂, —NH—CO—N[C(CH₃)₃]₂, —NH—CS—NH₂, —NH—CS—NHCH₃, —NH—CS—NHC₂H₅, —NH—CS—NHC₃H₇, —NH—CS—NH-CyClo-C₃H₅, —NH—CS—NH[CH(CHs)₂], —NH—CS—NH[C(CHs)₃], —NH—CS—N(CHs)₂, —NH—CS—N(C₂Hs)₂, —NH—CS—N(C₃H₇)₂> —NH—CS—N(cyclo-C₃Hs)₂, —NH—CS—N[CH(CH₃)₂]₂, —NH—CS—N[C(CH₃)₃]₂ₗ —NH—C(═NH)—NH₂, —NH—C(═NH)—NHCH₃, —NH—C(═NH)—NHC₂Hs, —NH—C(═NH)—NHC₃H₇, —NH—C(═NH)—N(C2H₅)₂, —NH—C(═NH)—NH-cyclo-C₃H5, —NH—C(═NH)—NH[CH(CH₃)2], —NH—C(═NH)—N(C₃H₇)₂> —NH—C(═NH)—NH[C(CH₃)s], —NH—C(═NH)—N(CH₃)₂, —NH—C(═NH)—N(cyclo-C₃H₅)₂, —NH—C(═NH)—N[CH(CH₃)₂]₂, —NH—C(═NH)—N[C(CH3)₃]2, —O—CO—NH₂, —O—CO—NHCH₃, —O—CO—NHC₂Hs, —O—CO—NHC₃H₇, —O—CO—NH-CyClO-C₃H₅, —O—CO—NH[CH(CH₃)₂], —O—CO—NH[C(CHs)₃], —O—CO—N(CH₃)₂, —O—CO—N(C₂H₂)₂, —O—CO—N(C₃H₇)₂, —O—CO—N(cyclo-C₃H₅)₂, —O—CO—N[CH(CH₃)₂]₂, —O—CO—N[C(CH₃)s]2, —O—CO—OCHs, —O—CO—OC₂H₅, —O—CO—OC₃H₇, —O—CO—O—CyClo-C₃H₅, —O—CO—OCH(CH₃)₂, —O—CO—OC(CHs)₃, —CH₂F —CHF₂, —CF₃, —CH₂Cl, —CHCl₂, —CCl₃, —CH₂Br—CHBr₂, —Cl₃, —CH₂—CH₂F—CH₂—CHF₂, —CH₂—CF₃, —CH₂—CH₂Cl, —CH₂—CHCl₂, —CH₂—CCl₃, —CH₂—CH₂Br—CH₂—CHBr₂, —CH₂—CBr₃, —CH₂—Cl₃, —CH₃, —C₂H₅, —C₃H₇, —CH(CHs)₂, —C4H9, —CH₂—CH(CH₃)₂, —CH(CHs)—C₂H₅, —C(CHs)₃, —C₅Hn, —CH(CH₃)—CsH₇ₗ—CH₂—CH(CHs)—C₂H₅, —CH(CHs)—CH(CHs)₂, —C(CHs)₂, —C₂H₅, —CH₂—C(CHs)₃, —CH(C₂Hs)₂, —C₂H—CH(CHs)₂, —C₆H₁3, —CsHes-CH(CHs)₂, —C₂H₄—CH(CH₃)—C₂H₅, —CH(CHg)—C₄H₉, —CH₂—CH(CHs)—C₃H₇, —CH(CH₃)—CH₂—CH(CH₃)₂, —CH(CHs)—CH(CHs)—C₂H₅, —CH₂—CH(CH₃)—CH(CH₃)₂, —CH₂—C(CHs)₂-C₂H₅, —C(CH₃)₂—C₃H₇, —C(CHs)₂-CH(CHs)₂, —C₂H₄—C(CH₃)₃, —CH(CH₃)—C(CH₃)₃, -Ph, —CH₂-Ph, —C₆H₄—CH₃, —CPh₃, —CH═CH₂, —CH₂—CH═CH₂, —C(CHs)═CH₂, —CH═CH—CH₃, —C₂H₄—CH═CH₂, —CH═C(CHs)₂, —CH₂—CH═CH—CH₃, —C≡CH, —C═C—CH₃, —CH₂—C═CH;

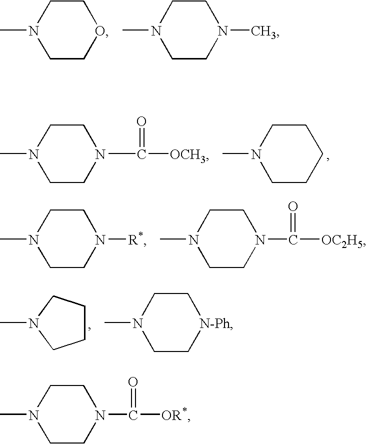

n, r are independently of each other integers from 0-8,
m, p, q are independently of each other integers from 1-8,
and stereoisomeric and regioisomeric forms and pharmaceutically acceptable salts of the compounds of general formula (I).

Yet another aspect of the present invention is related to compounds as described above wherein the following compounds are not encompassed:
4-[5-(4-Chloro-benzylamino)-pyridin-3-yl]-phenol,
4-[5-(3,4-Dichloro-benzylamino)-pyridin-3-yl]-phenol,
4-[5-(3,4-Dimethoxy-benzylamino)-pyridin-3-yl]-phenol,
3-[5-(4-Chloro-benzylamino)-pyridin-3-yl]-benzamide,
3-[5-(3,4-Difluoro-benzylamino)-pyridin-3-yl]-benzamide,
4-[5-(3,4-Difluoro-benzylamino)-pyridin-3-yl]-phenol,
3-{[5-(4-Hydroxy-phenyl)-pyridin-3-ylamino]-methyl}-phenol,
3-{[5-(3-Hydroxymethyl-phenyl)-pyridin-3-ylamino]-methyl}-phenol,
3-[5-(3-Hydroxy-benzylamino)-pyridin-3-yl]-N-(2-hydroxy-ethyl)-benzamide,
3-{5-[(3-Hydroxybenzyl)amino]pyridin-3-yl}phenol,
3-{[5-(4-Hydroxymethyl-phenyl)-pyridin-3-ylamino]-methyl}-phenol,
3-[5-(3-Hydroxy-benzylamino)-pyridin-3-yl]-benzamide,
3-{[5-(3-Amino-phenyl)-pyridin-3-ylamino]-methyl}-phenol, and
3-[(5-Phenyl-pyridin-3-ylamino)-methyl]-phenol.

Yet another aspect of the present invention is related to compounds as described above wherein additionally the following compounds are not encompassed:
3-[5-(4-Chloro-benzylamino)-pyridin-3-yl]-phenol,
{4-[5-(3,4-Difluoro-benzylamino)-pyridin-3-yl]-phenyl}-methanol,
{4-[5-(4-Chloro-benzylamino)-pyridin-3-yl]-phenyl}-methanol,
(4-Dimethylamino-benzyl)-[5-(2-methoxy-phenyl)-pyridin-3-yl]-amine,
N-{3-[5-(3,4-Difluoro-benzylamino)-pyridin-3-yl]-phenyl}-methanesulfonamide,
N-{3-[5-(3,4-Dichloro-benzylamino)-pyridin-3-yl]-phenyl}-methanesulfonamide,
N-{S-[δ-ĈChloro-benzylaminoJ-pyridin-S-yll-phenylJ-methanesulfonamide,
3-[5-(3,4-Dimethoxy-benzylamino)-pyridin-3-yl]-benzamide,
3-[5-(4-Chloro-benzylamino)-pyridin-3-yl]-N-(2-dimethylamino-ethyl)-benzamide,
N-(2-Dimethylamino-ethyl)-3-[5-(3-hydroxy-benzylamino)-pyridin-3-yl]-benzamide,
3-[5-(3,4-Dichloro-benzylamino)-pyridin-3-yl]-phenol,
3-[5-(3,4-Difluoro-benzylamino)-pyridin-3-yl]-phenol,
(3,4-Difluoro-benzyl)-[5-(3,4-dimethoxy-phenyl)-pyridin-3-yl]-amine,
(4-Chloro-benzyl)-[5-(3,4-dimethoxy-phenyl)-pyridin-3-yl]-amine,
3-{[5-(3,4-Dimethoxy-phenyl)-pyridin-3-ylamino]-methyl}-phenol,
N-{S-[δ-CŜ-Dimethoxy-benzylaminoJ-pyridin-S-ylj-phenylJ-acetamidθ,
N-{3-[5-(4-Chloro-benzylamino)-pyridin-3-yl]-phenyl}-acetamide,
N-{3-[5-(3,4-Difluoro-benzylamino)-pyridin-3-yl]-phenyl}-acetamide,
N-{3-[5-(3-Hydroxy-benzylamino)-pyridin-3-yl]-phenyl}-acetamide,
(3,4-Dimethoxy-benzyl)-[5-(3,4-dimethoxy-phenyl)-pyridin-3-yl]-amine,
3-{[5-(2-Hydroxymethyl phenyl)-pyridin-3-ylamino]-methyl}-phenol,
3-{[5-(2-Methoxy-phenyl)-pyridin-3-ylamino]-methyl}-phenol,
3-{[5-(4-Methanesulfonyl-phenyl)-pyridin-3-ylamino]-methyl}-phenol,
3-{[5-(3-Trifluoromethoxy-phenyl)-pyridin-3-ylamino]-methyl}-phenol,
3-{[5-(4-Morpholin-4-yl-phenyl)-pyridin-3-ylamino]-methyl}-phenol, and
N-{3-[5-(3-Hydroxy-benzylamino)-pyridin-3-yl]-phenyl}-methanesulfonamide.

Of the compounds of the invention as described above, a preferred group are those compounds of the general formula (II)

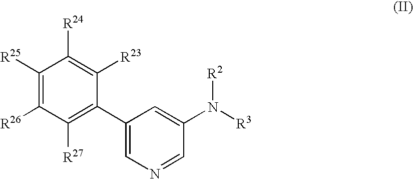

wherein
the substituents R², R³, R²³-R²⁷ have the meanings as defined above.

Another preferred group according to the present invention are those compounds of the general formula (III)

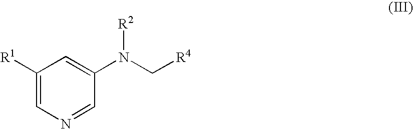

wherein
the substituents R¹, R², R⁴ have the meanings as defined above.

Another preferred group according to the present invention are those compounds of formulae (I), (II) or (III), wherein
R³ is a group —CHR⁵—R⁴, where R [symbol] is H;
R⁴ represents a group

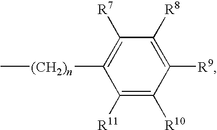

where n is zero;
R¹ represents a group

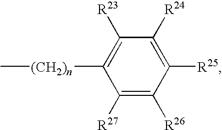

where n is zero;
R² is selected from the group consisting of
—H, —CH₃, —C₂H₅, —C₃H₇, -cyclo-C₃H₅, —CH(CH₃)₂, —C₄H₉, -cyclo-C₄H₇, —CH₂—CH(CHs)₂, —CH(CHs)—C₂H₅, —C(CH₃)₃, —C₅H₁₁, -cyclo-C₅H₉, —C₆H₁₃, -CyClo-C₆H₁₁, -Ph, —CH₂Ph, —C₆H₄—CH₃, —CHO, —COCH₃, —COC₂H₅, COC₃H₇, —COC₄H₉, —CO-cyclo-C₃H₅, —COCH(CHs)₂, —COC(CHs)₃, —COPh, —CO—CH₂Ph, —CO—C₆H₄—CH₃, —COOCHs, —COOC₂H₅, —COOC₃H₇, —COOC₄H₉, —COO-cyclo-C₃H₅, —COOCH(CHs)₂, —COOC(CHs)₃, —COOPh, —COO—CH₂Ph, —COO—C₆H₄—CH₃; and
R⁷-R¹¹ and R²³-R²⁷ have the meanings as defined above for compound of formula (I)—

Of this group of compounds, a more preferred subgroup according to the present invention are those compounds wherein
R² is —H, —CH₃, Or—COOC₄H₉;
each substituent R⁷-R¹¹ and R²³-R²⁷ is independently selected from the group consisting of —H, —F, —Cl, —Br, —OH, —CH₃, —C₂H₅, —C₃H₇, -cyclo-C₃H₅, —CH(CHs)₂, —C₄H₉, -cyclo-C₄H₇, —CH₂—CH(CHs)₂, —CH(CHs)—C₂H₅, —C(CHs)₃, —C₅H₁₁, —OCH₃, OCF₃, —NH₂, N(CHs)₂, —N(C₂Hg)₂, —NO₂, —COOH, —COOCH₃, —CONH₂, —CN, SO₂CH₃, NHSO₂CH₃,

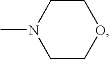

### —CR³⁵R³⁶R³⁷,

—X—(CH₂)ₘ—CR³²R³³R³⁴, Or—X—CH₂—R⁵¹;
X is —NHCO— or —CONH—;
R⁵¹ is H;
each of the substituents R³³-R³⁶ is H;
R³² is OH or N(CHs)₂;
R³⁷ is OH; and
m is O or 1.

Of the above subgroup of compounds, a more preferred class of compounds according to the present invention are those compounds wherein:
R⁷ is —CH₃, —C₂H₅, —C₃H₇, -CyClo-C₃H₅, —CH(CH₃)₂, —C₄H₉, -cyclo-C₄H₇, —CH₂—CH(CHs)₂, —CH(CHs)—C₂H₅, —C(CHs)₃, —C₅Hn, —OCH₃, —OH, —F, —Cl, or —Br.

A preferred subclass of compounds of the above class is that subclass wherein:
R⁷ is —CH₃; —OCH₃, —OH or —Cl;
R⁸ is —OH, —NH₂, —OCH₃, —CONH₂, or —SO₂NH₂;
R⁹-R¹¹ are each H;
R²³ is H;
R²⁴ is H, —OH, —NH₂, —COOH, —CONH₂, or —SO₂NH₂;
R²⁵ is H, —Cl, —OH, —OCH₃, —OCF₃, —CH₃, —CF₃, —NH₂, —COOH, —CONH₂, —COOCH₃, —CN, —SO₂CH₃, or —SO₂NH₂; and
R²⁶-R²⁷ are each H.

Of the above subclass even more preferred are compounds wherein
R⁷ is —CH₃; —OCH₃, or —Cl.

Of the above subgroup of compounds, another more preferred class of compounds according to the present invention are those compounds wherein:
R⁷ is —CH₃, —C₂H₅, —C₃H₇, -cyclo-C₃H₅, —CH(CH₃)₂, —C₄H₉, -cyclo-C₄H₇, —CH₂—CH(CH₃)₂, —CH(CH₃)—C₂H₅, —C(CH₃)₃—C₅Hn, —OCH₃, —F, —Cl or —Br.

Another preferred subgroup of compounds according to the present invention are those compounds of general formulae (I), (II) or (III) wherein:
R⁷ is H; and
R⁸ is selected from the group consisting of —CH₃, —C₂H₅, —C₃H₇, —CH(CHs)₂, —C₄H₉, cyclo-C₄H₇, —CH₂—CH(CHs)₂, —CH(CHs)—C₂H₅, —C(CH₃)_{S}, —C₅Hn, —OCF₃, NO₂, —COOH, —COOCH₃, —CONH₂, —CN, —SO₂CH₃, —NH₂, NHSO₂CH₃,

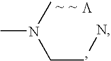

—CR³⁵R³⁶R³⁷, —X—(CH₂)ₘ—CR³²R³³R³⁴, or —X—CH₂—R⁵¹;
X is —NHCO— or —CONH—;
R⁵¹ is H;
each of the substituents R³³-R [symbol] ³⁶ is H;
R³² is OH or N(CH₃)₂;
R³⁷ is OH; and
m is 0 or 1.

Of this subgroup, a preferred class of compounds according to the invention are those compounds wherein:
R⁸ is —COOH, —COOCH₃, —CONH₂, or —CN.

Another preferred subgroup of compounds according to the present invention are those compounds of general formulae (I), (II) or (III) wherein:
R⁷ is H; and
R⁹ is selected from the group consisting of —OH, —CH₃, —H₂H₅, —C₃H₇, —CH(CHs)₂, —C₄H₉, cyclo-C₄H₇, —CH₂—CH(CHs)₂, —CH(CHs)—C₂H₅, —C(CHs)₃, —C₅Hn, —OCF₃, NO₂, —COOH, —COOCH₃, —CONH₂, —CN, —SO₂CH₃, —NH₂, NHSO₂CH₃,

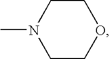

—CR— [symbol] R [symbol] ³⁰R [symbol] , —X—(CH₂)ₘ—CR³²R³³R³⁴, or —X—CH₂—R⁵¹;
X is —NHCO— or —CONH—;
R⁵¹ is H;
each of the substituents R³³-R³⁶ is H;
R³² is OH or N(CH₃)₂;
R³⁷ is OH; and
m is O or 1.

Of the above subgroup of compounds, a more preferred class of compounds according to the present invention are those compounds wherein:
R⁹ is selected from the group consisting of —CH₃, —C₂H₅, —C₃H₇, —CH(CHs)₂, —C₄H₉, cyclo-C₄H₇, —CH₂—CH(CHs)₂, —CH(CHa)-C₂H₅, —C(CH₃)_{S}, —C₅H₁₁, —OCF₃, NO₂, —COOH, —COOCH₃, —CONH₂, —CN, —SO₂CH₃, —NHSO₂CH₃,

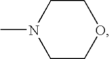

—CR³⁵R³⁶R³⁷, —X—(CH₂)ₘ—CR³²R³³R³⁴, or —X—CH₂—R⁵¹.

Another preferred subgroup of compounds according to the present invention are those compounds of general formulae (I), (II) or (III) wherein:
R²³ is H, —F, —Cl, —Br, —OH, —CH₃, —C₂H₅, —C₃H₇, -cyclo-C₃H₅, —CH(CH₃)₂, —C₄H₉, -cyclo-C₄H₇, —CH₂—CH(CH₃)₂ₗ—CH(CHs)—C₂H₅, —C(CH₃)₃, —C₅H₁₁, OCF₃, NH₂, N(CHs)₂, —N(C₂Hs)₂, —NO₂, —COOH, —COOCH₃, —CONH₂, —CN, —SO₂CHS₁NHSO₂CH₃,

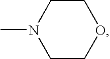

or —X—(CH₂)ₘ—CR³²R³³R³⁴, —X—CH₂—R⁵¹;
X is —NHCO— or —CONH—;
R⁵¹ is H;
each of the substituents R³³-R³⁴ is H;
R³² is OH or N(CHs)₂; and
m is O or 1.

Of the above subgroup of compounds, a more preferred class of compounds according to the present invention are those compounds wherein:
R²³ is H, —F, —Cl, —Br, —OH, —CH₃, —C₂H₅, —C₃H₇, -cyclo-C₃H₅ₗ—CH(CH₃)₂, —C₄H₉, -cyclo-C₄H₇, —CH₂—CH(CHs)₂, —CH(CHs)—C₂H₅, —C(CHs)₃, —C₅H₁₁, OCF₃, NH₂, N(CHs)₂, —N(C₂Hg)₂, —NO₂, —COOH, —COOCH₃, —CONH₂, —CN, —SO₂CH₃, NHSO₂CH₃,

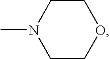

or —X—(CH₂)ₘ—CR³²R³³R³⁴, —X—CH₂—R⁵¹.

Another preferred subgroup of compounds according to the present invention are those compounds of general formulae (I), (II) or (III) wherein:
R²⁵ is H, —F, —Cl, —Br, —CH₃, —C₂H₅, —C₃H₇, -cyclo-C₃H₅, —CH(CH₃)₂, —C₄H₉, -CyClO—C₄H₇, —CH₂—CH(CH₃)₂, —CH(CHs)—C₂H₅, —C(CH₃)₃, —C₅H₁₁, —OCF₃, NH₂, N(CHs)₂, —N(C₂Hg)₂, —NO₂, —COOH, —COOCH₃, —CONH₂, —CN, —NHSO₂CH₃, —X—(CH₂)ₘ—CR³²R³³R³⁴, or —X—CH₂—R⁵¹;
X is —NHCO— or —CONH—;
R⁵¹ is H;
each of the substituents R³³-R³⁴ is H;
R³² is OH or N(CH₃)₂; and
m is O or 1.

Of the above subgroup of compounds, a more preferred class of compounds according to the present invention are those compounds wherein:
R²⁵ is —F, —Cl, —Br₁—CH₃, —C₂H₅, —C₃H₇, -cyclo-C₃H₅, —CH(CHs)₂, —C₄H₉, -cyclo-C₄H₇, —CH₂—CH(CHs)₂, —CH(CHs)—C₂H₅, —C(CH₃)₃, —C₅Hi₁, OCF₃, —NH₂, N(CHs)₂, —N(C₂Hs)₂, —NO₂, —COOH, —COOCHs, —CN, NHSO₂CH₃.

In another aspect of the invention, those compounds of the formulae (I), (II), or (III), or according to any one of the above groups, subgroups, classes or subclasses are preferred wherein R⁷ is not hydrogen.

In another aspect of the invention, those compounds of the formulae (I), (II), or (III), or according to any one of the above groups, subgroups, classes or subclasses are preferred wherein R⁷ is not hydrogen and not hydroxy.

In yet another aspect of the invention, those compounds of the formulae (I), (II), or (III), or according to any one of the above groups, subgroups, classes or subclasses are preferred wherein R²³ is not hydrogen.

In yet another aspect of the invention, those compounds of the formulae (I), (II), or (III), or according to any one of the above groups, subgroups, classes or subclasses are preferred wherein R²³ is not hydrogen, not methoxy and not hydroxymethyl.

In yet another aspect of the invention, those compounds of the formulae (I), (II), or (III), or according to any one of the above groups, subgroups, classes or subclasses are preferred wherein R⁸ is not —F, —Cl, —OH, or —OCH₃.

In yet another aspect of the invention, those compounds of the formulae (I), (II), or (III), or according to any one of the above groups, subgroups, classes or subclasses are preferred wherein R⁸ is not —F, —Cl, —Br, —NH₂, —NO₂, —OH, —OCH₃, Or-OCF₃.

In yet another aspect of the invention, those compounds of the formulae (I), (II), or (III), or according to any one of the above groups, subgroups, classes or subclasses are preferred wherein R⁹ is not —F, —Cl, or —OCH₃.

In yet another aspect of the invention, those compounds of the formulae (I), (II), or (III), or according to any one of the above groups, subgroups, classes or subclasses are preferred wherein R⁹ is not —F, Cl, —Br, —NH₂, —NO₂, —OH, or —OCH₃.

In yet another aspect of the invention, those compounds of the formulae (I), (II), or (III), or according to any one of the above groups, subgroups, classes or subclasses are preferred wherein R⁹ is not —F, —Cl, —Br, —NH₂, —N(CH₃)₂, —NO₂, —OH, or —OCH₃.

Other preferred substructures are selected from the following formulas (IV-XVII):

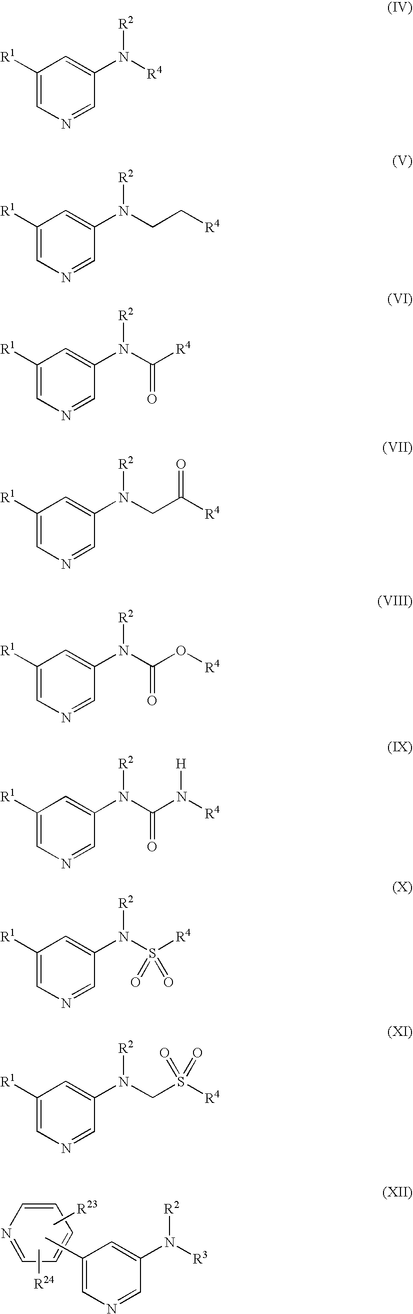

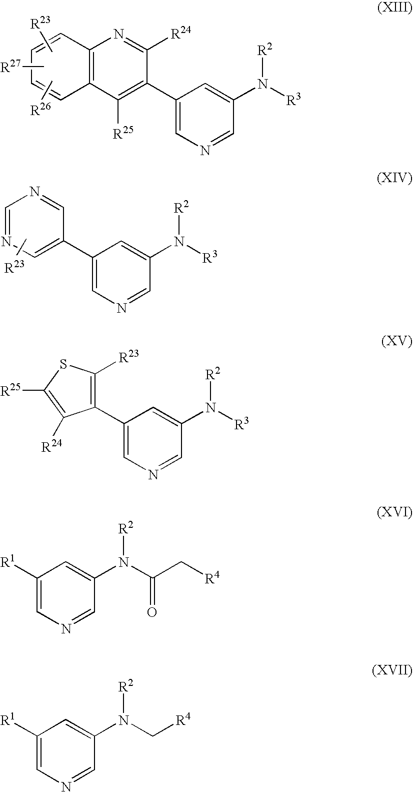

wherein
the substituents R¹-R⁴ and R²³-R²⁷ have the meanings as defined above.

Especially the following compounds are preferred:
Compound 1 (3,4-Difluoro-benzyl)-(5-thiophen-3-yl-pyridin-3-yl)-amine
Compound 2 N-(3-{5-[(Benzo[1,3]dioxol-5-ylmethyl)-amino]-pyridin-3-yl}-phenyl)-methanesulfonamide
Compound 3 3-{5-[(Benzo[1,3]dioxol-5-ylmethyl)-amino]-pyridin-3-yl}-phenol
Compound 4 [5-(4-Morpholin-4-yl-phenyl)-pyridin-3-yl]-pyridin-3-ylmethyl-amine
Compound 5 N-(2-Dimethylamino-ethyl)-3-{5-[(pyridin-3-ylmethyl)-amino]-pyridin-3-yl}-benzamide
Compound 6 (3,4-Difluoro-benzyl)-(5-pyrimidin-5-yl-pyridin-3-yl)-amine
Compound 7 (3-Chloro-phenyl)-(5-phenethyl-pyridin-3-yl)-amine
Compound 8 N-(2-Dimethylamino-ethyl)-3-[5-(4-methoxy-phenylamino)-pyridin-3-yl]-benzamide
Compound 9 4-(5-Phenylamino-pyridin-3-yl)-phenol
Compound 10 [5-(4-Methanesulfonyl-phenyl)-pyridin-3-yl]-phenyl-amine
Compound 11 (4-Chloro-benzyl)-(5^{l}-methoxy-[3,3′]bipyridinyl-5-yl)-amine
Compound 12 3-[5-(4-Chloro-benzylamino)-pyridin-3-yl]-phenol
Compound 13 {4-[5-(3,4-Difluoro-benzylamino)-pyridin-3-yl]-phenyl}-methanol
Compound 14 N-(2-Dimethylamino-ethyl)-3-{5-[(furan-3-ylmethyl)-amino]-pyridin-3-yl}-benzamide
Compound 15 [5-(4-Methanesulfonyl-phenyl)-pyridin-3-yl]-pyridin-3-ylmethyl-amine
Compound 16 (3-Bromo-phenyl)-[5-(4-dimethylamino-phenyl)-pyridin-3-yl]-amine
Compound 17 (6′-Methoxy-[3,3′]bipyridinyl-5-yl)-phenyl-amine
Compound 18 (3-Chloro-4-fluoro-phenyl)-[5-(4-dimethylamino-phenyl)-pyridin-3-yl]-amine
Compound 19 (4-Diethylamino-benzyl)-[5-(2-methoxy-phenyl)-pyridin-3-yl]-amine
Compound 20 Quinolin-3-ylmethyl-(5-quinolin-3-yl-pyridin-3-yl)-amine
Compound 21 {4-[5-(4-Chloro-benzylamino)-pyridin-3-yl]-phenyl}-methanol
Compound 22 [3,4′]Bipyridinyl-5-yl-(3,4-dimethoxy-benzyl)-amine
Compound 23 (3-Bromo-phenyl)-(5-quinolin-8-yl-pyridin-3-yl)-amine
Compound 24 N-(2-Dimethylamino-ethyl)-3-[5-(3-nitro-phenylamino)-pyridin-3-yl]-benzamide
Compound 25 Furan-S-ylmethy|̂δ′-methoxy-P.S′lbipyridinyl-δ-yO-amine
Compound 26 N-(2-Dimethylamino-ethyl)-4-[5-(3-nitro-phenylamino)-pyridin-3-yl]-benzamide
Compound 27 [3,3′]Bipyridinyl-5-yl-quinolin-3-ylmethyl-amine
Compound 28 [3,3′]Bipyridinyl-5-yl-(3,4-dichloro-benzyl)-amine
Compound 29 4-[5-(4-Chloro-benzylamino)-pyridin-3-yl-phenol
Compound 30 3-{5-[(Naphthalen-2-ylmethyl)-amino]-pyridin-3-yl}-phenol
Compound 31 N-{3-[5-(3,4-Difluoro-benzylamino)-pyridin-3-yl]-phenyl}-methanesulfonamide
Compound 32 [3,3′]Bipyridinyl-5-yl-furan-3-ylmethyl-amine
Compound 33 4-[5-(3,4-Dichloro-benzylamino)-pyridin-3-yl]-phenol
Compound 34 4-[5-(3,4-Dimethoxy-benzylamino)-pyridin-3-yl]-phenol
Compound 35 (Ŝ-Difluoro-benzylH 6′-methoxy-tS.Ŝbipyridinyl-δ-yO-amine
Compound 36 [((E)-δ-Hex-1-enyl)-pyridin<5-yl]-(3A5-trimethoxy-phenyl)-amine
Compound 37 3-[δ-(Naphthalen-2-ylamino)-pyridin-3-yl]-phenol
Compound 38 (4-Chloro-phenyl)-(5-pyrimidin-5-yl-pyridin-3-yl)-amine
Compound 39 N-{3-[5-(3,4-Dichloro-benzylamino)-pyridin-3-yl]-phenyl}-methanesulfonamide
Compound 40 3-[δ-(3,4-Dimethoxy-benzylamino)-pyridin-3-yl]-benzamide
Compound 41 5-Bromo-2-{5-[(furan-3-ylmethyl)-amino]-pyridin-3-yl}-indol θ-1-carboxylic acid tert-butyl ester
Compound 42 [3,3′]Bipyridinyl-8-yl-pyridin-3-ylmethyl-amine
Compound 43 {2-[6-(3-Chloro-phenylamino)-pyridin-3-yl]-phenyl}-methanol
Compound 44 3-(5-Phenylamino-pyridin-3-yl)-benzamide
Compound 45 (4-Chloro-phenyl)-(5′-methoxy-[3,3]bipyridinyl-5-yl)-amine
Compound 46 4-[5-(4-Chloro-phenylamino)-pyridin-3-yl]-N-(2-dimethylamino-ethyl)-benzamide
Compound 47 {4-[5-(3-Nitro-phenylamino)-pyridin-3-yl]-phenyl}-methanol
Compound 48 (5′-Methoxy-[3,3′]bipyridinyl-5-yl)-naphthalen-2-ylmethyl-amine
Compound 49 3-[5-(4-Chloro-benzylamino)-pyridin-3-yl]-N-(2-dimethylamino-ethyl)-benzamide
Compound 50 [3,3′]Bipyridinyl-5-yl-(3,4-difluoro-benzyl)-amine
Compound 51 (Ŝ-Difluoro-benzylH δ′-methoxy-P.S′lbipyridinyl-5-yO-amine
Compound 52 3-[5-(4-Trifluoromethoxy-phenylamino)-pyridin-3-yl]-phenol
Compound 53 [5-(3,4-Dimethoxy-phenyl)-pyridin-3-yl]-naphthalen-2-yl-amine
Compound 54 N-(2-Dimethylamino-ethyl)-3-[5-(naphthalen-2-ylamino)-pyridin-3-yl]-benzamide
Compound 55 (4-Chloro-phenyl)-[5-(3-trifluoromethoxy-phenyl)-pyridin-3-yl]-amine
Compound 56 (4-Chloro-phenyl)-[5-(2-methoxy-phenyl)-pyridin-3-yl]-amine
Compound 57 3-{5-[(Quinolin-3-ylmethyl)-amino]-pyridin-3-yl}-benzamide
Compound 58 4-[5-(2,4-Dimethoxy-pyrimidin-5-yl)-pyridin-3-ylamino]-benzonitrile
Compound 59 3-[5-(3,4-Dichloro-benzylamino)-pyridin-3-yl]-phenol
Compound 60 [5-(2,4-Dimethoxy-pyrimidin-5-yl)-pyridin-3-ylH⁴-isopropyl-phenyl)-amine
Compound 61 (5′-Methoxy-[33]bipyridinyl-5-yl)-(3-nitro-phenyl)-amine
Compound 62 3-[5-(3-Chloro-4-fluoro-phenylamino)-pyridin-3-yl]-benzamide
Compound 63 (3-Chloro-4-fluoro-phenyl)-(5-quinolin-3-yl-pyridin-3-yl)-amine
Compound 64 {2-[5-(3-Chloro-4-fluoro-phenylamino)-pyridin-3-yl]-phenyl}-methanol
Compound 65 [5-(2,4-Dimethoxy-pyrimidin-5-yl)-pyridin-3-yl]-naphthalen-2-yl-amine
Compound 66 (4-Chloro-phenyl)-[5-(4-methanesulfonyl-phenyl)-pyridin-3-yl]-amine
Compound 67 N-{3-[5-(4-Chloro-phenylamino)-pyridin-3-yl]-phenyl}-acetamide
Compound 68 {2-[5-(4-Chloro-phenylamino)-pyridin-3-yl]-phenyl}-methanol
Compound 69 3-[5-(4-Chloro-phenylamino)-pyridin-3-yl]-N-(2-dimethylamino-ethyl)-benzamide
Compound 70 N-(3-{5-[(Quinolin-3-ylmethyl)-amino]-pyridin-3-yl}-phenyl)-acetamide
Compound 71 (3,4-Difluoro-benzyl)-[5-(3,4-dimethoxy-phenyl)-pyridin-3-yl]-amine
Compound 72 [5-(2,4-Dimethoxy-pyrimidin-5-yl)-pyridin-3-yl]-furan-3-ylmethyl-amine
Compound 73 {4-[5-(4-Chloro-phenylamino)-pyridin-3-yl]-phenyl}-methanol
Compound 74 Furan-3-ylmethyl-(6′-methoxy-[3,3′]bipyridinyl-5-yl)-amine
Compound 75 N-(2-Hydroxy-ethyl)-3-{5-[(pyridin-3-ylmethyl)-amino]-pyridin-3-yl}-benzamide
Compound 76 Pyridin-3-ylmethyl-(5-thiophen-3-yl-pyridin-3-yl)-amine
Compound 77 {3-[5-(4-Trifluoromethoxy-phenylamino)-pyridin-3-yl]-phenyl}-methanol
Compound 78 {3-[5-(Naphthalen-2-ylamino)-pyridin-3-yl]-phenyl}-methanol
Compound 79 (Ŝ-Dichloro-benzylHS′-methoxy-P.S′lbipyridinyl-5-ylJ-amine
Compound 80 (3-Nitro-phenyl)-(5-thiophen-3-yl-pyridin-3-yl)-amine
Compound 81 (3-Chloro-4-fluoro-phenyl)-[5-(2-methoxy-phenyl)-pyridin-3-yl]-amine
Compound 82 3-[5-(4-Chloro-benzylamino)-pyridin-3-yl]-benzamide
Compound 83 (3-Chloro-4-fluoro-phenyl)-[5-(2,4-dimethoxy-pyrimidin-5-yl)-pyridin-3-yl]-amine
Compound 84 N-(2-Dimethylamino-ethyl)-4-[5-(naphthalen-2-ylamino)-pyridin-3-yl]-benzamide
Compound 85 (5′-Methoxy-[3,3′]bipyridinyl-5-yl)-(4-trifluoromethoxy-phenyl)-amine
Compound 86 (4-Chloro-phenyl)-(6′-methoxy-[3,3^{l}]bipyridinyl-5-yl)-amine
Compound 87 [3,4′]Bipyridinyl-5-yl-naphthalen-2-ylmethyl-amine
Compound 88 [3,4′]Bipyridinyl-5-yl-(4-chloro-benzyl)-amine
Compound 89 Benzo[1,3]dioxol-5-ylmethyl-[5-(3,4-dimethoxy-phenyl)-pyridin-3-yl]-amine
Compound 90 (3-{5-[(Furan-3-ylmethyl)-amino]-pyridin-3-yl}-phenyl)-methanol
Compound 91 N-{3-[5-(3,4-Dimethoxy-benzylamino)-pyridin-3-yl]-phenyl}-acetamide
Compound 92 3-{5-[(Pyridin-3-ylmethyl)-amino]-pyridin-3-yl}-phenol
Compound 93 [5-(3,4-Dimethoxy-phenyl)-pyridin-3-yl]-phenyl-amine
Compound 94 (3-Chloro-4-fluorôhenyl)-(5-pyrimidin-5-yl̂yridin-3-yl)-amine
Compound 95 N-{3-[5-(4-Chloro-benzylamino)-pyridin-3-yl]-phenyl}-methanesulfonamide
Compound 96 (3,4-Dimethoxy-benzyl)-[5-(3,4-dimethoxy-phenyl)-pyridin-3-yl]-amine
Compound 97 3-{5-[(Furan-3-ylmethyl)-amino]-pyridin-3-yl}-N-(2-hydroxy-ethyl)-benzamide
Compound 98 (5-Benzo[1,3]dioxol-5-yl-pyridin-3-yl)-furan-3-ylmethyl-amine
Compound 99 (3-Bromo-phenyl)-[5-(4-morpholin-4-yl-phenyl)-pyridin-3-yl]-amine
Compound 100 [3,3′]Bipyridinyl-5-yl-(3-bromo-phenyl)-amine
Compound 101 4-(5-Thiophen-3-yl-pyridin-3-ylamino)-benzonitril θ
Compound 102 N-(3-{5-[(Furan-3-ylmethyl)-amino]-pyridin-3-yl}-phenyl)-methanesulfonamide
Compound 103 (3-Bromo-phenyl)-[5-(2-methoxy-phenyl)-pyridin-3-yl]-amine
Compound 104 N-(2-Hydroxy-θthyl)-3-[5-(4-mθthoxy-phenylamino)-pyridin-3-yl]-benzamide
Compound 105 3-[5-(4-Chloro-phenylamino)-pyridin-3-yl]-benzamide
Compound 106 N-(3-{5-[(Naphthalen-2-ylmethyl)-amino]-pyridin-3-yl}-phenyl)-acetamide
Compound 107 Benzo[1,3]dioxol-5-ylmethyl-[3,4′]bipyridinyl-5-yl-amine
Compound 108 5-Bromo-2-[5-(3-hydroxy-benzylamino)-pyridin-3-yl]-indole-1-carboxylic acid tert-butyl ester
Compound 109 Furan-3-ylmethyl-(5-thiophen-3-yl-pyridin-3-yl)-amine
Compound 110 [3,4′]Bipyridinyl-5-yl-furan-3-ylmethyl-amine
Compound 111 [5-(3,4-Dimethoxy-phenyl)-pyridin-3-yl]-quinolin-3-ylmethyl-amine
Compound 112 N-{3-[5-(4-Chloro-benzylamino)-pyridin-3-yl]-phenyl}-acetamide
Compound 113 3-[5-(3,4-Difluoro-benzylamino)-pyridin-3-yl]-phenol
Compound 114 (3-Chloro-4-fluoro-phenylM6′-methoxy43,3]bipyridinyl-5-yl)-amine
Compound 115 (5′-Methoxy-[3,3′]bipyridinyl-5-yl)-(4-methoxy-phenyl)-amine
Compound 116 (4-{5-[(Furan-3-ylmethyl)-amino]-pyridin-3-yl}-phenyl)-methanol
Compound 117 3-[5-(3-Chloro-phenylamino)-pyridin-3-yl]-benzamide
Compound 118 N-{3-[5-(4-Isopropyl-phenylamino)-pyridin-3-yl]-phenyl}-acetamide
Compound 119 Phenyl-(5-quinolin-3-yl-pyridin-3-yl)-amine
Compound 120 4-(5-Pyrimidin-5-yl-pyridin-3-ylamino)-benzonitrile
Compound 121 (5′-Methoxy-[3,3′]bipyridinyl-5-yl)-(3,4,5-trimethoxy-phenyl)-amine
Compound 122 (3-Chloro-phenyl)-(5-quinolin-8-yl-pyridin-3-yl)-amine
Compound 123 [3,4′]Bipyridinyl-5-yl-(3,4-dichloro-benzyl)-amine
Compound 124 3-[5-(3,4-Difluoro-benzylamino)-pyridin-3-yl]-benzamide
Compound 125 3-{5-[(Benzo[1,3]dioxol-5-ylmethyl)-amino]-pyridin-3-yl}-benzamide
Compound 126 4-{5-[(Benzo[1,3]dioxol-5-ylmethyl)-amino]-pyridin-3-yl}-phenol
Compound 127 4-[5-(3,4-Difluoro-benzylamino)-pyridin-3-yl]-phenol
Compound 128 (5′-Methoxy-[3,3′]bipyridinyl-5-yl)-pyridin-3-ylmethyl-amine
Compound 129 (S-Bromo-phenylH δ′-methoxy-[3.S′lbipyridinyl-δ-yO-amine
Compound 130 N-(3-{5-[(Benzo[1,3]dioxol-5-ylmethyl)-amino]-pyridin-3-yl}-phenyl)-acetamide
Compound 131 [5-(3,4-Dimethoxy-phenyl)-pyridin-3-yl]-(4-isopropyl-phenyl)-amine
Compound 132 N-(2-Hydroxy-ethyl)-3-[5-(3-nitro-phenylamino)-pyridin-3-yl]-benzamide
Compound 133 [5-(2,4-Dimethoxy-pyrimidin-5-yl)-pyridin-3-yl]-phenyl-amine
Compound 134 5-Bromo-2-{5-[(pyridin-3-ylmethyl)-amino]-pyridin-3-yl}-indole-1-carboxylic acid tert-butyl ester
Compound 135 3-{5-[(Pyridin-3-ylmethyl)-amino]-pyridin-3-yl}-benzamide
Compound 136 (3-Bromo-phenyl)-[5-(2,4-dimethoxy-pyrimidin-5-yl)-pyridin-3-yl]-amine
Compound 137 [3-(5-Phenylamino-pyridin-3-yl)-phenyl]-methanol
Compound 138 N-{3-[5-(4-Methoxy-phenylamino)-pyridin-3-yl]-phenyl}-methanesulfonamide
Compound 139 [3,4′]Bipyridinyl-5-yl-(3,4-difluoro-benzyl)-amine
Compound 140 N-{3-[5-(3-Nitro-phenylamino)-pyridin-3-yl]-phenyl}-methanesulfonamide
Compound 141 3-{5-[(Furan-3-ylmethyl)-amino]-pyridin-3-yl}-phenol
Compound 142 3-[5-(4-Isopropyl-phenylamino)-pyridin-3-yl]-phenol
Compound 143 4-[((E)-5-Hex-1-enyl)-pyridin-3-ylamino]-benzonitrile
Compound 144 N-{S-[δ-CNaphthalen̂-ylaminoJ-pyridin-5-yll-phenylJ-acetamide
Compound 145 N-{3-[5-(3,4-Difluoro-benzylamino)-pyridin-3-yl]-phenyl}-acetamide
Compound 146 (3-Bromo-phenyl)-(5-quinolin-3-yl-pyridin-3-yl)-amine
Compound 147 {4-[5-(3-Bromo-phenylamino)-pyridin-3-yl]-phenyl}-methanol
Compound 148 4-[5-(4-Isopropyl-phenylamino)-pyridin-3-yl]-phenol
Compound 149 3-[5-(4-Cyano-phenylamino)-pyridin-3-yl]-N-(2-dimethylamino-ethyl)-benzamide
Compound 150 4-(6^{l}-Methoxy-[3,3′]bipyridinyl-5-ylamino)-benzonitrile
Compound 151 3-[5-(Naphthalen-2-ylamino)-pyridin-3-yl]-benzamide
Compound 152 3-[5-(4-Methoxy-phenylamino)-pyridin-3-yl]-benzamide
Compound 153 [3,4′]Bipyridinyl-5-yl-(3-bromo-phenyl)-amine
Compound 154 (4-Chloro-benzyl)-[5-(3,4-dimethoxy-phenyl)-pyridin-3-yl]-amine
Compound 155 3-{[5-(2-Hydroxymethyl-phenyl)-pyridin-3-ylamino]-methyl}-phenol
Compound 156 [3,3′]Bipyridinyl-5-yl-(3-nitro-phenyl)-amine
Compound 157 N-{3-[5-(3-Nitro-phenylamino)-pyridin-3-yl]-phenyl}-acetamide
Compound 158 N-(2-Hydroxy-ethyl)-3-(5-phenylamino-pyridin-3-yl)-benzamide
Compound 159 3-([3,3′]Bipyridinyl-5-ylaminomethyl)-phenol
Compound 160 3-{5-[(Furan-3-ylmethyl)-amino]-pyridin-3-yl}-benzamide
Compound 161 (e′-Methoxy-IS.S′lbipyridinyl-δ-ylH 3,4,δ-trimethoxy-phenyl)-amine
Compound 162 (3-Chloro-4-fluoro-phenyl)-[5-(4-methanesulfonyl-phenyl)-pyridin-3-yl]-amine
Compound 163 N-(3-{5-[(Furan-3-ylmethyl)-amino]-pyridin-3-yl}-phenyl)-acetamide
Compound 164 [3,4′]Bipyridinyl-5-yl-(3-nitro-phenyl)-amine
Compound 165 {3-[5-(4-Chloro-phenylamino)-pyridin-3-yl]-phenyl}-methanol
Compound 166 [3,4′]Bipyridinyl-5-yl-(3-chloro-phenyl)-amine
Compound 167 3-[5-(3-Nitro-phenylamino)-pyridin-3-yl]-benzamide
Compound 168 3-[5-(4-Chloro-phenylamino)-pyridin-3-yl]-phenol
Compound 169 N-{3-[5-(4-Chloro-phenylamino)-pyridin-3-yl]-phenyl}-methanesulfonamide
Compound 170 3-[5-(3-Bromo-phenylamino)-pyridin-3-yl]-benzamide
Compound 171 4-(5^{l}-Methoxy-[3,3′]bipyridinyl-5-ylamino)-benzonitrile
Compound 172 3-{[5-(2-Methoxy-phenyl)-pyridin-3-ylamino]-methyl}-phenol
Compound 173 4-[5-(3-Chloro-4-fluoro-phenylamino)-pyridin-3-yl]-N-(2-dimethylamino-ethyl)-benzamide
Compound 174 3-[5-(3-Nitro-phenylamino)-pyridin-3-yl]-phenol
Compound 175 {4-[5-(3-Chloro-4-fluoro-phenylamino)-pyridin-3-yl]-phenyl}-methanol
Compound 176 4-{5-[(Furan-3-ylmethyl)-amino]-pyridin-3-yl}-phenol
Compound 177 [5-(3,4-Dimethoxy-phenyl)-pyridin-3-yl]-furan-3-ylmethyl-amine
Compound 178 3-[5-(3-Bromo-phenylamino)-pyridin-3-yl]-N-(2-hydroxy-ethyl)-benzamide
Compound 179 N-(2-Dimethylamino-ethyl)-3-[5-(3,4,5-trimethoxy-phenylamino)-pyridin-3-yl]-benzamide
Compound 180 (3-Chloro-4-fluoro-phenyl)-(5′-methoxy-[3,3]bipyridinyl-5-yl)-amine
Compound 181 3-{[5-(2,4-Dimethoxy-pyrimidin-5-yl)-pyridin-3-ylamino]-methyl}-phenol
Compound 182 (5-Thiophen-3-yl-pyridin-3-yl)-(3,4,5-trimethoxy-phenyl)-amine
Compound 183 N-{3-[5-(4-Cyano-phenylamino)-pyridin-3-yl]-phenyl}-methanesulfonamide
Compound 184 N-[3-(5-Phenylamino-pyridin-3-yl)-phenyl]-methanesulfonamide
Compound 185 [3,3′]Bipyridinyl-5-yl-(3-chloro-phenyl)-amine
Compound 186 4-[5-(3-Chloro-4-fluoro-phenylamino)-pyridin-3-yl]-phenol
Compound 187 N-{3-[5-(3-Chloro-phenylamino)-pyridin-3-yl]-phenyl}-acetamide
Compound 188 3-[5-(3,4,5-Trimethoxy-phenylamino)-pyridin-3-yl]-benzamide
Compound 189 3-[5-(3,4,5-Trimethoxy-phenylamino)-pyridin-3-yl]-phenol
Compound 190 3-(5-Phenylamino-pyridin-3-yl)-phenol
Compound 191 {3-[5-(3-Chloro-4-fluoro-phenylamino)-pyridin-3-yl]-phenyl}-methanol
Compound 192 N-{3-[5-(3-Chloro-phenylamino)-pyridin-3-yl]-phenyl}-methanesulfonamide
Compound 193 N-(2-Hydroxy-ethyl)-3-[5-(naphthalen-2-ylamino)-pyridin-3-yl]-benzamide
Compound 194 N-[3-(5-Phenylamino-pyridin-3-yl)-phenyl]-acetamide
Compound 195 3-[(5-Pyrimidin-5-yl-pyridin-3-ylamino)-methyl]-phenol
Compound 196 N-(3-[5-(3,4,5-Trimethoxy-phenylamino)-pyridin-3-yl]-phenyl)-methanesulfonamide
Compound 197 4-[5-(3-Hydroxymethyl-phenyl)-pyridin-3-ylamino]-benzonitrile
Compound 198 3-[5-(3-Chloro-phenylamino)-pyridin-3-yl]-N-(2-hydroxy-ethyl)-benzamide
Compound 199 [5-(3,4-Dimethoxy-phenyl)-pyridin-3-yl]-(3-nitro-phenyl)-amine
Compound 200 N-{3-[5-(3,4,5-Trimethoxy-phenylamino)-pyridin-3-yl]-phenyl}-acetamido
Compound 201 3-[5-(4-Cyano-phenylamino)-pyridin-3-yl]-benzamide
Compound 202 N-(2-Dimethylamino-ethyl)-4-[5-(3-hydroxy-benzylamino)-pyridin-3-yl]-benzamide
Compound 203 4-[5-(4-Methanesulfonyl-phenyl)-pyridin-3-ylamino]-benzonitrile
Compound 204 N-{3-[5-(4-Trifluoromethoxy-phenylamino)-pyridin-3-yl]-phenyl}-acetamide
Compound 205 N-{3-[5-(4-Cyano-phenylamino)-pyridin-3-yl]-phenyl}-acetamide
Compound 206 3-{[5-(4-Methanesulfonyl-phenyl)-pyridin-3-ylamino]-methyl}-phenol
Compound 207 S—K6′-Methoxy-fS.S′lbipyridinyl-δ-ylaminoJ-mθthyll-phenol
Compound 208 3-[5-(3-Chloro-4-fluoro-phenylamino)-pyridin-3-yl]-phenol
Compound 209 3-([3,4′]Bipyridinyl-5-ylaminomethyl)-phenol
Compound 210 N-{3-[5-(3-Chloro-4-fluoro-phenylamino)-pyridin-3-yl]-phenyl}-methanesulfonamide
Compound 211 N-{3-[5-(Naphthalen-2-ylamino)-pyridin˜3-yl]-phenyl}-methanesulfonamide
Compound 212 3-{[5-(4-Hydroxy-phenyl)-pyridin-3-ylamino]-methyl}-phenol
Compound 213 N-{3-[5-(3-Chloro-4-fluoro-phenylamino)-pyridin-3-yl]-phenyl}-acetamide
Compound 214 3-[(5-Benzo[1,3]dioxol-5-yl-pyridin-3-ylamino)-methyl]-phenol
Compound 215 3-{[5-(3-Trifluoromethoxy-phenyl)-pyridin-3-ylamino]-methyl}-phenol
Compound 216 3-{[5-(3-Hydroxymethyl-phenyl)-pyridin-3-ylamino]-methyl}-phenol
Compound 217 3-[5-(3-Hydroxy-benzylamino)-pyridin-3-yl]-N-(2-hydroxy-ethyl)-benzamide
Compound 218 3-{[5-(4-Morpholin-4-yl-phenyl)-pyridin-3-ylamino]-methyl}-phenol
Compound 219 4-[5-(3-Hydroxy-phenyl)-pyridin-3-ylamino]-benzonitrile
Compound 220 4-[5-(3-Nitro-phenylamino)-pyridin-3-yl]-phenol
Compound 221 N-{3-[5-(3-Hydroxy-benzylamino)-pyridin-3-yl]-phenyl}-acetamide
Compound 222 3-[(5′-Methoxy-[3,3′]bipyridinyl-5-ylamino)-methyl]-phenol
Compound 223 3-{[5-(3,4-Dimethoxy-phenyl)-pyridin-3-ylamino]-methyl}-phenol
Compound 224 N-(2-Dimethylamino-ethyl)-3-[5-(3-hydroxy-benzylamino)-pyridin-3-yl]-benzamide
Compound 225 3-{5-[(3-Hydroxybenzyl)amino]pyridin-3-yl}phenol
Compound 226 N-{3-[5-(3-Hydroxy-benzylamino)-pyridin-3-yl]-phenyl}-methanesulfonamide
Compound 227 N-{3-[5-(4-Methoxy-phenylamino)-pyridin-3-yl]-phenyl}-acetamide
Compound 228 3-[(5-Thiophen-3-yl-pyridin-3-ylamino)-methyl]-phenol
Compound 229 3-{[5-(4-Hydroxymethyl-phenyl)-pyridin-3-ylamino]-methyl}-phenol
Compound 230 3-[5-(3-Hydroxy-benzylamino)-pyridin-3-yl]-benzamide
Compound 231 2-Fluoro-3-[5-(3-hydroxy-benzylamino)-pyridin-3-yl]-phenol
Compound 232 3-{[5-(3-Amino-phenyl)-pyridin-3-ylamino]-methyl}-phenol
Compound 233 3-{[5-(3-Hydroxy-phenyl)-pyridin-3-ylamino]-methyl}-2-methyl-phenol
Compound 234 3-Hydroxy-N-[5-(3-hydroxy-phenyl)-pyridin-3-yl]-benzamide
Compound 235 N-{3-[5-(4-Fluoro-phenylamino)-pyridin-3-yl]-phenyl}-acetamide
Compound 236 3-{[5-(2-Fluoro-3-methoxy-phenyl)-pyridin-3-ylamino]-methyl}-phenol
Compound 237 N-{3-[5-(2-Fluoro-phenylamino)-pyridin-3-yl]-phenyl}-acetamide
Compound 238 3-[(5-Phenyl-pyridin-3-ylamino)-methyl]-phenol
Compound 239 3-{[5-(3-Methoxy-phenyl)-pyridin-3-ylamino]-methyl}-phenol
Compound 240 N-{3-[5-(2-Methoxy-phenylamino)-pyridin-3-yl]-phenyl}-acetamide
Compound 241 3-{5-[(3-Hydroxy-benzyl)-methyl-amino]-pyridin-3-yl}-phenol
Compound 242 5-{[5-(3-Hydroxy-phenyl)-pyridin-3-ylamino]-methyl}-benzene-1,3-diol
Compound 243 3-[5-(3-Hydroxy-benzylamino)-pyridin-3-yl]-benzoic acid methyl ester
Compound 244 3-{5-[2-(3-Hydroxy-phenyl)-ethylamino]-pyridin-3-yl}-phenol
Compound 245 3-[5-(3-Amino-benzylamino)-pyridin-3-yl]-phenol
Compound 246 3-[5-(3-Hydroxy-benzylamino)-pyridin-3-yl]-benzoic acid
Compound 247 5-{[5-(3-Hydroxy-phenyl)-pyridin-3-ylamino]-methyl}-2-methyl-phenol
Compound 248 3-[(5-Bromo-pyridin-3-ylamino)-methyl]-phenol
Compound 249 3-{[5-(2-Hydroxy-phenyl)-pyridin-3-ylamino]-methyl}-phenol
Compound 250 N-{3-[5-(Methyl-phenyl-amino)-pyridin-3-yl]-phenyl}-acetamide
Compound 251 2-{[5-(3-Hydroxy-phenyl)-pyridin-3-ylamino]-methyl}-phenol
Compound 252 [5-(3-Amino-phenyl)-pyridin-3-yl]-phenyl-amine
Compound 253 N-[3-(5-Amino-pyridin-3-yl)-phenyl]-acetamide
Compound 254 3-(5-Benzylamino-pyridin-3-yl)-phenol
Compound 255 3-[5-(2-Fluoro-5-methoxy-benzylamino)-pyridin-3-yl]-phenol
Compound 256 2-(3-Hydroxy-phenyl)-N-[5-(3-hydroxy-phenyl)-pyridin-3-yl]-acetamide
Compound 257 3-(Pyridin-3-ylaminomethyl)-phenol
Compound 258 3-[5-(3-Methoxy-benzylamino)-pyridin-3-yl]-phenol
Compound 259 3-[5-(4-Fluoro-3-methoxy-benzylamino)-pyridin-3-yl]-phenol
Compound 260 3-(5-Amino-pyridin-3-yl)-phenol
Compound 261 3-{5-[(3-Methoxy-benzyl)-methyl-amino]-pyridin-3-yl}-phenol
Compound 262 3-{[5-(3-Hydroxy-phenyl)-pyridin-3-ylamino]methyl}-benzoic acid methyl ester
Compound 263 3-{[5-(3-Hydroxy-phenyl)-pyridin-3-ylamino]methyl}-benzoic acid
Compound 264 3-{[5-(3-Hydroxy-phenyl)-pyridin-3-ylamino]mθthyl}-benzoic acid methyl ester
Compound 265 3-{[5-(3-Hydroxy-phenyl)-pyridin-3-ylamino]-methyl}-benzamide
Compound 266 3-[5-(3-Nitro-benzylamino)-pyridin-3-yl]-phenol
Compound 267 N-[5-(3-Hydroxy-phenyl)-pyridin-3-yl]-3-methoxy-4-methyl-benzamide
Compound 268 3-[5-(3-Methoxy-4-methyl-benzylamino)-pyridin-3-yl]-phenol
Compound 269 3-Hydroxy-N-[5-(3-hydroxy-phenyl)-pyridin-3-yl]-2-methyl-benzamide
Compound 270 (3-Methoxy-benzyl)-[5-(3-methoxy-benzyl)-pyridin-3-yl]-carbamic acid tert-butyl ester
Compound 271 (3-methoxy-benzyl)-[5-(3-methoxy-benzyl)-pyridin-3-yl]-amine
Compound 272 (3-hydroxy-benzyl)-[5-(3-hydroxy-benzyl)-pyridin-3-yl]-amine
Compound 273 3-{[5-(3-Hydroxy-phenyl)-pyridin-3-ylamino]-methyl}-benzoic acid methyl ester
Compound 274 (5-Phenyl-pyridin-3-yl)-phenyl-amine.

Most of the compounds of the invention are basic and form pharmaceutically acceptable salts with organic and inorganic acids.

Examples of suitable acids for such acid addition salt formation are hydrochloric acid, hydrobromic acid, sulfuric acid, phosphoric acid, acetic acid, citric acid, oxalic acid, malonic acid, salicylic acid, p-aminosalicylic acid, malic acid, fumaric acid, succinic acid, ascorbic acid, maleic acid, sulfonic acid, phosphonic acid, perchloric acid, nitric acid, formic acid, propionic acid, gluconic acid, lactic acid, tartaric acid, hydroxymaleic acid, pyruvic acid, phenylacetic acid, benzoic acid, p-aminobenzoic acid, p-hydroxybenzoic acid, methanesulfonic acid, ethanesulfonic acid, nitrous acid, hydroxyethanesulfonic acid, ethylenesulfonic acid, p-toluenesulfonic acid, naphthylsulfonic acid, sulfanilic acid, camphersulfonic acid, china acid, mandelic acid, o-methylmandelic acid, hydrogen-benzenesulfonic acid, picric acid, adipic acid, D-o-tolyltartaric acid, tartronic acid, α-toluic acid, (o, m, p)-toluic acid, naphthylamine sulfonic acid, and other mineral or carboxylic acids well known to those skilled in the art. The salts are prepared by contacting the free base form with a sufficient amount of the desired acid to produce a salt in the conventional manner.

The free base forms may be regenerated by treating the salt with a suitable dilute aqueous base solution such as dilute aqueous sodium hydroxide, potassium carbonate, ammonia and sodium bicarbonate. The free base forms differ from their corresponding salt forms somewhat in certain physical properties, such as solubility in polar solvents, but the salts are otherwise equivalent to their corresponding free base forms for purposes of this invention.

The present invention also comprises pharmaceutically active salts of these compounds, all stereoisomeric forms and regioisomeric forms of these compounds or prodrugs thereof.

Other aspects of the present invention relate to the pyridinylamines as outlined above in the general formula (I), for use as new pharmaceutically active agents, particularly for the prophylaxis and/or treatment of prion diseases, immunological diseases, autoimmune diseases, bipolar and clinical disorders, cardiovascular diseases, cell proliferative diseases, diabetes, inflammation, transplant rejections, erectile dysfunction, neurodegenerative diseases, stroke, hair loss, obesity, polycystic ovary syndrome, ischaemia leukopenia, Down's syndrome, Lewy body disease, Crohns disease, periodontal diseases, corneal ulceration, proteinuria, myelodysplastic syndromes, biliary cirrhosis, virally or bacterially induced diseases or infections, mycobateria-induced infections (including opportunistic infections) and diseases, pharmaceutical compositions comprising these pyridinylamines as active ingredients and methods for treating prion diseases, immunological diseases, autoimmune diseases, bipolar and clinical disorders, cardiovascular diseases, cell proliferative diseases, diabetes, inflammation, transplant rejections, erectile dysfunction, neurodegenerative diseases, stroke, viral infections, virally and/or bacterially induced diseases, in mammals, including humans.

Surprisingly, it was found that the compounds according to general formula (I) as well as pharmaceutically acceptable salts of these compounds can be used for prophylaxis and/or treatment of prion diseases, immunological diseases, autoimmune diseases, bipolar and clinical disorders, cardiovascular diseases, cell proliferative diseases, diabetes, inflammation, transplant rejections, erectile dysfunction, neurodegenerative diseases, stroke, hair loss, obesity, polycystic ovary syndrome, ischaemia leukopenia, Down's syndrome, Lewy body disease, periodontal diseases, corneal ulceration, proteinuria, myelodysplastic syndromes and biliary cirrhosis, virally and/or bacterially induced diseases, especially mycobacteria-induced infections and diseases at pharmaceutically acceptable concentrations while exhibiting enhanced metabolitic stability. It shall be stressed that the compounds which are excluded from the claims by disclaimer are herewith explicitly claimed for any pharmaceutical use thereof as described herein.

Furthermore, it was found the pyridinylamines of the present invention are kinase inhibitors, especially of tyrosine kinases and tyrosine-like kinases.

Protein kinases form a large family of structurally related enzymes that control a variety of different cell processes including proliferation, differentiation, apoptosis, motility, transcription, translation and other signaling processes by adding phosphate groups to target proteins (Hardie, G. and Hanks, S. (1995) The Protein Kinase Facts Book, I and II, Academic Press, San Diego, Calif.). The protein kinase family can conveniently be classified into two classes with regard to substrate specificity: protein tyrosine kinases (PTKs) phosphorylate their substrates on tyrosine residues, whereas serine/threonine kinases (STKs) phosphorylate proteins on serine or threonine residues.

PTKs can be further subdivided into receptor tyrosine kinases (RTKs) and intracellular tyrosine kinases. Upon binding of a ligand like a growth factor or hormone, RTKs are activated and, in turn, affect numerous cellular responses such as cell division (proliferation), cell differentiation, cell growth, expression of metabolic enzymes, effects to the extracellular microenvironment, etc. An example of a RTKs is the “HER” family of RTKs, which include EGFR (epithelial growth factor receptor), HER2, HER3 and HER4. Further examples include the PDGFR family, c-Kit, and others.

Intracellular tyrosine kinases do not contain extracellular and transmembrane domains. One example of this group is the Abl tyrosine kinase, whose fusion with the BCR-gene is the cause for chronic myelogenous leukaemia (Semin Hematol. 2003 April; 40(2 Suppl 2):4-10).

Related to ABL is the Src family of intracellular tyrosine kinases. These kinases are implicated in cancer, immune system dysfunction and bone remodeling diseases (For general reviews, see Thomas and Brugge, Annu. Rev. Cell Dev. Biol. (1997) 13, 513; Lawrence and Niu, Pharmacol. Then (1998) 77, 81; Tatosyan and Mizenina, Biochemistry (Moscow) (2000) 65, 49; Boschelli et al., Drugs of the Future 2000, 25(7), 717, (2000)).

Members of the Src family include the following eight kinases in mammals: Src, Fyn, Yes, Fgr, Lyn, Hck, Lck, and Blk. Based on published studies, Src kinases are considered as potential therapeutic targets for various human diseases. Mice that are deficient in Src develop osteoporosis, or bone build-up, because of depressed bone resorption by osteoclasts. This suggests that osteoporosis resulting from abnormally high bone resorption can be treated by inhibiting Src (Soriano et al., Cell, 69, 551 (1992) and Soriano et al., Cell, 64, 693 (1991)).

Src also plays a role in the replication of hepatitis B virus. The virally encoded transcription factor HBx activates Src in a step required for propagation of the virus (Klein et al., EMBO J., 18, 5019, (1999) and Klein et al., Mol. Cell. Biol., 17, 6427 (1997)).

A number of studies have linked Src expression to cancers such as colon, breast, hepatic and pancreatic cancer, certain B-cell leukemias and lymphomas (Curr Pharm Des. 2003; 9(25):2043-59; Front Biosci. 2003 Sep. 1; 8:s1068-73).

Other Src family kinases are also potential therapeutic targets. The function of Lck as a positive activator of T-cell signaling suggests that Lck inhibitors may be useful for treating autoimmune disease such as rheumatoid arthritis (Molina et al., Nature, 357, 161 (1992)). Hck, Fgr and Lyn have been identified as important mediators of integrin signaling in myeloid leukocytes (Lowell et al., J. Leukoc. Diol., 65, 313 (1999)). Inhibition of these kinase mediators may therefore be useful for treating inflammation (Boschelli et al., Drugs of the Future 2000, 25(7), 717, (2000)).

An example for a STK family kinase is RICK (RIP2, Cardiak, CARD3). RICK belongs to the RIP family of protein kinases, including the kinases RICK, RIP, Rip3 and RIP4, which have been implemented in NF-kB activation. RICK is central part of the innate and adaptive immune response and involved in host response to intracellular infections as well as in inflammatory processes (Eickhoff et al. JBC March 2003; Current Biology, 8, p. 885-8; Nature 416, p. 194-9; Nature 416, p. 190-3.). Inhibition of RICK has been described to modulate the innate and adaptive immune response (WO03059285). Inhibitors of RICK and RIP kinase activity have been described to block human Cytomegalovirus replication (US20030082519). The inventive compounds are explicitly suitable as RICK inhibitors.

ROCK1 and 2 constitute a family of kinases that have been shown to be involved in cellular functions including apoptosis, cell migration, transcriptional activation, fibrosis, cytokinesis, inflammation and cell proliferation (Nat Rev Mol Cell Biol. 2003 June; 4(6):446-56). Moreover, ROCK plays a critical role in smooth muscle contraction and in the inhibition of axonal growth in neurons. Therefore, ROCK1 and 2 have been implicated to be important for a number of diseases (Curr Opin Investig Drugs. 2003 September; 4(9):1065-75; Int J Impot Res. 2003 October; 15 Suppl 5:S20-4.). Inhibition of Rho kinase activity in animal models has demonstrated a number of benefits of Rho kinase inhibitors for atherosclerosis, cardiovascular diseases such as hypertension, penile erectile dysfunction, central nervous system disorders, neoplasias, thrombotic disorders such as platelet aggregation, leukocyte aggregation and bone resorption.

Glycogen synthase kinase-3 (GSK-3) is a serine/threonine protein kinase, comprised of alpha and beta isoforms, that has been linked to various diseases including diabetes, Alzheimer's disease, CNS disorders such as manic depressive disorder and neurodegenerative diseases, and cardiomyocyte hypertrophy [see, e.g., WO 99/65897; WO 00/38675; Kaytor and Orr, Curr. Opin. Neurobiol., 12, 275-8 (2000); Haq et al., J. Cell Biol., 151, 117-30 (2000); Eldar-Finkelman, Trends Mol. Med., 8, 126-32 (2002)].

Another example for a serine/threonine kinase is Inhibitor of NF-kappa B kinase beta (IKK beta). Included in the genes regulated by NF-kappa B are a number of cytokines and chemokines, cell adhesion molecules, acute phase proteins, immunoregulatory proteins, eicosanoid metabolizing enzymes and anti-apoptotic genes (Cell. 2002 April; 109 Suppl:S81-96). It is well-known that NF-kappa B plays a key role in the regulated expression of a large number of pro-inflammatory mediators including cytokines such as TNF, IL-1 beta, IL-6 and IL-8, cell adhesion molecules, such as ICAM and VCAM, and inducible nitric oxide synthase (iNOS). Several IKK beta inhibitors are currently being in development for the treatment of a variety of inflammatory and autoimmune diseases (Nat Rev Drug Discov. 2004 January; 3(1): 17-26).

Among the kinases, the cyclin-dependent kinases (CDKs) play a major role in the control of the cell cycle. To date, nine kinase subunits (cdk 1-9) have been identified along with several regulatory subunits (cyclins A-H) (A. M. Senderowicz and E. A. Sausville Journal of the National Cancer Institute (2000), 92 (5), 376-387; and S. Mani; C. Wang; K. Wu; R. Francis; R. Pestell ‘Exp. Opin. Invest. Drugs (2000) 9 (8), 1849-1870). An increasing body of evidence has shown a link between tumour development and cdk related malfunctions. CDKs play a role in the regulation of cellular proliferation. Therefore, CDK inhibitors could be useful in the treatment of cell proliferative disorders (Lancet Oncol. 2004 January; 5(1):27-36. Review, Oncogene. 2003 Sep. 29; 22(42):6609-20, Curr Opin Pharmacol. 2003 August; 3(4):362-70.). Other indications include neurodegenerative disorders such as Alzheimer's disease and amyotrophic lateral sclerosis, which have been linked to Cdk5 (J Mol Neurosci. 2002 December; 19(3):267-73). Several host cell kinases have been shown to be important for virus replication like human cytomegalovirus, herpes simplex virus, human immune deficiency virus and VCV varicella zoster virus (WO2004/043467).

p38 is another example for a protein kinase with serine/threonine specificity. It is also known as cytokine suppressive anti-inflammatory drug binding protein (CSBP). Inhibition of p38 kinase leads to a blockade in the production of both IL-1 and TNF. Based upon this finding it is believed that p38, along with other MAPKs, has a role in mediating cellular responses to inflammatory stimuli, such as leukocyte accumulation, macrophage/monocyte activation, tissue resorption, fever, acute phase responses and neutrophilia. In addition, p38 has been implicated in acute and chronic inflammatory diseases, in cancer, thrombin-induced platelet aggregation, immunodeficiency disorders, autoimmune diseases, cell death, allergies, osteoporosis and neurodegenerative disorders (WO9621654; Current review: p38 MAP kinases: key signaling molecules as therapeutic targets for inflammatory diseases. Nat Rev Drug Discov. 2003 September; 2(9):717-26).

The human cytomegalovirus-encoded protein kinase pUL97 is belonging to a group of homologous protein kinase C (PKC)-like protein kinases with serine/threonine-specificity. Several studies have shown that pUL97 is particularly important for efficient replication (Marschall et al., 2001; Michel et al., 1996; Prichard et al., 1999; Wolf et al., 2001). Inhibitors of pUL97 should therefore be useful for treatment of HCMV associated diseases.

It has been clearly demonstrated that kinases play an important role in disease states associated with, but not limited to, disregulated cell signaling, inflammation, cancer, allergy/asthma, disease and conditions of the immune system, disease and conditions of the central nervous system, and angiogenesis. The development of selective protein kinase inhibitors that can block the disease pathologies and/or symptoms resulting from aberrant protein kinase activity has therefore generated much interest (Current review: Protein kinases—the major drug targets of the twenty-first century? Nat Rev Drug Discov. 2002 April; 1(4):309-15). Attempts have been made to identify small organic molecules which inhibit protein kinases. For example, imidazoles, oxazoles and thiazoles (WO2004/005283), purines (2003/0199534) and bisindolyl-maleimids (WO9718809) have been described as kinase inhibitors. 3-(cycloalkano-heteroarylidenyl)-2-indolinone (U.S. Pat. No. 6,579,897), pyrimido-pyrimidines (US20040019210) and bis-monocylic, bicyclic and heterocyclic aryl compounds (WO 92/20642) have been described as specific PTK inhibitors. Some companies have begun to develop Inhibitors that specifically inhibit p38. For example, PCT publication WO02/14281 describes purines, PCT publication WO95/31451 describes pyrazoles and US 2004/0023992 describes pyrazolo-pyrimidine aniline compounds as p38 inhibitors. PCT publication WO 98/27098 also describes substituted nitrogen-containing heterocycles as p38 inhibitors. Heteroaryls, covering substituted 3-aminopyridines amongst others, are described as Akt kinase inhibitor agents (WO 03/051366) with no biological activity shown on other kinases.

The following list represents a certain number of kinases which can be inhibited by the inventive compounds:

TABLE 1
List of all protein kinases

| No. | Accession Number | Gene |
|---|---|---|
| 1 | NM_001105 | ACVR1 (activin A receptor, type I) |
| 2 | NM_004302 | ACVR1B (activin A receptor, type IB) |
| 3 | NM_145259 | ACVR1C, ALK7 |
| 4 | NM_001616 | ACVR2, activin A receptor, type II |
| 5 | NM_001106 | ACVR2B, activin A receptor, type IIB |
| 6 | NM_000020 | ACVRL1 (activin A receptor type II-like 1) |
| 7 | NM_004612 | TGFBR1 (transforming growth factor, beta receptor I (activin A receptor |
| | | type II-like kinase, 53 kD)) |
| 8 | NM_003242 | TGFBR2 (transforming growth factor, beta receptor II) |
| 9 | NM_004329 | BMPR1A (bone morphogenetic protein receptor, type IA) |
| 10 | NM_001203 | BMPR1B (bone morphogenetic protein receptor, type IB) |
| 11 | NM_001204 | BMPR2 (bone morphogenetic protein receptor, type II (serine/threonine |
| | | kinase)) |
| 12 | NM_006251 | PRKAA1 (protein kinase, AMP-activated, alpha 1 catalytic subunit) |
| 13 | NM_006252 | PRKAA2 (protein kinase, AMP-activated, alpha 2 catalytic subunit) |
| 14 | NM_002929 | GRK1; rhodopsin kinase |
| 15 | NM_001619 | GRK2 |
| 16 | NM_005160 | GRK3 |
| 17 | NM_005307 | GRK4 |
| 18 | NM_005308 | GRK5 |
| 19 | NM_002082 | GRK6 |
| 20 | NM_139209 | GRK7 (G protein-coupled receptor kinase 7) |
| 21 | NM_017572 | MKNK2, GPRK7 |
| 22 | NM_001654 | ARAF1 (v-raf murine sarcoma 3611 viral oncogene homolog 1) |
| 23 | NM_004333 | BRAF (v-raf murine sarcoma viral oncogene homolog B1) |
| 24 | NM_002880 | RAF1 (v-raf-1 murine leukemia viral oncogene homolog 1) |
| 25 | NM_021574 | BCR1 |
| 26 | NM_003656 | CAMK1 (calcium/calmodulin-dependent protein kinase I) |
| 27 | NM_015981 | CAMK2A (calcium/calmodulin-dependent protein kinase (CaM kinase) II |
| | | alpha) |
| 28 | NM_001220 | CAMK2B (calcium/calmodulin-dependent protein kinase (CaM kinase) II |
| | | beta) |
| 29 | NM_001221 | CAMK2D (calcium/calmodulin-dependent protein kinase (CaM kinase) II |
| | | delta) |
| 30 | NM_020439 | CAMK1G (calcium/calmodulin-dependent protein kinase IG) |
| 31 | NM_001222 | CAMK2G (calcium/calmodulin-dependent protein kinase (CaM kinase) II |
| | | gamma) |
| 32 | NM_001744 | CAMK4 (calcium/calmodulin-dependent protein kinase IV) |
| 33 | NM_001786 | CDC2 (cell division cycle 2) |
| 34 | NM_001798 | CDK2 (cyclin-dependent kinase 2) |
| 35 | NM_001258 | CDK3 (cyclin-dependent kinase 3) |
| 36 | NM_000075 | CDK4 (cyclin-dependent kinase 4) |
| 37 | NM_004935 | CDK5 (cyclin-dependent kinase 5) |
| 38 | NM_001259 | CDK6 (cyclin-dependent kinase 6) |
| 39 | NM_001799 | CDK7 (cyclin-dependent kinase 7) |
| 40 | NM_001260 | CDK8 (cyclin-dependent kinase 8) |
| 41 | NM_001261 | CDK9 (cyclin-dependent kinase 9 (CDC2-related kinase)) |
| 42 | NM_003674 | CDK10 (cyclin-dependent kinase (CDC2-like) 10) |
| 43 | NM_015076 | CDK11, DPK |
| 44 | NM_004196 | CDKL1 (cyclin-dependent kinase-like 1); KKIALRE |
| 45 | NM_003948 | CDKL2 (cyclin-dependent kinase-like 2); KKIAMRE |
| 46 | NM_016508 | CDKL3 (cyclin-dependent kinase-like 3); NKIAMRE |
| 47 | XM_293029 | CDKL4, similar to cyclin-dependent kinase-like 1 |
| 48 | NM_033489 | CDC2L1 (cell division cycle 2-like 1); PITSLRE B |
| 49 | NM_024011 | CDC2L1 (cell division cycle 2-like 1); PITSLRE A |
| 50 | NM_003718 | CDC2L5 (cell division cycle 2-like 5) |
| 51 | NM_006201 | PCTK1 (PCTAIRE protein kinase 1) |
| 52 | NM_002595 | PCTK2 (PCTAIRE protein kinase 2) |
| 53 | NM_002596 | PCTK3 (PCTAIRE protein kinase 3) |
| 54 | NM_012395 | PFTK1 (PFTAIRE protein kinase 1) |
| 55 | NM_001278 | IKK-alpha; CHUK |
| 56 | NM_001556 | IKK-beta; IKK2 |
| 57 | NM_001892 | CSNK1A1 (casein kinase 1, alpha 1) |
| 58 | NM_001893 | CSNK1D (casein kinase 1, delta) |
| 59 | NMJJ01894 | CSNK1E (casein kinase 1, epsilon) |
| 60 | NM_004384 | CSNK1G3 (casein kinase 1, gamma 3) |
| 61 | NM_001319 | CSNK1G2 (casein kinase 1, gamma 2) |
| 62 | NM_001895 | CSNK2A1 (casein kinase 2, alpha 1) |
| 63 | NM_001896 | CSNK2A2 (casein kinase 2, alpha prime) |
| 64 | NM_022048 | CSNK1G1 (casein kinase 1, gamma 1) |
| 65 | NMJ304071 | CLK1 (CDC-like kinase 1) |
| 66 | NMJD03993 | CLK2 (CDC-like kinase 2) |
| 67 | NM_003992 | CLK3 (CDC-like kinase 3) |
| 68 | NM_020666 | CLK4 (CDC-like kinase 4) |
| 69 | NM_004938 | DAPK1 (death-associated protein kinase 1) |
| 70 | NM_014326 | DAPK2 (death-associated protein kinase 2) |
| 71 | NM_001348 | DAPK3 (death-associated protein kinase 3) |
| 72 | NM_004954 | EMK1 (ELKL motif kinase) |
| 73 | NM_002746 | MAPK3; ERK1 |
| 74 | NM_002745 | MAPK1, ERK2 |
| 75 | NM_002748 | MAPK6; ERK3 |
| 76 | NM_002747 | MAPK4; ERK3-related |
| 77 | NM_002749 | MAPK7; ERK5 |
| 78 | NM_001315 | MAPK14; CSBP1 |
| 79 | NM_002751 | MAPK11; p38beta |
| 80 | NM_002969 | MAPK12; ERK6, p38g |
| 81 | NM_002754 | MAPK13; p38delta |
| 82 | AY065978 | ERK8 |
| 83 | NM_002750 | MAPK8; JNK1 |
| 84 | NM_002752 | MAPK9; JNK2 |
| 85 | NM_002753 | MAPK10; JNK3 |
| 86 | NM_006712 | FASTK (Fas-activated protein kinase) |
| 87 | NM_004579 | MAP4K2; GCK |
| 88 | NM_019884 | GSK3A (glycogen synthase kinase 3 alpha) |
| 89 | NM_002093 | GSK3B (glycogen synthase kinase 3 beta) |
| 90 | NM_002576 | PAK1 |
| 91 | NM_002577 | PAK2 |
| 92 | NM_002578 | PAK3 |
| 93 | NM_005884 | PAK4 |
| 94 | NM_020341 | PAK5 (PAK7) |
| 95 | NM_020168 | PAK6 |
| 96 | NM_007181 | MAP4K1; HPK1 |
| 97 | NM_004517 | ILK (integrin-linked kinase) |
| 98 | NM_001569 | IRAK1 (interleukin-1 receptor-associated kinase 1) |
| 99 | NM_001570 | IRAK2 (interleukin-1 receptor-associated kinase 2) |
| 100 | NM_007199 | IRAK-M |
| 101 | NM_016123 | IRAK4 |
| 102 | NM_006575 | MAP4K5 |
| 103 | NM_002314 | LIMK1 (LIM domain kinase 1) |
| 104 | NM_005569 | LIMK2 (LIM domain kinase 2) |
| 105 | NM_000455 | STK11; LKB1 |
| 106 | NM_005906 | MAK (male germ cell-associated kinase) |
| 107 | NM_002755 | MAP2K1; MEK1 |
| 108 | NM_030662 | MAP2K2; MEK2 |
| 109 | NM_002756 | MAP2K3; MEK3 |
| 110 | NM_003010 | MAP2K4; MEK4 |
| 111 | NM_002757 | MAP2K5; MEK5 |
| 112 | NM_002758 | MAP2K6; MEK6 |
| 113 | NM_005043 | MAP2K7; MKK7 |
| 114 | XM_042066 | MAP3K1; MEKK1 |
| 115 | NM_006609 | MAP3K2; MEKK2 |
| 116 | NM_002401 | MAP3K3; MEKK3 |
| 117 | NM_005922 | MAP3K4; MEKK4 |
| 118 | NM_005923 | MAP3K5; ASK1 |
| 119 | NM_004672 | MAP3K6 |
| 120 | NM_003188 | MAP3K7; TAK1 |
| 121 | NM_005204 | MAP3K8; Tpl-2 |
| 122 | XM_027237 | MAP3K9; MLK1 |
| 123 | NM_002446 | MAP3K10; MST; MLK2 |
| 124 | NM_002419 | MAP3K11; MLK3 |
| 125 | NM_006301 | MAP3K12; DLK |
| 126 | NM_004721 | MAP3K13; LZK |
| 127 | NM_003954 | MAP3K14; NIK |
| 128 | AX282911 | MAP3K7, similar to MAP/ERK kinase kinase 5; apoptosis sigma |
| | | regulating kinase |
| 129 | AX504239 | MAP3K8 |
| 130 | NM_015112 | MAST205 |
| 131 | NM_005965 | MYLK (myosin, light polypeptide kinase) |
| 132 | NM_033118 | MYLK2 (myosin light chain kinase 2) |
| 133 | NM_005372 | MOS (v-mos Moloney murine sarcoma viral oncogene homolog) |
| 134 | NM_006282 | STK4; MST1 |
| 135 | NM_006281 | STK3; MST2 |
| 136 | NM_003576 | STK24; MST3 |
| 137 | NM_012224 | NEK1 (NIMA (never in mitosis gene a)-related kinase 1) |
| 138 | NM_002497 | NEK2 (NIMA (never in mitosis gene a)-related kinase 2) |
| 139 | NM_002498 | NEK3 (NIMA (never in mitosis gene a)-related kinase 3) |
| 140 | AX394707 | NEK5 |
| 141 | NM_014397 | NEK6 (NIMA (never in mitosis gene a)-related kinase 6) |
| 142 | NM_133494 | NEK7 |
| 143 | NM_178170 | NEK8, NEK12A |
| 144 | NM_033116 | NEK9 |
| 145 | AX250157 | NEK10 |
| 146 | NM_024800 | NEK11 |
| 147 | NM_003157 | STK2 |
| 148 | NM_005406 | ROCK1 (Rho-associated, coiled-coil containing protein kinase 1); |
| | | p160ROCK |
| 149 | NM_004850 | ROCK2 (Rho-associated, coiled-coil containing protein kinase 2) |
| 150 | NM_007271 | STK38; NDR |
| 151 | NM_015000 | STK38L, NDR2 |
| 152 | NM_004409 | DMPK1 (dystrophia myotonica-protein kinase) |
| 153 | XM_290516 | DMPK2, HSMDPKIN |
| 154 | NM_003607 | MRCKalpha (PK428) |
| 155 | NM_007174 | Citron |
| 156 | NM_002613 | PDPK1 (3-phosphoinositide dependent protein kinase-1) |
| 157 | NM_006213 | PHKG1 (phosphorylase kinase, gamma 1) |
| 158 | NM_000294 | PHKG2 (phosphorylase kinase, gamma 2) |
| 159 | NM_002648 | PIM1 |
| 160 | NM_006875 | PIM2 |
| 161 | AR208686 | PIM3 |
| 162 | NM_014791 | KIAA0175 |
| 163 | NM_002730 | PRKACA (protein kinase, cAMP-dependent, alpha) |
| 164 | NM_002731 | PRKACB (protein kinase, cAMP-dependent, beta) |
| 165 | NM_002732 | PRKACG (protein kinase, cAMP-dependent, gamma) |
| 166 | NM_002742 | PRKCM (protein kinase C, mu) |
| 167 | NM_002737 | PRKCA (protein kinase C, alpha) |
| 168 | NM_002738 | PRKCB1 (protein kinase C, beta 1) |
| 169 | NM_006254 | PRKCD (protein kinase C, delta) |
| 170 | NM_005400 | PRKCE (protein kinase C, epsilon) |
| 171 | NM_002739 | PRKCG (protein kinase C, gamma) |
| 172 | NM_006255 | PRKCH (protein kinase C, eta) |
| 173 | NM_002740 | PRKCI (protein kinase C, iota) |
| 174 | NM_006257 | PRKCQ (protein kinase C, theta) |
| 175 | NM_002744 | PRKCZ (protein kinase C, zeta) |
| 176 | NM_002741 | PRKCL1 (protein kinase C-like 1) |
| 177 | NM_006256 | PRKCL2 (protein kinase C-like 2) |
| 178 | NM_006258 | PRKG1 (protein kinase, cGMP-dependent, type I) |
| 179 | NM_006259 | PRKG2 (protein kinase, cGMP-dependent, type II); cGKII |
| 180 | NM_002759 | PRKR (protein kinase, interferon-inducible double stranded RNA |
| | | dependent) |
| 181 | NM_006852 | TLK2 (tousled-like kinase 2) |
| 182 | NM_012290 | TLK1 (tousled-like kinase 1) |
| 183 | NM_005044 | PRKX (protein kinase, X-linked) |
| 184 | NM_005030 | PLK (polo-like kinase) |
| 185 | NM_004073 | CNK (cytokine-inducible kinase) |
| 186 | NM_003913 | PRPF4B |
| 187 | NM_006742 | PSKH1 (protein serine kinase H1) |
| 188 | NM_005163 | AKT1 (v-akt murine thymoma viral oncogene homolog 1) |
| 189 | NM_001626 | AKT2 (v-akt murine thymoma viral oncogene homolog 2) |
| 190 | NM_005465 | AKT3 (v-akt murine thymoma viral oncogene homolog 3 (protein kinase |
| | | B, gamma)) |
| 191 | NM_014264 | STK18; Sak |
| 192 | NM_005627 | SGK (serum/glucocorticoid regulated kinase) |
| 193 | NM_002376 | MARK3 (MAP/microtubule affinity-regulating kinase 3) |
| 194 | NM_006374 | STK25; YSK1 |
| 195 | NM_003137 | SRPK1 (SFRS protein kinase 1) |
| 196 | NM_182692 | SRPK2 (SFRS protein kinase 2) |
| 197 | NM_003319 | Titin |
| 198 | NM_003318 | TTK protein kinase |
| 199 | NM_003384 | VRK1 (vaccinia related kinase 1) |
| 200 | NM_006296 | VRK2 (vaccinia related kinase 2) |
| 201 | NM_003390 | WEE1 |
| 202 | NM_018650 | MARK1 (MAP/microtubule affinity-regulating kinase 1) |
| 203 | NM_003160 | STK13; (aurora/IPL1-like), AIE2, aurora kinase C |
| 204 | NM_004759 | MAPKAPK2 |
| 205 | NM_004635 | MAPKAPK3 |
| 206 | NM_003668 | MAPKAPK5 |
| 207 | NM_005734 | HIPK3 (homeodomain interacting protein kinase 3), DYRK6 |
| 208 | NM_003503 | CDC7L1 (CDC7 cell division cycle 7-like 1) |
| 209 | NM_016231 | NLK |
| 210 | NM_003565 | ULK1 (unc-51-like kinase 1) |
| 211 | NM_014683 | ULK2 (unc-51-like kinase 2) |
| 212 | AX056454 | DKFZP434C131 protein, ULK3 |
| 213 | NM_017886 | hypothetical protein FLJ20574, ULK4 |
| 214 | NM_053006 | STK22B; TSSK2 |
| 215 | NM_003684 | MKNK1 (MAP kinase-interacting serine/threonine kinase 1); MNK1 |
| 216 | NM_003804 | RIPK1 (receptor (TNFRSF)-interacting serine-threonine kinase 1); RIP |
| 217 | NM_003821 | RIPK2 (receptor-interacting serine-threonine kinase 2); RICK |
| 218 | NM_006871 | RIPK3 (receptor-interacting serine-threonine kinase 3); RIP3 |
| 219 | NM_003600 | STK6; BTAK, AIK |
| 220 | NM_004217 | STK12; IPL1, aurora kinase B |
| 221 | NM_006549 | CAMKK2 (calcium/calmodulin-dependent protein kinase kinase 2, beta) |
| 222 | NM_017719 | SNRK (SNF-1 related kinase) |
| 223 | NM_001433 | ERN1 (ER to nucleus signalling 1) |
| 224 | NM_004336 | BUB1 (BUB1 budding uninhibited by benzimidazoles 1 homolog) |
| 225 | NM_001211 | BUB1B (BUB1 budding uninhibited by benzimidazoles 1 homolog beta) |
| 226 | NM_006622 | SNK (serum-inducible kinase) |
| 227 | NM_001274 | CHEK1 (CHK1 checkpoint homolog) |
| 228 | NM_003957 | STK29; PEN11B |
| 229 | NM_013233 | STK39; SPAK |
| 230 | NM_003691 | STK16; PKL12 |
| 231 | XM_290796 | TAO1/KIAA1361 |
| 232 | NM_003159 | STK9 |
| 233 | NM_014586 | HUNK (hormonally upregulated Neu-associated kinase) |
| 234 | NM_004834 | MAP4K4; NIK; HGK |
| 235 | NM_002953 | RPS6KA1 = ribosomal protein S6 kinase, 90 kD, polypeptide 1 |
| 236 | NM_021135 | RPS6KA2 (ribosomal protein S6 kinase, 90 kD, polypeptide 2); RSK3 |
| 237 | NM_003161 | RPS6KB1 (ribosomal protein S6 kinase, 70 kD, polypeptide 1) |
| 238 | NM_004586 | RPS6KA3 = ribosomal protein S6 kinase, 90 kD, polypeptide 3; RSK2 |
| 239 | NM_004755 | RPS6KA5 (ribosomal protein S6 kinase, 90 kD, polypeptide 5); MSK1 |
| 240 | NM_003942 | RPS6KA4 (ribosomal protein S6 kinase, 90 kD, polypeptide 4); MSK2 |
| 241 | NM_003952 | RPS6KB2 (ribosomal protein S6 kinase, 70 kD, polypeptide 2) |
| 242 | NM_004760 | STK17A; DRAK1 |
| 243 | NM_014413 | HRI (heme-regulated initiation factor 2-alpha kinase) |
| 244 | NM_007194 | CHEK2 (CHK2 checkpoint homolog) |
| 245 | NM_012119 | CCRK (cell cycle related kinase) |
| 246 | NM_014370 | STK23; MSSK1 |
| 247 | NM_005990 | STK10; LOK |
| 248 | MM_004836 | EIF2AK3 (eukaryotic translation initiation factor 2-alpha kinase 3) |
| 249 | MM_003618 | MAP4K3; GLK |
| 250 | NMJD14720 | SLK (SNF1 sucrose nonfermenting like kinase) |
| 251 | NM_014602 | PIK3R4 (phosphoinositide-3-kinase, regulatory subunit 4, p150) |
| 252 | NM_006285 | TESK1 (testis-specific kinase 1) |
| 253 | NMJ321643 | GS3955 protein |
| 254 | NM_004203 | PKMYT1 |
| 255 | NM_015148 | PASK (PAS domain containing serine/threonine kinase) |
| 256 | NM_014002 | IKKE (IKK-related kinase epsilon; Inducible IkappaB kinase) |
| 257 | NM_007118 | TRIO (triple functional domain (PTPRF interacting)) |
| 258 | NM_001396 | DYRK1A (dual-specificity tyrosine-(Y)-phosphorylation regulated kinase |
| | | 1A) |
| 259 | NM_004714 | DYRK1B (dual-specificity tyrosine-(Y)-phosphorylation regulated kinase |
| | | 1B) |
| 260 | NM_003583 | DYRK2 (dual-specificity tyrosine-(Y)-phosphorylation regulated kinase 2) |
| 261 | NM_003582 | DYRK3 (dual-specificity tyrosine-(Y)-phosphorylation regulated kinase 3) |
| 262 | NM_003845 | DYRK4 (dual-specificity tyrosine-(Y)-phosphorylation regulated kinase 4) |
| 263 | NM_031417 | MARKL1 (MAP/microtubule affinity-regulating kinase like 1) |
| 264 | NM_014840 | KIAA0537 gene product |
| 265 | XM_039796 | TNIK (Traf2 and NCK interacting kinase) |
| 266 | XM_038150 | MAST3, KIAA0561 protein |
| 267 | XM_291141 | MAST4, KIAA0303 protein |
| 268 | NM_015375 | DustyPK |
| 269 | NM_002760 | PRKY (protein kinase, Y-linked) |
| 270 | NM_003688 | CASK (calcium/calmodulin-dependent serine protein kinase (MAGUK |
| | | family)) |
| 271 | NM_004734 | DCAMKL1 (doublecortin and CaM kinase-like 1) |
| 272 | NMJ 52619 | hypothetical protein MGC45428, DCAMKL2 |
| 273 | AX504237 | DCAMKL3, KIAA1765 protein |
| 274 | NM_004226 | STK17B; DRAK2 |
| 275 | NM_005813 | PRKCN (protein kinase C, nu) |
| 276 | NM_005255 | GAK (cyclin G associated kinase) |
| 277 | NM_032294 | hypothetical protein DKFZp761M0423 |
| 278 | NM_014226 | RAGE1 (renal tumor antigen) |
| 279 | NM_006035 | CDC42BPB (CDC42 binding protein kinase beta (DMPK-like)) |
| 280 | NM_007170 | TESK2 (testis-specific kinase 2) |
| 281 | NMJ 52696 | Nbak2, KIAA0630 protein |
| 282 | NM_016151 | PSK |
| 283 | NMJ 73354 | SNF1LK, SIK |
| 284 | AB023190 | SAST (syntrophin associated serine/threonine kinase) |
| 285 | NMJ322740 | HIPK2 (homeodomain interacting protein kinase 2) |
| 286 | AX236110 | GCN2, elF2alpha kinase |
| 287 | NM_013355 | PKNbeta |
| 288 | NMJ 98465 | NRK/ZC4 (NIK-related kinase) |
| 289 | NM_013257 | SGKL (serum/glucocorticoid regulated kinase-like) |
| | NM_016276 | SGK2 (serum/glucocorticoid regulated kinase 2) |
| | NM_012424 | RPS6KC1 (ribosomal protein S6 kinase, 52 kD, polypeptide 1) |
| | NM_014496 | RPS6KA6 (ribosomal protein S6 kinase, 90 kD, polypeptide 6); RSK4 |
| 293 | NM_013254 | TBK1 (TANK-binding kinase 1) |
| 294 | NM_016281 | JIK |
| 295 | NM_016440 | VRK3 for vaccinia related kinase 3 |
| 296 | NM_015716 | MINK (Misshapen/NIK-related kinase) |
| 297 | AX166520 | similar to Ca2+/Calmodulin-dependent protein kinase I, CAMK1b |
| 298 | NM_006410 | HTATIP2 (HIV-1 Tat interactive protein 2, 30 kD) |
| 299 | NM_016542 | MST4 |
| 300 | NM_016653 | ZAK (sterile-alpha motif and leucine zipper containing kinase AZK) |
| 301 | NMJ 73575 | PKE, YANK3 |
| 302 | NM_018979 | PRKWNK1 (protein kinase, lysine deficient 1); WNK1 |
| 303 | NM_006648 | PRKWNK2 (protein kinase, lysine deficient 2) |
| 304 | NM_020922 | PRKWNK3 (protein kinase, lysine deficient 3) |
| 305 | NM_032387 | PRKWNK4 (protein kinase, lysine deficient 4) |
| 306 | NM_018492 | TOPK (T-LAK cell-originated protein kinase) |
| 307 | AL359916 | (longer at STK35, CLIK1 |
| | | 5′) |
| 308 | NM_020680 | NTKL (N-terminal kinase-like) |
| 309 | NM_032844 | MASTL, hypothetical protein FLJ14813 |
| 310 | NM_020397 | CKLIK, CamKI-like protein kinase |
| 311 | AX224725 | SCYL2 |
| 312 | NM_153335 | STLK5, LYK5 |
| 313 | NM_174944 | TSSK4 |
| 314 | NM_052841 | STK22C; TSSK3 |
| 315 | XM_166453 | TTBK1 |
| 316 | AR004796 | KSR1 (kinase suppressor of ras) |
| 317 | NM_032037 | SSTK |
| 318 | NM_016457 | PKD2 (polycystic kidney disease 2) |
| 319 | NM_025195 | C8FW, Trb1 |
| 320 | NM_033266 | ERN2 (ER to nucleus signalling 2) |
| 321 | NM_020423 | PACE-1 |
| 322 | NM_033550 | PRPK |
| 323 | NM_018401 | serine/thronine kinase HSA250839, YANK2 |
| 324 | NM_020639 | ANKRD3 (ankyrin repeat domain 3); DIK |
| 325 | NM_015690 | STK36 |
| 326 | NM_014572 | LATS2 (LATS, large tumor suppressor, homolog 2) |
| 327 | AX056397 | SPEG, KIAA1297 protein |
| 328 | AX504253 | Wee1B |
| 329 | AX766335 | QSK, KIAA0999 protein |
| 330 | NM_007064 | TRAD |
| 331 | NM_004690 | LATS1, (LATS, large tumor suppressor, homolog 1) |
| 332 | NM_014911 | AAK1 |
| 333 | NM_014920 | ICK, MAK-related kinase |
| 334 | NM_198892 | BMP2K, BIKE |
| 335 | NM_033126 | PSKH2 |
| 336 | NM_031464 | hypothetical protein MGC11287 similar to ribosomal protein S6 kinase |
| 337 | NM_032409 | PINK1 (PTEN induced putative protein kinase 1) |
| 338 | NM_013392 | NRBP (nuclear receptor binding protein |
| 339 | NM_016507 | CrkRS |
| 340 | NM_005109 | OSR1 (oxidative-stress responsive 1) |
| 341 | NM_139158 | ALS2CR7 |
| 342 | NM_032028 | STK22D, TSSK1 |
| 343 | NM_017771 | PXK (PX domain-containing protein kinase), Slob |
| 344 | NM_018571 | ALS2CR2 (amyotrophic lateral sclerosis 2 (juvenile) chromosome region, |
| | | candidate 2), STLK6 |
| 345 | NM_031965 | GSG2, haspin |
| 346 | NM_015191 | SIK2, QIK |
| 347 | AX039412 | KIAA1639, Obscn |
| 348 | AX207388 | YANK1 |
| 349 | AX394712 | similar to MLCK, hypothetical protein LOC340156 |
| 350 | NM_178510 | ANKK1 |
| 351 | NM_021158 | C20orf97 (chromosome 20 open reading frame 97), Trb3 |
| 352 | NM_152649 | MLKL, hypothetical protein FLJ34389 |
| 353 | AX250159 | SgK223, DKFZp761P0423 |
| 354 | XM_370878 | KIAA2002 |
| 355 | NM_024652 | LRRK1 |
| 356 | NM_033115 | TBCK, hypothetical portein MGC16169 |
| 357 | AX250163 | SgK424, similar to testis expressed gene 14 (LOC126392) |
| 358 | NM_031272 | TEX14 (testis expressed sequence 14) |
| 359 | NM_024046 | hypothetical protein MGC8407, VACAMKL |
| 360 | NM_014916 | LMTK2, KIAA1079 protein, LMR2, KPI-2 |
| 361 | NM_017433 | MYO3A |
| 362 | NM_138995 | MYO3B |
| 363 | NM_030952 | SNARK |
| 364 | NM_030906 | STK33 |
| 365 | NM_182493 | similar to myosin light chain kinase (MLCK) |
| 366 | NM_032430 | BRSK1, KIAA1811 |
| 367 | XM_370948 | SBK, similar to SH3-binding kinase (LOC388228) |
| 368 | NM_032017 | SINK-homologous serine/threonine kinase, MGC4796 |
| 369 | NM_020547 | AMHR2 (anti-Mullerian hormone receptor, type II) |
| 370 | NM_031414 | STK31 |
| 371 | NM_032237 | hypothetical protein FLJ23356 |
| 372 | NM_021133 | RNASEL (ribonuclease L (2′,5′-oligoisoadenylate synthetase-dependent |
| 373 | AX166516 | similar to protein kinase Bsk146 |
| 374 | NMJ53361 | NIM1, MGC42105, similar to serine/threonine kinase (KIN1/SNF1/Nim1 |
| | | subfamily) |
| 375 | NMJ45203 | casein kinase 1 alpha S-like, CKIa2 |
| 376 | NM_173500 | TTBK2 |
| 377 | NMJ 44685 | HIPK4 |
| 378 | NMJ 75866 | KIS |
| 379 | AX166547 | KSR2 |
| 380 | AX056416 | NRBP2 |
| 381 | AX540378 | SgK494, hypothetical protein FLJ25006 |
| 382 | NMJ 52835 | CLIK1L |
| 383 | AX540373 | SgK071, similar to MGC43306 protein (LOC401568) |
| 384 | AX056460 | SgK493, hypothetical protein BC007901 (LOC91461) |
| 385 | NM_005157 | ABL1 |
| 386 | NM_005158 | ABL2, ARG |
| 387 | NM_005781 | ACK1 |
| 388 | NM_000061 | BTK |
| 389 | NM_005246 | FER |
| 390 | NM_002005 | FES |
| 391 | NM_002031 | FRK (fyn-related kinase) |
| 392 | NM_002037 | FYN |
| 393 | NM_002110 | HCK |
| 394 | NM_005248 | FGR |
| 395 | NM_005356 | LCK |
| 396 | NM_002344 | LTK |
| 397 | NM_002350 | LYN |
| 398 | NM_004383 | CSK |
| 399 | NM_005546 | ITK |
| 400 | NM_005417 | SRC |
| 401 | NM_003215 | TEC |
| 402 | NM_005433 | YES |
| 403 | NM_003328 | TXK |
| 404 | NM_080823 | SRMS |
| 405 | NM_001715 | BLK |
| 406 | NM_001721 | BMX |
| 407 | NM_005975 | PTK6 |
| 408 | NM_002821 | PTK7 |
| 409 | NM_002822 | PTK9 |
| 410 | NM_007284 | PTK9L |
| 411 | NM_000222 | KIT |
| 412 | NM_005211 | CSF1R |
| 413 | NM_005232 | EphA1 |
| 414 | NM_004431 | EphA2 |
| 415 | NM_005233 | EphA3 |
| 416 | NM_004438 | EphA4 |
| 417 | NM_004439 | EphA5 |
| 418 | AX250164 | EphA6 |
| 419 | NM_004440 | EphA7 |
| 420 | NM_020526 | EphAδ |
| 421 | AX166562 | EphAIO |
| 422 | NM_004441 | EphB1 |
| 423 | NM_004442 | EphB2 |
| 424 | NM_004443 | EphB3 |
| 425 | NM_004444 | EphB4 |
| 426 | NM_004445 | EphB6 |
| 427 | NM_000604 | FGFR1 |
| 428 | NM_000141 | FGFR2 |
| 429 | NM_000142 | FGFR3 |
| 430 | NM_002011 | FGFR4 |
| 431 | NM_002253 | KDR |
| 432 | NM_002019 | FLT1 |
| 433 | NM_004119 | FLT3 |
| 434 | NM_002020 | FLT4 |
| 435 | NM_005228 | EGFR |
| 436 | NM_004448 | HER2 |
| 437 | NM_001982 | HER3 |
| 438 | NM_005235 | HER4 |
| 439 | NM_002378 | MATK |
| 440 | NM_000875 | IGF1R |
| 441 | NM_000208 | INSR |
| 442 | NM_014215 | INSRR |
| 443 | NM_002227 | JAK1 |
| 444 | NM_004972 | JAK2 |
| 445 | NM_000215 | JAK3 |
| 446 | NM_003331 | TYK2 |
| 447 | NM_006343 | MER |
| 448 | NM_021913 | AXL |
| 449 | NM_006293 | TYRO3 |
| 450 | NM_000245 | MET |
| 451 | NM_002447 | MST1R, RON |
| 452 | NM_002958 | RYK |
| 453 | NM_006206 | PDGFRalpha |
| 454 | NM_002609 | PDGFRbeta |
| 455 | NM_020630 | RET |
| 456 | NM_005012 | ROR1 |
| 457 | NM_004560 | ROR2 |
| 458 | NM_002944 | ROS1 |
| 459 | NM_005607 | PTK2, FAK |
| 460 | NM_004103 | PTK2B, PYK2 |
| 461 | NM_003177 | SYK |
| 462 | NM_001079 | ZAP70 |
| 463 | NM_005424 | TIE1 |
| 464 | NM_000459 | TEK, TIE2 |
| 465 | NM_005592 | MUSK |
| 466 | NM_002529 | NTRK1 |
| 467 | NM_006180 | NTRK2 |
| 468 | NM_002530 | NTRK3 |
| 469 | NM_013994 | DDR1 |
| 470 | NM_006182 | DDR2 |
| 471 | NM_004920 | AATK/LMR1 |
| 472 | XM_055866 | LMTK3 |
| 473 | NM_003985 | TNK1 |
| 474 | L08961 | HUMSPRMTK |
| 475 | NM_004304 | ALK |
| 476 | NM_015978 | CARK |
| 477 | NM_018423 | DKFZp761P1010 |
| 478 | NM_032435 | KIAA1804, MLK4 |
| 479 | AJ277481 | ILK-2 |
| 480 | NM_000906 | NPR1 |
| 481 | NM_000907 | NPR2 |
| 482 | NM_004963 | GUCY2C |
| 483 | NM_000180 | GUCY2D |
| 484 | NM_001522 | GUCY2F |
| 485 | XM_058513 | DKFZp434H2111 |
| 486 | NM_006218 | PIK3CA (phosphoinositide-3-kinase, catalytic, alpha polypeptide) |
| 487 | NM_006219 | PIK3CB (phosphoinositide-3-kinase, catalytic, beta polypeptide) |
| 488 | NM_002649 | PIK3CG (phosphoinositide-3-kinase, catalytic, gamma polypeptide) |
| 489 | NM_005026 | PIK3CD (phosphoinositide-3-kinase, catalytic, delta polypeptide |
| 490 | NM_014006 | SMG1 |
| 491 | NM_000051 | ATM (ataxia telangiectasia mutated) |
| 492 | NM_001184 | ATR (ataxia telangiectasia and Rad3 related) |
| 493 | NM_014216 | ITPK1 |
| 494 | NM_004958 | FRAP1 (FK506 binding protein 12-rapamycin associated protein 1) |
| 495 | NM_002645 | PIK3C2A (phosphoinositide-3-kinase, class 2, alpha polypeptide) |
| 496 | NM_002647 | PIK3C3 (phosphoinositide-3-kinase, class 3); Vps34 |
| 497 | NM_002651 | PIK4CB (phosphatidylinositol 4-kinase, catalytic, beta polypeptide) |
| 498 | NM_002650 | PIK4CA (phosphatidylinositol 4-kinase, catalytic, alpha polypeptide) |
| 499 | NM_003496 | TRRAP (transformation/transcription domain-associated protein) |
| 500 | NM_002646 | PIK3C2B (phosphoinositide-3-kinase, class 2, beta polypeptide) |
| 501 | NM_004570 | PIK3C2G (phosphoinositide-3-kinase, class 2, gamma polypeptide) |
| 502 | NM_006904 | PRKDC (protein kinase, DNA-activated) |
| 503 | NM_013302 | elongation factor-2 kinase |
| 504 | NM_025144 | LAK (lymphocyte alpha-kinase) |
| 505 | NM_017662 | TRPM6 |
| 506 | NM_052947 | HAK |
| 507 | NM_020778 | MIDORI |
| 508 | NM_005881 | BCKDK |
| 509 | NM_002610 | PDK1 |
| 510 | NM_002611 | PDK2 |
| 511 | NM_005391 | PDK3 |
| 512 | NM_002612 | PDK4 |
| 513 | NM_018343 | RIOK2 |
| 514 | NM_031480 | RIOK1 |
| 515 | NM_003831 | RIOK3 |
| 516 | BC017459 | ADCK1 |
| 517 | NM_052853 | ADCK2 |
| 518 | NM_020247 | CABC1 |
| 519 | NM_024876 | ADCK4 |
| 520 | NM_174922 | ADCK5 |
| 521 | NM_032454 | STK19 |
| 522 | NM_001726 | BRDT |
| 523 | NM_005104 | BRD2 |
| 524 | NM_007371 | BRD3 |
| 525 | NM_058243 | BRD4, var. long |
| 526 | NM_014299 | BRD4, var. Short |
| 527 | NM_004606 | TAF1 |
| 528 | NM_153809 | TAF1L |
| 529 | NM_003852 | TIF1 |
| 530 | NM_005762 | TRIM28 |
| 531 | NM_015906 | TRIM33 |

Accession Numbers were obtained from the public data bank NCBI (http://www.ncbi.nlm.nih.gov/).

Additionally, the present invention relates to the use of the compounds of the present invention for the manufacturing of a pharmaceutical composition for the prophylaxis and/or treatment of prion diseases, immunological diseases, autoimmune diseases, bipolar and clinical disorders, cardiovascular diseases, cell proliferative diseases, diabetes, inflammation, transplant rejections, erectile dysfunction, neurodegenerative diseases, stroke, virally and/or bacterially induced diseases.

Further aspects of the present invention relate to the use of the compounds of general formula (I) for the preparation of a pharmaceutical composition useful for prophylaxis and/or treatment of infectious diseases including opportunistic diseases, prion diseases, immunological diseases, autoimmune diseases, bipolar and clinical disorders, cardiovascular diseases, cell proliferative diseases, diabetes, inflammation, osteoporosis, transplant rejections, erectile dysfunction, neurodegenerative diseases, stroke, hair loss, obesity, polycystic ovary syndrome, ischaemia leukopenia, Down's syndrome, Lewy body disease, periodontal diseases, corneal ulceration, proteinuria, myelodysplastic syndromes and biliary cirrhosis.

### Infectious Diseases Including Opportunistic Infections

In yet another aspect of the present invention, the compounds according to the general formula (I) are for the preparation of a pharmaceutical composition for the prophylaxis and/or treatment of infectious diseases, including opportunistic diseases and opportunistic infections. The term infectious diseases comprises infections caused by viruses, bacteria, prions, fungi, and/or parasites.

Especially, virally induced infectious diseases, including opportunistic diseases are addressed. In a preferred embodiment of this aspect, the virally induced infectious diseases, including opportunistic diseases, are caused by retroviruses, human endogenous retroviruses (HERVs), hepadnaviruses, herpesviruses, flaviviridae, and/or adenoviruses. Preferably, the retroviruses are selected from lentiviruses or oncoretroviruses, wherein the lentivirus is preferably selected from the group comprising: HIV-1, HIV-2, feline immunodeficiency virus (FIV), bovine immunodeficiency virus (BIV), sivian immunodeficiency viruses (SIVs), chimeras of HIV and SIV (SHIV), caprine arthritis encephalitis virus (CAEV), visna/maedi virus (VMV) or equine infectious anemia virus (EIAV), preferably HIV-1 and HIV-2, and the oncoretrovirus is preferably selected from HTLV-I, HTLV-II or bovine leukemia virus (BLV), preferably HTLV-I and HTLV-II.

The hepadnavirus is preferably selected from HBV, ground squirrel hepatitis virus (GSHV) or woodchuck hepatitis virus (WHV), preferably HBV, the herpesvirus is selected from the group comprising: Herpes simplex virus I (HSV I), herpes simplex virus II (HSV II), Epstein-Barr virus (EBV), varicella zoster virus (VZV), human cytomegalovirus (HCMV), human herpesvirus 6 (HHV-6), human herpesvirus 7 (HHV-7) or human herpesvirus 8 (HHV-8), preferably HCMV, and the flaviviridae is selected from HCV, West nile or Yellow Fever.

It is to be understood, that all the viruses mentioned above, also comprise drug resistant virus strains.

Examples of infective diseases are AIDS, Alveolar Hydatid Disease (AHD, Echinococcosis), Amebiasis (Entamoeba histolytica Infection), Angiostrongylus Infection, Anisakiasis, Anthrax, Babesiosis (Babesia Infection), Balantidium Infection (Balantidiasis), Baylisascaris Infection (Raccoon Roundworm), Bilharzia (Schistosomiasis), Blastocystis hominis Infection (Blastomycosis), Boreliosis, Botulism, Brainerd Diarrhea, Brucellosis, BSE (Bovine Spongiform Encephalopathy), Candidiasis, Capillariasis (Capillaria Infection), CFS (Chronic Fatigue Syndrome), Chagas Disease (American Trypanosomiasis), Chickenpox (Varicella-Zoster virus), Chlamydia pneumoniae Infection, Cholera, Chronic Fatigue Syndrome, CJD (Creutzfeldt-Jakob Disease), Clonorchiasis (Clonorchis Infection), CLM (Cutaneous Larva Migrans, Hookworm Infection), Coccidioidomycosis, Conjunctivitis, Coxsackievirus A16 (Hand, Foot and Mouth Disease), Cryptococcosis, Cryptosporidium Infection (Cryptosporidiosis), Culex mosquito (Vector of West Nile Virus), Cutaneous Larva Migrans (CLM), Cyclosporiasis (Cyclospora Infection), Cysticercosis (Neurocysticercosis), Cytomegalovirus Infection, Dengue/Dengue Fever, Dipylidium Infection (Dog and Cat Flea Tapeworm), Ebola Virus Hemorrhagic Fever, Echinococcosis (Alveolar Hydatid Disease), Encephalitis, Entamoeba coli Infection, Entamoeba dispar Infection, Entamoeba hartmanni Infection, Entamoeba histolytica Infection (Amebiasis), Entamoeba polecki Infection, Enterobiasis (Pinworm Infection), Enterovirus Infection (Non-Polio), Epstein-Barr Virus Infection, Escherichia coli Infection, Foodborne Infection, Foot and mouth Disease, Fungal Dermatitis, Gastroenteritis, Group A streptococcal Disease, Group B streptococcal Disease, Hansen's Disease (Leprosy), Hantavirus Pulmonary Syndrome, Head Lice Infestation (Pediculosis), Heliobacter pylori Infection, Hematologic Disease, Hendra Virus Infection, Hepatitis (HCV, HBV), Herpes Zoster (Shingles), HIV Infection, Human Ehrlichiosis, Human Parainfluenza Virus Infection, Influenza, Isosporiasis (Isospora Infection), Lassa Fever, Leishmaniasis, Kala-azar (Kala-azar, Leishmania Infection), Leprosy, Lice (Body lice, Head lice, Pubic lice), Lyme Disease, Malaria, Marburg Hemorrhagic Fever, Measles, Meningitis, Mosquito-borne Diseases, Mycobacterium avium Complex (MAC) Infection, Naegleria Infection, Nosocomial Infections, Nonpathogenic Intestinal Amebae Infection, Onchocerciasis (River Blindness), Opisthorciasis (Opisthorcis Infection), Parvovirus Infection, Plague, PCP (Pneumocystis carinii Pneumonia), Polio, Q Fever, Rabies, Respiratory Syncytial Virus (RSV) Infection, Rheumatic Fever, Rift Valley Fever, River Blindness (Onchocerciasis), Rotavirus Infection, Roundworms Infection, Salmonellosis, Salmonella Enteritidis, Scabies, Shigellosis, Shingles, Sleeping Sickness, Smallpox, Streptococcal Infection, Tapeworm Infection (Taenia Infection), Tetanus, Toxic Shock Syndrome, Tuberculosis, Ulcers (Peptic Ulcer Disease), Valley Fever, Vibrio parahaemolyticus Infection, Vibrio vulnificus Infection, Viral Hemorrhagic Fever, Warts, Waterborne infectious Diseases, West Nile Virus Infection (West Nile Encephalitis), Whooping Cough, Yellow Fever, Charga's disease, effects of Shiga-like toxin resulting from Staphylococcus infection, meningococcal infection, infections from Borrelia burgdorferi, Treponema pallidum.

### Bacterial Infections

As described above, the compounds according to the general formula (I) are also useful for the preparation of a pharmaceutical composition for prophylaxis and/or treatment of bacterially induced infectious diseases, including opportunistic diseases and opportunistic infections, wherein the bacterially induced infectious diseases, including opportunistic diseases, are selected from tuberculosis, leprosy or mycobacteria-induced meningitis. One advantage of the inventive compounds disclosed herein is there use against drug resistant bacterial strains.

### Prion Diseases

Another aspect of the present invention is directed to the use of at least one compound of the general formula (I) and/or pharmaceutically acceptable salts thereof for prophylaxis and/or treatment of prion diseases.

Prions are infectious agents, which do not have a nucleic acid genome. It seems that a protein alone is the infectious agent. A prion has been defined as “small proteinaceous infectious particle, which resists inactivation, by procedures that modify nucleic acids”. The discovery that proteins alone can transmit an infectious disease has come as a considerable surprise to the scientific community. Prion diseases are often called “transmissible spongiform encephalopathies”, because of the post mortem appearance of the brain with large vacuoles in the cortex and cerebellum. Probably most mammalian species develop these diseases. Prion diseases are a group of neurodegenerative disorders of humans and animals and the prion diseases can manifest as sporadic, genetic or infectious disorders.

As used herein the term “prion diseases” refers to transmissible spongiform encephalopathies. Examples for prion diseases acquired by exogenous infection are the Bovine spongiform encephalitis (BSE) of cattle and cows and the new variant of Creutzfeld-Jakob disease (vCJD) caused by BSE as well as scrapie (sheep, goat), TME (transmissible mink encephalopathy; mink), and CWD (chronic wasting disease; muledeer, deer, elk) of animals. Examples of human prion diseases include kuru, Alpers Syndrome, sporadic Creutzfeldt-Jakob disease (sCJD), familial CJD (fCJD), iatrogenic CJD (iCJD), Gerstmann-Straussler-Scheinker (GSS) disease, fatal familial insomnia (FFI), and especially the new variant CJD (nvCJD or vCJD). Preferred are BSE, vCJD, and CJD.

The name “prion” is used to describe the causative agents, which underlie the transmissible spongiform encephalopathies. A prion is proposed to be a novel infectious particle that differs from viruses and viroids. It is composed solely of one unique protein that resists most inactivation procedures such as heat, radiation, and proteases. The latter characteristic has led to the term protease-resistant isoform of the prion protein. The protease-resistant isoform has been proposed to slowly catalyze the conversion of the normal prion protein into the abnormal form.

The term “isoform” in the context of prions means two proteins with exactly the same amino acid sequence, that are folded into molecules with dramatically different tertiary structures. The normal cellular isoform of the prion protein (PrP^{c}) has a high a-helix content, a low b-sheet content, and is sensitive to protease digestion. The abnormal, disease-causing isoform (PrP^{Sc}) has a lower a-helix content, a much higher b-sheet content, and is much more resistant to protease digestion.

### Immunological Diseases

Another aspect of the present invention is directed to the use of at least one compound of the general formula (I) and/or pharmaceutically acceptable salts thereof for prophylaxis and/or treatment of immunological diseases, neuroimmunological diseases, and autoimmune diseases.

Immunological diseases are, for instance, asthma and diabetes, rheumatic and autoimmune diseases, AIDS, rejection of transplanted organs and tissues (cf. below), rhinitis, chronic obstructive pulmonary diseases, ulcerative colitis, sinusitis, lupus erythematosus, recurrent infections, atopic dermatitis/eczema and occupational allergies, food allergies, drug allergies, severe anaphylactic reactions, anaphylaxis, and other manifestations of allergic disease, as well as uncommon problems such as primary immunodeficiencies, including antibody deficiency states, cell mediated immunodeficiencies (e.g., severe combined immunodeficiency, DiGeorge syndrome, Hyper-IgE syndrome, Wiskott-Aldrich syndrome, ataxia-telangiectasia), immune mediated cancers, and white cell defects.

“Autoimmune disease” refers to a category of more than 80 chronic illnesses, each very different in nature, that can affect everything from the endocrine glands (like the thyroid) to organs like the kidneys, as well as to the digestive system.

In autoimmune diseases, such as systemic lupus erythematosus, rheumatoid arthritis (RA), multiple sclerosis (MS), immune-mediated or type 1 diabetes mellitus, immune mediated glomerulonephritis, scleroderma, pernicious anemia, alopecia, pemphigus, pemphigus vulgaris, myasthenia gravis, inflammatory bowel diseases, Crohn's disease, psoriasis, autoimmune thyroid diseases, and Hashimoto's disease, Hashimoto's thyroiditis, dermatomyositis, goodpasture syndrome, myasthenia gravis pseudoparalytica, ophtalmia sympatica, phakogene uveitis, chronical aggressive hepatitis, primary billiary cirrhosis, autoimmune hemolytic anemy, Werlof disease, specific cells uncontrollably attack the body's own tissues and organs (autoimmunity), producing inflammatory reactions and other serious symptoms and diseases.

There are many different autoimmune diseases, and they can each affect the body in different ways. For example, the autoimmune reaction is directed against the brain in multiple sclerosis and the gut in Crohn's disease. In other autoimmune diseases such as systemic lupus erythematosus (lupus), affected tissues and organs may vary among individuals with the same disease. One person with lupus may have affected skin and joints whereas another may have affected skin, kidney, and lungs. Ultimately, damage to certain tissues by the immune system may be permanent, as with destruction of insulin-producing cells of the pancreas in Type 1 diabetes mellitus.

### Bipolar and Clinical Disorders

Another aspect of the present invention is directed to the use of at least one compound of the general formula (I) and/or pharmaceutically acceptable salts thereof for prophylaxis and/or treatment of bipolar and clinical disorders.

The term “bipolar and clinical disorders” shall refer to adjustment disorders, anxiety disorders, delirium, dementia, amnestic and other cognitive disorders, disorders usually first diagnosed in infancy (e.g.), childhood, or adolescence, dissociative disorders (e.g. dissociative amnesia, depersonalization disorder, dissociative fugue and dissociative identity disorder), eating disorders, factitious disorders, impulse-control disorders, mental disorders due to a general medical condition, mood disorders, other conditions that may be a focus of clinical attention, personality disorders, schizophrenia and other psychotic disorders, sexual and gender identity disorders, sleep disorders, somatoform disorders, substance-related disorders, generalized anxiety disorder (e.g. acute stress disorder, posttraumatic stress disorder), panic disorder, phobia, agoraphobia, obsessive-compulsive disorder, stress, acute stress disorder, anxiety neurosis, nervousness, phobia, posttraumatic stress disorder, posttraumatic stress disorder (PTSD), abuse, obsessive-compulsive disorder (OCD), manic depressive psychosis, specific phobias, social phobia, adjustment disorder with anxious features.

Examples for disorders usually first diagnosed in infancy, childhood, or adolescence are: mental retardation, learning disorders, mathematics disorder, reading disorder, disorder of written expression, motor skills disorders, developmental coordination disorder, communication disorders, expressive language disorder, phonological disorder, mixed receptive-expressive language disorder, stuttering, pervasive developmental disorders, Asperger's disorder, autistic disorder, childhood disintegrative disorder, Rett's disorder, pervasive developmental disorder, attention-deficit/hyperactivity disorder (ADHD), conduct disorder, oppositional defiant disorder, feeding disorder of infancy or early childhood, pica, rumination disorder, tic disorders, chronic motor or vocal tic disorder, Tourette's syndrome, elimination disorders, encopresis, enuresis, selective mutism, separation anxiety disorder, reactive attachment disorder of infancy or early childhood, stereotypic movement disorder.

Examples for substance-related disorders are: alcohol related disorders, amphetamine related disorders, caffeine related disorders, cannabis related disorders, cocaine related disorders, hallucinogen related disorders, inhalant related disorders, nicotine related disorders, opioid related disorders, psychotic disorder, psychotic disorder, phencyclidine-related disorder, abuse, persisting amnestic disorder, anxiety disorder, persisting dementia, dependence, intoxication, intoxication delirium, mood disorder, psychotic disorder, withdrawal, withdrawal delirium, sexual dysfunction, sleep disorder.

### Cardiovascular Diseases

The inventive compounds are also useful for prophylaxis and/or treatment of cardiovascular diseases such as adult congenital heart disease, aneurysm, stable angina, unstable angina, angina pectoris, angioneurotic edema, aortic valve stenosis, aortic aneurysm, arrhythmia, arrhythmogenic right ventricular dysplasia, arteriosclerosis, arteriovenous malformations, atrial fibrillation, Behcet syndrome, bradycardia, cardiac tamponade, cardiomegaly, congestive cardiomyopathy, hypertrophic cardiomyopathy, restrictive cardiomyopathy, cardiovascular disease prevention, carotid stenosis, cerebral hemorrhage, Churg-Strauss syndrome, diabetes, insulin resistance and diabetes including non-insulin-dependent diabetes mellitus (NIDDM), Ebstein's Anomaly, Eisenmenger complex, cholesterol embolism, bacterial endocarditis, fibromuscular dysplasia, congenital heart defects, heart diseases, congestive heart failure, heart valve diseases, heart attack, epidural hematoma, hematoma, subdural, Hippel-Lindau disease, hyperemia, hypertension, pulmonary hypertension, hypertrophic growth, left ventricular hypertrophy, right ventricular hypertrophy, hypoplastic left heart syndrome, hypotension, intermittent claudication, ischemic heart disease, Klippel-Trenaunay-Weber syndrome, lateral medullary syndrome, long QT syndrome mitral valve prolapse, moyamoya disease, mucocutaneous lymph node syndrome, myocardial infarction, myocardial ischemia, myocarditis, pericarditis, peripheral vascular diseases, phlebitis, polyarteritis nodosa, pulmonary atresia, Raynaud disease, chronic renal failure, restenosis, Sneddon syndrome, stenosis, superior vena cava syndrome, syndrome X, tachycardia, Takayasu's arteritis, hereditary hemorrhagic telangiectasia, telangiectasis, temporal arteritis, tetralogy of fallot, thromboangiitis obliterans, thrombosis, thromboembolism, tricuspid atresia, varicose veins, vascular diseases, vasculitis, vasospasm, ventricular fibrillation, Williams syndrome, peripheral vascular disease, varicose veins and leg ulcers, deep vein thrombosis, Wolff-Parkinson-White syndrome.

Preferred are adult congenital heart disease, aneurysms, angina, angina pectoris, arrhythmias, cardiovascular disease prevention, cardiomyopathies, congestive heart failure, myocardial infarction, pulmonary hypertension, hypertrophic growth, restenosis, stenosis, thrombosis and arteriosclerosis.

### Proliferative Disease

In yet another preferred embodiment, the cell proliferative disease is cancer, which is preferably selected from the group comprising:

The proliferation disorders and cancers are preferably selected from the group comprising advanced cancers, lymphoid malignancies and tumor metastases, especially adenocarcinoma, choroidal melanoma, acute leukemia, acoustic neurinoma, ampullary carcinoma, anal carcinoma, astrocytoma, basal cell carcinoma, pancreatic cancer, desmoid tumor, bladder cancer, bronchial carcinoma, breast cancer, Burkitt's lymphoma, corpus cancer, CUP-syndrome (carcinoma of unknown primary), colorectal cancer, small intestine cancer, small intestinal tumors, ovarian cancer, endometrial carcinoma, ependymoma, epithelial cancer types, Ewing's tumors, gastrointestinal tumors, gastric cancer, gallbladder cancer, gall bladder carcinomas, uterine cancer, cervical cancer, cervix, glioblastomas, gynecologic tumors, ear, nose and throat tumors, hematologic neoplasias, hairy cell leukemia, urethral cancer, skin cancer, skin testis cancer, brain tumors (gliomas), brain metastases, testicle cancer, hypophysis tumor, carcinoids, Kaposi's sarcoma, laryngeal cancer, germ cell tumor, bone cancer, colorectal carcinoma, head and neck tumors (tumors of the ear, nose and throat area), colon carcinoma, craniopharyngiomas, oral cancer (cancer in the mouth area and on lips), cancer of the central nervous system, liver cancer, liver metastases, leukemia, eyelid tumor, lung cancer, lymph node cancer (Hodgkin's/Non-Hodgkin's), lymphomas, stomach cancer, malignant melanoma, malignant neoplasia, malignant tumors gastrointestinal tract, breast carcinoma, rectal cancer, medulloblastomas, melanoma, meningiomas, Hodgkin's disease, mycosis fungoides, nasal cancer, neurinoma, neuroblastoma, kidney cancer, renal cell carcinomas, non-Hodgkin's lymphomas, oligodendroglioma, esophageal carcinoma, osteolytic carcinomas and osteoplastic carcinomas, osteosarcomas, ovarial carcinoma, pancreatic carcinoma, penile cancer, plasmocytoma, prostate cancer, pharyngeal cancer, rectal carcinoma, retinoblastoma, vaginal cancer, thyroid carcinoma, Schneeberger disease, esophageal cancer, spinalioms, T-cell lymphoma (mycosis fungoides), thymoma, tube carcinoma, eye tumors, urethral cancer, urologic tumors, urothelial carcinoma, vulva cancer, wart appearance, soft tissue tumors, soft tissue sarcoma, Wiim's tumor, cervical carcinoma and tongue cancer.

Preferred are the following cancer types: bladder, breast, central nervous system, colon, gastric, lung, kidney, melanoma, head and neck, ovarian, cervix, glioblastoma, pancreas, prostate, stomach, skin testis, leukemia, Hodgkin's lymphoma, liver and renal cancer.

### Diabetes

In yet another preferred embodiment, said diabetes is selected from Type I diabetes or Type II diabetes and non-insulin-dependent diabetes mellitus (NIDDM).

### Inflammation

In yet another preferred embodiment, said inflammation is mediated preferably by the cytokines TNF-α, IL-1β, GM-CSF, IL-6 and/or IL-8.

As described above, the compounds according to general formula (I) are pharmaceutically active agents for prophylaxis and/or treatment of inflammatory diseases. Thus, these compounds are used for the manufacture of a pharmaceutical formulation for prophylaxis and/or treatment of inflammations and inflammatory diseases in mammals, including humans.

Inflammatory diseases can emanate from infectious and non-infectious inflammatory conditions which may result from infection by an invading organism or from irritative, traumatic, metabolic, allergic, autoimmune, or idiopathic causes as shown in the following list.

I. Acute infections
A. Viral
B. Bacterial
   II. Noninfectious causes
   III. Chronic (granulomatous) diseases
A. Bacterial
B. Spirochetal
C. Mycotic (Fungal)
D. Idiopathic
   IV. Allergic, immune, and idiopathic disorders
A. Hypersensitivity reactions
B. Immune and idiopathic disorders
   V. Miscellaneous inflammatory conditions
A. Parasitic infections
B. Inhalation causes:
   Acute (thermal) injury
   Pollution and inhalant allergy
   Carcinogens
C. Radiation injury:
   Radionecrosis

Thus, the compounds disclosed herein can be used for prophylaxis and/or treatment of inflammations caused by invading organisms such as viruses, bacteria, prions, and parasites as well as for prophylaxis and/or treatment of inflammations caused by irritative, traumatic, metabolic, allergic, autoimmune, or idiopathic reasons.

Consequently, the disclosed compounds are useful for prophylaxis and/or treatment of inflammatory diseases which are initiated or caused by viruses, parasites, and bacteria which are connected to or involved in inflammations.

The following bacteria are known to cause inflammatory diseases: mycoplasma pulmonis (causes e.g. chronic lung diseases (CLD), murine chronic respiratory disease), ureaplasma urealyticum (causes pneumonia in newborns), mycoplasma pneumoniae and chlamydia pneumoniae (cause chronic asthma), C. pneumoniae (causes atherosclerosis, pharyngitis to pneumonia with empyema, human coronary heart disease), Heliobacter pylori (human coronary heart disease, stomach ulcers).

The following viruses are known to cause inflammatory diseases: herpes viruses especially cytomegalovirus (causes human coronary heart disease).

The compounds disclosed herein are useful for prophylaxis and/or treatment of inflammatory diseases caused and/or induced and/or initiated and/or enhanced by the afore-mentioned bacteria or viruses.

Furthermore, the compounds of formula (I) are useful for prophylaxis and/or treatment of inflammatory diseases of the central nervous system (CNS), inflammatory rheumatic diseases, inflammatory diseases of blood vessels, inflammatory diseases of the middle ear, inflammatory bowel diseases, inflammatory diseases of the skin, inflammatory disease uveitis, inflammatory diseases of the larynx. Examples are osteoarthritis, septic arthritis, bone resorption, postmenopausal osteoperosis, sepsis, gram negative sepsis, septic shock, endotoxin shock, systemic inflammatory response syndrome, irritable bowel syndrome, Jarisch Heryheimer reactions, adult respiratory distress syndrome, acute pulmonary fibrotic diseases, pulmonary sarcoidosis, allergic respiratory diseases, COPD (chronic obstructive pulmonary disease), silicosis, coal worker's pneumoconiosis, alveolar injury, hepatic failure, liver disease during acute inflammation, immunedeficiency and fibrotic diseases, dermatosis, including psoriasis, atopic dermatitis and ultraviolet radiation (UV)-induced skin damage.

Examples for inflammatory diseases of the central nervous system (CNS) are algal disorders, protothecosis, bacterial disorders, abscessation, bacterial meningitis, idiopathic inflammatory disorders, eosinophilic meningoencephalitis, feline polioencephalomyelitis, granulomatous meningoencephalomyelitis, meningitis, steroid responsive meningitis-arteritis, miscellaneous meningitis/meningoencephalitis, necrotizing encephalitis, pyogranulomatous meningoencephalomyelitis, shaker dog disease, mycotic diseases of the CNS, parasitic encephalomyelitis, prion protein induced diseases, feline spongiform encephalopathy, protozoal encephalitis-encephalomyelitis, toxoplasmosis, neosporosis, sarcocystosis, encephalitozoonosis, trypanosomiasis, acanthamebiasis, babesiosis, leishmaniasis, rickettsial disorders, rocky mountain spotted fever, canine ehrlichiosis, viral disorders, aujeszky's disease, borna disease, canine herpes virus encephalomyelitis, canine distemper encephalomyelitis, canine distemper encephalomyelitis in immature animals, multifocal distemper encephalomyelitis in mature animals, old dog encephalitis, chronic relapsing encephalomyelitis, post-vaccinal canine distemper encephalitis, feline immunodeficiency virus, feline infectious peritonitis, feline leukemia virus, infectious canine hepatitis, La Crosse virus encephalitis, parvovirus encephalitis, rabies, post-vaccinal rabies, tick-borne encephalitis in dogs.

Examples for inflammatory rheumatic diseases are rheumatoid arthritis, scleroderma, lupus, polymyositis, dermatomyositis, psoriatic arthritis, ankylosing spondylitis, Reiters's syndrome, juvenile rheumatoid arthritis, bursitis, tendinitis (tendonitis), and fibromyositis.

Examples for inflammatory diseases of blood vessels are vasculitis, autoantibodies in vasculitis, microscopic polyangiitis, giant cell arteritis, Takayasu's arteritis, vasculitis of the central nervous system, thromboangiitis obliterans (Buerger's Disease), vasculitis secondary to bacterial, fungal, and parasitic infection, vasculitis and rheumatoid arthritis, vasculitis in systemic lupus erythematosus, vasculitis in the idiopathic inflammatory myopathies, relapsing polychondritis, systemic vasculitis in sarcoidosis, vasculitis and malignancy, and drug-induced vasculitis.

Examples for inflammatory diseases of the middle ear are acute suppurative otitis media, bullous myringitis, granular myringitis, and chronic suppurative otitis media, which can manifest as mucosal disease, cholesteatoma, or both.

Examples for inflammatory bowel diseases are ulcerative colitis, Crohn's disease.

Examples for inflammatory diseases of the skin are acute inflammatory dermatoses, urticaria (hives), spongiotic dermatitis, allergic contact dermatitis, irritant contact dermatitis, atopic dermatitis, erythemal multiforme (EM minor), Stevens-Johnson syndrome (SJS, EM major), toxic epidermal necrolysis (TEN), chronic inflammatory dermatoses, psoriasis, lichen planus, discoid lupus erythematosus, and acne vulgaris

Uveitis are inflammations located in and/or on the eye and may be associated with inflammation elsewhere in the body. In most circumstances, patients who have uveitis as part of a disease elsewhere in the body are aware of that illness. The majority of patients with uveitis do not have an apparent associated systemic illness. Causes of uveitis can be infectious causes, masquerade syndromes, suspected immune-mediated diseases, and/or syndromes confined primarily to the eye.

The following viruses are associated with inflammations: human immunodeficiency virus-I, herpes simplex virus, herpes zoster virus, and cytomegalovirus.

Bacterial or spirochetal caused, induced, initiated and/or enhanced inflammations are tuberculosis, leprosy, proprionobacterium, syphilis, Whipple's disease, leptospirosis, brucellosis, and lyme disease.

Parasitic (protozoan or helminthic) caused, induced, initiated and/or enhanced inflammations are toxoplasmosis, acanthameba, toxocariasis, cysticercosis, onchocerciasis.

Examples of inflammatory diseases caused, induced, initiated and/or enhanced by fungi are histoplasmosis, coccidioidomycosis, candidiasis, aspergillosis, sporotrichosis, blastomycosis, and cryptococcosis.

Masquerade syndromes are, for instance, leukemia, lymphoma, retinitis pigmentosa, and retinoblastoma.

Suspected immune-mediated diseases can be selected from the group comprising ankylosing spondylitis, Behcet's disease, Crohn's disease, drug or hypersensitivity reaction, interstitial nephritis, juvenile rheumatoid arthritis, Kawasaki's disease, multiple sclerosis, psoriatic arthritis, Reiter's syndrome, relapsing polychondritis, sarcoidosis, Sjogren's syndrome, systemic lupus erythematosus, ulcerative colitis, vasculitis, vitiligo, Vogt Koyanagi Harada syndrome.

Syndromes confined primarily to the eye are, for instance, acute multifocal placoid pigmentary epitheliopathy, acute retinal necrosis, birdshot choroidopathy, Fuch's heterochromic cyclitis, glaucomatocyclitic crisis, lens-induced uveitis, multifocal choroiditis, pars planitis, serpiginous choroiditis, sympathetic ophthalmia, and trauma.

Examples for inflammatory diseases of the larynx are gastroesophageal (laryngopharyngeal) reflux disease, pediatric laryngitis, acute laryngeal infections of adults, chronic (granulomatous) diseases, allergic, immune, and idiopathic disorders and miscellaneous inflammatory conditions.

Pediatric laryngitis is known as acute (viral or bacterial) infection such as laryngotracheitis (croup), supraglottis (epiglottitis), diphtheria, and noninfectious causes are for example spasmodic croup and traumatic laryngitis.

Acute laryngeal infections of adults are, for instance, viral laryngitis, common upper respiratory infection, laryngotracheitis, herpes simplex, bacterial laryngitis, supraglottis, laryngeal abscess, and gonorrhea.

Chronic (granulomatous) diseases can be selected from the group comprising bacterial diseases, tuberculosis, leprosy, scleroma, actinomycosis, tularemia, glanders, spirochetal (syphilis) diseases, mycotic (fungal) diseases, candidiasis, blastomycosis, histoplasmosis, coccidiomycosis, aspergillosis, idiopathic diseases, sarcoidosis, and Wegener's granulomatosis.

Allergic, immune, and idiopathic disorders are, for example, hypersensitivity reactions, angioedema, Stevens-Johnson syndrome, immune and idiopathic disorders, infections of the immunocompromised host, rheuatoid arthritis, systeic lupus erythematosus, cicatricial pemphigoid, relapsing polychondritis, Sjogren's syndrome, and amyloidosis.

Miscellaneous inflammatory conditions are, for instance, parasitic infections, trichinosis, leishmaniasis, schistosomiasis, syngamus laryngeus, inhalation laryngitis, acute (thermal) injury, pollution and inhalant allergy, carcinogens, radiation injury, radiation laryngitis, radionecrosis, vocal abuse, vocal-cord hemorrhage, muscle tension dysphonias, and contact ulcer and granuloma.

### Transplant Rejection

Transplant rejection is when a transplant recipient's immune system attacks a transplanted organ or tissue. No two people (except identical twins) have identical tissue antigens. Therefore, in the absence of immunosuppressive drugs, organ and tissue transplantation would almost always cause an immune response against the foreign tissue (rejection), which would result in destruction of the transplant. Though tissue typing ensures that the organ or tissue is as similar as possible to the tissues of the recipient, unless the donor is an identical twin, no match is perfect and the possibility of organ/tissue rejection remains.

The inventive compounds of general formula (I) are used as immunosuppressive drugs and/or anti-rejection drugs in order to prevent transplant rejection such as systemic lupus erythematosis, host-versus-graft reactions, ischemia reperfusion injury and allograft rejection including chronic lung, kidney and heart allograft rejection, complications due to total hip replacement, and ankylosing spondylitis.

One example of transplant rejection is the graft-versus-host-disease (GVHD) that can occur following bone marrow transplant. The donor's immune cells in the transplanted marrow make antibodies against the host's (transplant patient's) tissues and attack the patient's vital organs. Transplant rejections (also known as graft rejection or tissue/organ rejection) may commonly occur when tissue or organs, which need blood supply, are transplanted. Said organs comprise especially inner organs such as heart, heart-lungs, lungs, liver, kidney, pancreas, spleen, skin, tissue, bone marrow, spinal marrow, hormone producing glands, gonads and gonadal glands.

### Neurodegenerative Diseases

Another aspect of the present invention is directed to the use of at least one compound of the general formula (I) and/or pharmaceutically acceptable salts thereof for prophylaxis and/or treatment of neurodegeneration and neurodegenerative disorders.

Among the hundreds of different neurodegenerative disorders, the attention has been given only to a handful, including Alzheimer disease, Parkinson disease, Huntington disease, and amyotrophic lateral sclerosis.

It is worth to mention that the same neurodegenerative process can affect different areas of the brain, making a given disease appear very different from a symptomatic standpoint.

Neurodegenerative disorders of the central nervous system (CNS) can be grouped into diseases of the cerebral cortex (Alzheimer disease), the basal ganglia (Parkinson disease), the brain-stem and cerebellum, or the spinal cord (amyotrophic lateral sclerosis).

Examples for neurodegeneration and neurodegenerative disorders are Alzheimer disease, Parkinson disease, Huntington disease, amyotrophic lateral sclerosis, AIDS-related dementia, retinitis pigmentosa, spinal muscular atrophy and cerebrellar degeneration, fragile X-associated tremor/ataxia syndrome (FXTAS), progressive supranuclear palsy (PSP), and striatonigral degeneration (SND), which is included with olivopontocerebellear degeneration (OPCD), and Shy Drager syndrome (SDS) in a syndrome known as multiple system atrophy (MSA), acute encephalitis, brain injury, amyotrophic lateral sclerosis and inflammatory pain, regenerative (recovery) treatment of CNS disorders such as spinal cord injury, acute neuronal injury (stroke, traumatic brain injury) progressive supranuclear palsy, subacute sclerosing panencephalitic parkinsonism, postencephalitic, pugilistic encephalitis, guam parkinsonism-dementia complex, corticobasal degeneration, frontotemporal dementia, AIDS associated dementia, mood disorders.

According to a still further aspect, the present invention refers to pharmaceutical compositions comprising at least one compound according to the present invention as an active ingredient together with at least one pharmaceutically acceptable (i.e. non-toxic) carrier, excipient and/or diluent. The pharmaceutical compositions of the present invention can be prepared in a conventional solid or liquid carrier or diluent and a conventional pharmaceutically-made adjuvant at suitable dosage level in a known way. The preferred preparations are adapted for oral application. These administration forms include, for example, pills, tablets, film tablets, coated tablets, capsules, micro- and nano formulations, liposomal formulations, powders and deposits.

Furthermore, the present invention also includes pharmaceutical preparations for parenteral application, including dermal, intradermal, intragastral, intracutan, intravasal, intravenous, intramuscular, intraperitoneal, intranasal, intravaginal, intrabuccal, percutan, rectal, subcutaneous, sublingual, topical, or transdermal application, which preparations in addition to typical vehicles and/or diluents contain at least one compound according to the present invention and/or a pharmaceutical acceptable salt thereof as active ingredient.

The pharmaceutical compositions according to the present invention containing at least one compound according to the present invention and/or a pharmaceutical acceptable salt thereof as active ingredient will typically be administered together with suitable carrier materials selected with respect to the intended form of administration, i.e. for oral administration in the form of tablets, capsules (either solid filled, semi-solid filled or liquid filled), powders for constitution, gels, elixirs, dispersable granules, syrups, suspensions, and the like, and consistent with conventional pharmaceutical practices. For example, for oral administration in the form of tablets or capsules, the active drug component may be combined with any oral non-toxic pharmaceutically acceptable carrier, preferably with an inert carrier like lactose, starch, sucrose, cellulose, magnesium stearate, dicalcium phosphate, calcium sulfate, talc, mannitol, ethyl alcohol (liquid filled capsules) and the like. Moreover, suitable binders, lubricants, disintegrating agents and coloring agents may also be incorporated into the tablet or capsule. Powders and tablets may contain about 5 to about 95 weight % of the pyridinylamines and/or the respective pharmaceutically active salt as active ingredient.

Suitable binders include starch, gelatin, natural sugars, corn sweeteners, natural and synthetic gums such as acacia, sodium alginate, carboxymethylcellulose, polyethylene glycol and waxes. Among suitable lubricants there may be mentioned boric acid, sodium benzoate, sodium acetate, sodium chloride, and the like. Suitable disintegrants include starch, methylcellulose, guar gum, and the like. Sweetening and flavoring agents as well as preservatives may also be included, where appropriate.

Moreover, the pharmaceutical compositions of the present invention may be formulated in sustained release form. Suitable dosage forms for sustained release include tablets having layers of varying disintegration rates or controlled release polymeric matrices impregnated with the active components and shaped in tablet form or capsules containing such impregnated or encapsulated porous polymeric matrices. Liquid form preparations include solutions, suspensions, and emulsions. Liquid form preparations may also include solutions for intranasal administration.

Aerosol preparations suitable for inhalation may include solutions and solids in powder form, which may be present in combination with a pharmaceutically acceptable carrier such as an inert, compressed gas, e.g. nitrogen.

For preparing suppositories, a low melting wax, such as a mixture of fatty acid glycerides like cocoa butter is melted first, and the active ingredient is then dispersed homogeneously therein e.g. by stirring. The molten, homogeneous mixture is then poured into conveniently sized moulds, allowed to cool, and thereby solidified.

Under tablet a compressed or moulded solid dosage form is understood which comprises the active ingredients with suitable diluents. The tablet may be prepared by compression of mixtures or granulations obtained by wet granulation, dry granulation, or by compaction well known to a person of ordinary skill in the art.

Powders for constitution refers to powder blends containing the active ingredients and suitable diluents which can be suspended e.g. in water or in juice.

Another aspect of the present invention is directed to combination therapies wherein at least one compound according to any formula (I) to (III) is administered together with a known or commonly used drug against infectious diseases, prion diseases, immunological diseases, autoimmune diseases, bipolar and clinical disorders, cardiovascular diseases, cell proliferative diseases, diabetes, inflammation, transplant rejections, erectile dysfunction, neurodegenerative diseases and stroke. Especially preferred are combination therapies including systemic combination therapies of at least one compound of the present invention together with known or commonly used HIV drugs, antibiotics or anticancer drugs. Furthermore, the inventive compounds can also be applied in addition to chemotherapy or any other radiotherapy such as hyperthermia for cancer treatment.

### Determination of the Inhibitory Effect of Representative Compounds of the Present Invention on Various Target Enzymes

In order to determine the inhibitory effect of the compounds of the subject invention on various target enzymes a generic kinase assay was established.

### Generic Kinase Assay:

Reactions were performed in 96-Well U-bottom microtiter plates (Greiner Bio-One; Frickenhausen/Germany, Cat. No. 650161), hereinafter designated “Assay Plates”. 10 μl of a solution comprising 40 μM Myelin Basic Protein (Invitrogen; Carlsbad, Calif./USA; Cat. No. 13228-010) and 4 μM ATP in three-fold concentrated Reaction Buffer (60 mM Tris-HCl, pH 7.5; 30 mM MgCl2; 3 mM dithiothreitol) were added into each well of the Assay Plate. 10 μl of serial dilutions of the compounds of the subject invention, dissolved in 4% DMSO, were then added into each well, except for Positive Control. Wells (C+ wells) and Negative Control Wells (C− wells). 10 μl of 4% DMSO without compounds were added to C+ and C− wells. 10 μl of a 500 mM solution of EDTA in water was then added to C− wells. Then 10 μl of a solution containing 50 μCi/ml Adenosine 5′-[γ-³³P]triphosphate in water was added to each well. To start the reaction 10 μl of the kinase to be assayed was added to each well. The optimal amount of kinase in the assay was determined to be the amount which yields to a turn-over of about 10% of ATP. Assay Plates were incubated for one hour at room temperature. Then 10 μl of a 500 mM solution of EDTA in water was added to each well except C− wells. Samples were now ready for measurement.

Measurements were preformed in 96-Well MAPH-Filter Plates (Millipore; Billerica, Mass./United States; Cat. No. MAPHNOB50), hereinafter designated “Measurement Plates”. Measurement Plates were washed with 200 μl of a 0.75% H₃PO₄ solution per well. The H₃PO₄ solution was exhausted using a Millipore vacuum station. 60 μl of a 0.75% H₃PO₄ solution was then added into each well, followed by the transfer of 30 μl of each well from the Assay Plate into the corresponding wells of the Measurement Plate. The Measurement Plate was incubated for 30 minutes at room temperature. Thereafter each well was washed three times with 200 μl of a 0.75% H₃PO₄ solution using a Millipore vacuum station. 20 μl of scintillation liquid (Supermix Liquid Szintillator; Perkin Elmer, Wellesley. Mass./United States; Cat. No. 1200-439) was then added to each well of the Measurement Plate. The plate was sealed and stored for 30 minutes in the darkness before radioactivity was quantified in a MicroBeta Scintillation Counter (Perkin Elmer, Wellesley. Mass./United States).

The following Table 2a shows the inhibitory effect of representative compounds of the present invention on various target enzymes.

TABLE 2a
Inhibitory effect of the compounds of the present invention on
different targets (A: 50-90% inhibition at 10 μM enzyme concentration;
B: >90% inhibition at 10 μM enzyme concentration; C: IC50 measured:
<1 μM)

| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
|---|---|---|---|---|---|---|---|---|---|---|---|---|---|---|---|---|---|
| 126 | | | | | | | | A | | | | | | A | | | |
| 127 | | | | | | | | A | | | | | | A | | | |
| 206 | | | A | | A | | | C | | | | | | | | | |
| 226 | | | B | A | A | A | A | C | | | | | | | | | |
| 217 | | | B | A | B | A | A | C | | | | | | | | | |
| 230 | A | | B | B | B | A | A | C | | | | | | C | | | |
| 228 | | | A | | | | A | C | | | | | | | | | |
| 218 | | | B | | A | A | A | C | | | | | | | | | |
| 159 | | | A | A | A | A | A | C | | | | | | | | | |
| 222 | | | A | A | A | | A | C | | | | | | | | | |
| 229 | A | | B | A | A | A | A | C | | | | | | A | | | |
| 221 | A | | B | A | B | A | A | C | | | | | | | A | | |
| 212 | A | A | B | A | B | A | A | C | | | | | | C | | | B |
| 223 | A | | B | A | A | A | A | C | | | | | | | | | |
| 216 | A | | B | A | A | A | A | C | | | | | | | | | |
| 224 | | | A | A | A | A | A | C | | | | | | | | | |
| 207 | | | | | A | | | C | | | | | | | | | |
| 225 | A | | B | A | B | A | A | C | | | | | | A | | | |
| 195 | A | | A | | A | | | C | | | | | | | | | |
| 166 | | | | | | | | A | | | | | A | | | | |
| 194 | | | A | | | | A | C | | | | | | | | | |
| 205 | | | | | | | A | C | A | A | | A | | | | | |
| 227 | | | A | | | | A | C | | | | | | | | | |
| 238 | A | | B | A | A | A | A | C | | | | | | | | | |
| 239 | A | | | A | A | A | B | C | B | A | B | | | A | | | C |
| 232 | A | | B | B | B | A | A | C | | | | | | | | | |
| 243 | A | | A | A | B | | | C | | | | | | A | | | B |
| 246 | B | | A | A | A | A | A | C | | | | | | | | | |
| 234 | A | | A | A | B | B | B | C | | | B | A | | | | | |
| 241 | | | | | | | | C | | | | | | A | | C | |
| 245 | A | | | | | | | C | | | | | | | | C | |
| 244 | | | A | A | A | B | A | C | | | | | | | | | |
| 236 | A | | B | A | A | A | A | C | A | | A | | | | | | |
| 231 | A | | A | A | A | A | A | C | A | A | B | | | | | | |
| 240 | | | | | A | | A | C | A | | | | | | | | |
| 237 | | | | | | A | A | C | A | | | A | | | | | |
| 235 | | | | | | A | A | C | A | | | | | | | | |
| 247 | | | A | | | A | A | C | | | | | | A | | | |
| 264 | A | | | | A | A | A | A | | | | | | | | | |
| 272 | | | | | | | | | | A | | | | A | | | B |

® Compound number [symbol]
Target cRaf
® Target GSK-3beta [symbol]
Target c-kit [symbol]
Target Abl
© Target p56Lck
® Target EGFR
® Target PDGFR [symbol]
Target RICK [symbol]
Target CDK1/CyclinB
© Target CDK5/p35
© Target c-Src
© Target IKKb
θ Target RIP
© Target ROCK 2
θ Target p38
© Target UL97
© Target CDK2/CyclinA

### In Vitro Activity of Compounds of the Present Invention Against a Range of Cancer Cell Lines

We observed the surprising finding that compounds of the present invention were useful in inhibiting or killing a large variety of tumor cells. Tumor cell lines tested included:

| | | Order | | | |
|---|---|---|---|---|---|
| Cell line | Depository | Number | Source/disease | Reference(s) | Medium |
| A2780 | ECACC | 93112519 | Human ovarian | Semin Oncol | RPMI + 10% |
| | | | carcinoma | (1984) 11: 285; | FCS + 2 mM |
| | | | | Cancer Res | glutamine |
| | | | | (1987) 47: 414. | |
| A-549 | ATCC | CCL-185 | Lung carcinoma | J Natl Cancer | DMEM + 10% |
| | | | | Inst (1973) | FCS + 2 mM |
| | | | | 51: 417; | glutamine |
| | | | | Int J Cancer | |
| | | | | (1976) 17: 62. | |
| B16-F1 | ATCC | 6323 | Skin melanoma | Nat New Biol | DMEM + 10% |
| | | | | (1973) 242: 148. | FCS + 2 mM |
| | | | | Cancer Res | glutamine |
| | | | | (1975) 35: 218. | |
| HT-29 | ATCC | HTB-38 | Colorectal | J Natl Cancer | McCoy 5A + |
| | | | adenocarcinoma | Inst (1977) | 10% FCS + 2 mM |
| | | | | 59: 221. | glutamine |
| | | | | Cancer Genet | |
| | | | | Cytogenet | |
| | | | | (1987) 27: 125. | |

Cells were exposed to the test compounds at various concentration in 384 well plates. Experiments were performed in triplicates. The following cell numbers were plated in the respective media (see above) in a volume of 25 μl: cell lines A2780 and A549 at 200 cells per well, cell line B 16-F1 at 250 cells per well and cell line HT-29 at 100 cells per well. Cell were incubated for 24 hours at 37° C. and 7% CO₂ before the compounds of the subject invention, i.e. the test compounds, were added to yield final concentrations of 30, 10, 3.3, 1.1, 0.37 and 0.12 μM. Test Compounds were added from 30O× concentrated stock solutions in DMSO. Plates were then incubated for 72 hours at the conditions described above. Then 5 μl of a alamar blue solution (Biozol, Eching/Germany, Cat. No. BZL 00727) was added and the plates were incubated for 4 hours at the conditions described above. Then fluorescence was measured at an optical density of 560/590 nm (excitation at 560 nm, emission at 590 nm) in a Wallac Victor² multilabel counter (Perkin Elmer, Wellesley. Mass./United States). Inhibitory activity of the compounds was calculated as % inhibition compared to cells treated with DMSO (negative control). As a positive control cells were treated with doxurubicin (final concentrations of doxorubicin: 1 μM, 0.3 μM and 0.1 μM; experimental set up and dilutions for the positive and the negative control were identical to the wells treated with test compounds).

Table 2b shows the level of inhibition of four tumor cell lines after incubation with compounds of the present invention. All compounds demonstrated a clear and pronounced anti-proliferative activity towards a this panel of cancer cell lines. This surprising effect over various different cancer cell lines indicates that the subject compounds have strong anti-cancer activity.

TABLE 2b
Inhibitory effect of the compounds of the present invention on
various cancer cell lines

| | | Cell lines | | | |
|---|---|---|---|---|---|
| | | A2780 | A549 | B16-F1 | HT-29 |
| Compounds | 230 | | <15 μM | | |
| | 212 | <5 μM | <5 μM | <5 μM | <15 μM |
| | 145 | | <15 μM | | <15 μM |
| | 239 | | <15 μM | <15 μM | |
| | 243 | <15 μM | <15 μM | | |

Clonogenic Survival Assay with HCT-116 Cells.

With this assay we determine the concentration of a compound which leads to the irreversible loss of viability after a specified period of exposure. All steps are performed using aseptic techniques.

### Protocol:

(1) Incubate and grow cells at 37° C. 5% CO₂. Pre-warm media (RPMI-1640, 10% FCS, pen/strep) to 37° C. by placing in water bath. Rinse bottle with 70% ethanol prior to use.
(2) Recover cells by trypsinization from sub-confluent plates and count using a hemocytometer.
(3) Plate 1×10⁴ cells in 25 ml of media in a 15 cm tissue culture dish. Set up 14 plates for each compound to be tested. Incubate overnight at 37° C.
(4) Dilute the compound into media at the appropriate concentrations and replace the medium on the cells with the medium containing compound. Set up two plates for each concentration of the compound to be tested, as well as two without compound.
(5) Incubate plates for 24 hours at 37° C. 5% CO₂.
(6) Remove media from cells and replace with fresh media.
(7) Incubate for 7 days as above.
(8) Wash with PBS.
(9) Stain colonies with crystal violet solution (0.4% crystal violet, 20% ethanol) for 5 minutes.
(10) Wash twice with dH₂o.
(11) Count colonies.

Compounds of the present invention lead to an irreversible loss of viability of HCT-116 cells after 24 hours of exposure to the compounds of the present invention. Said compounds not only lead to an growth arrest, but cause an irreversible loss of viability.

### Activity of Compounds in Xenograft Tumor Models.

With this assay we demonstrate in-vivo activity of compounds of the present invention.

### Mice/Husbandry.

Mice are obtained from Charles River, housed in static microisolators, and provided ad libitum with water and an irradiated standard rodent diet (Purina Pico-Lab Rodent Diet 20).

### Determination of Maximum Tolerated Dose (MTD).

Mice at 8 weeks of age are pair-matched into groups of 5-8 animals and preliminary toxicity studies are performed with unknown test compounds. Animals are treated i.v. daily for 10 consecutive days with test compound and are weighed twice weekly. Mice are examined frequently for clinical signs of any adverse drug-related effects. Acceptable toxicity for anti-cancer drugs in mice is defined by the NCI as no mean group weight loss of over 20% and not more than 10% toxic death in treated animals.

### Standard Protocol.

### Experiments in Athymic Mice.

Athymic nude mice (male or female, 6-7 weeks; athymic nude mice are hairless, lack a normal thymus gland, and have a defective immune system because of a genetic mutation) are implanted s.c. with single 1 mm³ tumor fragments (tumor brie) or alternatively, 5-10×10⁶ tissue culture-derived cells into the flank. Animals are initially monitored twice weekly for tumor growth and then daily as the implants approach the desired size of approximately 100 mm³. When the tumors grow to between 50-250 mg in calculated tumor weight, the animals are pair-matched into appropriate experimental treatment groups (8-10 animals/group) and treatment with test compounds is initiated. A positive control is dosed according to historical controls. Tumor weights are calculated and body weights are taken twice weekly and animals are observed frequently for adverse drug effects. The protocol calls for any animal whose tumor mass reaches 1,000 mg to be immediately euthanized.

Tumors are measured by determining the length and width of the tumor with a digital caliper. Tumor weight is estimated using the following formula:

Tumor Weight (mg)=(w²×l)/2

where w=width and l=length in mm of the tumor. These values can also be expressed in volumetric units (mm³).

Experimental treatment may cause partial regression (PR) or complete regression (CR) of tumors. PR is where the tumor size is 50% or less of the starting (day 1) size but greater than 0.0 mg for three consecutive days during the course of the study, whereas a CR occurs when there is no measurable tumor mass for three consecutive days. Cures are defined as animals whose tumor shrinks to 0 mg and remains that way until the completion of the experiment.

Log cell kill (LCK) is a calculation that determines the percentage of tumor cells that are killed after the initiation of treatment and can be used as a quantitative measure of efficacy:

Log Cell Kill (LCK)=(T−C)/(3.32)(Td)

where T=is the mean time required for the treatment group of mice to reach 1,000 mg in size, C=the mean time for the control group tumors to reach 1,000 mg in size, Td=is the tumor doubling time estimated from the linear regression analysis from a semi-log growth plot of the control group tumors during exponential growth and 3.32=the number of doublings required for a population to increase 1-log 10 unit. Each LCK unit represents 1-log 10 unit of cell killing (e.g. 1 LCK=90% kill, 2 LCK=99% kill, etc.). We consider compounds to be significantly active when they have LCK values >1, which corresponds to >90% tumor cell kill.

Tumor growth inhibition (TGI) is a calculation that describes the amount of tumor growth that is inhibited by treatment with a compound over a defined period of time. It is expressed as:

% TGI=100(1−T/C)

where T is the mean tumor size of a compound treated group on a given day, and C is the mean tumor size of the vehicle control group on the same day.

Toxic deaths are defined as deaths caused by compound treatment and not by advanced disease state. A death is considered toxic if the animal dies within 1 week after the final compound treatment and the tumor size has not reached 1,000 mg. Non-tumor related deaths after this point are recorded, but not considered toxic deaths.

Tumor regression is defined according the following conventions: a regression is defined as partial (PR) if the tumor weight decreases to <50% of the starting weight (<50 mg). A regression is defined as complete (CR) if the tumor weight decreases below measurable weight during the experimental period. A cure is defined as a tumor-free animal at end of the observation period.

Similarly, experiments are performed in a syngeneic ip/ip mouse model.

Results. Compounds of the present invention show the following effects in the describe xenograft mouse model: (1) weight and size of tumors are smaller for compound treated animals as compared to the control groups, (2) Log cell kill (LCK) is higher for compound treated animals as compared to the control groups, and (3) Tumor growth inhibition (TGI) is higher for compound treated animals as compared to the control groups.

### Selection and Development of Drug Candidates.

In order to select the most appropriate compound to enter further experiments and to assess its suitability for use in a therapeutic composition for the treatment of disorders and diseases, such as cancers, additional data are collected. Such data can include the in vitro killing efficiency as measured by IC50 and cytotoxicity across a panel of tumor cell lines, the percentage cell killing as estimated in vitro, and tumor reduction data and mouse survival data from in vivo animal models. Furthermore, such experiments may also include the elucidation and/or determination of the mechanism of action of the subject compound, the target of the subject compound, and other characteristics of the subject compound, such as the binding affinity of the compound to the target or the binding site of the compound on the target. Such experiments may also include molecular modelling of the drug-target interaction.

The compound that shows the lowest IC50 for killing, the highest percentage cell killing and broadest across various tumor cell lines, the best tumor reduction data and/or the best mouse-survival data may be chosen to enter further experiments. Such experiments may include, for example, therapeutic profiling and toxicology in animals, phase I clinical trials in humans and other clinical trails.

### Synthesis of the Compounds of the Present Invention

In the following section, general procedures are described for the synthesis of the compounds of the present invention.

The pyridinylamines of the present invention can be synthesized by the conversion of 3-amino-5-bromo pyridine with suitable aldehydes in the presence of sodium triacetoxyborohydride. In a subsequent reaction step the intermediate compound is reacted in a Suzuki like coupling reaction with a suitable aryl boronic acid or alkyl boronic acid or ester in order to obtain a compound according to general formula (I). The secondary amino residue can be converted to a tertiary amino residue by deprotonation with a suitable base such as sodium hydride or butyl lithium and subsequent reaction with an alkylating agent such as alkyl iodides or alkyl bromides. It is also possible to carry out the alkylating step before the Suzuki like coupling reaction. In this case, step 2 and step 3 are replaced with each other as indicated by the backslash arrow.

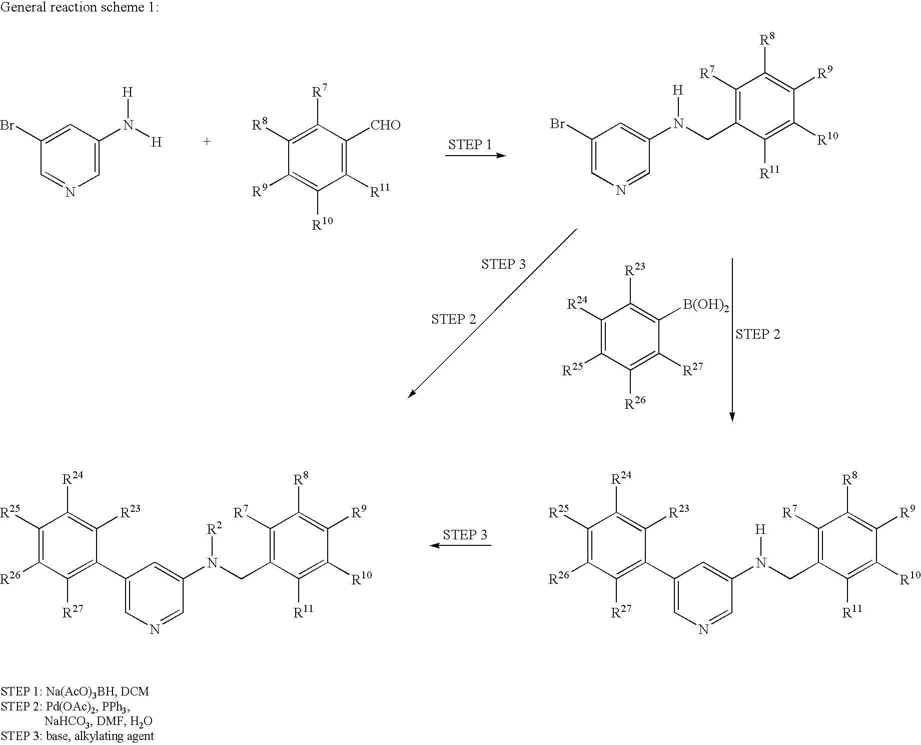

Another general method for the synthesis of the inventive compounds comprises the conversion of 3-amino-5-bromo pyridine with suitably substituted aryl boronic acids or alkyl boronic acids. Thereafter, the intermediate product is reacted in a Suzuki like coupling reaction with a second aryl boronic acid or alkyl boronic acid or ester in order to give compounds of the general formula (I).

The invention will now be illustrated by a series of examples which are intended to set forth typical and preferred procedures to be utilized in practice, but which shall not limit the ambit of the claims and the scope of protection.

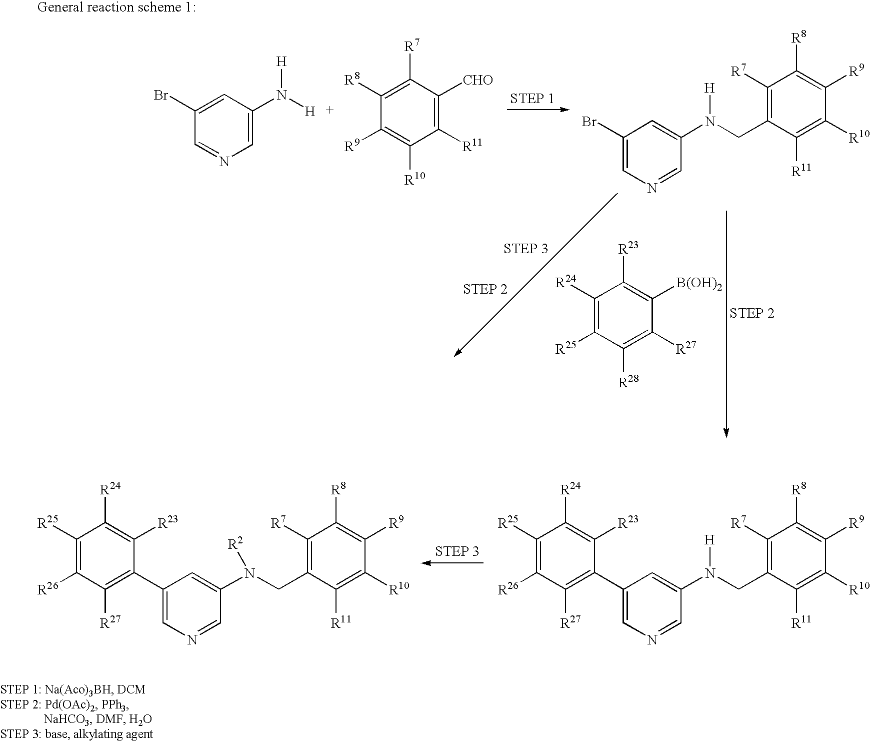

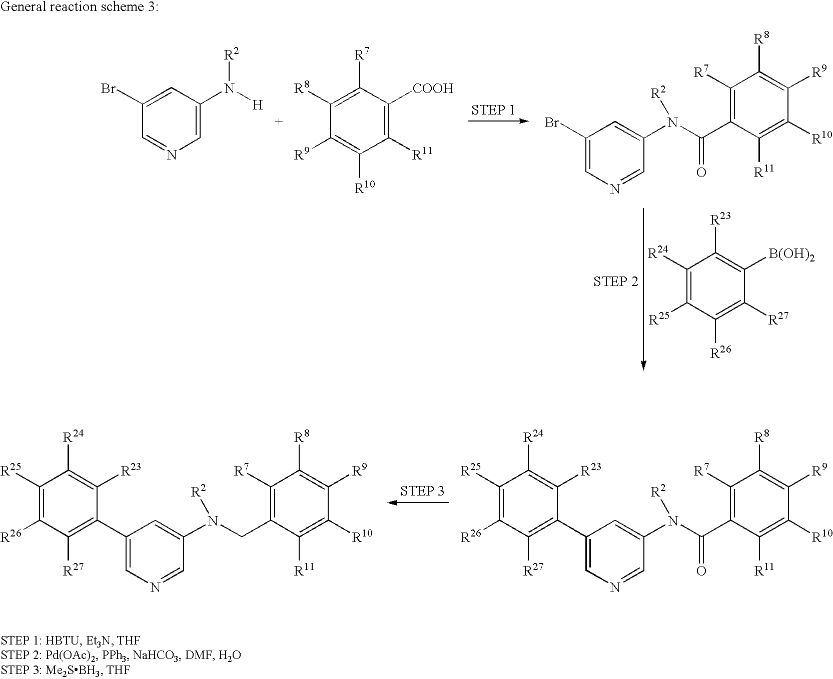

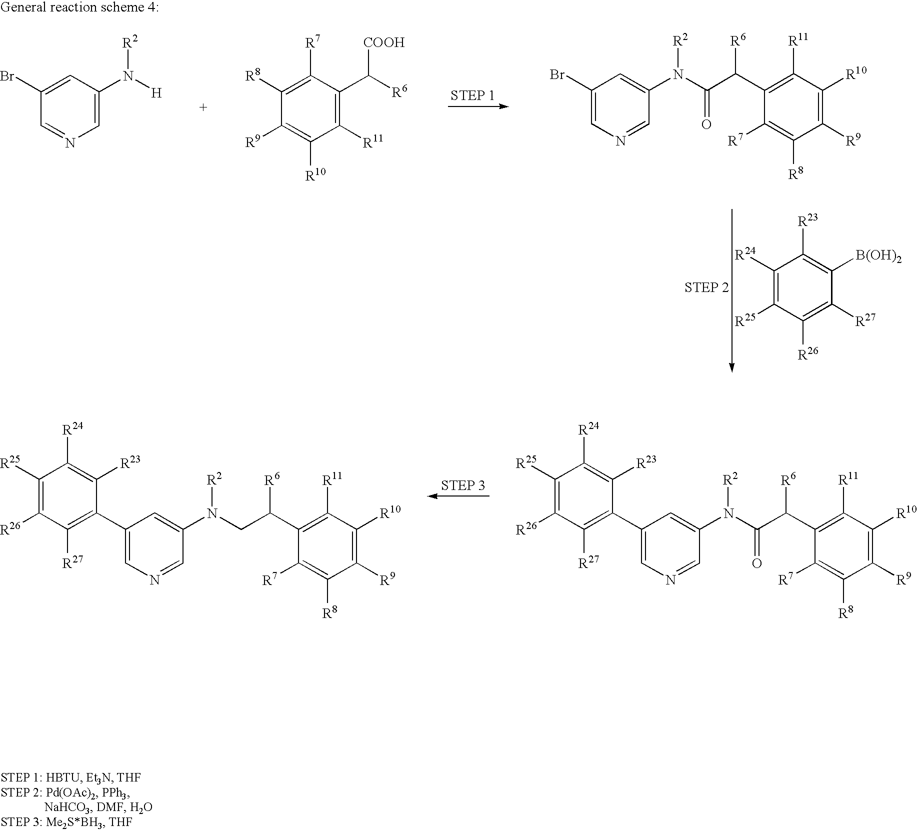

In a first step according to scheme 3 and 4, a suitable carboxylic acid was reacted with 3-amino-5-bromo pyridine under formation of an amid bond in order to result in an intermediate product which was converted in a second step with an aryl boronic acid or alkyl boronic acid or ester in a Suzuki like coupling reaction. Compounds of general formula (I) were obtained having an amid residue which could in a third step be reduced to a methylene group be means of a suitable reducing agent such as boranes.

The Suzuki like coupling reaction is not limited to aryl boronic acids. It can also be carried out with heteroaryl boronic acids, phenetyl boronic acids, alkinyl boronic acids, or alkenyl boronic acids. Thus, the group R¹ can be introduced by means of said Suzuki like coupling reaction as outlined in the following scheme 5.

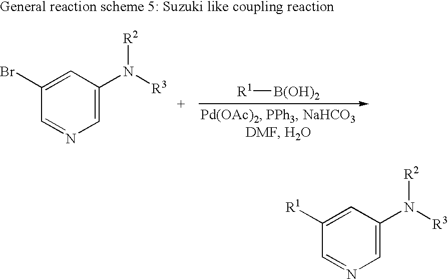

The following compounds can be prepared according to scheme 1 and/or 3:

1-5, 11-15, 19-22, 25, 27-35, 39-42, 48-51, 57, 59, 70-72, 74-76, 79, 82, 87-92, 95-98, 102, 106-113, 116, 123-128, 130, 134, 135, 139, 141, 145, 154, 155, 159, 160, 163, 172, 176, 177, 181, 195, 202, 206, 207, 209, 212, 214-218, 221-226, 228-234, 236, 238, 239, 241-243, 245-249, 251, 254, 255, 257-259, 261-273.

The following compounds can be prepared according to scheme 2:

6-10, 16-18, 23, 24, 26, 36-38, 43-47, 52-56, 58, 60-69, 73, 77, 78, 80, 81, 83-86, 93, 94, 99-101, 103-105, 114, 115, 117-122, 129, 131-133, 136-138, 140, 142-144, 146-153, 156-158, 161, 162, 164-171, 173-175, 178-180, 182-194, 196-201, 203-205, 208, 210, 211, 213, 219, 220, 227, 235, 237, 240, 250, 252, 274.

The following compounds can be prepared according to scheme 4:

224, 256.

### DESCRIPTION OF FIGURES

FIG. 1 shows representative examples of the inventive compounds

FIG. 2 shows representative examples of the inventive compounds

FIG. 3 shows the general scaffold of the inventive compounds

FIG. 4 shows the inhibition of human Cytomegalovirus replication

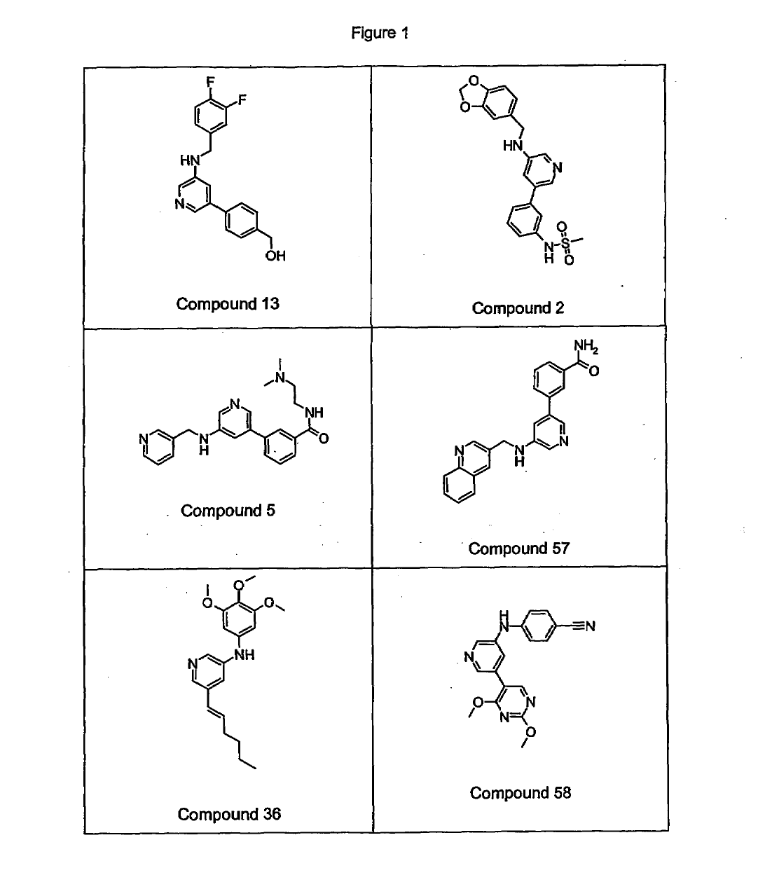

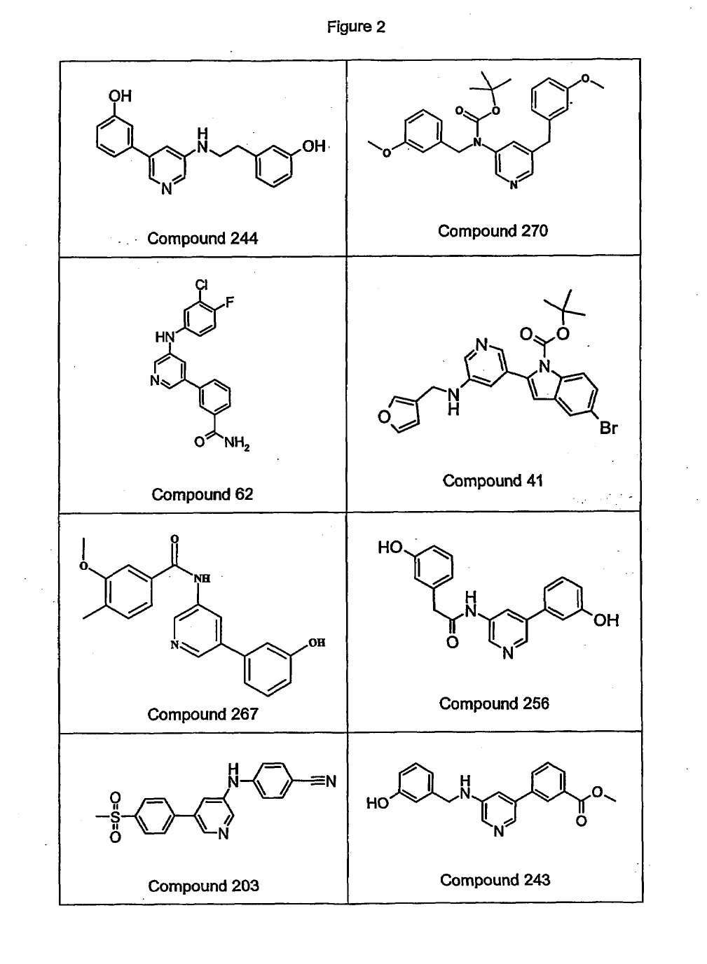

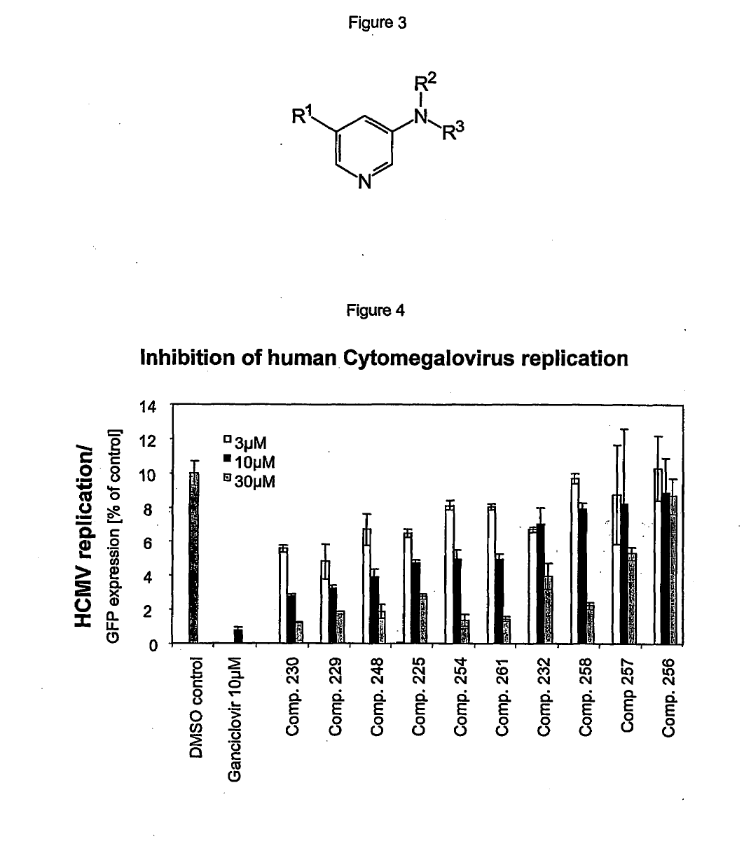

For HCMV-replication assays, subconfluent monolayers were infected with an HCMV strain AD169 producing EGFP. 1 h post infection, the culture medium was replaced with fresh one containing the indicated concentrations of the substances, DMSO control or 10 μM Ganciclovir, and cultured for 7d. Cells were lysed (in 25 mM Tris, pH 7.5, 2 mM DTT, 1% Triton X100 and 10% glycerol) and analysed for EGFP content in a Wallac Victor fluorescence detector

### EXAMPLES

### Materials and Methods

### Analytical Methods:

LC/MS data were obtained using a Micromass ZQ instrument with atmospheric pressure chemical ionisation or electrospray ionisation under the conditions described below.

### Standard Acidic LC-MS Conditions (Method A)

### HPLC Setup

| Solvents: | Acetonitrile (Far UV grade) with 0.1% (VTV) formic acid | |
|---|---|---|
| | Water (High purity via Elga UHQ unit) with 0.1% formic | |
| | acid | |
| Column: | Phenomenex Luna 5μ C 18 (2), 30 × 4.6 mm. | |
| Flow Rate: | 2 ml/min | |
| Gradient: | A: Water/formic acid | B: MeCN/formic acid |
| Time | A % | B % |
| 0.00 | 80 | 20 |
| 2.50 | 0.00 | 100 |
| 3.50 | 0.00 | 100 |
| 3.60 | 80 | 20 |
| 4.50 | 80 | 20 |

### UV Detection Via HP or Waters DAD

Purity is assessed as the integral over the window 210-400 nm.

If necessary, specific wavelength traces are extracted from the DAD data. Optional ELS detection was conducted using Polymer Labs ELS-1000.

MS Detection: Either Micromass Platform or ZQ, both Single Quadrapole LC-MS Instruments.
Scan range for MS Data (m/z)
Start (m/z) 100
End (m/z) 650
With +ve/−ve switching
Ionisation is either electrospray or APCI dependent on compound types.

### Standard Basic LC-MS Conditions (Method B)

### HPLC Setup

| | |
|---|---|
| Solvents: | Acetonitrile (Far UV grade) |
| | Water (High purity via Elga UHQ unit) with 1OmM |
| | ammonium bicarbonate (ammonium hydrogen carbonate) |
| Column: | Waters Xterra MS 5μ C 18, 50 × 4.6 mm. |
| Flow Rate: | 2 ml/min |

| Gradient: | A: Water/NH4HCO3 | B: MeCN/NH4HCO3 |
|---|---|---|
| Time | A % | B % |
| 0.00 | 80 | 20 |
| 2.50 | 0 | 100 |
| 3.50 | 0 | 100 |
| 3.60 | 80 | 20 |
| 4.50 | 80 | 20 |

### UV Detection Via HP or Waters DAD

Purity is assessed as the integral over the window 210-400 nm.

If necessary, specific wavelength traces are extracted from the DAD data. Optional ELS detection was conducted using Polymer Labs ELS-1000.

MS Detection: Either Micromass Platform or ZQ, both Single Quadrapole LC-MS Instruments.
Scan range for MS Data (m/z)
Start (m/z) 100
End (m/z) 650
With +ve/−ve switching
Ionisation is either electrospray or APCI dependent on compound types.

All reagents were obtained commercially and used directly. DMF and THF were dried over 4A molecular sieves (Fisher Scientific). Column chromatography employed Silica Gel 60 (Fluka). TLC was carried out using pre-coated plastic sheets Polygram SIL G/UV254 (Macherey-Nagel).

### Standard Basic LC-MS Conditions (Method C)

The conditions for the standard basic LC-MS conditions for Method C1 are the same as for Method A1, with the distinction that for method C1 no buffer like ammonium bicarbonate (ammonium hydrogen carbonate) or formic acid was used.

### Preparation of 3-[(5-bromo-pyridin-3-ylamino)-methyl]-phenol (248)

Sodium triacetoxyborohydride (1.03 g, 4.87 mmol) was added to a mixture of 3-hydroxy benzaldehyde (425 mg, 3.48 mmol) and 3-amino-5-bromo pyridine (600 mg, 3.48 mmol) in DCM (10 ml). The reaction was stirred at room temperature for 18 hours. Reaction diluted with DCM (30 ml) and washed with de-ionised water (2×20 ml). Aqueous combined and extracted with EtOAc (3×30 ml). Organics combined, dried over MgSO₄, filtered and evaporated to dryness. Residue triturated in petroleum ether 40/60 to give product (248) in 48% yield.

LC-MS, m/z [MH]⁺ 279. Retention time, 1.06 minutes. Method A.

¹H NMR (DMSO-cfe, 400 MHz): δ=4.40 (d, 2H, CH₂), 6.81 (d, 1H, Ar—H), 6.96 (m, 3H, Ar—H N—H)₁ 7.23 (d, 1H, Ar—H), 7.30 (t, 1H, Ar—H), 7.98 (s, 1H, Ar—H), 8.13 (s, 1H₁Ar—H), 9.54 (s, 1H₁OH).

### Preparation of 3-{[5-(2-fluoro-3-methoxy-phenyl)-pyridin-3-ylamino]-methyl}-phenol (236)

To a solution of 3-[(5-bromo-pyridin-3-ylamino)-methyl]-phenol (248) (204 mg, 0.74 mmol) in de-gassed DMF (5 ml) under a N₂ atmosphere, 2-fluoro-3-methoxyphenyl boronic acid (250 mg, 1.47 mmol), NaHCO₃ (247 mg, 2.94 mmol), de-gassed de-ionised water (2 ml), triphenylphosphine (30 mg, O.Hmmol) and palladium acetate (9 mg, 0.07 mmol) were added. Reaction stirred at 80° C. for 18 hours. Reaction cooled and evaporated to dryness. Residue dissolved in EtOAc (40 ml) and washed with Na₂CO₃ (30 ml) and de-ionised water (30 ml), dried over MgSO₄, filtered and evaporated to dryness. Residue triturated in DCM to give product (236) in 52% yield.

LC-MS, m/z [MH]⁺ 325. Retention time, 1.82 minutes. Method B.

¹H NMR (DMSO-cfe, 400 MHz): δ=3.91 (s, 3H, CH₃), 4.30 (d, 2H, CH₂), 6.69-7.28 (9H, Ar—H, N—H), 7.90 (s, 1H, Ar—H), 8.05 (s, 1H, Ar—H), 9.38 (s, 1H, OH).

The following analogues of 3-{[5-(2-fluoro-3-methoxy-phenyl)-pyridin-3-ylamino]-methyl}-phenol (236), were prepared using the experimental procedures described above.

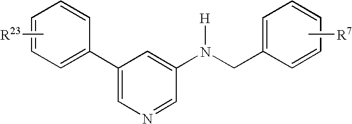

| | | | LC-MS, | Retention | |
|---|---|---|---|---|---|
| Com- | | | m/z | Time | |
| pound | R7 | R23 | [MH]⁺ | (minutes) | Method |
| 225 | 3-OH | 3-OH | 293 | 1.30 | C |
| 221 | 3-OH | 3-NHCOCH₃ | 334 | 1.24 | C |
| 230 | 3-OH | 3-CONH₂ | 321 | 1.09 | C |
| 229 | 3-OH | 4-CH₂OH | 307 | 1.19 | C |
| 239 | 3-OH | 3-OCH₃ | 307 | 1.83 | B |
| 254 | H | 3-OH | 277 | 1.81 | B |
| 258 | 3-OCH₃ | 3-OH | 307 | 1.80 | B |
| 249 | 3-OH | 2-OH | 293 | 1.58 | B |
| 212 | 3-OH | 4-OH | 293 | 1.47 | B |
| 232 | 3-OH | 3-NH₂ | 292 | 1.53 | B |
| 251 | 2-OH | 3-OH | 293 | 1.58 | B |
| 243 | 3-OH | 3-COOCH₃ | 225 | 1.82 | B |
| 273 | 3-COOCH₃ | 3-OH | 225 | 1.70 | B |
| 266 | 3-NO₂ | 3-OH | 322 | 1.73 | B |
| 238 | 3-OH | H | 277 | 1.82 | B |
| 242 | 3,5-OH | 3-OH | 309 | 1.31 | B |
| 259 | 3-OCH₃, | 3-OH | 325 | 1.84 | B |
| | 4-F | | | | |
| 255 | 2-F, | 3-OH | 325 | 1.87 | B |
| | 5-OCH₃ | | | | |

Furthermore, the following analogues are prepared using the same experimental procedures:

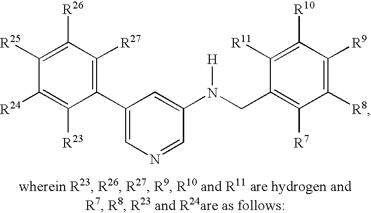

| Compound | R7 | R8 | R24 | R25 |
|---|---|---|---|---|
| 263 | CH₃ | OH | NH₂ | H |
| 264 | CH₃ | OH | COOH | H |
| 265 | CH₃ | OH | CONH₂ | H |
| 266 | CH₃ | OH | SO₂NH₂ | H |
| 267 | CH₃ | NH₂ | CONH₂ | H |
| 268 | CH₃ | OCH₃ | CONH₂ | H |
| 269 | CH₃ | CONH₂ | CONH₂ | H |
| 270 | CH₃ | SO₂NH₂ | CONH₂ | H |
| 271 | CH₃ | NH₂ | OH | H |
| 272 | CH₃ | OCH₃ | OH | H |
| 273 | CH₃ | CONH₂ | OH | H |
| 274 | CH₃ | SO₂NH₂ | OH | H |
| 275 | CH₃ | OH | H | OH |
| 276 | CH₃ | OH | H | NH₂ |
| 277 | CH₃ | OH | H | COOH |
| 278 | CH₃ | OH | H | COOCH₃ |
| 279 | CH₃ | OH | H | CONH₂ |
| 280 | CH₃ | OH | H | SO₂CH₃ |
| 281 | CH₃ | OH | H | SO₂NH₂ |
| 282 | CH₃ | OH | H | Cl |
| 283 | CH₃ | OH | H | OCH₃ |
| 284 | CH₃ | OH | H | CH₃ |
| 285 | CH₃ | OH | H | CN |
| 286 | CH₃ | OH | H | OCF₃ |
| 287 | CH₃ | OH | H | CF₃ |
| 288 | OH | OH | CONH₂ | H |
| 289 | OCH₃ | OH | CONH₂ | H |
| 290 | Cl | OH | CONH₂ | H |
| 291 | OH | OH | OH | H |
| 292 | OCH₃ | OH | OH | H |
| 293 | Cl | OH | OH | H |

The following analogue is prepared as well using the same experimental procedure:

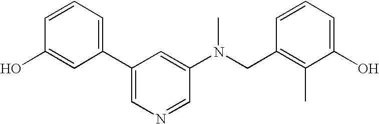

(compound 294)

The following derivatisations/transformations were also conducted:

### Preparation of 2-fluoro-3-[5-(3-hydroxy-benzylamino)-pyridin-3-yl]-phenol (231)

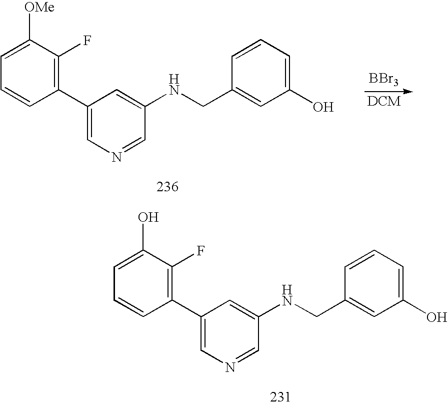

To a solution of 3-{[5-(2-Fluoro-3-methoxy-phenyl)-pyridin-3-ylamino]-methyl}-phenol (236) (97 mg, 0.30 mmol) in DCM (15 ml) at −78° C. under a N₂ atmosphere, a 1M solution of borontribromide in DCM (6 ml, 5.99 mmol) was added. Reaction was allowed to warm to room temperature and stirred for 18 hours. Reaction was quenched with addition of de-ionised water and pH adjusted to 6 by addition of 2M NaOH. Mixture was extracted with EtOAc (2×40 ml). The organic phases were combined, dried over MgSO₄ and evaporated to dryness. Residue was purified by Prep-HPLC. Compound 231 was isolated in 37% yield.

LC-MS, m/z [MH]⁺ 311. Retention time, 1.44 minutes. Method B.

¹H NMR (DMSO-de, 400 MHz): δ=4.38 (d, 2H, CH₂), 6.72-7.25 (9H, Ar—H, N—H), 8.00 (s, 1H, Ar—H), 8.10 (s, 1H, Ar—H), 9.8 (br s, 2H, 2(OH)).

### Preparation of 3-{[5-(3-hydroxy-phenyl)-pyridin-3-ylamino]methyl}-benzoic acid (263)

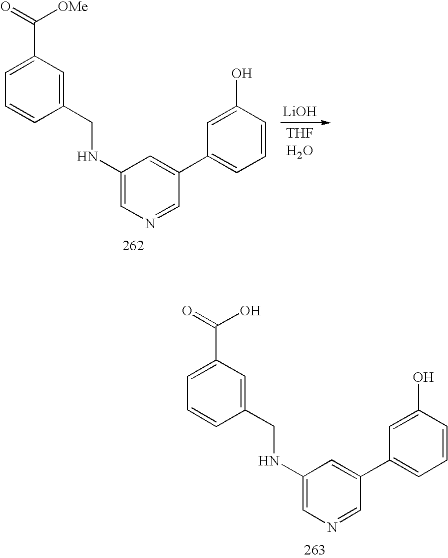

To a solution of 3-{[5-(3-hydroxy-phenyl)-pyridin-3-ylamino]methyl}-benzoic acid methyl ester (262) (53 mg, 0.16 mmol) in THF (2 ml) and de-ionised water (2 ml), lithium hydroxide. H₂O (33 mg, 0.60 mmol) was added. Reaction was stirred for 18 hours at room temperature.

THF was evaporated and aqueous phase acidified to pH 3-4 with acetic acid then extracted with EtOAc (4×40 ml). The organic phases were combined, dried over MgSO₄, filtered and evaporated. Residue was triturated in de-ionised water. Compound 263 could be isolated in 63% yield.

LC-MS, m/z [MH]⁺ 321. Retention time, 1.03 minutes. Method B.

¹H NMR (DMSO-CZ₆, 400 MHz): δ=4.62 (d, 2H, CH₂), 6.86 (t, 1H, N—H), 6.95 (d, 1H, Ar—H)₁ 7.10 (s, 1H, Ar—H), 7.15 (d, 1H, Ar—H), 7.22 (s, 1H, Ar—H), 7.40 (t, 1H, Ar—H), 7.65 (t, 1H, Ar—H), 7.82 (d, 1H, Ar—H), 8.00 (d, 1H, Ar—H), 8.18 (m, 3H, Ar—H), 9.72 (br s, 1H, OH) 13.10 (br s, 1H, COOH).

### Preparation of 3-{[5-(3-hydroxy-phenyl)-pyridin-3-ylamino]-methyl}-benzamide (265)

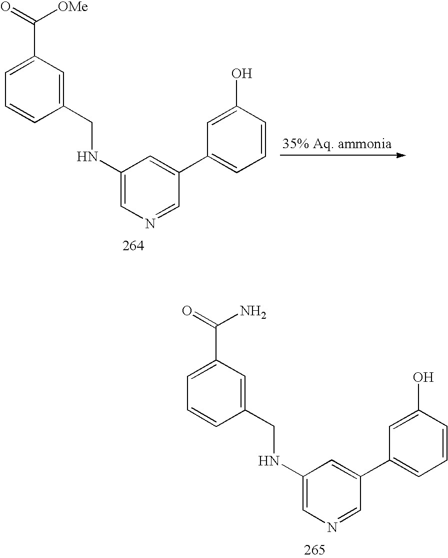

A mixture of 264 3-{[5-(3-hydroxy-phenyl)-pyridin-3-ylamino]methyl}-benzoic acid methyl ester (63 mg, 0.19 mmol) in 35% Aq.ammonia (10 ml) was heated for 7 hours under reflux. Reaction was cooled and partitioned between EtOAc (30 ml) and de-ionised water (30 ml). Layers were separated and aqueous phase extracted with EtOAc (30 ml). The organic phases were combined, washed with brine (30 ml) dried over MgSO₄, filtered and evaporated. Residue was purified by column chromatography. Mixture was pre absorbed onto flash silica and eluted with 10% MeOH/DCM. Compound 265 was isolated in 40% yield.

LC-MS, m/z [MH]⁺ 320. Retention time, 1.32 minutes. Method B.

¹H NMR (DMSO-c/e, 400 MHz): δ=4.30 (d, 2H, CH₂), 6.51 (t, 1H, N—H), 6.65-7.83 (13H, Ar—H, N—H) 9.40 (s, 1H, OH).

### Preparation of 3-[5-(3-hydroxy-benzylamino)-pyridin-3-yl]-benzoic acid (246)

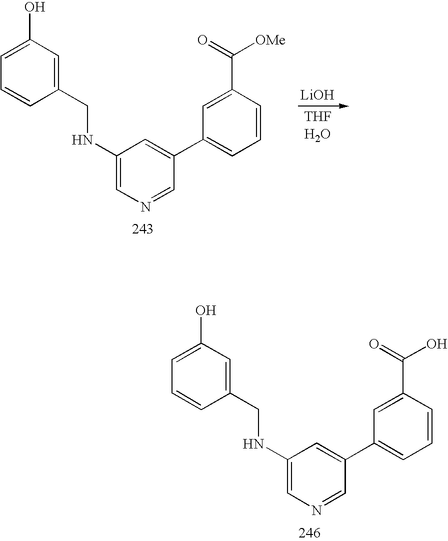

To a solution of 3-[5-(3-hydroxy-benzylamino)-pyridin-3-yl]-benzoic acid methyl ester (243) (83 mg, 0.25 mmol) in THF (2 ml) and de-ionised water (2 ml), lithium hydroxide. H₂O (52 mg, 1.25 mmol) was added. Reaction was stirred for 8 hours at room temperature.

THF was evaporated and reaction mixture was acidified to pH 4-5 with acetic acid. Precipitate was collected by filtration, washing with diethyl ether (50 ml).

Compound 246 was isolated in 46% yield.

LC-MS, m/z [MH]⁺ 321. Retention time, 0.97 minutes. Method B.

¹H NMR (DMSO-c/e, 400 MHz): δ=4.55 (d, 2H, CH₂), 6.88 (d, 2H, Ar—H), 7.20-7.38 (4H, Ar—H, N—H), 7.85 (t, 1H, Ar—H), 8.10 (d, 1H, Ar—H), 8.20 (d, 1H, Ar—H), 8.26 (s, 1H, Ar—H), 8.30 (s, 1H, Ar—H), 8.36 (s, 1H, Ar—H), 9.60 (br s, 1H, OH), 13.35 (br s, 1H₁COOH).

### Preparation of 3-[5-(3-amino-benzylamino)-pyridin-3-yl]-phenol (245)

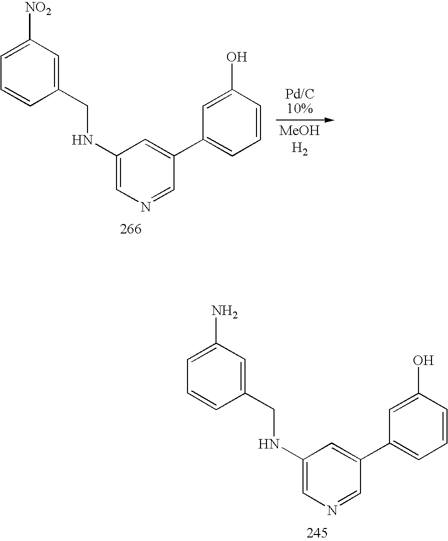

A solution of 3-[5-(3-nitro-benzylamino)-pyridin-3-yl]-phenol (266) (113 mg, 0.35 mmol) was hydrogenated over 10% Pd/C (20 mg) in a H₂ atmosphere for 48 hours. Reaction was filtered through celite and evaporated to dryness. Residue was purified by column chromatography. Mixture was pre absorbed onto flash silica and eluted with 5% sat NH₃ in MeOH/DCM. Compound 245 was isolated in 45% yield.

LC-MS, m/z[MH]⁺ 192. Retention time, 1.44 minutes. Method B.

¹H NMR (DMSO-CZ₆, 400 MHz): δ=4.10 (d, 2H, CH₂), 4.98 (s, 2H, NH₂), 6.30 (d, 1H, Ar—H), 6.40-6.90 (9H, Ar—H, N—H), 7.15 (t, 1H, Ar—H), 7.85 (d, 2H, Ar—H), 9.45 (s, 1H₁O—H).

### Preparation of N-[3-(5-phenylamino-pyridin-3-yl)-phenyl]-acetamide (194) according to general scheme 2

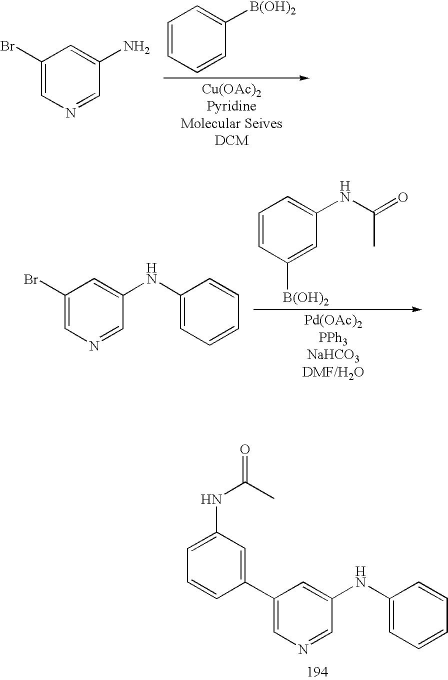

### Preparation of (5-bromo-pyridin-3-yl)-phenyl-amine

To a solution of 3-amino-5-bromo pyridine (300 mg, 1.74 mmol) in DCM (20 ml), phenyl boronic acid (424 mg, 3.48 mmol), pyridine (281 μl, 3.48 mmol), 4A mol sieves (200 mg) and copper(II) acetate (158 mg, 0.87 mmol) were added. Reaction was stirred for 18 hours under atmosphere.

Reaction was filtered, washing cake with MeOH and evaporated to dryness. Residue was purified by flash chromatography. Mixture was pre absorbed onto flash silica, loaded onto a 10 g isolute flash Si cartridge and eluted using CombiFlash™ instrumentation, with a gradient of 0-60% EtOAc/petroleum ether 40/60 (v:v). (5-Bromo-pyridin-3-yl)-phenyl-amine was isolated in 29% yield.

LC-MS, m/z [MH]⁺ 249. Retention time, 1.84 minutes. Method A.

¹H NMR (CDCl₃, 400 MHz): δ=5.75 (br s, 1H, NH), 7.10 (m, 3H, Ar—H), 7.35 (m, 2H, Ar—H), 7.55 (s, 1H, Ar—H), 8.20 (s, 1H, Ar—H), 8.25 (s, 1H, Ar—H).

### Preparation of N-[3-(5-phenylamino-pyridin-3-yl)-phenyl]-acetamide (194)

To a solution of (5-bromo-pyridin-3-yl)-phenyl-amine (120 mg, 0.48 mmol) in de-gassed DMF (5 ml) under a N₂ atmosphere, 3-acetamidophenylboronic acid (173 mg, 0.97 mmol), NaHCO₃ (162 mg, 1.93 mmol), de-gassed de-ionised water (2 ml), triphenylphosphine (19 mg, 0.071 mmol) and palladium acetate (5 mg, 0.024 mmol) were added. Reaction was stirred at 80° C. for 18 hours. Reaction was cooled and evaporated to dryness. Residue was dissolved in EtOAc (40 ml) and washed with de-ionised water (30 ml), dried over MgSO₄, filtered and evaporated to dryness. Residue was purified by flash chromatography. Mixture was pre absorbed onto flash silica and eluted with 5% MeOH/DCM. Compound 194 was isolated in 37% yield.

LC-MS, m/z [MH]⁺ 304. Retention time, 1.72 minutes. Method B.

¹H NMR (DMSO-de, 400 MHz): δ=2.11 (s, 3H, CH₃), 7.00 (t, 1H, Ar—H), 7.21 (d, 2H, Ar—H), 7.39-7.94 (7H, Ar—H, N—H), 8.32 (s, 1H, Ar—H), 8.41 (s, 1H, Ar—H), 8.60 (s, 1H, Ar—H), 10.11 (s, 1H, N—H).

The following analogues of N-[3-(5-phenylamino-pyridin-3-yl)-phenyl]-acetamide (194), were prepared using the experimental procedures described above.

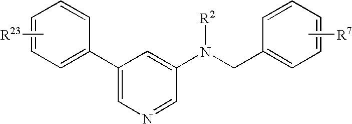

| | | | | LC-MS, | Retention | |
|---|---|---|---|---|---|---|
| Com- | | | | m/z | Time | |
| pound | R23 | R7 | R2 | [MH]⁺ | (minutes) | Method |
| 227 | 3-NHCOCH₃ | 4-OMe | H | 334 | 1.68 | B |
| 240 | 3-NHCOCH₃ | 2-OMe | H | 334 | 1.63 | B |
| 237 | 3-NHCOCH₃ | 2-F | H | 322 | 1.67 | B |
| 235 | 3-NHCOCH₃ | 4-F | H | 322 | 1.68 | B |
| 252 | 3-NH₂ | H | H | 262 | 1.74 | B |
| 205 | 3-NHCOCH₃ | 4-CN | H | 329 | 1.64 | B |
| 274 | H | H | H | 247 | 2.15 | B |
| 250^{#} | 3-NHCOCH₃ | H | Me | 318 | 1.81 | B |

^{#}prepared using N-methyl aniline in preparation of analogue

### Preparation of 3-(5-amino-pyridin-3-yl)-phenol (260)

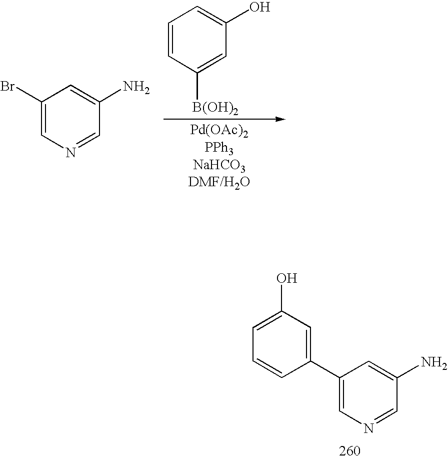

To a solution of 3-amino-5-bromopyridine (150 mg, 0.87 mmol) in de-gassed DMF (5 ml) under a N₂ atmosphere, 3-hydroxyphenyl boronic acid (240 mg, 1.75 mmol), NaHCO₃ (293 mg, 3.5 mmol), de-gassed de-ionised water (2 ml), triphenylphosphine (34 mg, 0.131 mmol) and palladium acetate (10 mg, 0.436 mmol) were added. Reaction was stirred at 80° C. for 18 hours, then cooled and evaporated to dryness. Residue was dissolved in EtOAc (40 ml) and washed with de-ionised water (30 ml), dried over MgSO₄, filtered and evaporated to dryness. Residue was triturated in ether to afford product. Compound was isolated in 63% yield.

LC-MS, m/z[MH]⁺ 187. Retention time, 1.08 minutes. Method B.

¹H NMR (DMSO-CZ₆, 400 MHz): δ=5.56 (br s, 2H, NH₂), 6.94 (d, 1H, Ar—H), 7.10 (d, 1H, Ar—H), 7.15 (d, 1H, Ar—H) 7.21 (t, 1H, Ar—H), 7.40 (t, 1H Ar—H,), 8.05 (d, 1H, Ar—H), 8.10 (d, 1H, Ar—H), 9.73 (s, 1H, OH).

N-[3-(5-amino-pyridin-3-yl)phenyl]-acetamide (253) was prepared using the experimental procedure above. LC-MS, m/z [MH]⁺ 228. Retention time, 0.99 minutes. Method B.

### Preparation of 3-hydroxy-N-[5-(3-hydroxy-phenyl)-pyridin-3-yl]-benzamide (234)

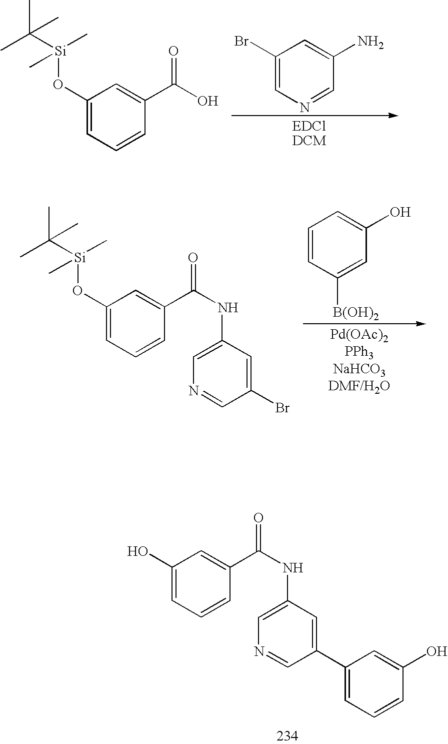

### Preparation of a N-(5-bromo-pyridin-3-yl)-3-(tert-butyl-dimethyl-silanyloxy)-benzamide

To a solution of 3-(tert-butyl-dimethyl-silanyloxy)-benzoic acid (725 mg, 2.57 mmol) in DCM (25 ml) at room temperature, EDCI (1.28 g, 6.68 mmol) was added and reaction was stirred for 30 minutes. 3-Amino-5-bromopyridine (421 mg, 2.45 mmol) was then added and reaction was stirred at 30° C. for 24 hours. Reaction was cooled, diluted with DCM (30 ml) and washed with de-ionised water (30 ml), NaHCO₃ (30 ml), de-ionised water (30 ml) and brine (30 ml)₁ dried over MgSO₄, filtered and evaporated. Residue was purified by flash chromatography. Mixture was pre absorbed onto flash silica, loaded onto a 20 g isolute flash Si cartridge and eluted using CombiFlash™ instrumentation, with a gradient of 0-100% EtOAc/petroleum ether 40/60 (v:v). N-(5-Bromo-pyridin-3-yl)-3-(tert-butyl-dimethyl-silanyloxy)-benzamide was isolated in 30% yield.

LC-MS, m/z [MH]⁺ 407. Retention time, 2.95 minutes. Method B.

¹H NMR (DMSO-de, 400 MHz): δ=0.27 (s, 6H, (CH₃)₂), 1-00 (s, 9H, (CH₃)₃), 7.15 (d, 1H, Ar—H), 7.42 (s, 1H, Ar—H) 7.50 (t, 1H, Ar—H) 7.62 (d, 1H, Ar—H) 8.48 (s, 1H, Ar—H), 8.52 (s, 1H, Ar—H), 8.95 (s, 1H, Ar—H), 10.51 (s, 1H, N—H).

### Preparation of 3-hydroxy-N-[5-(3-hydroxy-phenyl)-pyridin-3-yl]-benzamide (234)

To a solution of N-(5-bromo-pyridin-3-yl)-3-(tert-butyl-dimethyl-silanyloxy)-benzamide (290 mg, 0.71 mmol) in de-gassed DMF (5 ml) under a N2 atmosphere, 3-hydroxyphenyl boronic acid (197 mg, 1.43 mmol), NaHCO₃ (240 mg, 2.85 mmol), de-gassed de-ionised water (2 ml), triphenylphosphine (28 mg, 0.107 mmol) and palladium acetate (8 mg, 0.036 mmol) were added. Reaction was stirred at 80° C. for 18 hours. Reaction was cooled and evaporated to dryness. Residue was dissolved in EtOAc (40 ml) and washed with de-ionised water (30 ml), dried over MgSO₄, filtered and evaporated to dryness. Residue was purified by flash chromatography. Mixture was pre absorbed onto flash silica and eluted with 5% MeOH/DCM. Product was triturated in diethyl ether. Compound 234 was isolated in 49% yield.

LC-MS, m/z [MH]⁺ 307. Retention time, 1.43 minutes. Method B.

¹H NMR (DMSO-de, 400 MHz): δ=6.90 (d, 1H, Ar—H), 7.05 (d, 1H, Ar—H), 7.10 (s, 1H, Ar—H), 7.15 (d, 1H, Ar—H), 7.40 (m, 3H, Ar—H) 7.50 (d, 1H, Ar—H) 8.49 (s, 1H, Ar—H) 8.60 (s, 1H, Ar—H), 9.00 (s, 1H, Ar—H), 9.70 (s, 1H, O—H), 9.87 (s, 1H, O—H), 10.50 (s, 1H, N—H).

### Preparation of 3-{5-[2-(3-hydroxy-phenyl)-ethylamino]-pyridin-3-yl}-phenol (244)

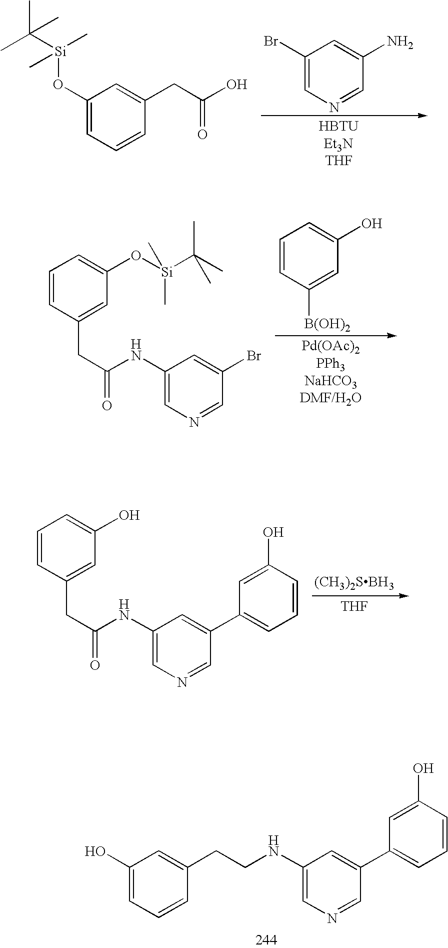

### Preparation of N-(5-bromo-pyridin-3-yl)-2-[3-(tert-butyl-dimethyl-silanyloxy)-phenylj-acetamide

To a solution of [3-(tert-butyl-dimethyl-silanyoxy)-phenyl]acetic acid (1.70 g, 6.39 mmol) in THF (10 ml) and DMF (0.5 ml) at room temperature under a N₂ atmosphere, Et₃N (1.86 ml, 13.44 mmol) and 3-amino-5-bromo pyridine (1.15 g, 6.72 mmol) were added. Reaction was cooled to 0° C. and HBTU (2.55 g, 6.72 mmol), was added. Reaction was stirred at room temperature for 2 hours and then warmed to 50° C. and stirred for 18 hours. Reaction was cooled, diluted with EtOAc (30 ml) and washed with citric acid (30 ml), NaHCO₃ (30 ml), de-ionised water (30 ml) and brine (30 ml), dried over MgSO₄, filtered and evaporated. Residue was purified by flash chromatography. Mixture was pre-absorbed onto flash silica and eluted with 20-40% EtOAc/petroleum ether 40/60 (v:v). N-(5-Bromo-pyridin-3-yl)-2-[3-(tert-butyl-dimethyl-silanyloxy)-phenyl]-acetamide was isolated in 48% yield.

LC-MS, m/z [MH]⁺ 421. Retention time, 2.86 minutes. Method B.

¹H NMR (DMSO-Cf₆, 400 MHz): δ=0.00 (s, 6H, (CHa)₂), 0.78 (s, 9H₁ (CH₃)₃), 3.47 (s, 2H, CH₂), 6.57 (d, 1H, Ar—H), 6.68 (s, 1H, Ar—H), 6.75 (d, 1H, Ar—H), 7.03 (t, 1H₁ Ar—H)₁8.21 (s s, 2H, Ar—H), 8.50 (s, 1H, Ar—H), 10.42 (s, 1H, N—H).

### Preparation of 2-(3-hydroxy-phenyl)-N-[5-(3-hydroxy-phenyl)-pyridin-3-yl]-acetamide (256)

To a solution of N-(5-bromo-pyridin-3-yl)-2-[3-(tert-butyl-dimethyl-silanyloxy)-phenyl]-acetamide 1.29 g, 3.07 mmol) in de-gassed DMF (15 ml) under a N₂ atmosphere, 3-hydroxyphenyl boronic acid (846 mg, 6.14 mmol), NaHCO₃ (1-03 g, 12.28 mmol), de-gassed de-ionised water (5 ml), triphenylphosphine (121 mg, 0.46 mmol) and palladium acetate (35 mg, 0.15 mmol) were added. Reaction was stirred at 80° C. for 18 hours. Reaction was cooled and evaporated to dryness. Residue was dissolved in EtOAc (40 ml) and washed Na₂CO₃ (30 ml) and de-ionised water (30 ml), dried over MgSO₄, filtered and evaporated to dryness. Residue was purified by flash chromatography. Mixture was pre absorbed onto flash silica and eluted with 5-10% MeOH/DCM. Product was triturated in diethyl ether. Compound 256 was isolated in a 46% yield.

LC-MS, m/z [MH]^{▪} 381. Retention time, 1.41 minutes. Method B.

¹H NMR (DMSO-de, 400 MHz): δ=3.43 (s, 2H, CH₂), 6.50 (d, 1H, Ar—H), 6.60 (m, 3H, Ar—H), 6.68 (d, 1H, Ar—H)₁ 6.84 (s, 1H₁Ar—H), 6.95 (m, 2H, Ar—H)₁ 8.14 (s, 1H, Ar—H)₁ 8.32 (s, 1H, Ar—H), 8.53 (s, 1H, Ar—H), 9.21 (s, 1H₁OH), 9.50 (s, 1H, OH)₁ 10.31 (s₁ I H₁N—H).

### Preparation of 3-{5-[2-(3-Hydroxy-phenyl)-ethylamino]-pyridin-3-yl}-phenol (244)

To a solution of 256, 2-(3-hydroxy-phenyl)-N-[5-(3-hydroxy-phenyl)-pyridin-3-yl]-acetamide (190 mg, 0.593 mmol) in THF (10 ml) under a N₂ atmosphere, a 2M solution of borane-methyl sulfide complex in THF (1.5 ml, 2.96 mmol) was added in one portion. Reaction was heated under reflux for 2 hours. Mixture was cooled and evaporated to dryness. Residue was dissolved in EtOAc (30 ml) and washed with 10% citric acid (30 ml), NaHCO₃ (30 ml) and de-ionised water (30 ml), dried over MgSO₄, filtered and evaporated under vacuum. Residue was dissolved in EtOH (30 ml) and heated under reflux for 3 hours. Mixture was cooled and evaporated to dryness. Residue was purified by flash chromatography. Mixture was pre absorbed onto flash silica and eluted with 5-10% MeOH/DCM. Compound 244 was isolated in a 31% yield.

LC-MS, m/z [MH]⁺ 307. Retention time, 1.62 minutes. Method B.

¹H NMR (DMSO-c/e, 400 MHz): δ=2.70 (t, 2H, CH₂), 3.20 (t, 2H, CH₂), 5.90 (t, 1H, N—H), 6.49 (d, 1H, Ar—H), 6.58 (s, 1H, Ar—H), 6.70 (d, 1H₁Ar—H), 6.78 (d, 1H, Ar—H), 6.90 (s, 1H, Ar—H), 6.94 (m, 2H, Ar—H), 7.00 (t, 1H, Ar—H), 7.15 (t, 1H, Ar—H) 7.88 (d, 2H), 9.15 (s, 1H, OH), 9.41 (s, 1H, OH).

### Preparation of 5-{[5-(3-hydroxy-phenyl)-pyridin-3-ylamino]-methyl>−2-methyl phenol (247)

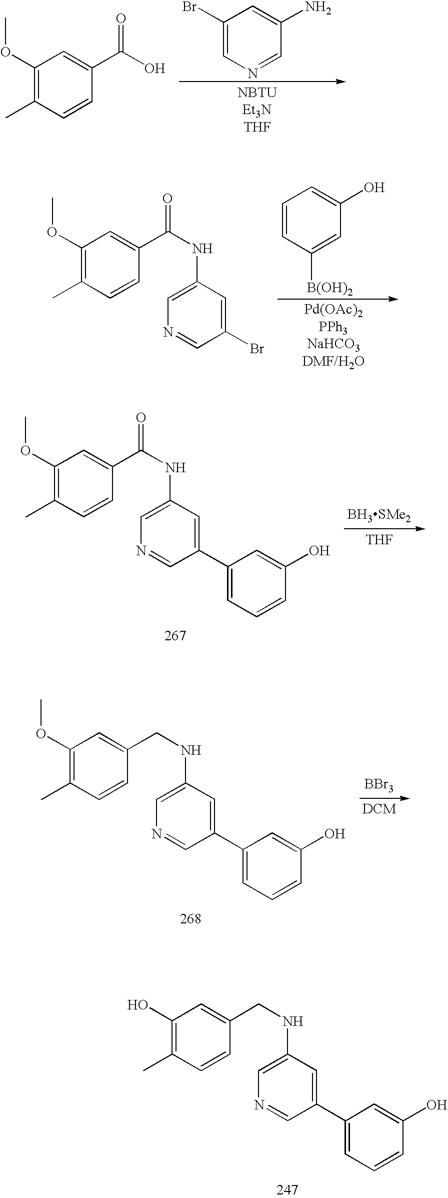

### Preparation of N-(5-bromo-pyridin-3-yl)-3-methoxy-4-methyl-benzamide

To a suspension of 4-methyl-3-methoxy benzoic acid (480 mg, 2.9 mmol), 3-bromo-5-amino pyridine (500 mg, 2.9 mmol) and NEt₃ (604 μl, 3.44 mmol) in THF (10 ml) at room temperature under a N₂ atmosphere, HBTU (1.10 g, 2.90 mmol) was added. Reaction was stirred for 30 minutes before being warmed to 50° C. and stirred for 18 hours. Solvent was removed under vacuum, residue was dissolved in EtOAc (30 ml) and washed with 10% citric acid (30 ml), Na₂CO₃ (30 ml), de-ionised water (30 ml) and brine (30 ml), dried over MgSO₄, filtered and evaporated. Residue was purified by column chromatography. Mixture was pre absorbed onto flash silica and eluted with 40% EtOAc/petroleum ether 40/60 (v:v). N-(5-Bromo-pyridin-3-yl)-3-methoxy-4-methyl-benzamide was isolated in 52% yield.

LC-MS, m/z[MH]⁺ 321. Retention time, 2.14 minutes, Method B.

¹H NMR (DMSO-Of₆, 400 MHz): δ=2.02 (s, 3H, CH₃), 3.67 (s, 3H, CH₃), 7.11 (d, 1H, Ar—H), 7.27 (s, 1H₁ Ar—H), 7.30 (d, 1H, Ar—H), 8.21 (s, 1H, Ar—H), 8.30 (s, 1H, Ar—H), 8.70 (s, 1H, Ar—H), 10.29 (s, 1H, NH).

### Preparation of N-[5-(3-hydroxy-phenyl)-pyridin-3-yl]-3-methoxy-4-methyl-benzamide 267

To a solution of N-(5-bromo-pyridin-3-yl)-3-methoxy-4-methyl-benzamide (482 mg, 1.50 mmol) in de-gassed DMF (8 ml) under a N₂ atmosphere, 3-hydroxyphenyl boronic acid (414 mg, 3.8 mmol), NaHCO₃ (504 mg, 6.00 mmol), de-gassed de-ionised water (4 ml), triphenylphosphine (59 mg, 0.22 mmol) and palladium acetate (17 mg, 0.073 mmol) were added. Reaction was stirred at 80° C. for 18 hours. Reaction was cooled and evaporated to dryness. Residue was dissolved in EtOAc (40 ml) and washed with Na₂CO₃ (30 ml) and de-ionised water (30 ml), dried over MgSO₄, filtered and evaporated to dryness. Residue was purified by flash chromatography. Mixture was pre absorbed onto flash silica and eluted with 5% MeOH/DCM. Compound 267 was isolated in a 95% yield.

LC-MS, m/z [MH]⁺ 335. Retention time, 1.89 minutes. Method B.

¹H NMR (DMSO-CZ₆, 400 MHz): δ=3.42 (s, 3H, CH₃), 4.10 (s, 3H, CH₃), 7.05 (d, 1H, Ar—H), 7.25 (s, 1H, Ar—H), 7.32 (d, 1H, Ar—H), 7.53 (t, 2H, Ar—H) 7.72 (s, 1H, Ar—H), 7.78 (d, 1H, Ar—H), 8.62 (s, 1H, Ar—H), 8.87 (s, 1H, Ar—H), 9.12 (s, 1H, Ar—H) 9.85 (s, 1H, OH), 10.65 (s, 1H, NH).

### Preparation of 3-[5-(3-methoxy-4-methyl-benzylamino)-pyridin-3-yl]-phenol 268

To a solution of N-[5-(3-hydroxy-phenyl)-pyridin-3-yl]-3-methoxy-4-methyl-benzamide (267) (474 mg, 1.42 mmol) in THF (10 ml) under a N₂ atmosphere, a 2M solution of borane-methyl sulfide complex in THF (3.54 ml, 7.10 mmol) was added in one portion. Reaction was heated under reflux for 2 hours. Mixture was cooled and evaporated to dryness. Residue was dissolved in EtOAc (30 ml) and washed with 10% citric acid (30 ml), NaHCO₃ (30 ml) and de-ionised water (30 ml), dried over MgSO₄, filtered and evaporated under vacuum. Residue was dissolved in EtOH (30 ml) and heated under reflux for 3 hours. Mixture was cooled and evaporated under vacuum. Residue was purified by flash chromatography. Mixture was pre absorbed onto flash silica and eluted with 5% MeOH/DCM. Compound 268 was isolated in a 37% yield.

LC-MS₁ m/z [MH]⁺ 321. Retention time, 2.32 minutes. Method B.

¹H NMR (DMSO-c/e, 400 MHz): δ=2.15 (s, 3H, CH₃), 3.81 (s, 3H, CH₃), 4.35 (d, 2H, CH₂) 6.60 (t, 1H, N—H), 6.84 (d, 1H₁Ar—H), 6.95 (d, 1H, Ar—H), 7.00 (s, 1H, Ar—H) 7.04 (m, 2H₁ Ar—H), 7.10 (m, 2H, Ar—H), 7.28 (t, 1H, Ar—H), 8.00 (s, 2H, Ar—H), 9.60 (s, 1H, OH).

### Preparation of 5-{[5-(3-hydroxy-phenyl)-pyridin-3-ylamino]-methyl}-2-methyl phenol (247)

To a solution of 3-[5-(3-methoxy-4-methyl-benzylamino)-pyridin-3-yl]-phenol (268) (163 mg, 0.51 mmol) in DCM (10 ml) at −78° C. under a N₂ atmosphere, borontribromide (800 μl, 1.17 mmol) was added. Reaction was allowed to warm to room temperature and stirred for 18 hours. Reaction was quenched with addition of de-ionised water and adjusted to pH 6 by addition of 2M NaOH. Mixture was extracted with EtOAc (2×40 ml). The organic phases were combined, dried over MgSO₄ and evaporated to dryness. Residue was purified by Prep-HPLC. Compound 247 was isolated in 43% yield.

LC-MS, m/z [MH]⁺ 307. Retention time, 1.58 minutes. Method B.

¹H NMR (DMSO-CZ₆, 400 MHz): δ=2.22 (s, 3H, CH₃), 4.41 (d, 2H, CH₂) 6.75 (t, 1H, N—H), 6.91 (d, 1H, Ar—H), 7.70 (s, d, 2H, Ar—H)₁ 7.11 (s, 1H, Ar—H) 7.20 (m, 3H, Ar—H), 7.42 (t, 1H₁Ar—H), 8.12 (d, 2H₁Ar—H)₁9.35 (s, 1H₁O—H), 9.70 (s, 1H₁O—H).

### Preparation of 3-{[5-(3-hydroxy-phenyl)-pyridin-3-ylamino]-methyl}2-methyl-phenol (233)

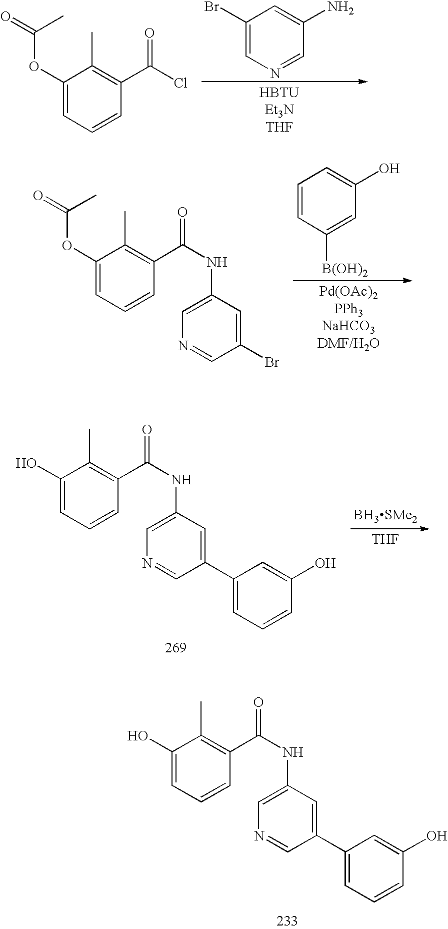

### Preparation of 3-(5-bromo-pyridin-3-yl-carbamoyl)-2-methyl-phenyl-acetate

To a solution of 3-amino-5-bromo pyridine (500 mg, 2.89 mmol) and NEt₃ in THF (5 ml), a solution of acetic acid 3-chlorocarbonyl-2-methyl-phenyl ester (614 mg, 2.89 mmol) in THF (5 ml) was added dropwise. Reaction was stirred for 18 hours at room temperature. Solvent was removed under vacuum. Residue was dissolved in EtOAc (30 ml) and extracted with 10% citric acid (30 ml), de-ionised water (30 ml), 1M NaOH (30 ml) and brine (30 ml), dried over MgSO₄ filtered and evaporated. Residue was purified by flash chromatography. Mixture was pre absorbed onto flash silica and eluted with 50% EtOAc/petroleum ether 40/60 (v:v). 3-(5-Bromo-pyridin-3-yl-carbamoyl)-2-methyl-phenyl-acetate was isolated in a 51% yield.

LC-MS, m/z [MH]⁺ 349. Retention time, 1.89 minutes. Method B.

¹H NMR (CDCl₃, 400 MHz): δ=2.26 (s, 3H, CH₃), 2.29 (s, 3H, CH₃), 7.11 (d, 1H, Ar—H), 7.25 (m, 2H, Ar—H), 8.18 (s, 1H, Ar—H), 8.41 (s, 1H, Ar—H), 8.72 (d, 2H, Ar—H, N—H).

### Preparation of 3-hydroxy-N-[5-(3-hydroxy-phenyl)-pyridin-3-yl]-2-methyl-benzamide (269)

To a solution of 3-(5-bromo-pyridin-3-yl carbamoyl)-2-methyl phenyl acetate (542 mg, 1.55 mmol) in de-gassed DMF (8 ml) under a N₂ atmosphere, 3-hydroxyphenyl boronic acid (428 mg, 3.10 mmol), NaHCO₃ (522 mg, 6.20 mmol), de-gassed de-ionised water (4 ml), triphenylphosphine (61 mg, 0.23 mmol) and palladium acetate (17 mg, o.oδmmol) were added. Reaction was stirred at 80° C. for 18 hours. Reaction was cooled and evaporated to dryness. Residue was dissolved in EtOAc (40 ml) and washed Na₂CO₃ (30 ml) and de-ionised water (30 ml), dried over MgSO₄, filtered and evaporated to dryness. Residue was purified by flash chromatography. Mixture was pre absorbed onto flash silica and eluted with 10% MeOH/DCM. Compound 269 was isolated in a 75% yield.

LC-MS, m/z [MH]⁺ 321. Retention time, 1.45 minutes. Method B.

¹H NMR (DMSO-Gf₆, 400 MHz): δ=2.09 (s, 3H, CH₃), 6.85 (d, 1H, Ar—H), 6.89 (d, 2H, Ar—H), 6.98 (s, 1H, Ar—H), 7.04 (m, 2H₁ Ar—H), 7.24 (t, 1H, Ar—H), 8.32 (s, 1H, Ar—H), 8.45 (s, 1H, Ar—H), 8.75 (s, 1H, Ar—H), 9.51 (d, 1H, O—H), 9.53 (s, 1H, O—H), 10.45 (s, 1H₁N—H).

### Preparation of 3-{[5-(3-hydroxy-phenyl)-pyridin-3-ylamino]-methyl}2-methyl-phenol (233)

To a solution of 3-hydroxy-N-[5-(3-hydroxy-phenyl)-pyridin-3-yl]-2-methyl-benzamide (269) (373 mg, 1.16 mmol) in THF (10 ml) under a N₂ atmosphere, a 2M solution of borane-methyl sulfide complex in THF (2.91 ml, 5.80 mmol) was added in one portion. Reaction was heated under reflux for 2 hours. Mixture was cooled and evaporated to dryness. Residue was dissolved in EtOAc (30 ml) and washed with 10% citric acid (30 ml), NaHCO₃ (30 ml) and de-ionised water (30 ml), dried over MgSO-t, filtered and evaporated under vacuum. Residue was dissolved in EtOH (30 ml) and heated under reflux for 3 hours. Mixture was cooled and evaporated. Residue was purified by flash chromatography. Mixture was pre absorbed onto flash silica and eluted with 5% MeOH/DCM. Compound 233 was isolated in a 31% yield.

LC-MS, m/z [MH]⁺ 307 Retention time, 1.63 minutes, Method B.

¹H NMR (DMSO-CZ₆, 400 MHz): δ=2.20 (s, 3H, CH₃), 4.35 (d, 2H, CH₂), 6.42 (t, 1H, N—H), 6.80 (d, 1H, Ar—H), 6.90 (d, 1H, Ar—H), 7.02 (s, 1H, Ar—H), 7.08 (d, 1H, Ar—H), 7.12 (s, 1H, Ar—H), 7.32 (t, 1H, Ar—H), 8.05 (s, 2H, Ar—H), 9.30 (s, 1H₁O—H), 9.62 (s, 1H, O—H).

### Preparation of 3-{5-[(3-hydroxy-benzyl)-methyl-amino]-pyridin-3-yl}-phenol (241)

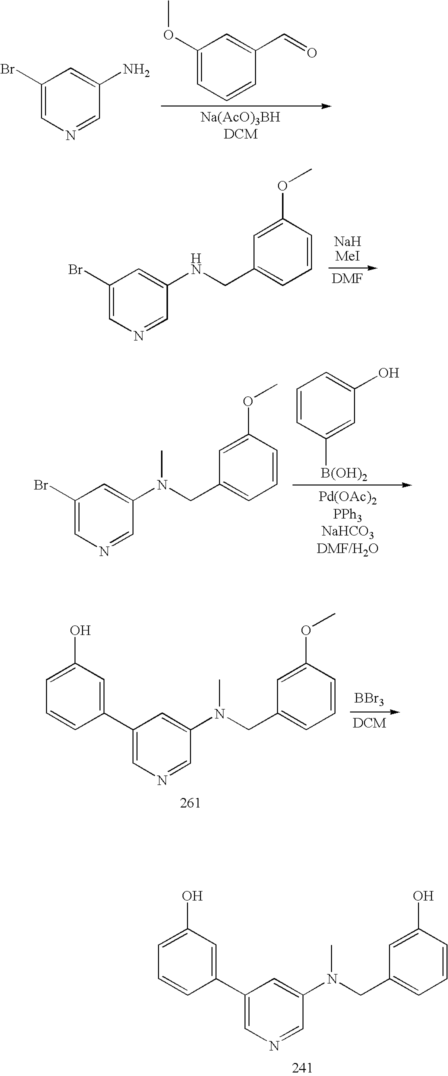

### Preparation of (5-bromo-pyridin-3-yl)-(3-methoxy-benzyl)-amine

Sodium triacetoxyborohydride (1.72 g, 8.15 mmol) was added to a mixture of 3-methoxy benzaldehyde (707 μl, 5.28 mmol) and 3-amino-5-bromo pyridine (1 g, 5.82 mmol) in DCM (20 ml). The reaction was stirred at room temperature for 18 hours. Reaction was diluted with DCM (60 ml) and washed with de-ionised water (2×60 ml). Aqueous phases were combined and extracted with EtOAc (3×60 ml). Organic phases were combined, dried over MgSO₄, filtered and evaporated to dryness. Residue was purified by flash chromatography. Mixture was pre absorbed onto flash silica, loaded onto a 10 g isolute flash Si cartridge and eluted using CombiFlash™ instrumentation, with a gradient of 0-100% EtOAc/petroleum ether 40/60 (v:v). (5-Bromo-pyridin-3-yl)-(3-methoxy-benzyl)-amine was isolated in 78% yield.

LC-MS, m/z [MH]⁺ 293. Retention time, 2.07 minutes. Method B.

¹H NMR (DMSO-Cf₆, 400 MHz): δ=3.60 (s, 3H, CH₃), 4.15 (d, 2H, CH₂), 6.70-6.98 (5H, Ar—H, NH), 7.12 (t, 1H, Ar—H), 7.65 (s, 1H, Ar—H), 7.80 (s, 1H, Ar—H).

### Preparation of (5-bromo-pyridin-3-yl)-(3-methoxy-benzyl)-methyl-amine

To a solution of (5-bromo-pyridin-3-yl)-(3-methoxy-benzyl)-amine (450 mg, 1.54 mmol) in DMF (5 ml) under a N₂ atmosphere at 0° C., sodium hydride (60% dispersed in mineral oil, 74 mg, 1.85 mmol) was added. Reaction was stirred at 0° C. for 30 minutes. Methyl iodide (210 μl, 3.28 mmol) was added and reaction allowed to warm to room temperature and stirred for 2 hours. Reaction was evaporated to dryness. Residue was dissolved in EtOAc (40 ml) and washed with de-ionised water (40 ml), dried over MgSO₄, filtered and evaporated. Residue was purified by flash chromatography. Mixture was pre absorbed onto flash silica, loaded onto a 10 g isolute flash Si cartridge and eluted using CombiFlash™ instrumentation, with a gradient of 0-65% EtOAc/petroleum ether 40/60 (v:v). (5-bromo-pyridin-3-yl)-(3-methoxy-benzyl)-methyl-amine was isolated in 37% yield.

LC-MS, m/z[MH]⁺ 307. Retention time, 2.28 minutes. Method B.

¹H NMR (DMSO-CZ₆, 400 MHz): δ=2.88 (s, 3H, N—CH₃), 3.51 (s, 3H, OCH₃), 4.41 (s, 2H, CH₂), 6.55 (m, 2H, Ar—H), 6.63 (d, 1H, Ar—H), 7.05 (m, 2H, Ar—H), 7.68 (s, 1H₁Ar—H), 7.85 (s, 1H₁Ar—H).

### Preparation of 3-{5-[(3-methoxy-benzyl)-methyl-amino]-pyridin-3-yl}-phenol (261)

To a solution of (5-bromo-pyridin-3-yl)-(3-methoxy-benzyl)-methyl-amine (160 mg, 0.52 mmol) in de-gassed DMF (10 ml) under a N₂ atmosphere, 3-hydroxyphenyl boronic acid (144 mg, 1.04 mmol), NaHCO₃ (175 mg, 2.10 mmol), de-gassed de-ionised water (5 ml), triphenylphosphine (21 mg, 0.078 mmol) and palladium acetate (6 mg, 0.026 mmol) were added. Reaction was stirred at 80° C. for 18 hours. Reaction was cooled and evaporated to dryness. Residue was dissolved in EtOAc (40 ml) and washed with Na₂CO₃ (30 ml) and de-ionised water (30 ml), dried over MgSO₄, filtered and evaporated to dryness. Residue was purified by flash chromatography. Mixture was pre absorbed onto flash silica, loaded onto a 10 g isolute flash Si cartridge and eluted using CombiFlash™ instrumentation, with a gradient of 0-100% EtOAc/petroleum ether 40/60 (v:v). Compound 261 was isolated in 75% yield.

LC-MS, m/z[MH]⁺ 321. Retention time, 2.00 minutes. Method B.

¹H NMR (DMSO-cfe, 400 MHz): δ=3.10 (s, 3H, N—CH₃), 3.70 (s, 3H, O—CH₃), 4.65 (s, 2H, CH₂), 6.79 (m, 4H, Ar—H), 6.98 (s, 1H, Ar—H), 7.01 (d, 1H, Ar—H), 7.13 (s, 1H₁Ar—H), 7.21 (m, 2H, Ar—H), 8.03 (s, 2H, Ar—H), 9.52 (s, 1H, OH).

### Preparation of 3-{5-[(3-hydroxy-benzyl)-methyl-amino]-pyridin-3-yl}-phenol (241)

To a solution of 3-{5-[(3-methoxy-benzyl)-methyl-amino]-pyridin-3-yl}-phenol (261) (75 mg, 0.23 mmol) in DCM (15 ml) at −78° C. under a N₂ atmosphere, boron tribromide (800 μl, 1.17 mmol) was added. Reaction was allowed to warm to room temperature and stirred for 18 hours. Reaction was quenched with addition of de-ionised water and adjusted to pH6 by addition of 2M NaOH. Mixture was extracted with EtOAc (2×40 ml). The organic phases were combined, dried over MgSO₄ and evaporated to dryness. Compound 241 was isolated in 57% yield.

LC-MS, m/z [MH]⁺ 305. Retention time, 1.60 minutes. Method B.

¹H NMR (DMSO-d₆, 400 MHz): δ=3.07 (s, 3H, N—CH₃), 4.58 (s, 2H, CH₂), 6.56 (d, 2H, Ar—H), 6.60 (d, 1H, Ar—H), 6.74 (d, 1H, Ar—H)₁ 6.97 (s, 1H, Ar—H), 7.01 (d, 1H, Ar—H), 7.10 (m, 2H, Ar—H)₁ 7.21 (t, 1H, Ar—H), 8.02 (s, 2H₁Ar—H)₁ 9.30 (s, 1H, OH)₁ 9.52 (S₁ 1H₁OH).

### Preparation of 3-(pyridin-3-ylanninomethyl)-phenol (257)

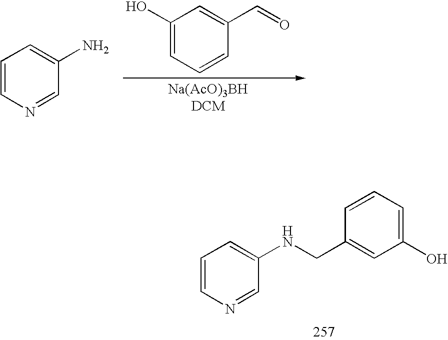

Sodium triacetoxyborohydride (664 mg, 2.97 mmol) was added to a mixture of 3-hydroxy benzaldehyde (285 mg, 2.34 mmol) and 3-amino pyridine (200 mg, 2.13 mmol) in DCM (10 ml). The reaction was stirred at room temperature for 18 hours. Reaction mixture was diluted with DCM (30 ml) and washed with de-ionised water (2×20 ml). Aqueous phase was combined and extracted with EtOAc (3×30 ml). The organic phases were combined, dried over MgSO₄, filtered and evaporated. Residue was purified by column chromatography. Mixture was pre absorbed onto flash silica and eluted with 80% EtOAc/petroleum ether 40/60 (v:v). Compound 257 was isolated in 52% yield.

LC-MS₁ m/z [MH]⁺ 201. Retention time, 1.32 minutes. Method B.

¹H NMR (DMSO-de, 400 MHz): δ=4.35 (d, 2H₁CH₂), 6.62 (t, 1H₁N—H)₁ 6.75 (d, 1H, Ar—H), 6.90 (s, 1H₁Ar—H)₁ 6.95 (s, 1H, Ar—H)₁ 7.00 (d, 1H₁Ar—H)₁ 7.19 (d, 1H, Ar—H)₁ 7.30 (t, 1H₁Ar—H), 7.90 (d, 1H, Ar—H)₁ 8.11 (s, 1H, Ar—H)₁ 9.49 (s, 1H₁OH).

### Preparation of (3-hydroxy-benzyl)-[5-(3-hydroxy-benzyl)-pyridin-3-yl]-amine (272)

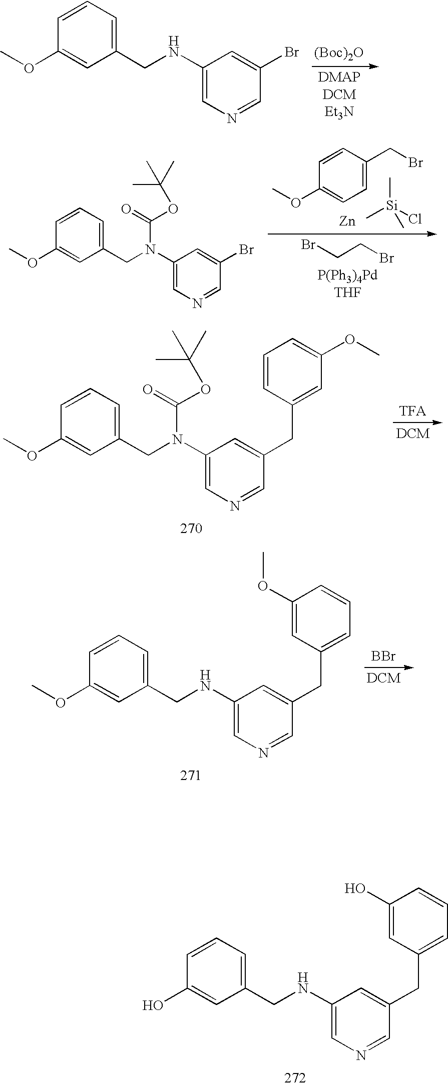

### Preparation of (5-bromo-pyridin-3-yl)-(3-methoxy-benzyl)-carbamic acid tert-butyl ester

To a solution of (5-bromo-pyridin-3-yl)-(3-methoxy-benzyl)-amine (1.35 g, 4.62 mmol) in dry DCM (20 ml), DMAP (135 mg), and Et₃N (966 μl, 6.93 mmol) was added followed by dropwise addition of a solution of di-tert-butyl dicarbonate (2.26 g, 10.35 mmol), in dry DCM (20 ml). Reaction was stirred at room temperature for 24 hours. Mixture was evaporated, dissolved in EtOAc (30 ml) and washed with 10% citric acid (30 ml), 1M NaOH (2×30 ml), de-ionised water (30 ml) and brine (30 ml). The organic phases were dried over MgSO₄, filtered and evaporated. (5-Bromo-pyridin-3-yl)-(3-methoxy-benzyl)-carbamic acid tert-butyl ester was isolated in 67% yield.

LC-MS, m/z [MH]⁺ 393. Retention time, 2.56 minutes. Method B.

¹H NMR (DMSO-de, 400 MHz): δ=1.45 (s, 9H, t-butyl), 3.79 (s, 3H, O—CH₃), 4.81 (s, 2H, CH₂), 6.80 (m, 3H, Ar—H), 7.25 (t, 1H, Ar—H), 7.74 (s, 1H, Ar—H), 8.40 (s, 1H, Ar—H), 7.48 (s, 1H₁Ar—H).

### Preparation of (3-methoxy-benzyl)-[5-(3-methoxy-benzyl)-pyridin-3-yl]-carbamic acid tert-butyl ester (270)

To a suspension of Zinc (218 mg, 3.2 mmol) in dry THF (5 ml) under a N₂ atmosphere, dibromoethane (19.2 μl, 0.22 mmol) was added. Reaction was heated at 60° C. for 5 minutes then allowed to cool to 35° C. Chlorotrimethylsilane (58 μl, 0.45 mmol) was added and mixture was stirred for 30 minutes followed by addition of 3-methoxybenzylbromide (234 μl, 1.67 mmol). Reaction was allowed to stir for 30 minutes. A solution of (δ-bromo-pyridin-S-ylHS-methoxy-benzyO-carbamic acid tert-butyl ester (219 mg, 0.56 mmol) and tetrakis(triphenylphosphine) palladium (0) (16 mg 0.014 mmol) in dry THF (3 ml) was added and reaction was stirred for 40 minutes at 50° C. Reaction was cooled, filtered through celite, diluted with EtOAc (20 ml) and washed with NH₄Cl (15 ml), and brine (15 ml), dried over MgSO₄, filtered and evaporated. Residue was purified by column chromatography. Mixture was pre absorbed onto flash silica and eluted with 5% MeOH/DCM. Compound 270 was isolated in 20% yield.

LC-MS, m/z[MH]⁺ 435. Retention time, 2.17 minutes. Method A.

¹H NMR (CDCl₃, 400 MHz): δ=1.38 (s, 9H, t-butyl), 3.75 (s, 3H, O—CH₃), 3.78 (s, 3H, O—CH₃), 3.88 (s, 2H, CH₂), 4.77 (s, 2H, CH₂), 6.70 (m, 6H, Ar—H), 7.20 (m, 3H, Ar—H), 8.28 (s, 1H, Ar—H), 8.30 (s, 1H, Ar—H).

### Preparation of (3-methoxy-benzyl)-[5-(3-methoxy-benzyl)-pyridin-3-yl]-amine (271)

To a solution of (3-methoxy-benzyl)-[5-(3-methoxy-benzyl)-pyridin-3-yl]-carbamic acid tert-butyl ester (270) (48 mg, 0.011 mmol) in DCM (2 ml) and de-ionised water (0.5 ml), TFA (2 ml) was added. Reaction was stirred for 1 hour at room temperature. Reaction was evaporated to dryness and partitioned between NaHCO₃ (30 ml) and EtOAc (30 ml). Aqueous phase was removed and further extracted with EtOAc (2×30 ml). The organic phases were combined, dried over MgSO₄, filtered and evaporated. Compound 271 was isolated in 71% yield.

LC-MS, m/z[MH]⁺ 335. Retention time, 2.18 minutes, Method B.

¹H NMR (CDCl₃, 400 MHz): δ=3.78 (s, 3H, O—CH₃), 3.80 (s, 2H, O—CH₃), 3.82 (s, 2H, CH₂), 4.03 (br s, 1H, N—H), 4.26 (d, 2H, CH₂), 5.99 (s, 1H, Ar—H), 6.73 (s, 1H, Ar—H), 6.80 (d, 2H, Ar—H), 6.83 (d, 1H, Ar—H), 6.90 (s, 1H, Ar—H), 6.93 (d, 1H, Ar—H), 7.24 (m, 2H, Ar—H), 7.91 (s, 1H, Ar—H), 7.95 (s, 1H, Ar—H).

### Preparation of (3-hydroxy-benzyl)-[5-(3-hydroxy-benzyl)-pyridin-3-yl]-amine (272)

To a solution of (3-methoxy-benzyl)-[5-(3-methoxy-benzyl)-pyridin-3-yl]-amine (271) (29 mg, 0.086 mmol) in DCM (2 ml) at −78° C. under a N₂ atmosphere, a 2M solution of boron tribromide in DCM (2.72 ml, 2.72 mmol) was added dropwise. Reaction was allowed to rise to room temperature and stirred for 1 hour. Reaction was quenched with NaHCO₃ (5 ml) and extracted with EtOAc (3×30 ml). The organic phases were combined, washed with brine (50 ml), dried over MgSO₄, filtered and evaporated. Residue was purified by column chromatography. Mixture was pre absorbed onto flash silica and eluted with 10% MeOH/DCM. Compound 272 was isolated in 40% yield.

LC-MS, m/z [MH]⁺ 307. Retention time, 1.34 minutes. Method B.

¹H NMR (DMSO-de, 400 MHz): δ=3.79 (s, 2H₁ CH₂), 4.25 (s, 2H, CH₂), 6.68-7.10 (9H, Ar—H, N—H).

### Characterization of Inventive Compounds Via LCMS Methods:

| Comp | LCMS | Retention | | | Comp | LCMS | Retention | | |
|---|---|---|---|---|---|---|---|---|---|
| No. | Method | Time | M⁺ | M − H | No. | Method | Time | M⁺ | M⁻ |
| 1 | C | | | | 2 | C | 1.9 | 398 | |
| 3 | C | 1.9 | 321 | | 4 | C | | 347 | |
| 5 | C | | | | 6 | C | 1.8 | 299 | |
| 7 | C | 2.2 | 309 | | 8 | C | | | |
| 9 | C | 1.8 | 263 | | 10 | C | 1.9 | 325 | |
| 11 | C | 2.0 | 326 | | 12 | C | 2.0 | 311 | |
| 13 | C | 1.9 | 327 | | 14 | C | | | |
| 15 | C | | 340 | | 16 | C | | | |
| 17 | C | | | | 18 | C | 2.8 | 342 | |
| 19 | C | | | | 20 | C | | 313 | |
| 21 | C | 1.9 | 325 | | 22 | C | | | |
| 23 | C | 2.2 | 377 | | 24 | C | | 406 | |
| 25 | C | 1.7 | 282 | | 26 | C | | 406 | |
| 27 | C | | | | 28 | C | 2.1 | 331 | |
| 29 | C | 2.0 | 311 | | 30 | C | 2.0 | 327 | |
| 31 | C | 2.0 | 390 | | 32 | C | 1.7 | 252 | |
| 33 | C | 2.0 | 346 | | 34 | C | 1.8 | 337 | |
| 35 | C | 2.0 | 328 | | 36 | C | 2.1 | 343 | |
| 37 | C | 2.1 | 313 | | 38 | C | 2.0 | 282 | |
| 39 | C | 2.1 | 423 | | 40 | C | 1.7 | 364 | |
| 41 | C | | 263 | | 42 | C | | | |
| 43 | C | 2.1 | 311 | | 44 | C | 1.8 | 290 | |
| 45 | C | 2.3 | 312 | | 46 | C | | | |
| 47 | C | 2.0 | 322 | | 48 | C | 2.1 | 342 | |
| 49 | C | | | | 50 | C | 1.9 | 298 | |
| 51 | C | 1.9 | 328 | | 52 | C | 2.2 | 347 | |
| 53 | C | 2.2 | 357 | | 54 | C | | | |
| 55 | C | | 365 | | 56 | C | 2.2 | 311 | |
| 57 | C | | | | 58 | C | 2.1 | 334 | |
| 59 | C | 2.0 | 346 | | 60 | C | 2.2 | 351 | |
| 61 | C | 2.1 | 323 | | 62 | C | 2.0 | 342 | |
| 63 | C | 2.4 | 350 | | 64 | C | 2.1 | 329 | |
| 65 | C | 2.3 | 359 | | 66 | C | 2.2 | 359 | |
| 67 | C | 2.0 | 338 | | 68 | C | 2.0 | 311 | |
| 69 | C | | | | 70 | C | | | |
| 71 | C | 2.0 | 357 | | 72 | C | 1.8 | 313 | |
| 73 | C | 2.0 | 311 | | 74 | C | 1.8 | 282 | |
| 75 | C | | | | 76 | C | | 268 | |
| 77 | C | 2.1 | 361 | | 78 | C | 2.1 | 327 | |
| 79 | C | 2.1 | 361 | | 80 | C | 2.2 | 298 | |
| 81 | C | 2.3 | 329 | | 82 | C | 1.9 | 338 | |
| 83 | C | 2.2 | 361 | | 84 | C | | | |
| 85 | C | 2.3 | 362 | | 86 | C | 2.2 | 312 | |
| 87 | C | | 312 | | 88 | C | | 296 | |
| 89 | C | 1.9 | 365 | | 90 | C | 1.7 | 281 | |
| 91 | C | 1.8 | 378 | | 92 | C | | 278 | |
| 93 | C | 2.0 | 307 | | 94 | C | 2.1 | 301 | |
| 95 | C | | 388 | | 96 | C | 1.9 | 381 | |
| 97 | C | 1.6 | 338 | | 98 | C | 1.9 | 295 | |
| 99 | C | 2.2 | 411 | | 100 | C | 2.0 | 327 | |
| 101 | C | 2.2 | 278 | | 102 | C | 1.8 | 344 | |
| 103 | C | 2.3 | 356 | | 104 | C | 1.7 | 364 | |
| 105 | C | 1.9 | 324 | | 106 | C | 2.0 | 368 | |
| 107 | C | | 306 | | 108 | C | | | |
| 109 | C | 1.8 | 257 | | 110 | C | | 252 | |
| 111 | C | | 372 | | 112 | C | 1.9 | 352 | |
| 113 | C | 1.9 | 313 | | 114 | C | 2.3 | 330 | |
| 115 | C | 2.2 | 308 | | 116 | C | 1.7 | 281 | |
| 117 | C | 2.0 | 324 | | 118 | C | 2.1 | 346 | |
| 119 | C | 2.1 | 298 | | 120 | C | 1.9 | 274 | |
| 121 | C | 1.9 | 368 | | 122 | C | 2.2 | 332 | |
| 123 | C | | 331 | | 124 | C | 1.8 | 340 | |
| 125 | C | 1.7 | 348 | | 126 | C | 1.8 | 321 | |
| 127 | C | 1.9 | 313 | | 128 | C | | 293 | |
| 129 | C | 2.2 | 357 | | 130 | C | 1.8 | 362 | |
| 131 | C | 2.2 | 349 | | 132 | C | 1.9 | 379 | |
| 133 | C | 1.9 | 309 | | 134 | C | | 480 | |
| 135 | C | | | | 136 | C | 2.3 | 388 | |
| 137 | C | 1.8 | 277 | | 138 | C | 1.9 | 370 | |
| 139 | C | | 298 | | 140 | C | 2.1 | 385 | |
| 141 | C | 1.7 | 267 | | 142 | C | 2.1 | 305 | |
| 143 | C | 2.5 | 278 | | 144 | C | 2.1 | 354 | |
| 145 | C | 1.9 | 354 | | 146 | C | 2.4 | 377 | |
| 147 | C | 2.0 | 356 | | 148 | C | 2.0 | 305 | |
| 149 | C | | 386 | | 150 | C | 2.2 | 303 | |
| 151 | C | 2.0 | 340 | | 152 | C | 1.7 | 320 | |
| 153 | C | 1.9 | 327 | | 154 | C | 2.0 | 355 | |
| 155 | C | 1.7 | 307 | | 156 | C | 2.0 | 293 | |
| 157 | C | 2.0 | 349 | | 158 | C | | | |
| 159 | C | | 278 | | 160 | C | 1.6 | 294 | |
| 161 | C | 1.9 | 367 | | 162 | C | 2.3 | 377 | |
| 163 | C | 1.7 | 308 | | 164 | C | 2.4 | 293 | |
| 165 | C | 2.0 | 311 | | 166 | C | 2.0 | 282 | |
| 167 | C | 1.9 | 335 | | 168 | C | 2.1 | 297 | |
| 169 | C | 2.1 | 374 | | 170 | C | 2.0 | 367 | |
| 171 | C | 2.0 | 303 | | 172 | C | 1.9 | 307 | |
| 173 | C | | 413 | | 174 | C | 2.0 | 308 | |
| 175 | C | 2.0 | 329 | | 176 | C | 1.7 | 267 | |
| 177 | C | 1.8 | 311 | | 178 | C | 1.9 | 413 | |
| 179 | C | | | | 180 | C | 2.2 | 330 | |
| 181 | C | 1.5 | 339 | | 182 | C | 1.9 | 343 | |
| 183 | C | 2.1 | 365 | | 184 | C | 1.9 | 340 | |
| 185 | C | 2.0 | 282 | | 186 | C | 2.1 | 315 | |
| 187 | C | 2.0 | 338 | | 188 | C | 1.7 | 380 | |
| 189 | C | 1.9 | 353 | | 190 | C | 1.9 | 263 | |
| 191 | C | 2.0 | 329 | | 192 | C | 2.2 | 374 | |
| 193 | C | 2.0 | 384 | | 194 | C | 1.9 | 304 | |
| 195 | C | 1.5 | 279 | | 196 | C | 1.9 | 430 | |
| 197 | C | 1.9 | 302 | | 198 | C | 1.9 | 368 | |
| 199 | C | 2.1 | 352 | | 200 | C | 1.9 | 394 | |
| 201 | C | 1.9 | 315 | | 202 | C | | | |
| 203 | C | 2.1 | 350 | | 204 | C | 2.2 | 388 | |
| 205 | C | 2.0 | 329 | | 206 | C | 1.7 | 355 | |
| 207 | C | 1.8 | 308 | | 208 | C | 2.1 | 315 | |
| 209 | C | | 278 | | 210 | C | 2.1 | 392 | |
| 211 | C | 2.2 | 390 | | 212 | C | 1.7 | 293 | |
| 213 | C | 2.0 | 356 | | 214 | C | 1.8 | 321 | |
| 215 | C | 2.1 | 361 | | 216 | C | 1.7 | 307 | |
| 217 | C | 1.6 | 364 | | 218 | C | 1.9 | 362 | |
| 219 | C | 2.0 | 288 | | 220 | C | 1.9 | 308 | |
| 221 | C | 1.7 | 334 | | 222 | C | | 308 | |
| 223 | C | 1.8 | 337 | | 224 | C | | | |
| 225 | C | 1.7 | 293 | | 226 | C | | | |
| 227 | C | 1.9 | 334 | | 228 | C | 1.8 | 283 | |
| 229 | C | 1.6 | 307 | | 230 | C | 1.6 | 320 | |
| 231 | C | 1.4 | 311 | | 232 | C | 1.5 | 292 | |
| 233 | C | 1.6 | 307 | | 234 | C | 1.4 | 307 | |
| 235 | C | 1.8 | 322 | | 236 | C | 1.8 | 325 | |
| 237 | C | 1.7 | 322 | | 238 | C | 1.8 | 277 | |
| 239 | C | 1.8 | 307 | | 240 | C | 1.6 | 334 | |
| 241 | C | 1.6 | | 305 | 242 | C | 1.3 | 309 | |
| 243 | C | 1.8 | 225 | | 244 | C | 1.3 | 293 | |
| 245 | C | 1.4 | 192 | | 246 | C | 1.0 | 321 | |
| 247 | C | 1.6 | 307 | | 248 | C | 1.1 | 279 | |
| 249 | C | 1.6 | 293 | | 250 | C | 1.8 | 318 | |
| 251 | C | 1.6 | 293 | | 252 | C | 1.7 | 262 | |
| 253 | C | | | | 254 | C | 1.8 | 277 | |
| 255 | C | 1.9 | 325 | | 256 | C | 1.4 | 381 | |
| 257 | C | 1.3 | 201 | | 258 | C | 1.8 | 307 | |
| 259 | C | 1.8 | 325 | | 260 | C | 1.1 | 187 | |
| 261 | C | 1.6 | 305 | | 262 | C | | | |

LCMS method C:
Instrument: Waters ZQ MS system + Binary HPLC system with Diode Array UV Detector
HPLC:
Column: Phenomenex - Luna C18(2) 30 × 4.6 mm ID, 5um
Mobile Phase: Acetonitrile (ACN)/UHQ water/0.1% formic acid - HPLC grade 5% ACN (O.δ min) to 95% ACN in 2.5 min, hold for 1.5 min
Flow Rate: 2.0 ml/min
Detector: DAD 210-400 nm
MS: Electospray +/−ve ionisation
Cone Voltage: 25 V
Source Temp.: 120° C.
Mass Range: 100-1000 amu

One skilled in the art readily appreciates that the present invention is well adapted to carry out the objects and obtain the ends and advantages mentioned, as well as those inherent therein. The methods and reagents described herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

Those skilled in the art will recognize, or be able to ascertain using no more than routine experimentation, many equivalents to the specific embodiments of the invention described herein. Such equivalents are intended to be encompassed by the following claims. Those skilled in the art will also recognize that all combinations of embodiments, combination of aspects or features of the claims described herein are within the scope of the invention.

1. A compound having a structure of general formula (I): 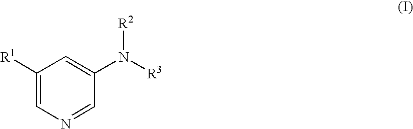
wherein:
R¹ represents —CR²³(R²⁴)R²⁵, —CR²⁸(R²⁹)—CR²⁶(R²⁷)—CR²³(R²⁴)R²⁵, —CR²⁶(R²⁷)—CR²³(R²⁴)R²⁵, —(CH₂)ₙ—CR²⁸(R²⁹)—CR²⁶(R²⁷)—CR²³(R²⁴)R²⁵, —(CH₂)ₙ—CH═CH—(CH₂)ᵣ—CR²³(R²⁴)R²⁵, 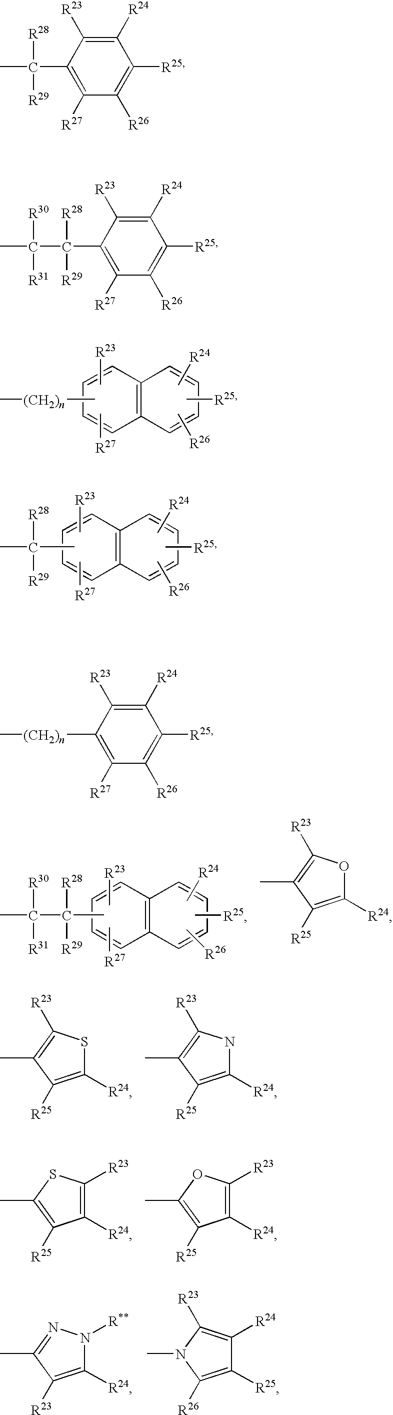 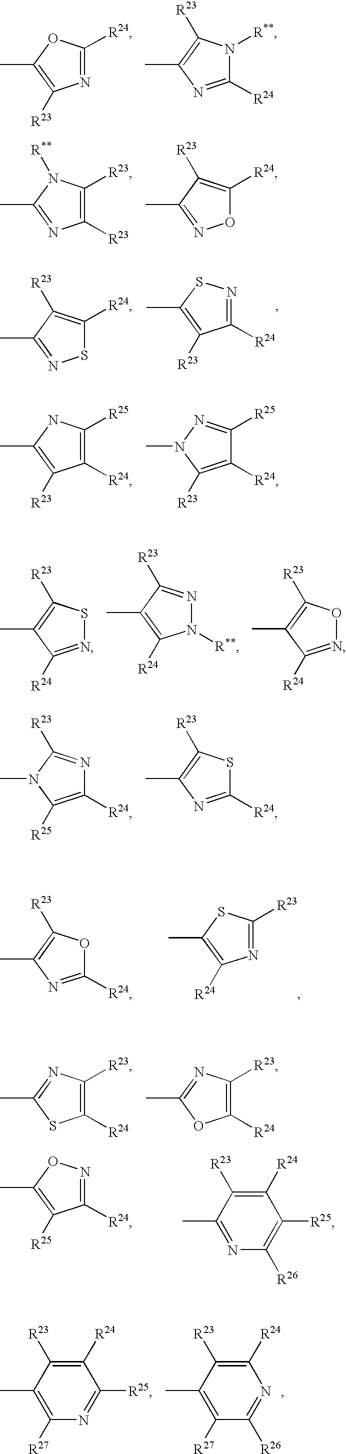 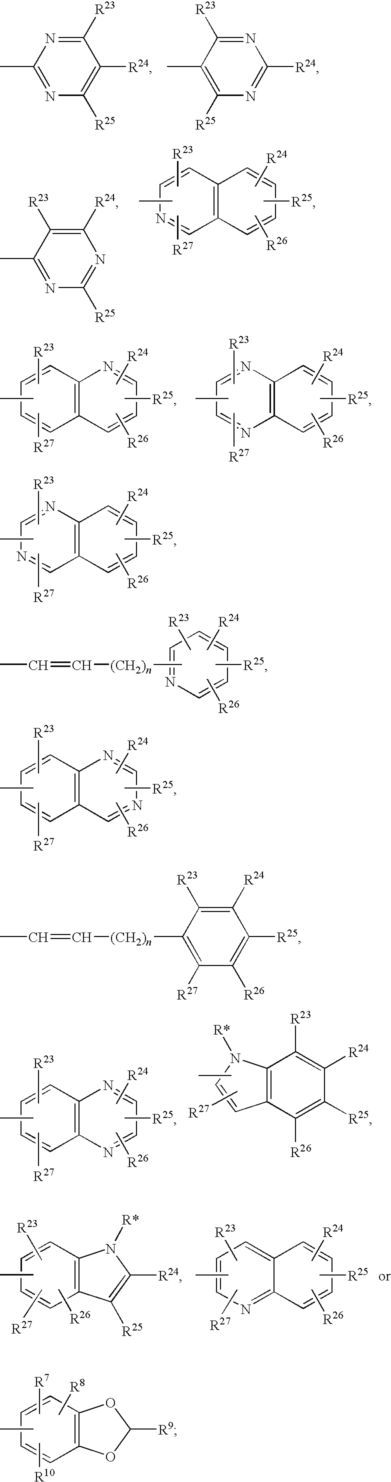
R², R* and R** represent independently of each other —H, —CH₃, —C₂H₅, —CH═CH₂, —C≡CH, —C₃H₇, -cyclo-C₃H₅, —CH(CH₃)₂, —CH₂—CH═CH₂, —C(CH₃)═CH₂, —CH═CH—CH₃, —C≡C—CH₃, —CH₂—C═CH, —C₄H₉, -cyclo-C₄H₇, —CH₂—CH(CH₃)₂, —CH(CH₃)—C₂H₅, —C(CH₃)₃, —C₅H₁₁, —R′, —R″, —R′″, -cyclo-C₅H₉, —C₆H₁₃, -cyclo-C₆H₁₁, -Ph, —CH₂Ph, —C₆H₄—CH₃, —CR′(R″)R′″, —C₂(R′)₅, —CH₂—CR′(R″)R′″, —CHR′—CH(R″)R′″, —C(R′)R″, —CH₂—R′″, —C₃(R′)₇, —C₂H₄—C(R′)₃, —CHO, —COCH₃, —COC₂H₅, —COC₃H₇, —COC₄H₉, —CO-cyclo-C₃H₅, —COCH(CH₃)₂, —COC(CH₃)₃, —COPh, —CO—CH₂Ph, —CO—C₆H₄—CH₃, —COOCH₃, —COOC₂H₅, —COOC₃H₇, —COOC₄H₉, —COO-cyclo-C₃H₅, —COOCH(CH₃)₂, —COOC(CH₃)₃, —COOPh, —COO—CH₂Ph, or —COO—C₆H₄—CH₃;
R′, R″ and R′″ represent independently of each other —H, —F, —Cl, —Br, —I, —CN, —SO₃H, —CONH₂, —OH, —SH, —OCH₃, —OC₂H₅, —SCH₃, —SC₂H₅, —NH₂, —NO₂, —NH(CH₃), —N(CH₃)₂, —NH(C₂H₅), —N(C₂H₅)₂, —OCF₃, —CH₂F—CHF₂, —CF₃, —CH₂Cl, —CH₂Br, —CH₂I, —CH₂—CH₂F, —CH₂—CF₃, —CH₂—CH₂Cl, —CH₂—CH₂Br, —CH₂—CH₂J, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, -Ph, —CH₂-Ph, —C₆H₄—CH₃, —CPh₃, —CH═CH₂, —CH₂—CH═CH₂, —C(CH₃)═CH₂, —CH═CH—CH₃, —C₂H₄—CH═CH₂, —CH═C(CH₃)₂, —CH₂—CH═CH—CH₃, —C≡CH, —C≡C—CH₃, —CH₂—C≡CH, —CHO, —COCH₃, —COC₂H₅, —COC₃H₇, —CO-cyclo-C₃H₅, —COCH(CH₃)₂, —COC(CH₃)₃, —OOC—CH₃, —OOC—C₂H₅, —COOH, —COOCH₃, —COOC₂H₅, —COOC₃H₇, —COO-cyclo-C₃H₅, —COOCH(CH₃)₂, or —COOC(CH₃)₃;
R³ represents —R⁴, —CO—R⁴, —CO—CH(R⁵)—R⁴, —CH(R⁵)—R⁴, —CH(R⁵)—CH(R⁶)—R⁴, —CH(R⁵)—CO—R⁴, —CH(R⁵)—CH(R⁶)—CO—R⁴, —CH(R⁵)—O—CO—R⁴, —CH(R⁵)—CH(R⁶)—O—CO—R⁴, —CO—NH—R⁴, —CO—O—R⁴, —SO₂—R⁴, —CH(R⁵)—SO₂—R⁴, or —CH(R⁵)—CH(R⁶)—SO₂—R⁴;
R⁴ represents —CR¹⁶(R¹⁷)R¹⁸, —CR²¹(R²²)—CR¹⁹(R²⁰)—CR¹⁶(R¹⁷)R¹⁸, —CR¹⁹(R²⁰)—CR¹⁶(R¹⁷)R¹⁸, —(CH₂)ₙ—CR²¹(R²²)—CR¹⁹(R²⁰)—CR¹⁶(R¹⁷)R¹⁸, 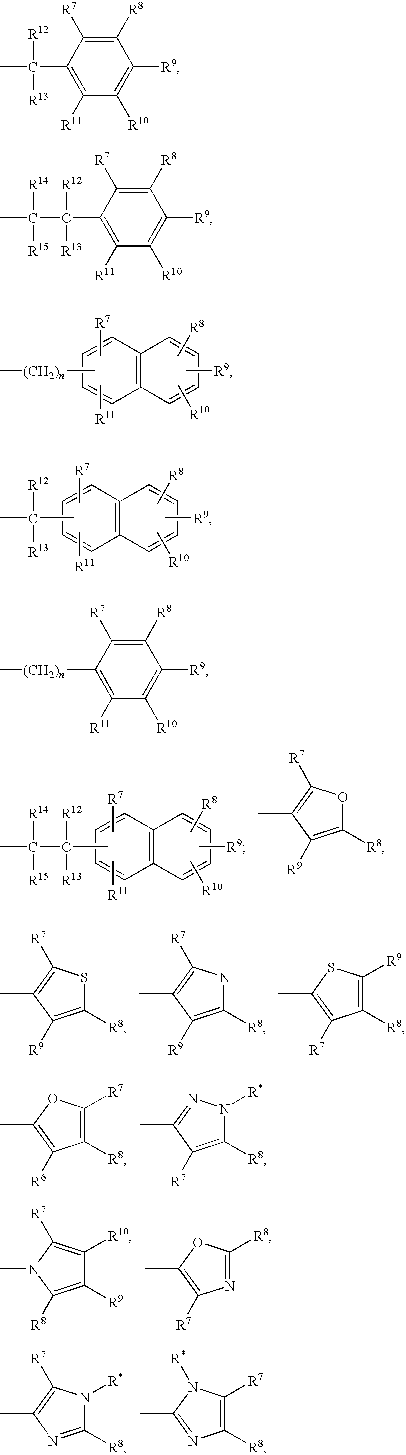 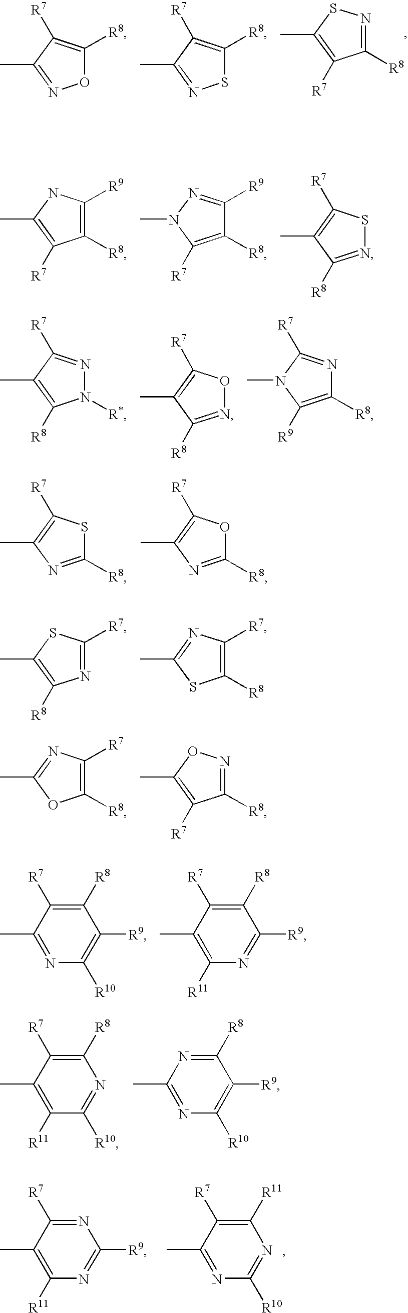 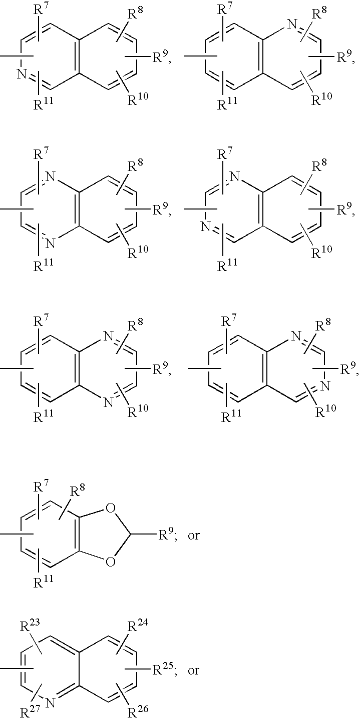
R² and R³ form together a heterocyclic ring such that the residue 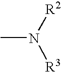
represents one of the following moieties: 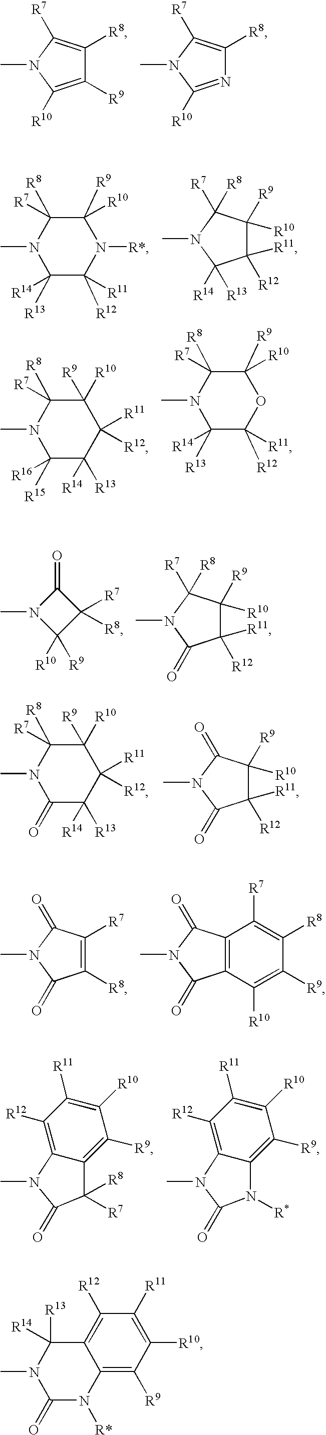 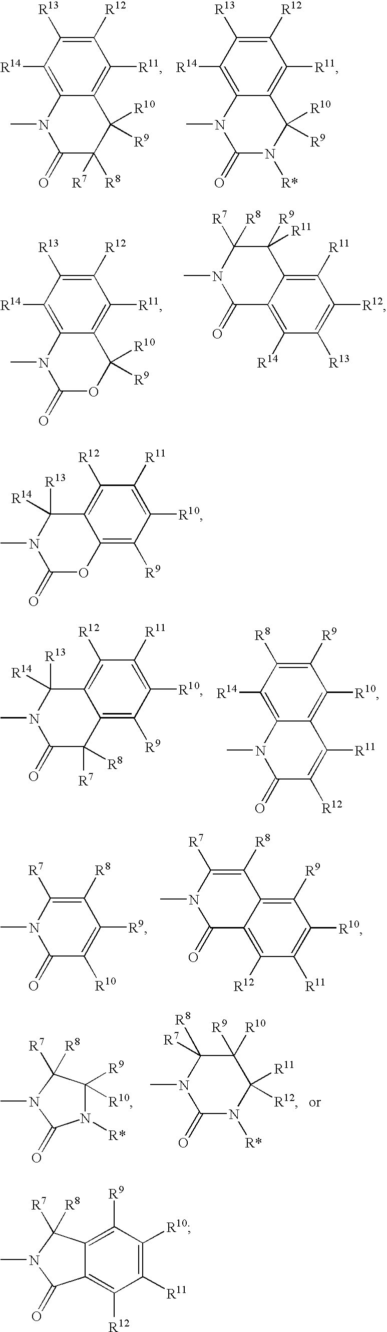
R⁵-R³¹ represent independently of each other
—(CH₂)ₘ—CR³²R³³R³⁴, —CR³⁵R³⁶R³⁷, —CR⁴³R⁴⁴—CR⁴⁵R⁴⁶—CR⁴⁷R⁴⁸R⁴⁹, —CR³⁸R³⁹—CR⁴⁰R⁴¹R⁴², —X—(CH₂)ₘ—CR³²R³³R³⁴, —X—CR³⁵R³⁶R³⁷, —X—CR³⁸R³⁹—CR⁴⁰R⁴¹R⁴², —X—CR⁴³R⁴⁴—CR⁴⁵R⁴⁶—CR⁴⁷R⁴⁸R⁴⁹, —CH₂R⁵⁰, —X—CH₂R⁵¹, —(CH₂)ₚ—R⁵³, —X—(CH₂)_{q}—R⁵⁴;
X represents —CO—, —O—, —S—, —NR*—, —NH—CO—, —CO—NH—, —O—CO—, —CO—O—, —SO₂—, —SO—, —SO₂—O—, —NH—SO₂—, —O—SO₂—, —O—CO—O—, —O—CO—NH—, —NH—CO—O—, —NH—CO—NH—, —NH—CS—NH—, —NH—C(═NH)—NH—, —CF₂—, —C₂F₄—, —C₃F₆—;
R⁵-R⁵⁴ represent independently of each other
—H, —OH, —OCH₃, —OC₂H₅, —OC₃H₇, —O-cyclo-C₃H₅, —OCH(CH₃)₂, —OC(CH₃)₃, —OPh, —OCH₂-Ph, —SH, —SCH₃, —SC₂H₅, —SC₃H₇, —S-cyclo-C₃H₅, —SCH(CH₃)₂, —SC(CH₃)₃, —NO₂, —F, —Cl, —Br, —I, —N₃, —CN, —CHO, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —COO-cyclo-C₃H₅, —COOCH(CH₃)₂, —COOC(CH₃)₃, —OOC—CH₃, —OOC—C₂H₅, —OOC—C₃H₇, —OOC-cyclo-C₃H₅, —OOC—CH(CH₃)₂, —OOC—C(CH₃)₃, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON[CH(CH₃)₂]₂, —NH₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(cyclo-C₃H₅)₂, —N[CH(CH₃)₂]₂, —N[C(CH₃)₃]₂, —SOCH₃, —SOC₂H₅, —SOC₃H₇, —SO-cyclo-C₃H₅, —SOCH(CH₃)₂, —SOC(CH₃)₃, —SO₂CH₃, —SO₂C₂H₅, —SO₂C₃H₇, —SO₂-cyclo-C₃H₅, —SO₂CH(CH₃)₂, —SO₂C(CH₃)₃, —SO₃H, —SO₃CH₃, —SO₃C₂H₅, —SO₃C₃H₇, —SO₃-cyclo-C₃H₅, —SO₃CH(CH₃)₂, —SO₃C(CH₃)₃, —NH—SO₂CH₃, —NH—SO₂C₂H₅, —NH—SO₂Ph, —NH—SO₂C₄H₆—CH₃, SO₂NH₂, —OCF₃, —OC₂F₅, —O—COOCH₃, —O—COOC₂H₅, —O—COOC₃H₇, —O—COO-cyclo-C₃H₅, —O—COOCH(CH₃)₂, —O—COOC(CH₃)₃, —NH—CO—NH₂, —NH—CO—NHCH₃, —NH—CO—NHC₂H₅, —NH—CO—NHC₃H₇, —NH—CO—NH-cyclo-C₃H₅, —NH—CO—NH[CH(CH₃)₂], —NH—CO—NH[C(CH₃)₃], —NH—CON(CH₃)₂, —NH—CON(C₂H₅)₂, —NH—CON(C₃H₇)₂, —NH—CO—N(cyclo-C₃H₅)₂, —NH—CO—N[CH(CH₃)₂]₂, —NH—CO—N[C(CH₃)₃]₂, —NH—CS—NH₂, —NH—CS—NHCH₃, —NH—CS—NHC₂H₅, —NH—CS—NHC₃H₇, —NH—CS—NH-cyclo-C₃H₅, —NH—CS—NH[CH(CH₃)₂], —NH—CS—NH[C(CH₃)₃], —NH—CS—N(CH₃)₂, —NH—CS—N(C₂H₅)₂, —NH—CS—N(C₃H₇)₂, —NH—CS—N(cyclo-C₃H₅)₂, —NH—CS—N[CH(CH₃)₂]₂, —NH—CS—N[C(CH₃)₃]₂, —NH—C(═NH)—NH₂, —NH—C(═NH)—NHCH₃, —NH—C(═NH)—NHC₂H₅, —NH—C(═NH)—NHC₃H₇, —NH—C(═NH)—NH-cyclo-C₃H₅, —NH—C(═NH)—NH[CH(CH₃)₂], —NH—C(═NH)—NH[C(CH₃)₃], —O—CO—NHCH₃, —NH—C(═NH)—N(CH₃)₂, —NH—C(═NH)—N(C₂H₅)₂, —O—CO—NHC₃H₇, —NH—C(═NH)—N(C₃H₇)₂, —NH—C(═NH)—N(cyclo-C₃H₅)₂, —O—CO—NH₂, —NH—C(═NH)—N[CH(CH₃)₂]₂, —NH—C(═NH)—N[C(CH₃)₃]₂, —O—CO—NHC₂H₅, —O—CO—NH-cyclo-C₃H₅, —O—CO—NH[CH(CH₃)₂], —O—CO—NH[C(CH₃)₃], —O—CO—N(CH₃)₂, —O—CO—N(C₂H₅)₂, —O—CO—N(C₃H₇)₂, —O—CO—N(cyclo-C₃H₅)₂, —O—CO—N[CH(CH₃)₂]₂, —O—CO—N[C(CH₃)₃]₂, —O—CO—OCH₃, —O—CO—OC₂H₅, —O—CO—OC₃H₇, —O—CO—O-cyclo-C₃H₅, —O—CO—OCH(CH₃)₂, —O—CO—OC(CH₃)₃, —CH₂F—CHF₂, —CF₃, —CH₂Cl, —CHCl₂, —CCl₃, —CH₂Br—CHBr₂, —CI₃, —CH₂—CH₂F—CH₂—CHF₂, —CH₂—CF₃, —CH₂—CH₂Cl, —CH₂—CHCl₂, —CH₂—CCl₃, —CH₂—CH₂Br—CH₂—CHBr₂, —CH₂—CBr₃, —CH₂—CI₃, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄H₉, —CH₂—CH(CH₃)—C₃H₇, —C(CH₃)₂—CH(CH₃)₂, —CH(CH₃)—CH₂—CH(CH₃)₂, —CH(CH₃)—CH(CH₃)—C₂H₅, —C(CH₃)₂—C₃H₇, —CH₂—CH(CH₃)—CH(CH₃)₂, —CH₂—C(CH₃)₂—C₂H₅, —C₂H₄—C(CH₃)₃, —CH(CH₃)—C(CH₃)₃, -Ph, —CH₂-Ph, —C₆H₄—CH₃, —CPh₃, —CH═CH₂, —CH₂—CH═CH₂, —C(CH₃)═CH₂, —CH═CH—CH₃, —C₂H₄—CH═CH₂, —CH═C(CH₃)₂, —CH₂—CH═CH—CH₃, —C≡CH, —C≡C—CH₃, —CH₂—C≡CH, 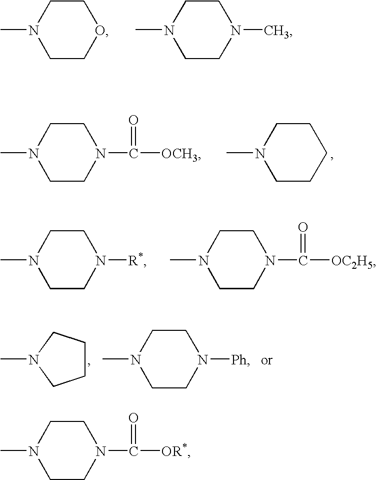
n and r are independently of each other integers from 0-8,
m, p, and q are independently of each other integers from 1-8,
and stereoisomeric and regioisomeric forms and pharmaceutically acceptable salts of the compounds of general formula (I).

2. A compound according to claim 1
wherein:
R¹ represents-CR²³(R²⁴)R²⁵, —CR²⁸(R²⁹)—CR²⁶(R²⁷)—CR²³(R²⁴)R²⁵, —CR²⁶(R²⁷)—CR²³(R²⁴)R²⁵, —(CH₂)ₙ—CR²⁸(R²⁹)—CR²⁶(R²⁷)—CR²³(R²⁴)R²⁵, —(CH₂)ₙ—CH═CH—(CH₂)ᵣ—CR²³(R²⁴)R²⁵, 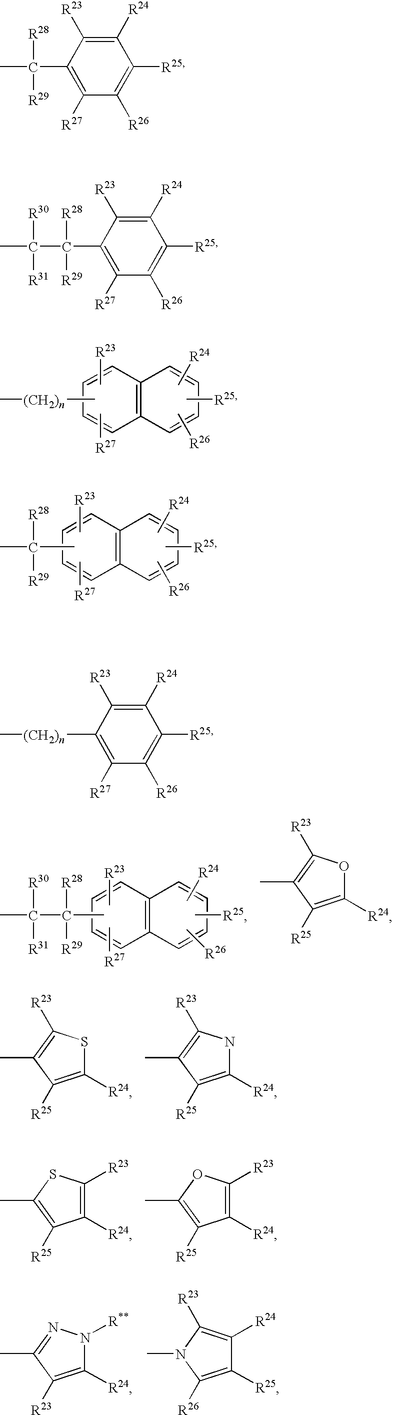 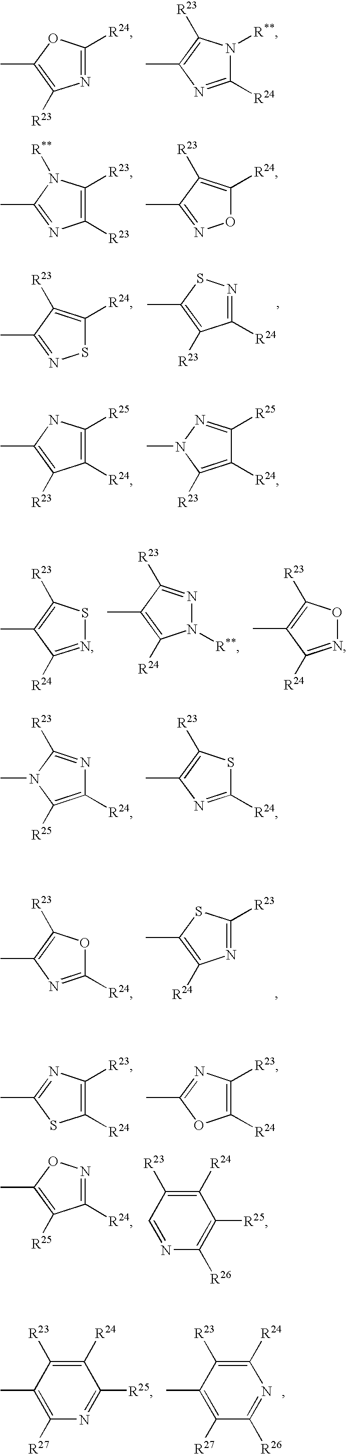 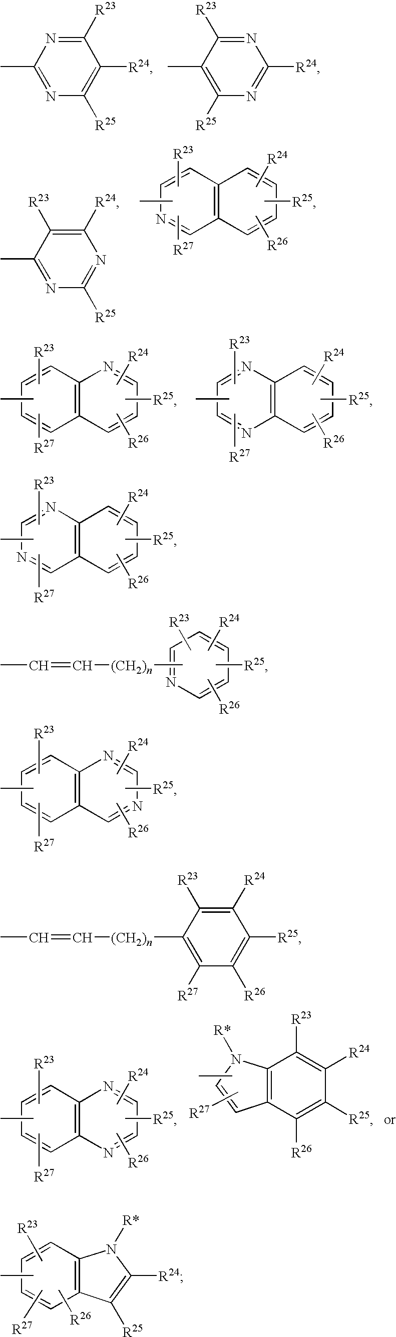
R², R* and R** represent independently of each other —H, —CH₃, —C₂H₅, —CH═CH₂, —C≡CH, —C₃H₇, -cyclo-C₃H₅, —CH(CH₃)₂, —CH₂—CH═CH₂, —C(CH₃)═CH₂, —CH═CH—CH₃, —C≡C—CH₃, —CH₂—C≡CH, —C₄H₉, -cyclo-C₄H₇, —CH₂—CH(CH₃)₂, —CH(CH₃)—C₂H₅, —C(CH₃)₃, —C₅H₁₁, —R′, —R″, —R′″, -cyclo-C₅H₉, —C₆H₁₃, -cyclo-C₆H₁₁, -Ph, —CH₂Ph, —C₆H₄—CH₃, —CR′(R″)R′″, —C₂(R′)₅, —CH₂—CR′(R″)R′″, —CHR′—CH(R″)R′″, —C(R′)R″—CH₂—R′″, —C₃(R′)₇, —C₂H₄—C(R′)₃, —CHO, —COCH₃, —COC₂H₅, —COC₃H₇, —COC₄H₉, —CO-cyclo-C₃H₅, —COCH(CH₃)₂, —COC(CH₃)₃, —COPh, —CO—CH₂Ph, —CO—C₆H₄—CH₃, —COOCH₃, —COOC₂H₅, —COOC₃H₇, —COOC₄H₉, —COO-cyclo-C₃H₅, —COOCH(CH₃)₂, —COOC(CH₃)₃, —COOPh, —COO—CH₂Ph, —COO—C₆H₄—CH₃;
R′, R″ and R′″ represent independently of each other —H, —F, —Cl, —Br, —I, —CN, —SO₃H, —CONH₂, —OH, —SH, —OCH₃, —OC₂H₅, —SCH₃, —SC₂H₅, —NH₂, —NO₂, —NH(CH₃), —N(CH₃)₂, —NH(C₂H₅), —N(C₂H₅)₂, —OCF₃, —CH₂F—CHF₂, —CF₃, —CH₂Cl, —CH₂Br, —CH₂I, —CH₂—CH₂F, —CH₂—CF₃, —CH₂—CH₂Cl, —CH₂—CH₂Br, —CH₂—CH₂I, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, -Ph, —CH₂-Ph, —C₆H₄—CH₃, —CPh₃, —CH═CH₂, —CH₂—CH═CH₂, —C(CH₃)═CH₂, —CH═CH—CH₃, —C₂H₄—CH═CH₂, —CH═C(CH₃)₂, —CH₂—CH═CH—CH₃, —C≡CH, —C≡C—CH₃, —CH₂—C≡CH, —CHO, —COCH₃, —COC₂H₅, —COC₃H₇, —CO-cyclo-C₃H₅, —COCH(CH₃)₂, —COC(CH₃)₃, —OOC—CH₃, —OOC—C₂H₅, —COOH, —COOCH₃, —COOC₂H₅, —COOC₃H₇, —COO-cyclo-C₃H₅, —COOCH(CH₃)₂, or —COOC(CH₃)₃;
R³ represents —R⁴, —CO—R⁴, —CO—CH(R⁵)—R⁴, —CH(R⁵)—R⁴, —CH(R⁵)—CH(R⁶)—R⁴, —CH(R⁵)—CO—R⁴, —CH(R⁵)—CH(R⁶)—CO—R⁴, —CH(R⁵)—O—CO—R⁴, —CH(R⁵)—CH(R⁶)—O—CO—R⁴, —CO—NH—R⁴, —CO—O—R⁴, —SO₂—R⁴, —CH(R⁵)—SO₂—R⁴, or —CH(R⁵)—CH(R⁶)—SO₂—R⁴;
R⁴ represents —CR¹⁶(R¹⁷)R¹⁸, —CR²¹(R²²)—CR¹⁹(R²⁰)—CR¹⁶(R¹⁷)R¹⁸, —CR¹⁹(R²⁰)—CR¹⁶(R¹⁷)R¹⁸, —(CH₂)ₙ—CR²¹(R²²)—CR¹⁹(R²⁰)—CR¹⁶(R¹⁷)R¹⁸, 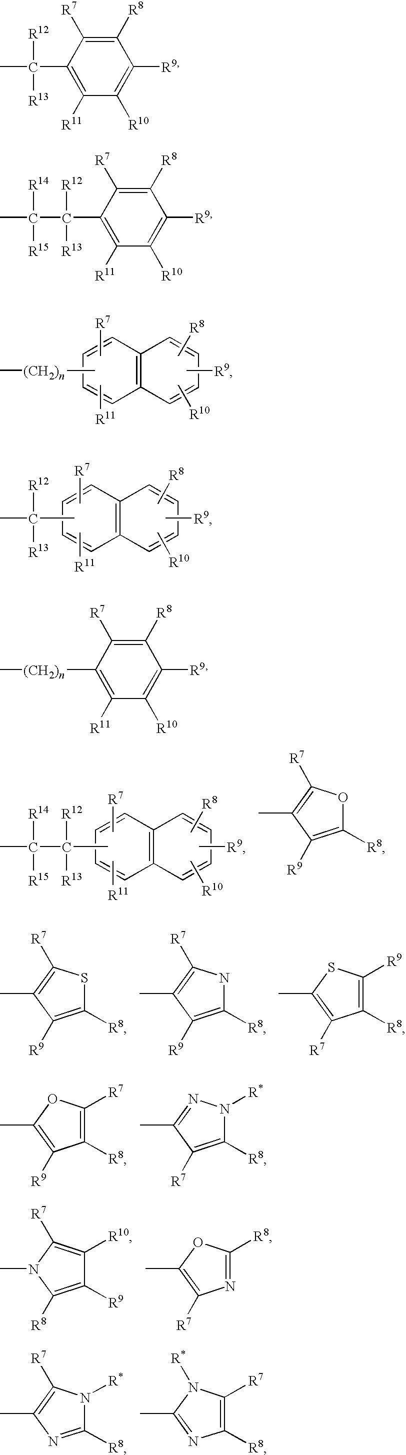 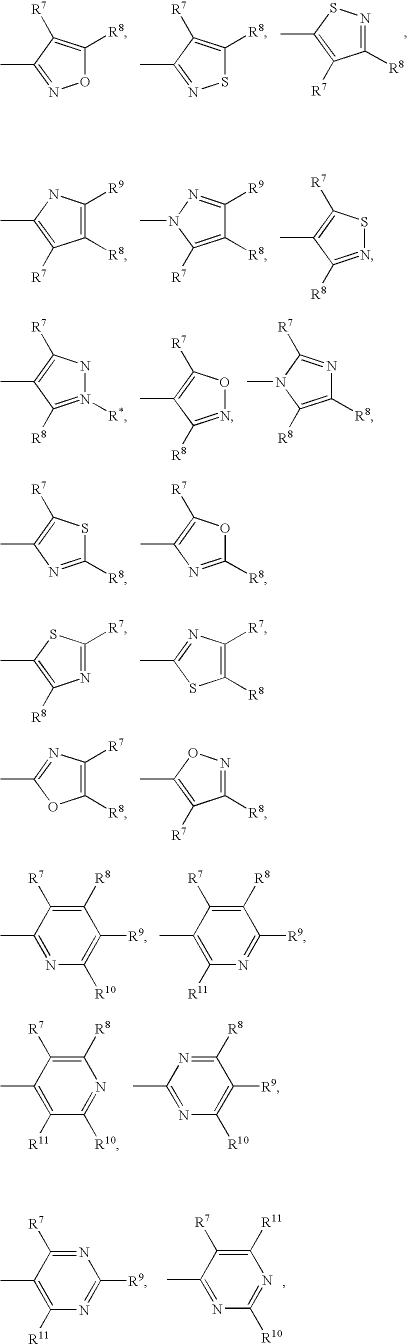 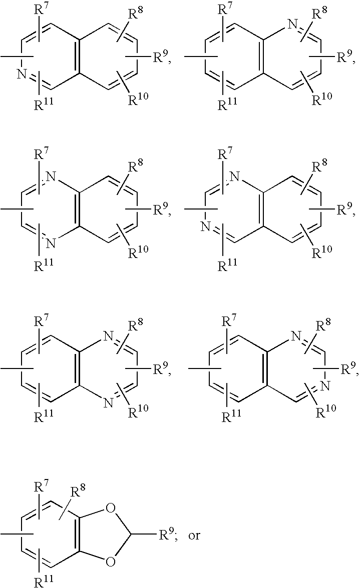
R² and R³ form together a heterocyclic ring such that the residue 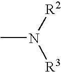
represents one of the following moieties: 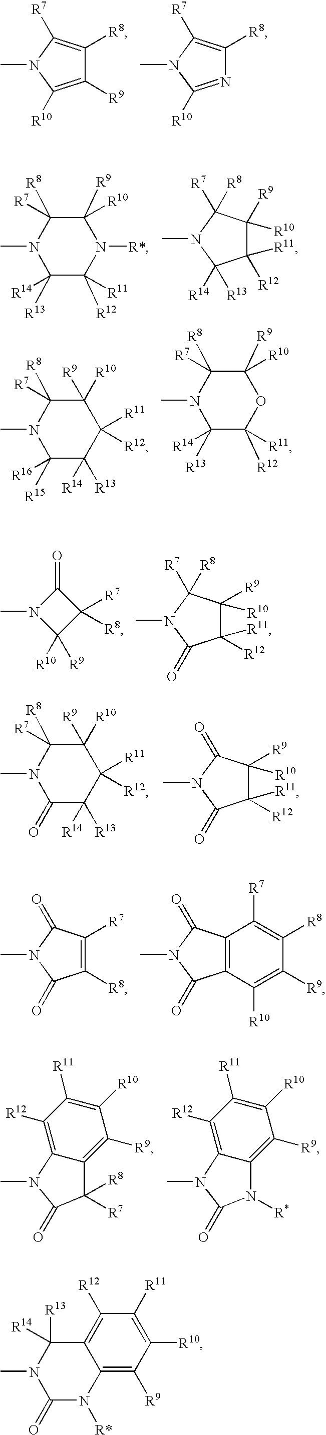 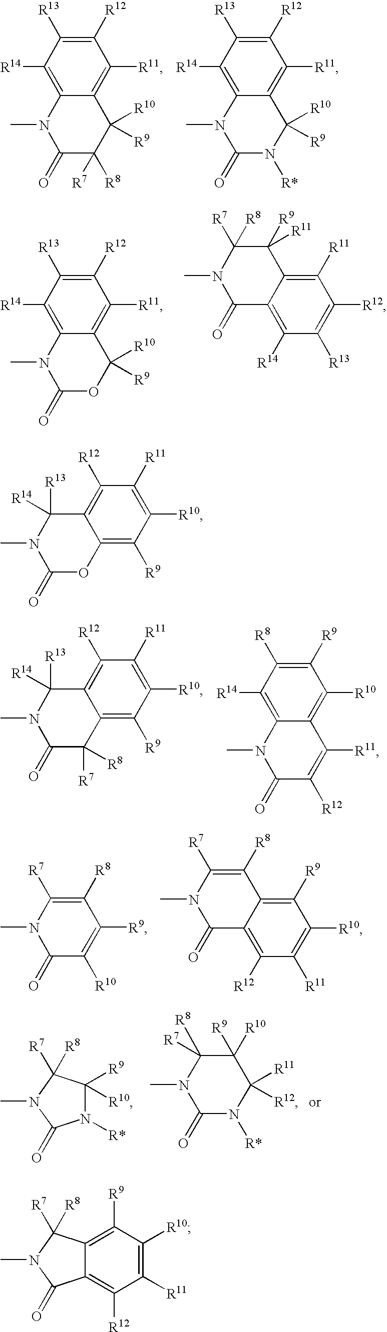
R⁵-R³¹ represent independently of each other
—(CH₂)ₘ—CR³²R³³R³⁴, —CR³⁵R³⁶R³⁷, —CR⁴³R⁴⁴—CR⁴⁵R⁴⁶—CR⁴⁷R⁴⁸R⁴⁹, —CR³⁸R³⁹—CR⁴⁰R⁴¹R⁴², —X—(CH₂)ₘ—CR³²R³³R³⁴, —X—CR³⁵R³⁶R³⁷, —X—CR³⁸R³⁹—CR⁴⁰R⁴¹R⁴², —X—CR⁴³R⁴⁴—CR⁴⁵R⁴⁶—CR⁴⁷R⁴⁸R⁴⁹, —CH₂R⁵⁰, —X—CH₂R⁵¹, —(CH₂)ₚ—R⁵³, or —X—(CH₂)_{q}—R⁵⁴;
X represents —CO—, —O—, —S—, —NR*—, —NH—CO—, —CO—NH—, —O—CO—, —CO—O—, —SO₂—, —SO—, —SO₂—O—, —NH—SO₂—, —O—SO₂—, —O—CO—O—, —O—CO—NH—, —NH—CO—O—, —NH—CO—NH—, —NH—CS—NH—, —NH—C(═NH)—NH—, —CF₂—, —C₂F₄—, or —C₃F₆—;
R⁵-R⁵⁴ represent independently of each other
—H, —OH, —OCH₃, —OC₂H₅, —OC₃H₇, —O-cyclo-C₃H₅, —OCH(CH₃)₂, —OC(CH₃)₃, —OPh, —OCH₂-Ph, —SH, —SCH₃, —SC₂H₅, —SC₃H₇, —S-cyclo-C₃H₅, —SCH(CH₃)₂, —SC(CH₃)₃, —NO₂, —F, —Cl, —Br, —I, —N₃, —CN, —CHO, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —COO-cyclo-C₃H₅, —COOCH(CH₃)₂, —COOC(CH₃)₃, —OOC—CH₃, —OOC—C₂H₅, —OOC—C₃H₇, —OOC-cyclo-C₃H₅, —OOC—CH(CH₃)₂, —OOC—C(CH₃)₃, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON[CH(CH₃)₂]₂, —NH₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(cyclo-C₃H₅)₂, —N[CH(CH₃)₂]₂, —N[C(CH₃)₃]₂, —SOCH₃, —SOC₂H₅, —SOC₃H₇, —SO-cyclo-C₃H₅, —SOCH(CH₃)₂, —SOC(CH₃)₃, —SO₂CH₃, —SO₂C₂H₅, —SO₂C₃H₇, —SO₂-cyclo-C₃H₅, —SO₂CH(CH₃)₂, —SO₂C(CH₃)₃, —SO₃H, —SO₃CH₃, —SO₃C₂H₅, —SO₃C₃H₇, —SO₃-cyclo-C₃H₅, —SO₃CH(CH₃)₂, —SO₃C(CH₃)₃, —NH—SO₂CH₃, —NH—SO₂C₂H₅, —NH—SO₂Ph, —NH—SO₂C₄H₆—CH₃, —OCF₃, —OC₂F₅, —O—COOCH₃, —O—COOC₂H₅, —O—COOC₃H₇, —O—COO-cyclo-C₃H₅, —O—COOCH(CH₃)₂, —O—COOC(CH₃)₃, —NH—CO—NH₂, —NH—CO—NHCH₃, —NH—CO—NHC₂H₅, —NH—CO—NHC₃H₇, —NH—CO—NH-cyclo-C₃H₅, —NH—CO—NH[CH(CH₃)₂], —NH—CO—NH[C(CH₃)₃], —NH—CO—N(CH₃)₂, —NH—CO—N(C₂H₅)₂, —NH—CO—N(C₃H₇)₂, —NH—CO—N(cyclo-C₃H₅)₂, —NH—CO—N[CH(CH₃)₂]₂, —NH—CO—N[C(CH₃)₃]₂, —NH—CS—NH₂, —NH—CS—NHCH₃, —NH—CS—NHC₂H₅, —NH—CS—NHC₃H₇, —NH—CS—NH-cyclo-C₃H₅, —NH—CS—NH[CH(CH₃)₂], —NH—CS—NH[C(CH₃)₃], —NH—CS—N(CH₃)₂, —NH—CS—N(C₂H₅)₂, —NH—CS—N(C₃H₇)₂, —NH—CS—N(cyclo-C₃H₅)₂, —NH—CS—N[CH(CH₃)₂]₂, —NH—CS—N[C(CH₃)₃]₂, —NH—C(═NH)—NH₂, —NH—C(═NH)—NHCH₃, —NH—C(═NH)—NHC₂H₅, —NH—C(═NH)—NHC₃H₇, —NH—C(═NH)—NH-cyclo-C₃H₅, —NH—C(═NH)—NH[CH(CH₃)₂], —NH—C(═NH)—NH[C(CH₃)₃], —O—CO—NHCH₃, —NH—C(═NH)—N(CH₃)₂, —NH—C(═NH)—N(C₂H₅)₂, —O—CO—NHC₃H₇, —NH—C(═NH)—N(C₃H₇)₂, —NH—C(═NH)—N(cyclo-C₃H₅)₂, —O—CO—NH₂, —NH—C(═NH)—N[CH(CH₃)₂]₂, —NH—C(═NH)—N[C(CH₃)₃]₂, —O—CO—NHC₂H₅, —O—CO—NH-cyclo-C₃H₅, —O—CO—NH[CH(CH₃)₂], —O—CO—NH[C(CH₃)₃], —O—CO—N(CH₃)₂, —O—CO—N(C₂H₅)₂, —O—CO—N(C₃H₇)₂, —O—CO—N(cyclo-C₃H₅)₂, —O—CO—N[CH(CH₃)₂]₂, —O—CO—N[C(CH₃)₃]₂, —O—CO—OCH₃, —O—CO—OC₂H₅, —O—CO—OC₃H₇, —O—CO—O-cyclo-C₃H₅, —O—CO—OCH(CH₃)₂, —O—CO—OC(CH₃)₃, —CH₂F—CHF₂, —CF₃, —CH₂Cl, —CHCl₂, —CCl₃, —CH₂Br—CHBr₂, —CI₃, —CH₂—CH₂F—CH₂—CHF₂, —CH₂—CF₃, —CH₂—CH₂Cl, —CH₂—CHCl₂, —CH₂—CCl₃, —CH₂—CH₂Br—CH₂—CHBr₂, —CH₂—CBr₃, —CH₂—CI₃, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄H₉, —CH₂—CH(CH₃)—C₃H₇, —C(CH₃)₂—CH(CH₃)₂, —CH(CH₃)—CH₂—CH(CH₃)₂, —CH(CH₃)—CH(CH₃)—C₂H₅, —C(CH₃)₂—C₃H₇, —CH₂—CH(CH₃)—CH(CH₃)₂, —CH₂—C(CH₃)₂—C₂H₅, —C₂H₄—C(CH₃)₃, —CH(CH₃)—C(CH₃)₃, -Ph, —CH₂-Ph, —C₆H₄—CH₃, —CPh₃, —CH═CH₂, —CH₂—CH═CH₂, —C(CH₃)═CH₂, —CH═CH—CH₃, —C₂H₄—CH═CH₂, —CH═C(CH₃)₂, —CH₂—CH═CH—CH₃, —C≡CH, —C≡C—CH₃, —CH₂—C≡CH, 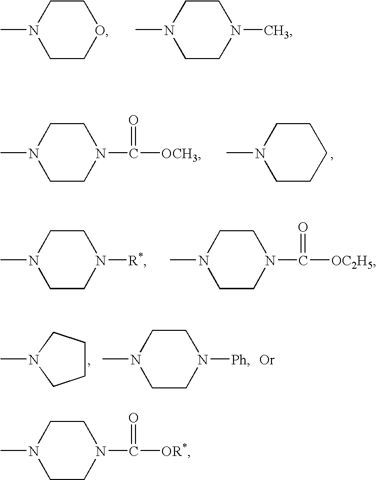
n and r are independently of each other integers from 0-8,
m, p, and q are independently of each other integers from 1-8,
and stereoisomeric and regioisomeric forms and pharmaceutically acceptable salts of the compounds of general formula (I).

3. A compound according to claim 1 having a structure of general formula (II): 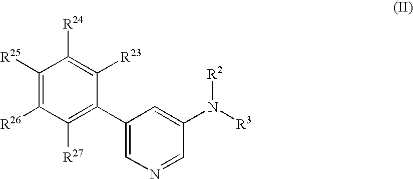
wherein
the substituents R², R³, R²³-R²⁷ are defined as in claim 1 or 2.

4. A compound according to claim 1 having a structure of general formula (III): 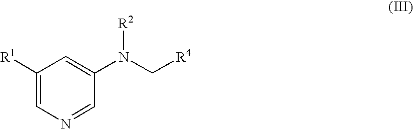
wherein
the substituents R¹, R², R⁴ are defined as in claim 1 or 2.

5. A compound according to claim 4 wherein
R³ is a group —CHR⁵—R⁴, where R⁵ is H;
R⁴ represents a group 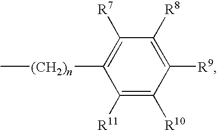
where n is zero;
R¹ represents a group 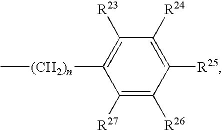
where n is zero;
R² is selected from
—H, —CH₃, —C₂H₅, —C₃H₇, -cyclo-C₃H₅, —CH(CH₃)₂, —C₄H₉, -cyclo-C₄H₇, —CH₂—CH(CH₃)₂, —CH(CH₃)—C₂H₅, —C(CH₃)₃, —C₅H₁₁, -cyclo-C₅H₉, —C₆H₁₃, -cyclo-C₆H₁₁, —Ph, —CH₂Ph, —C₆H₄—CH₃, —CHO, —COCH₃, —COC₂H₅, COC₃H₇, —COC₄H₉, —CO-cyclo-C₃H₅, —COCH(CH₃)₂, —COC(CH₃)₃, —COPh, —CO—CH₂Ph, —CO—C₆H₄—CH₃, —COOCH₃, —COOC₂H₅, —COOC₃H₇, —COOC₄H₉, —COO-cyclo-C₃H₅, —COOCH(CH₃)₂, —COOC(CH₃)₃, —COOPh, or —COO—CH₂Ph, —COO—C₆H₄—CH₃; and
R⁷-R¹¹ and R²³-R²⁷ are defined as in claim 1 or 2.

6. A compound according to claim 5, wherein
R² is —H, —CH₃, or —COOC₄H₉;
each substituent R⁷-R¹¹ and R²³-R²⁷ is independently selected from
—H, —F, —Cl, —Br, —OH, —CH₃, —C₂H₅, —C₃H₇, -cyclo-C₃H₅, —CH(CH₃)₂, —C₄H₉, -cyclo-C₄H₇, —CH₂—CH(CH₃)₂, —CH(CH₃)—C₂H₅, —C(CH₃)₃, —C₅H₁₁, —OCH₃, OCF₃, —NH₂, N(CH₃)₂, —N(C₂H₅)₂, —NO₂, —COOH, —COOCH₃, —CONH₂, —CN, SO₂CH₃, —NHSO₂CH₃, 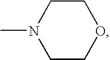
—CR³⁵R³⁶R³⁷, —X—(CH₂)ₘ—CR³²R³³R³⁴, or —X—CH₂—R⁵¹;
X is —NHCO— or —CONH—;
R⁵¹ is H;
each of the substituents R³³-R³⁶ is H;
R³² is OH or N(CH₃)₂;
R³⁷ is OH; and
m is 0 or 1.

7. A compound according to claim 6, wherein
R⁷ is —CH₃, —C₂H₅, —C₃H₇, -cyclo-C₃H₅, —CH(CH₃)₂, —C₄H₉, -cyclo-C₄H₇, —CH₂—CH(CH₃)₂, —CH(CH₃)—C₂H₅, —C(CH₃)₃, —C₅H₁₁, —OCH₃, —OH, —F, —Cl, or —Br.

8. A compound according to claim 7, wherein
R⁷ is —CH₃, —C₂H₅, —C₃H₇, -cyclo-C₃H₅, —CH(CH₃)₂, —C₄H₉, -cyclo-C₄H₇, —CH₂—CH(CH₃)₂, —CH(CH₃)—C₂H₅, —C(CH₃)₃, —C₅H₁₁, —OCH₃, —F, —Cl, or —Br.

9. A compound according to claim 7, wherein
R⁷ is —CH₃, —OCH₃, —OH or —Cl;
R⁸ is —OH, —NH₂, —OCH₃, —CONH₂, or —SO₂NH₂;
R⁹-R¹¹ are each H;
R²³ is H;
R²⁴ is H, —OH, —NH₂, —COOH, —CONH₂, or —SO₂NH₂;
R²⁵ is H, —Cl, —OH, —OCH₃, —OCF₃, —CH₃, —CF₃, —NH₂, —COOH, —CONH₂, COOCH₃, —CN, —SO₂CH₃, or —SO₂NH₂; and
R²⁶-R²⁷ are each H.

10. A compound according to claim 1
wherein
R⁷ is H; and
R⁸ is selected from —CH₃, —C₂H₅, —C₃H₇, —CH(CH₃)₂, —C₄H₉, cyclo-C₄H₇, —CH₂—CH(CH₃)₂, —CH(CH₃)—C₂H₅, —C(CH₃)₃, —C₅H₁₁, —OCF₃, NO₂, —COOH, —COOCH₃, —CONH₂, —CN, —SO₂CH₃, —NH₂, NHSO₂CH₃, 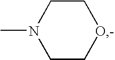
CR³⁵R³⁶R³⁷, —X—(CH₂)ₘ—CR³²R³³R³⁴, or —X—CH₂—R⁵¹;
X is —NHCO— or —CONH—;
R⁵¹ is H;
each of the substituents R³³-R³⁶ is H;
R³² is OH or N(CH₃)₂;
R³⁷ is OH; and
m is 0 or 1.

11. A compound according to claim 10 wherein
R⁸ is —COOH, —COOCH₃, —CONH₂, or —CN.

12. A compound according to claim 1, wherein
R⁷ is H; and
R⁹ is selected from —OH, —CH₃, —C₂H₅, —C₃H₇, —CH(CH₃)₂, —C₄H₉, cyclo-C₄H₇, —CH₂—CH(CH₃)₂, —CH(CH₃)—C₂H₅, —C(CH₃)₃, —C₅H₁₁, —OCF₃, NO₂, —COOH, —COOCH₃, —CONH₂, —CN, —SO₂CH₃, —NH₂, NHSO₂CH₃, 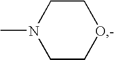
CR³⁵R³⁶R³⁷, —X—(CH₂)ₘ—CR³²R³³R³⁴, or —X—CH₂—R⁵¹;
X is —NHCO— or —CONH—;
R⁵¹ is H;
each of the substituents R³³-R³⁶ is H;
R³² is OH or N(CH₃)₂;
R³⁷ is OH; and
m is 0 or 1.

13. A compound according to claim 1, wherein
R²³ is H, —F, —Cl, —Br, —OH, —CH₃, —C₂H₅, —C₃H₇, -cyclo-C₃H₅, —CH(CH₃)₂, —C₄H₉, -cyclo-C₄H₇, —CH₂—CH(CH₃)₂, —CH(CH₃)—C₂H₅, —C(CH₃)₃, —C₅H₁₁, OCF₃, NH₂, —N(CH₃)₂, —N(C₂H₅)₂, —NO₂, —COOH, —COOCH₃, —CONH₂, —CN, SO₂CH₃, NHSO₂CH₃, 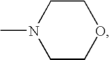
or —X—(CH₂)ₘ—CR³²R³³R³⁴, or —X—CH₂—R⁵¹;
X is —NHCO— or —CONH—;
R⁵¹ is H;
each of the substituents R³³-R³⁴ is H;
R³² is OH or N(CH₃)₂; and
m is 0 or 1.

14. A compound according to claim 1, wherein
R²⁵ is H, —F, —Cl, —Br, —CH₃, —C₂H₅, —C₃H₇, -cyclo-C₃H₅, —CH(CH₃)₂, —C₄H₉, -cyclo-C₄H₇, —CH₂—CH(CH₃)₂, —CH(CH₃)—C₂H₅, —C(CH₃)₃, —C₅H₁₁, OCF₃, NH₂, —N(CH₃)₂, —N(C₂H₅)₂, —NO₂, —COOH, —COOCH₃, —CONH₂, —CN, NHSO₂CH₃, —X—(CH₂)ₘ—CR³²R³³R³⁴, or —X—CH₂—R⁵¹;
X is —NHCO— or —CONH—;
R⁵¹ is H;
each of the substituents R³³-R³⁴ is H;
R³² is OH or N(CH₃)₂; and
m is 0 or 1.

15. A compound according to claim 1, wherein R⁷ is not hydrogen.

16. A compound according to claim 1, wherein R²³ is not hydrogen.

17. A compound according to claim 1, wherein R⁸ is not —F, Cl, —Br, —NH₂, —NO₂, —OH, —OCH₃, or —OCF₃.

18. A compound according to claim 1, wherein R⁹ is not —F, Cl, —Br, —NH₂, —N(CH₃)₂, —NO₂, —OH, or —OCH₃.

19. A compound according to claim 1, wherein the compound is selected from:
Compound 1 (3,4-Difluoro-benzyl)-(5-thiophen-3-yl-pyridin-3-yl)-amine,
Compound 2 N-(3-{5-[(Benzo[1,3]dioxol-5-ylmethyl)-amino]-pyridin-3-yl}-phenyl)-methanesulfonamide,
Compound 3 3-{5-[(Benzo[1,3]dioxol-5-ylmethyl)-amino]-pyridin-3-yl}-phenol,
Compound 4 [5-(4-Morpholin-4-yl-phenyl)-pyridin-3-yl]-pyridin-3-ylmethyl-amine,
Compound 5 N-(2-Dimethylamino-ethyl)-3-{5-[(pyridin-3-ylmethyl)-amino]-pyridin-3-yl}-benzamide,
Compound 6 (3,4-Difluoro-benzyl)-(5-pyrimidin-5-yl-pyridin-3-yl)-amine,
Compound 7 (3-Chloro-phenyl)-(5-phenethyl-pyridin-3-yl)-amine,
Compound 8 N-(2-Dimethylamino-ethyl)-3-[5-(4-methoxy-phenylamino)-pyridin-3-yl]-benzamide,
Compound 9 4-(5-Phenylamino-pyridin-3-yl)-phenol,
Compound 10 [5-(4-Methanesulfonyl-phenyl)-pyridin-3-yl]-phenyl-amine,
Compound 11 (4-Chloro-benzyl)-(5′-methoxy-[3,3′]bipyridinyl-5-yl)-amine,
Compound 12 3-[5-(4-Chloro-benzylamino)-pyridin-3-yl]-phenol,
Compound 13 {4-[5-(3,4-Difluoro-benzylamino)-pyridin-3-yl]-phenyl}-methanol,
Compound 14 N-(2-Dimethylamino-ethyl)-3-{5-[(furan-3-ylmethyl)-amino]-pyridin-3-yl}-benzamide,
Compound 15 [5-(4-Methanesulfonyl-phenyl)-pyridin-3-yl]-pyridin-3-ylmethyl-amine,
Compound 16 (3-Bromo-phenyl)-[5-(4-dimethylamino-phenyl)-pyridin-3-yl]-amine,
Compound 17 (6′-Methoxy-[3,3′]bipyridinyl-5-yl)-phenyl-amine,
Compound 18 (3-Chloro-4-fluoro-phenyl)-[5-(4-dimethylamino-phenyl)-pyridin-3-yl]-amine,
Compound 19 (4-Diethylamino-benzyl)-[5-(2-methoxy-phenyl)-pyridin-3-yl]-amine,
Compound 20 Quinolin-3-ylmethyl-(5-quinolin-3-yl-pyridin-3-yl)-amine,
Compound 21 {4-[5-(4-Chloro-benzylamino)-pyridin-3-yl]-phenyl}-methanol,
Compound 22 [3,4′]Bipyridinyl-5-yl-(3,4-dimethoxy-benzyl)-amine,
Compound 23 (3-Bromo-phenyl)-(5-quinolin-8-yl-pyridin-3-yl)-amine,
Compound 24 N-(2-Dimethylamino-ethyl)-3-[5-(3-nitro-phenylamino)-pyridin-3-yl]-benzamide,
Compound 25 Furan-3-ylmethyl-(5′-methoxy-[3,3′]bipyridinyl-5-yl)-amine,
Compound 26 N-(2-Dimethylamino-ethyl)-4-[5-(3-nitro-phenylamino)-pyridin-3-yl]-benzamide,
Compound 27 [3,3′]Bipyridinyl-5-yl-quinolin-3-ylmethyl-amine,
Compound 28 [3,3′]Bipyridinyl-5-yl-(3,4-dichloro-benzyl)-amine,
Compound 29 4-[5-(4-Chloro-benzylamino)-pyridin-3-yl]-phenol,
Compound 30 3-{5-[(Naphthalen-2-ylmethyl)-amino]-pyridin-3-yl}-phenol,
Compound 31 N-{3-[5-(3,4-Difluoro-benzylamino)-pyridin-3-yl]-phenyl}-methanesulfonamide,
Compound 32 [3,3′]Bipyridinyl-5-yl-furan-3-ylmethyl-amine,
Compound 33 4-[5-(3,4-Dichloro-benzylamino)-pyridin-3-yl]-phenol,
Compound 34 4-[5-(3,4-Dimethoxy-benzylamino)-pyridin-3-yl]-phenol,
Compound 35 (3,4-Difluoro-benzyl)-(6′-methoxy-[3,3′]bipyridinyl-5-yl)-amine,
Compound 36 [((E)-5-Hex-1-enyl)-pyridin-3-yl]-(3,4,5-trimethoxy-phenyl)-amine,
Compound 37 3-[5-(Naphthalen-2-ylamino)-pyridin-3-yl]-phenol,
Compound 38 (4-Chloro-phenyl)-(5-pyrimidin-5-yl-pyridin-3-yl)-amine,
Compound 39 N-{3-[5-(3,4-Dichloro-benzylamino)-pyridin-3-yl]-phenyl}-methanesulfonamide,
Compound 40 3-[5-(3,4-Dimethoxy-benzylamino)-pyridin-3-yl]-benzamide,
Compound 41 5-Bromo-2-{5-[(furan-3-ylmethyl)-amino]-pyridin-3-yl}-indole-1-carboxylic acid tert-butyl ester,
Compound 42 [3,3′]Bipyridinyl-5-yl-pyridin-3-ylmethyl-amine,
Compound 43 {2-[5-(3-Chloro-phenylamino)-pyridin-3-yl]-phenyl}-methanol,
Compound 44 3-(5-Phenylamino-pyridin-3-yl)-benzamide,
Compound 45 (4-Chloro-phenyl)-(5′-methoxy-[3,3′]bipyridinyl-5-yl)-amine,
Compound 46 4-[5-(4-Chloro-phenylamino)-pyridin-3-yl]-N-(2-dimethylamino-ethyl)-benzamide,
Compound 47 {4-[5-(3-Nitro-phenylamino)-pyridin-3-yl]-phenyl}-methanol,
Compound 48 (5′-Methoxy-[3,3′]bipyridinyl-5-yl)-naphthalen-2-ylmethyl-amine,
Compound 49 3-[5-(4-Chloro-benzylamino)-pyridin-3-yl]-N-(2-dimethylamino-ethyl)-benzamide,
Compound 50 [3,3′]Bipyridinyl-5-yl-(3,4-difluoro-benzyl)-amine,
Compound 51 (3,4-Difluoro-benzyl)-(5′-methoxy-[3,3′]bipyridinyl-5-yl)-amine,
Compound 52 3-[5-(4-Trifluoromethoxy-phenylamino)-pyridin-3-yl]-phenol,
Compound 53 [5-(3,4-Dimethoxy-phenyl)-pyridin-3-yl]-naphthalen-2-yl-amine,
Compound 54 N-(2-Dimethylamino-ethyl)-3-[5-(naphthalen-2-ylamino)-pyridin-3-yl]-benzamide,
Compound 55 (4-Chloro-phenyl)-[5-(3-trifluoromethoxy-phenyl)-pyridin-3-yl]-amine,
Compound 56 (4-Chloro-phenyl)-[5-(2-methoxy-phenyl)-pyridin-3-yl]-amine,
Compound 57 3-{5-[(Quinolin-3-ylmethyl)-amino]-pyridin-3-yl}-benzamide,
Compound 58 4-[5-(2,4-Dimethoxy-pyrimidin-5-yl)-pyridin-3-ylamino]-benzonitrile,
Compound 59 3-[5-(3,4-Dichloro-benzylamino)-pyridin-3-yl]-phenol,
Compound 60 [5-(2,4-Dimethoxy-pyrimidin-5-yl)-pyridin-3-yl]-(4-isopropyl-phenyl)-amine,
Compound 61 (5′-Methoxy-[3,3′]bipyridinyl-5-yl)-(3-nitro-phenyl)-amine,
Compound 62 3-[5-(3-Chloro-4-fluoro-phenylamino)-pyridin-3-yl]-benzamide,
Compound 63 (3-Chloro-4-fluoro-phenyl)-(5-quinolin-3-yl-pyridin-3-yl)-amine,
Compound 64 {2-[5-(3-Chloro-4-fluoro-phenylamino)-pyridin-3-yl]-phenyl}-methanol,
Compound 65 [5-(2,4-Dimethoxy-pyrimidin-5-yl)-pyridin-3-yl]-naphthalen-2-yl-amine,
Compound 66 (4-Chloro-phenyl)-[5-(4-methanesulfonyl-phenyl)-pyridin-3-yl]-amine,
Compound 67 N-{3-[5-(4-Chloro-phenylamino)-pyridin-3-yl]-phenyl}-acetamide,
Compound 68 {2-[5-(4-Chloro-phenylamino)-pyridin-3-yl]-phenyl}-methanol,
Compound 69 3-[5-(4-Chloro-phenylamino)-pyridin-3-yl]-N-(2-dimethylamino-ethyl)-benzamide,
Compound 70 N-(3-{5-[(Quinolin-3-ylmethyl)-amino]-pyridin-3-yl}-phenyl)-acetamide,
Compound 71 (3,4-Difluoro-benzyl)-[5-(3,4-dimethoxy-phenyl)-pyridin-3-yl]amine,
Compound 72 [5-(2,4-Dimethoxy-pyrimidin-5-yl)-pyridin-3-yl]-furan-3-ylmethyl-amine,
Compound 73 {4-[5-(4-Chloro-phenylamino)-pyridin-3-yl]-phenyl}-methanol,
Compound 74 Furan-3-ylmethyl-(6′-methoxy-[3,3′]bipyridinyl-5-yl)-amine,
Compound 75 N-(2-Hydroxy-ethyl)-3-{5-[(pyridin-3-ylmethyl)-amino]-pyridin-3-yl}-benzamide,
Compound 76 Pyridin-3-ylmethyl-(5-thiophen-3-yl-pyridin-3-yl)-amine,
Compound 77 {3-[5-(4-Trifluoromethoxy-phenylamino)-pyridin-3-yl]-phenyl}-methanol,
Compound 78 {3-[5-(Naphthalen-2-ylamino)-pyridin-3-yl]-phenyl}-methanol,
Compound 79 (3,4-Dichloro-benzyl)-(5′-methoxy-[3,3′]bipyridinyl-5-yl)-amine,
Compound 80 (3-Nitro-phenyl)-(5-thiophen-3-yl-pyridin-3-yl)-amine,
Compound 81 (3-Chloro-4-fluoro-phenyl)-[5-(2-methoxy-phenyl)-pyridin-3-yl]-amine,
Compound 82 3-[5-(4-Chloro-benzylamino)-pyridin-3-yl]-benzamide,
Compound 83 (3-Chloro-4-fluoro-phenyl)-[5-(2,4-dimethoxy-pyrimidin-5-yl)-pyridin-3-yl]-amine,
Compound 84 N-(2-Dimethylamino-ethyl)-4-[5-(naphthalen-2-ylamino)-pyridin-3-yl]-benzamide,
Compound 85 (5′-Methoxy-[3,3′]bipyridinyl-5-yl)-(4-trifluoromethoxy-phenyl)-amine,
Compound 86 (4-Chloro-phenyl)-(6′-methoxy-[3,3′]bipyridinyl-5-yl)-amine,
Compound 87 [3,4′]Bipyridinyl-5-yl-naphthalen-2-ylmethyl-amine,
Compound 88 [3,4′]Bipyridinyl-5-yl-(4-chloro-benzyl)-amine,
Compound 89 Benzo[1,3]dioxol-5-ylmethyl-[5-(3,4-dimethoxy-phenyl)-pyridin-3-yl]-amine,
Compound 90 (3-{5-[(Furan-3-ylmethyl)-amino]-pyridin-3-yl}-phenyl)-methanol,
Compound 91 N-{3-[5-(3,4-Dimethoxy-benzylamino)-pyridin-3-yl]-phenyl}-acetamide,
Compound 92 3-{5-[(Pyridin-3-ylmethyl)-amino]-pyridin-3-yl}-phenol,
Compound 93 [5-(3,4-Dimethoxy-phenyl)-pyridin-3-yl]-phenyl-amine,
Compound 94 (3-Chloro-4-fluoro-phenyl)-(5-pyrimidin-5-yl-pyridin-3-yl)-amine,
Compound 95 N-{3-[5-(4-Chloro-benzylamino)-pyridin-3-yl]-phenyl}-methanesulfonamide,
Compound 96 (3,4-Dimethoxy-benzyl)-[5-(3,4-dimethoxy-phenyl)-pyridin-3-yl]-amine,
Compound 97 3-{5-[(Furan-3-ylmethyl)-amino]-pyridin-3-yl}-N-(2-hydroxy-ethyl)-benzamide,
Compound 98 (5-Benzo[1,3]dioxol-5-yl-pyridin-3-yl)-furan-3-ylmethyl-amine,
Compound 99 (3-Bromo-phenyl)-[5-(4-morpholin-4-yl-phenyl)-pyridin-3-yl]-amine,
Compound 100 [3,3′]Bipyridinyl-5-yl-(3-bromo-phenyl)-amine,
Compound 101 4-(5-Thiophen-3-yl-pyridin-3-ylamino)-benzonitrile,
Compound 102 N-(3-{5-[(Furan-3-ylmethyl)-amino]-pyridin-3-yl}-phenyl)-methanesulfonamide,
Compound 103 (3-Bromo-phenyl)-[5-(2-methoxy-phenyl)-pyridin-3-yl]-amine,
Compound 104 N-(2-Hydroxy-ethyl)-3-[5-(4-methoxy-phenylamino)-pyridin-3-yl]benzamide,
Compound 105 3-[5-(4-Chloro-phenylamino)-pyridin-3-yl]-benzamide,
Compound 106 N-(3-{5-[(Naphthalen-2-ylmethyl)-amino]-pyridin-3-yl}-phenyl)-acetamide,
Compound 107 Benzo[1,3]dioxol-5-ylmethyl-[3,4′]bipyridinyl-5-yl-amine,
Compound 108 5-Bromo-2-[5-(3-hydroxy-benzylamino)-pyridin-3-yl]-indole-1-carboxylic acid tert-butyl ester,
Compound 109 Furan-3-ylmethyl-(5-thiophen-3-yl-pyridin-3-yl)-amine,
Compound 110 [3,4′]Bipyridinyl-5-yl-furan-3-ylmethyl-amine,
Compound 111 [5-(3,4-Dimethoxy-phenyl)-pyridin-3-yl]-quinolin-3-ylmethyl-amine,
Compound 112 N-{3-[5-(4-Chloro-benzylamino)-pyridin-3-yl]-phenyl}-acetamide,
Compound 113 3-[5-(3,4-Difluoro-benzylamino)-pyridin-3-yl]-phenol,
Compound 114 (3-Chloro-4-fluoro-phenyl)-(6′-methoxy-[3,3′]bipyridinyl-5-yl)-amine,
Compound 115 (5′-Methoxy-[3,3′]bipyridinyl-5-yl)-(4-methoxy-phenyl)-amine,
Compound 116 (4-{5-[(Furan-3-ylmethyl)-amino]-pyridin-3-yl}-phenyl)-methanol,
Compound 117 3-[5-(3-Chloro-phenylamino)-pyridin-3-yl]-benzamide,
Compound 118 N-{3-[5-(4-Isopropyl-phenylamino)-pyridin-3-yl]-phenyl}-acetamide,
Compound 119 Phenyl-(5-quinolin-3-yl-pyridin-3-yl)-amine,
Compound 120 4-(5-Pyrimidin-5-yl-pyridin-3-ylamino)-benzonitrile,
Compound 121 (5′-Methoxy-[3,3′]bipyridinyl-5-yl)-(3,4,5-trimethoxy-phenyl)-amine,
Compound 122 (3-Chloro-phenyl)-(5-quinolin-8-yl-pyridin-3-yl)-amine,
Compound 123 [3,4′]Bipyridinyl-5-yl-(3,4-dichloro-benzyl)-amine,
Compound 124 3-[5-(3,4-Difluoro-benzylamino)-pyridin-3-yl]-benzamide,
Compound 125 3-{5-[(Benzo[1,3]dioxol-5-ylmethyl)-amino]-pyridin-3-yl}-benzamide,
Compound 126 4-{5-[(Benzo[1,3]dioxol-5-ylmethyl)-amino]-pyridin-3-yl}-phenol,
Compound 127 4-[5-(3,4-Difluoro-benzylamino)-pyridin-3-yl]-phenol,
Compound 128 (5′-Methoxy-[3,3′]bipyridinyl-5-yl)-pyridin-3-ylmethyl-amine,
Compound 129 (3-Bromo-phenyl)-(5′-methoxy-[3,3′]bipyridinyl-5-yl)-amine,
Compound 130 N-(3-{5-[(Benzo[1,3]dioxol-5-ylmethyl)-amino]-pyridin-3-yl}-phenyl)-acetamide,
Compound 131 [5-(3,4-Dimethoxy-phenyl)-pyridin-3-yl]-(4-isopropyl-phenyl)-amine,
Compound 132 N-(2-Hydroxy-ethyl)-3-[5-(3-nitro-phenylamino)-pyridin-3-yl]-benzamide,
Compound 133 [5-(2,4-Dimethoxy-pyrimidin-5-yl)-pyridin-3-yl]-phenyl-amine,
Compound 134 5-Bromo-2-{5-[(pyridin-3-ylmethyl)-amino]-pyridin-3-yl}-indole-1-carboxylic acid tert-butyl ester,
Compound 135 3-{5-[(Pyridin-3-ylmethyl)-amino]-pyridin-3-yl}-benzamide,
Compound 136 (3-Bromo-phenyl)-[5-(2,4-dimethoxy-pyrimidin-5-yl)-pyridin-3-yl]-amine,
Compound 137 [3-(5-Phenylamino-pyridin-3-yl)-phenyl]-methanol,
Compound 138 N-{3-[5-(4-Methoxy-phenylamino)-pyridin-3-yl]-phenyl}-methanesulfonamide,
Compound 139 [3,4′]Bipyridinyl-5-yl-(3,4-difluoro-benzyl)-amine,
Compound 140 N-{3-[5-(3-Nitro-phenylamino)-pyridin-3-yl]-phenyl}-methanesulfonamide,
Compound 141 3-{5-[(Furan-3-ylmethyl)-amino]-pyridin-3-yl}-phenol,
Compound 142 3-[5-(4-Isopropyl-phenylamino)-pyridin-3-yl]-phenol,
Compound 143 4-[((E)-5-Hex-1-enyl)-pyridin-3-ylamino]-benzonitrile,
Compound 144 N-{3-[5-(Naphthalen-2-ylamino)-pyridin-3-yl]-phenyl}-acetamide,
Compound 145 N-{3-[5-(3,4-Difluoro-benzylamino)-pyridin-3-yl]-phenyl}-acetamide,
Compound 146 (3-Bromo-phenyl)-(5-quinolin-3-yl-pyridin-3-yl)-amine,
Compound 147 {4-[5-(3-Bromo-phenylamino)-pyridin-3-yl]-phenyl}-methanol,
Compound 148 4-[5-(4-Isopropyl-phenylamino)-pyridin-3-yl]-phenol,
Compound 149 3-[5-(4-Cyano-phenylamino)-pyridin-3-yl]-N-(2-dimethylamino-ethyl)-benzamide,
Compound 150 4-(6′-Methoxy-[3,3′]bipyridinyl-5-ylamino)-benzonitrile,
Compound 151 3-[5-(Naphthalen-2-ylamino)-pyridin-3-yl]-benzamide,
Compound 152 3-[5-(4-Methoxy-phenylamino)-pyridin-3-yl]-benzamide,
Compound 153 [3,4′]Bipyridinyl-5-yl-(3-bromo-phenyl)-amine,
Compound 154 (4-Chloro-benzyl)-[5-(3,4-dimethoxy-phenyl)-pyridin-3-yl]-amine,
Compound 155 3-{[5-(2-Hydroxymethyl-phenyl)-pyridin-3-ylamino]-methyl}-phenol,
Compound 156 [3,3′]Bipyridinyl-5-yl-(3-nitro-phenyl)-amine,
Compound 157 N-{3-[5-(3-Nitro-phenylamino)-pyridin-3-yl]-phenyl}-acetamide,
Compound 158 N-(2-Hydroxy-ethyl)-3-(5-phenylamino-pyridin-3-yl)-benzamide,
Compound 159 3-([3,3′]Bipyridinyl-5-ylaminomethyl)-phenol,
Compound 160 3-{5-[(Furan-3-ylmethyl)-amino]-pyridin-3-yl}-benzamide,
Compound 161 (6′-Methoxy-[3,3′]bipyridinyl-5-yl)-(3,4,5-trimethoxy-phenyl)-amine,
Compound 162 (3-Chloro-4-fluoro-phenyl)-[5-(4-methanesulfonyl-phenyl)-pyridin-3-yl]-amine,
Compound 163 N-(3-{5-[(Furan-3-ylmethyl)-amino]-pyridin-3-yl}-phenyl)-acetamide,
Compound 164 [3,4′]Bipyridinyl-5-yl-(3-nitro-phenyl)-amine,
Compound 165 {3-[5-(4-Chloro-phenylamino)-pyridin-3-yl]-phenyl}-methanol,
Compound 166 [3,4′]Bipyridinyl-5-yl-(3-chloro-phenyl)-amine,
Compound 167 3-[5-(3-Nitro-phenylamino)-pyridin-3-yl]-benzamide,
Compound 168 3-[5-(4-Chloro-phenylamino)-pyridin-3-yl]-phenol,
Compound 169 N-{3-[5-(4-Chloro-phenylamino)-pyridin-3-yl]-phenyl}-methanesulfonamide,
Compound 170 3-[5-(3-Bromo-phenylamino)-pyridin-3-yl]-benzamide,
Compound 171 4-(5′-Methoxy-[3,3′]bipyridinyl-5-ylamino)-benzonitrile,
Compound 172 3-{[5-(2-Methoxy-phenyl)-pyridin-3-ylamino]-methyl}-phenol,
Compound 173 4-[5-(3-Chloro-4-fluoro-phenylamino)-pyridin-3-yl]-N-(2-dimethylamino-ethyl)-benzamide,
Compound 174 3-[5-(3-Nitro-phenylamino)-pyridin-3-yl]-phenol,
Compound 175 {4-[5-(3-Chloro-4-fluoro-phenylamino)-pyridin-3-yl]-phenyl}-methanol,
Compound 176 4-{5-[(Furan-3-ylmethyl)-amino]-pyridin-3-yl}-phenol,
Compound 177 [5-(3,4-Dimethoxy-phenyl)-pyridin-3-yl]-furan-3-ylmethyl-amine,
Compound 178 3-[5-(3-Bromo-phenylamino)-pyridin-3-yl]-N-(2-hydroxy-ethyl)-benzamide,
Compound 179 N-(2-Dimethylamino-ethyl)-3-[5-(3,4,5-trimethoxy-phenylamino)-pyridin-3-yl]-benzamide,
Compound 180 (3-Chloro-4-fluoro-phenyl)-(5′-methoxy-[3,3′]bipyridinyl-5-yl)-amine,
Compound 181 3-{[5-(2,4-Dimethoxy-pyrimidin-5-yl)-pyridin-3-ylamino]-methyl}-phenol,
Compound 182 (5-Thiophen-3-yl-pyridin-3-yl)-(3,4,5-trimethoxy-phenyl)-amine,
Compound 183 N-{3-[5-(4-Cyano-phenylamino)-pyridin-3-yl]-phenyl}-methanesulfonamide,
Compound 184 N-[3-(5-Phenylamino-pyridin-3-yl)-phenyl]-methanesulfonamide,
Compound 185 [3,3′]Bipyridinyl-5-yl-(3-chloro-phenyl)-amine,
Compound 186 4-[5-(3-Chloro-4-fluoro-phenylamino)-pyridin-3-yl]-phenol,
Compound 187 N-{3-[5-(3-Chloro-phenylamino)-pyridin-3-yl]-phenyl}-acetamide,
Compound 188 3-[5-(3,4,5-Trimethoxy-phenylamino)-pyridin-3-yl]-benzamide,
Compound 189 3-[5-(3,4,5-Trimethoxy-phenylamino)-pyridin-3-yl]-phenol,
Compound 190 3-(5-Phenylamino-pyridin-3-yl)-phenol,
Compound 191 {3-[5-(3-Chloro-4-fluoro-phenylamino)-pyridin-3-yl]-phenyl}-methanol,
Compound 192 N-{3-[5-(3-Chloro-phenylamino)-pyridin-3-yl]-phenyl}-methanesulfonamide,
Compound 193 N-(2-Hydroxy-ethyl)-3-[5-(naphthalen-2-ylamino)-pyridin-3-yl]-benzamide,
Compound 194 N-[3-(5-Phenylamino-pyridin-3-yl)-phenyl]-acetamide,
Compound 195 3-[(5-Pyrimidin-5-yl-pyridin-3-ylamino)-methyl]-phenol,
Compound 196 N-{3-[5-(3,4,5-Trimethoxy-phenylamino)-pyridin-3-yl]-phenyl}-methanesulfonamide,
Compound 197 4-[5-(3-Hydroxymethyl-phenyl)-pyridin-3-ylamino]-benzonitrile,
Compound 198 3-[5-(3-Chloro-phenylamino)-pyridin-3-yl]-N-(2-hydroxy-ethyl)-benzamide,
Compound 199 [5-(3,4-Dimethoxy-phenyl)-pyridin-3-yl]-(3-nitro-phenyl)-amine,
Compound 200 N-{3-[5-(3,4,5-Trimethoxy-phenylamino)-pyridin-3-yl]-phenyl}-acetamide,
Compound 201 3-[5-(4-Cyano-phenylamino)-pyridin-3-yl]-benzamide,
Compound 202 N-(2-Dimethylamino-ethyl)-4-[5-(3-hydroxy-benzylamino)-pyridin-3-yl]-benzamide,
Compound 203 4-[5-(4-Methanesulfonyl-phenyl)-pyridin-3-ylamino]-benzonitrile,
Compound 204 N-{3-[5-(4-Trifluoromethoxy-phenylamino)-pyridin-3-yl]-phenyl}-acetamide,
Compound 205 N-{3-[5-(4-Cyano-phenylamino)-pyridin-3-yl]-phenyl}-acetamide,
Compound 206 3-{[5-(4-Methanesulfonyl-phenyl)-pyridin-3-ylamino]-methyl}-phenol,
Compound 207 3-[(6′-Methoxy-[3,3′]bipyridinyl-5-ylamino)-methyl]-phenol,
Compound 208 3-[5-(3-Chloro-4-fluoro-phenylamino)-pyridin-3-yl]-phenol,
Compound 209 3-([3,4′]Bipyridinyl-5-ylaminomethyl)-phenol,
Compound 210 N-{3-[5-(3-Chloro-4-fluoro-phenylamino)-pyridin-3-yl]-phenyl}-methanesulfonamide,
Compound 211 N-{3-[5-(Naphthalen-2-ylamino)-pyridin-3-yl]-phenyl}-methanesulfonamide,
Compound 212 3-{[5-(4-Hydroxy-phenyl)-pyridin-3-ylamino]-methyl}-phenol,
Compound 213 N-{3-[5-(3-Chloro-4-fluoro-phenylamino)-pyridin-3-yl]-phenyl}-acetamide,
Compound 214 3-[(5-Benzo[1,3]dioxol-5-yl-pyridin-3-ylamino)-methyl]-phenol,
Compound 215 3-{[5-(3-Trifluoromethoxy-phenyl)-pyridin-3-ylamino]-methyl}-phenol,
Compound 216 3-{[5-(3-Hydroxymethyl-phenyl)-pyridin-3-ylamino]-methyl}-phenol,
Compound 217 3-[5-(3-Hydroxy-benzylamino)-pyridin-3-yl]-N-(2-hydroxy-ethyl)-benzamide,
Compound 218 3-{[5-(4-Morpholin-4-yl-phenyl)-pyridin-3-ylamino]-methyl}-phenol,
Compound 219 4-[5-(3-Hydroxy-phenyl)-pyridin-3-ylamino]-benzonitrile,
Compound 220 4-[5-(3-Nitro-phenylamino)-pyridin-3-yl]-phenol,
Compound 221 N-{3-[5-(3-Hydroxy-benzylamino)-pyridin-3-yl]-phenyl}-acetamide,
Compound 222 3-[(5′-Methoxy-[3,3′]bipyridinyl-5-ylamino)-methyl]-phenol,
Compound 223 3-{[5-(3,4-Dimethoxy-phenyl)-pyridin-3-ylamino]-methyl}-phenol,
Compound 224 N-(2-Dimethylamino-ethyl)-3-[5-(3-hydroxy-benzylamino)-pyridin-3-yl]-benzamide,
Compound 225 3-{5-[(3-Hydroxybenzyl)amino]pyridin-3-yl}phenol,
Compound 226 N-{3-[5-(3-Hydroxy-benzylamino)-pyridin-3-yl]-phenyl}-methanesulfonamide,
Compound 227 N-{3-[5-(4-Methoxy-phenylamino)-pyridin-3-yl]-phenyl}-acetamide,
Compound 228 3-[(5-Thiophen-3-yl-pyridin-3-ylamino)-methyl]-phenol.
Compound 229 3-{[5-(4-Hydroxymethyl-phenyl)-pyridin-3-ylamino]-methyl}-phenol,
Compound 230 3-[5-(3-Hydroxy-benzylamino)-pyridin-3-yl]-benzamide,
Compound 231 2-Fluoro-3-[5-(3-hydroxy-benzylamino)-pyridin-3-yl]-phenol,
Compound 232 3-{[5-(3-Amino-phenyl)-pyridin-3-ylamino]-methyl}-phenol,
Compound 233 3-{[5-(3-Hydroxy-phenyl)-pyridin-3-ylamino]-methyl}-2-methyl-phenol,
Compound 234 3-Hydroxy-N-[5-(3-hydroxy-phenyl)-pyridin-3-yl]-benzamide,
Compound 235 N-{3-[5-(4-Fluoro-phenylamino)-pyridin-3-yl]-phenyl}-acetamide,
Compound 236 3-{[5-(2-Fluoro-3-methoxy-phenyl)-pyridin-3-ylamino]-methyl}-phenol,
Compound 237 N-{3-[5-(2-Fluoro-phenylamino)-pyridin-3-yl]-phenyl}-acetamide,
Compound 238 3-[(5-Phenyl-pyridin-3-ylamino)-methyl]-phenol,
Compound 239 3-{[5-(3-Methoxy-phenyl)-pyridin-3-ylamino]-methyl}-phenol,
Compound 240 N-{3-[5-(2-Methoxy-phenylamino)-pyridin-3-yl]-phenyl}-acetamide,
Compound 241 3-{5-[(3-Hydroxy-benzyl)-methyl-amino]-pyridin-3-yl}-phenol,
Compound 242 5-{[5-(3-Hydroxy-phenyl)-pyridin-3-ylamino]-methyl}-benzene-1,3-diol,
Compound 243 3-[5-(3-Hydroxy-benzylamino)-pyridin-3-yl]-benzoic acid methyl ester,
Compound 244 3-{5-[2-(3-Hydroxy-phenyl)-ethylamino]-pyridin-3-yl}-phenol,
Compound 245 3-[5-(3-Amino-benzylamino)-pyridin-3-yl]-phenol,
Compound 246 3-[5-(3-Hydroxy-benzylamino)-pyridin-3-yl]-benzoic acid,
Compound 247 5-{[5-(3-Hydroxy-phenyl)-pyridin-3-ylamino]-methyl}-2-methyl-phenol,
Compound 248 3-[(5-Bromo-pyridin-3-ylamino)-methyl]-phenol,
Compound 249 3-{[5-(2-Hydroxy-phenyl)-pyridin-3-ylamino]-methyl}-phenol,
Compound 250 N-{3-[5-(Methyl-phenyl-amino)-pyridin-3-yl]-phenyl}-acetamide,
Compound 251 2-{[5-(3-Hydroxy-phenyl)-pyridin-3-ylamino]-methyl}-phenol,
Compound 252 [5-(3-Amino-phenyl)-pyridin-3-yl]-phenyl-amine,
Compound 253 N-[3-(5-Amino-pyridin-3-yl)-phenyl]-acetamide,
Compound 254 3-(5-Benzylamino-pyridin-3-yl)-phenol,
Compound 255 3-[5-(2-Fluoro-5-methoxy-benzylamino)-pyridin-3-yl]-phenol,
Compound 256 2-(3-Hydroxy-phenyl)-N-[5-(3-hydroxy-phenyl)-pyridin-3-yl]-acetamide,
Compound 257 3-(Pyridin-3-ylaminomethyl)-phenol,
Compound 258 3-[5-(3-Methoxy-benzylamino)-pyridin-3-yl]-phenol,
Compound 259 3-[5-(4-Fluoro-3-methoxy-benzylamino)-pyridin-3-yl]-phenol,
Compound 260 3-(5-Amino-pyridin-3-yl)-phenol,
Compound 261 3-{5-[(3-Methoxy-benzyl)-methyl-amino]-pyridin-3-yl}-phenol,
Compound 262 3-{[5-(3-Hydroxy-phenyl)-pyridin-3-ylamino]methyl}-benzoic acid methyl ester,
Compound 263 3-{[5-(3-Hydroxy-phenyl)-pyridin-3-ylamino]methyl}-benzoic acid,
Compound 264 3-{[5-(3-Hydroxy-phenyl)-pyridin-3-ylamino]methyl}-benzoic acid methyl ester,
Compound 265 3-{[5-(3-Hydroxy-phenyl)-pyridin-3-ylamino]-methyl}-benzamide,
Compound 266 3-[5-(3-Nitro-benzylamino)-pyridin-3-yl]-phenol,
Compound 267 N-[5-(3-Hydroxy-phenyl)-pyridin-3-yl]-3-methoxy-4-methyl-benzamide,
Compound 268 3-[5-(3-Methoxy-4-methyl-benzylamino)-pyridin-3-yl]-phenol,
Compound 269 3-Hydroxy-N-[5-(3-hydroxy-phenyl)-pyridin-3-yl]-2-methyl-benzamide,
Compound 270 (3-Methoxy-benzyl)-[5-(3-methoxy-benzyl)-pyridin-3-yl]-carbamic acid tert-butyl ester,
Compound 271 (3-methoxy-benzyl)-[5-(3-methoxy-benzyl)-pyridin-3-yl]-amine,
Compound 272 (3-hydroxy-benzyl)-[5-(3-hydroxy-benzyl)-pyridin-3-yl]-amine,
Compound 273 3-{[5-(3-Hydroxy-phenyl)-pyridin-3-ylamino]-methyl}-benzoic acid methyl ester,
Compound 274 (5-Phenyl-pyridin-3-yl)-phenyl-amine.

20-21. (canceled)

22. A method for the prophylaxis and/or treatment of an infectious disease, prion disease, immunological disease, autoimmune disease, bipolar or clinical disorder, cardiovascular disease, cell proliferative disease, diabetes, inflammation, osteoporosis, transplant rejection, erectile dysfunction, neurodegenerative disease, stroke, hair loss, obesity, polycystic ovary syndrome, ischaemia, leukopenia, Down's syndrome, Lewy body disease, periodontal disease, corneal ulceration, proteinuria, myelodysplastic syndrome, or biliary cirrhosis in a patient, comprising administering to the patient at least one compound according to claim 1.

23. A method according to claim 22, wherein the infectious disease is selected from AIDS, Alveolar Hydatid Disease (AHD, Echinococcosis), Amebiasis (Entamoeba histolytica Infection), Angiostrongylus Infection, Anisakiasis, Anthrax, Babesiosis (Babesia Infection), Balantidium Infection (Balantidiasis), Baylisascaris Infection (Raccoon Roundworm), Bilharzia (Schistosomiasis), Blastocystis hominis Infection (Blastomycosis), Boreliosis, Botulism, Brainerd Diarrhea, Brucellosis, BSE (Bovine Spongiform Encephalopathy), Candidiasis, Capillariasis (Capillaria Infection), CFS (Chronic Fatigue Syndrome), Chagas Disease (American Trypanosomiasis), Chickenpox (Varicella-Zoster virus), Chlamydia pneumoniae Infection, Cholera, Chronic Fatigue Syndrome, CJD (Creutzfeldt-Jakob Disease), Clonorchiasis (Clonorchis Infection), CLM (Cutaneous Larva Migrans, Hookworm Infection), Coccidioidomycosis, Conjunctivitis, Coxsackievirus A16 (Hand, Foot and Mouth Disease), Cryptococcosis, Cryptosporidium Infection (Cryptosporidiosis), Culex mosquito (Vector of West Nile Virus), Cutaneous Larva Migrans (CLM), Cyclosporiasis (Cyclospora Infection), Cysticercosis (Neurocysticercosis), Cytomegalovirus Infection, Dengue/Dengue Fever, Dipylidium Infection (Dog and Cat Flea Tapeworm), Ebola Virus Hemorrhagic Fever, Echinococcosis (Alveolar Hydatid Disease), Encephalitis, Entamoeba coli Infection, Entamoeba dispar Infection, Entamoeba hartmanni Infection, Entamoeba histolytica Infection (Amebiasis), Entamoeba polecki Infection, Enterobiasis (Pinworm Infection), Enterovirus Infection (Non-Polio), Epstein-Barr Virus Infection, Escherichia coli Infection, Foodborne Infection, Foot and mouth Disease, Fungal Dermatitis, Gastroenteritis, Group A streptococcal Disease, Group B streptococcal Disease, Hansen's Disease (Leprosy), Hantavirus Pulmonary Syndrome, Head Lice Infestation (Pediculosis), Heliobacter pylori Infection, Hematologic Disease, Hendra Virus Infection, Hepatitis (HCV, HBV), Herpes Zoster (Shingles), HIV Infection, Human Ehrlichiosis, Human Parainfluenza Virus Infection, Influenza, Isosporiasis (Isospora Infection), Lassa Fever, Leishmaniasis, Kala-azar (Kala-azar, Leishmania Infection), Lice (Body lice, Head lice, Pubic lice), Lyme Disease, Malaria, Marburg Hemorrhagic Fever, Measles, Meningitis, Mosquito-borne Diseases, Mycobacterium avium Complex (MAC) Infection, Naegleria Infection, Nosocomial Infections, Nonpathogenic Intestinal Amebae Infection, Onchocerciasis (River Blindness), Opisthorciasis (Opisthorcis Infection), Parvovirus Infection, Plague, PCP (Pneumocystis carinii Pneumonia), Polio, Q Fever, Rabies, Respiratory Syncytial Virus (RSV) Infection, Rheumatic Fever, Rift Valley Fever, River Blindness (Onchocerciasis), Rotavirus Infection, Roundworms Infection, Salmonellosis, Salmonella Enteritidis, Scabies, Shigellosis, Shingles, Sleeping Sickness, Smallpox, Streptococcal Infection, Tapeworm Infection (Taenia Infection), Tetanus, Toxic Shock Syndrome, Tuberculosis, Ulcers (Peptic Ulcer Disease), Valley Fever, Vibrio parahaemolyticus Infection, Vibrio vulnificus Infection, Viral Hemorrhagic Fever, Warts, Waterborne infectious Diseases, West Nile Virus Infection (West Nile Encephalitis), Whooping Cough, Yellow Fever, tuberculosis, leprosy, mycobacteria-induced meningitis, Chagas disease, effects of Shiga-like toxin resulting from Staphylococcus infection, meningococcal infection, and infections from Borrelia burgdorferi or Treponema pallidum.

24. Use according to claim 22, wherein the prion disease is selected from Scrapie, TME, CWD, BSE, CJD, vCJD, GSS, FFI, Kuru, and Alpers Syndrome.

25. A method according to claim 22, wherein the immunological disease and/or autoimmune disease is selected from:
asthma, diabetes, rheumatic diseases, AIDS, rejection of transplanted organs and tissues, rhinitis, chronic obstructive pulmonary diseases, ulcerative colitis, sinusitis, lupus erythematosus, recurrent infections, atopic dermatitis/eczema and occupational allergies, food allergies, drug allergies, severe anaphylactic reactions, anaphylaxis, manifestations of allergic diseases, primary immunodeficiencies, antibody deficiency states, cell mediated immunodeficiencies, severe combined immunodeficiency, DiGeorge syndrome, Hyper-IgE syndrome, Wiskott-Aldrich syndrome, ataxia-telangiectasia, immune mediated cancers, white cell defects, autoimmune diseases, systemic lupus erythematosus, rheumatoid arthritis (RA), multiple sclerosis (MS), immune-mediated or Type I Diabetes Mellitus, immune mediated glomerulonephritis, scleroderma, pernicious anemia, alopecia, pemphigus, pemphigus vulgaris, myasthenia gravis, inflammatory bowel diseases, Crohn's disease, psoriasis, autoimmune thyroid diseases, Hashimoto's disease, dermatomyositis, Goodpasture's syndrome, myasthenia gravis pseudoparalytica, ophtalmia sympatica, phakogene uveitis, chronic aggressive hepatitis, primary billiary cirrhosis, autoimmune hemolytic anemia, and Werlof disease.

26. A method according to claim 22, wherein the bipolar and/or clinical disorder is selected from: adjustment disorders, anxiety disorders, delirium, dementia, amnestic and other cognitive disorders, disorders usually first diagnosed in infancy, childhood, or adolescence, dissociative disorders, eating disorders, factitious disorders, impulse-control disorders, mental disorders due to a general medical condition, mood disorders, other conditions that may be a focus of clinical attention, personality disorders, schizophrenia and other psychotic disorders, sexual and gender identity disorders, sleep disorders, somatoform disorders, substance-related disorders, generalized anxiety disorder, panic disorder, phobia, agoraphobia, obsessive-compulsive disorder, stress, acute stress disorder, anxiety neurosis, nervousness, phobia, posttraumatic stress disorder, posttraumatic stress disorder (PTSD), abuse, obsessive-compulsive disorder (OCD), manic depressive psychosis, specific phobias, social phobia, and adjustment disorder with anxious features.

27. A method according to claim 26, wherein the bipolar and/or clinical disorder is selected from: acute stress disorder, agoraphobia without history of panic disorder, anxiety disorder due to general medical condition, generalized anxiety disorder, obsessive-compulsive disorder, panic disorder with agoraphobia, panic disorder without agoraphobia, posttraumatic stress disorder, specific phobia, social phobia, substance-induced anxiety disorder, delirium due to a general medical condition, substance intoxication delirium, substance withdrawal delirium, delirium due to multiple etiologies, Alzheimer's, Creutzfeldt-Jakob disease, head trauma, Huntington's disease, HIV disease, Parkinson's disease, Pick's disease, substance-induced persisting vascular dementia due to other general medical conditions, dementia due to multiple etiologies, amnestic disorder due to a general medical condition, substance-induced persisting amnestic disorder, mental retardation, learning disorders, mathematics disorder, reading disorder, disorder of written expression, learning disorder, motor skills disorders, developmental coordination disorder, communication disorders, expressive language disorder, phonological disorder, mixed receptive-expressive language disorder, stuttering, pervasive developmental disorders, Asperger's disorder, autistic disorder, childhood disintegrative disorder, Rett's disorder, pervasive developmental disorder, attention-deficit/hyperactivity disorder (ADHD), conduct disorder, oppositional defiant disorder, feeding disorder of infancy or early childhood, pica, rumination disorder, tic disorders, chronic motor or vocal tic disorder, Tourette's syndrome, elimination disorders, encopresis, enuresis, selective mutism, separation anxiety disorder, reactive attachment disorder of infancy or early childhood, stereotypic movement disorder, dissociative amnesia, depersonalization disorder, dissociative fugue, dissociative identity disorder, anorexia nervosa, bulimia nervosa, mood episodes, major depressive episode, hypomanic episode, manic episode, mixed episode, depressive disorders, dysthymic disorder, major depressive disorder, single episode, recurrent, bipolar disorders, bipolar I disorder, bipolar II disorder, cyclothymic disorder, mood disorder due to a general medical condition, substance-induced mood disorder, schizophreniform disorder, schizoaffective disorder, delusional disorder, brief psychotic disorder, shared psychotic disorder, psychotic disorder due to a general medical condition, delusions, hallucinations, substance-induced psychotic disorder, female sexual arousal disorder, orgasmic disorders, premature ejaculation, sexual pain disorders, dyspareunia, vaginismus, sexual dysfunction due to a general medical condition, female dyspareunia, female hypoactive sexual desire disorder, male erectile disorder, over-active bladder syndrome, male hypoactive sexual desire disorder, male dyspareunia, other female sexual dysfunction, other male sexual dysfunction, substance-induced sexual dysfunction, sexual dysfunction, exhibitionism, fetishism, frotteurism, pedophilia, masochism, sadism, transvestic fetishism, voyeurism, paraphilia, gender identity disorder, dyssomnias, breathing-related sleep disorder, circadian rhythm sleep disorder, hypersomnia, hypersomnia related to another mental disorder, insomnia, insomnia related to another mental disorder, narcolepsy, dyssomnia, parasomnias, nightmare disorder, sleep terror disorder, sleepwalking disorder, parasomnia, body dysmorphic disorder, conversion disorder, hypochondriasis, pain disorder, somatization disorder, undifferentiated somatoform disorder, alcohol related disorders, amphetamine related disorders, caffeine related disorders, cannabis related disorders, cocaine related disorders, hallucinogen related disorders, inhalant related disorders, nicotine related disorders, opioid related disorders, psychotic disorder, psychotic disorder, phencyclidine-related disorder, abuse, persisting amnestic disorder, anxiety disorder, persisting dementia, dependence, intoxication, intoxication delirium, mood disorder, psychotic disorder, withdrawal, withdrawal delirium, sexual dysfunction, and sleep disorder.

28. A method according to claim 22, wherein the cardiovascular disease is selected from:
adult congenital heart disease, aneurysm, stable angina, unstable angina, angina pectoris, angioneurotic edema, aortic valve stenosis, aortic aneurysm, arrhythmia, arrhythmogenic right ventricular dysplasia, arteriosclerosis, arteriovenous malformations, atrial fibrillation, Behcet syndrome, bradycardia, cardiac tamponade, cardiomegaly, congestive cardiomyopathy, hypertrophic cardiomyopathy, restrictive cardiomyopathy, cardiovascular disease prevention, carotid stenosis, cerebral hemorrhage, Churg-Strauss syndrome, diabetes, insulin resistance, non-insulin-dependent diabetes mellitus (NIDDM), Ebstein's Anomaly, Eisenmenger complex, cholesterol embolism, bacterial endocarditis, fibromuscular dysplasia, congenital heart defects, heart diseases, congestive heart failure, heart valve diseases, heart attack, epidural hematoma, hematoma, subdural hematoma, Hippel-Lindau disease, hyperemia, hypertension, pulmonary hypertension, hypertrophic growth, left ventricular hypertrophy, right ventricular hypertrophy, hypoplastic left heart syndrome, hypotension, intermittent claudication, ischemic heart disease, Klippel-Trenaunay-Weber syndrome, lateral medullary syndrome, long QT syndrome mitral valve prolapse, moyamoya disease, mucocutaneous lymph node syndrome, myocardial infarction, myocardial ischemia, myocarditis, pericarditis, peripheral vascular diseases, phlebitis, polyarteritis nodosa, pulmonary atresia, Raynaud disease, chronic renal failure, restenosis, Sneddon syndrome, stenosis, superior vena cava syndrome, syndrome X, tachycardia, Takayasu's arteritis, hereditary hemorrhagic telangiectasia, telangiectasis, temporal arteritis, tetralogy of fallot, thromboangiitis obliterans, thrombosis, thromboembolism, tricuspid atresia, varicose veins, vascular diseases, vasculitis, vasospasm, ventricular fibrillation, Williams syndrome, peripheral vascular disease, varicose veins and leg ulcers, deep vein thrombosis and, Wolff-Parkinson-White syndrome.

29. A method according to claim 22, wherein the proliferative disease is selected from:
advanced cancer, lymphoid malignancies and tumor metastases, adenocarcinoma, choroidal melanoma, acute leukemia, acoustic neurinoma, ampullary carcinoma, anal carcinoma, astrocytoma, basal cell carcinoma, pancreatic cancer, desmoid tumor, bladder cancer, bronchial carcinoma, breast cancer, Burkitt's lymphoma, corpus cancer, CUP-syndrome (carcinoma of unknown primary), colorectal cancer, small intestine cancer, small intestinal tumors, ovarian cancer, endometrial carcinoma, ependymoma, epithelial cancer types, Ewing's tumors, gastrointestinal tumors, gastric cancer, gallbladder cancer, gall bladder carcinomas, uterine cancer, cervical cancer, cervix, glioblastomas, gynecologic tumors, ear, nose and throat tumors, hematologic neoplasias, hairy cell leukemia, urethral cancer, skin cancer, skin testis cancer, brain tumors (gliomas), brain metastases, testicle cancer, hypophysis tumor, carcinoids, Kaposi's sarcoma, laryngeal cancer, germ cell tumor, bone cancer, colorectal carcinoma, head and neck tumors (tumors of the ear, nose and throat area), colon carcinoma, craniopharyngiomas, oral cancer (cancer in the mouth area and on lips), cancer of the central nervous system, liver cancer, liver metastases, leukemia, eyelid tumor, lung cancer, lymph node cancer (Hodgkin's/Non-Hodgkin's), lymphomas, stomach cancer, malignant melanoma, malignant neoplasia, malignant tumors gastrointestinal tract, breast carcinoma, rectal cancer, medulloblastomas, melanoma, meningiomas, Hodgkin's disease, mycosis fungoides, nasal cancer, neurinoma, neuroblastoma, kidney cancer, renal cell carcinomas, non-Hodgkin's lymphomas, oligodendroglioma, esophageal carcinoma, osteolytic carcinomas and osteoplastic carcinomas, osteosarcomas, ovarial carcinoma, pancreatic carcinoma, penile cancer, plasmocytoma, prostate cancer, pharyngeal cancer, rectal carcinoma, retinoblastoma, vaginal cancer, thyroid carcinoma, Schneeberger disease, esophageal cancer, spinalioms, T-cell lymphoma (mycosis fungoides), thymoma, tube carcinoma, eye tumors, urethral cancer, urologic tumors, urothelial carcinoma, vulva cancer, wart appearance, soft tissue tumors, soft tissue sarcoma, Wilm's tumor, cervical carcinoma and tongue cancer.

30. A method according to claim 22, wherein said diabetes is selected from Type I diabetes or Type II diabetes, non-insulin-dependent diabetes mellitus (NIDDM).

31. A method according to claim 22, wherein said inflammation is mediated by the cytokines TNF-α, IL-1β, GM-CSF, IL-6 and/or IL-8.

32. A method according to claim 22, wherein the inflammatory disease is caused, induced, initiated and/or enhanced by bacteria, viruses, prions, parasites, fungi, and/or caused by irritative, traumatic, metabolic, allergic, autoimmune, or idiopathic reasons.

33. A method according to claim 32, wherein the inflammatory disease is caused, induced, initiated, and/or enhanced by a virus and/or bacterium selected from human immunodeficiency virus-I, herpes viruses, herpes simplex virus (HSV-1 and HSV-2), HHV-6, HHV-7, HHV-8, Ebstein Barr virus (EBV), herpes zoster virus, varizella zoster virus (VZV), cytomegalovirus (HCMV), mycoplasma pulmonis, ureaplasma urealyticum, mycoplasma pneumoniae, chlamydia pneumoniae, C. pneumoniae, Heliobacter pylori, and propriono-bacterium.

34. A method according to claim 33,
wherein the inflammatory disease is selected from inflammatory diseases of the central nervous system (CNS), inflammatory rheumatic diseases, inflammatory diseases of blood vessels, inflammatory diseases of the middle ear, inflammatory bowel diseases, inflammatory diseases of the skin, inflammatory disease uveitis, and inflammatory diseases of the larynx.

35. A method according to claim 34, wherein the inflammatory disease is selected from:
abscessation, acanthameba, acanthamebiasis, acne vulgaris, actinomycosis, acute inflammatory dermatoses, acute laryngeal infections of adults, acute multifocal placoid pigmentary epitheliopathy, acute (thermal) injury, acute retinal necrosis, acute suppurative otitis media, algal disorders, allergic contact dermatitis, amyloidosis angioedema, ankylosing spondylitis, aspergillosis, atopic dermatitis, Aujeszky's disease, autoantibodies in vasculitis, babesiosis, bacterial disorders, bacterial laryngitis, bacterial meningitis, Behcet's disease, birdshot choroidopathy, blastomycosis, borna disease, brucellosis, bullous myringitis, bursitis, candidiasis, canine distemper encephalomyelitis, canine distemper encephalomyelitis in immature animals, canine ehrlichiosis, canine herpes virus encephalomyelitis, cholesteatoma, chronic (granulomatous) diseases, chronic inflammatory dermatoses, chronic relapsing encephalomyelitis, chronic suppurative otitis media, cicatricial pemphigoid, coccidiomycosis, coccidioidomycosis, common upper respiratory infection, contact ulcer and granuloma, Crohn's disease, cryptococcosis, cysticercosis, dermatomyositis, diphtheria, discoid lupus erythematosus, drug-induced vasculitis, drug or hypersensitivity reaction, encephalitozoonosis, eosinophilic meningoencephalitis, erythemal multiforme (EM minor), feline leukemia virus, feline immunodeficiency virus, feline infectious peritonitis, feline polioencephalomyelitis, feline spongiform encephalopathy, fibromyositis, Fuch's heterochromic cyclitis, gastroesophageal (laryngopharyngeal) reflux disease, giant cell arteritis, glanders, glaucomatocyclitic crisis, gonorrhea granular myringitis, granulomatous meningoencephalomyelitis, herpes simplex, histoplasmosis, idiopathic diseases, idiopathic inflammatory disorders, immune and idiopathic disorders, infections of the immunocompromised host, infectious canine hepatitis, inhalation laryngitis, interstitial nephritis, irritant contact dermatitis, juvenile rheumatoid arthritis, Kawasaki's disease, La Crosse virus encephalitis, laryngeal abscess, laryngotracheitis (croup), leishmaniasis, lens-induced uveitis, leprosy, leptospirosis, leukemia, lichen planus, lupus, lyme disease, lymphoma, meningitis, meningoencephalitis in greyhounds, miscellaneous meningitis/meningoencephalitis, microscopic polyangiitis, multifocal choroiditis, multifocal distemper encephalomyelitis in mature animals, multiple sclerosis, muscle tension dysphonias, mycotic (fungal) diseases, mycotic diseases of the CNS, necrotizing encephalitis, neosporosis, old dog encephalitis, onchocerciasis, parasitic encephalomyelitis, parasitic infections, pars planitis, parvovirus encephalitis, pediatric laryngitis, pollution and inhalant allergy, polymyositis, post-vaccinal canine distemper encephalitis, post-vaccinal rabies, prion protein induced diseases, protothecosis, protozoal encephalitis-encephalomyelitis, psoriasis, psoriatic arthritis, pug dog encephalitis, pyogranulomatous meningoencephalomyelitis, rabies, radiation injury, radiation laryngitis, radionecrosis, relapsing polychondritis, Reiters's syndrome, retinitis pigmentosa, retinoblastoma, rheumatoid arthritis, rickettsial disorders, rocky mountain spotted fever, salmon poisoning, sarcocystosis, sarcoidosis, schistosomiasis, scleroderma, scleroma, serpiginous choroiditis, shaker dog disease, Sjogren's syndrome, spasmodic croup, spirochetal (syphilis) diseases, spongiotic dermatitis, sporotrichosis, steroid responsive meningitis-arteritis, Stevens-Johnson syndrome (SJS, EM major), supraglottitis (epiglottitis), sympathetic ophthalmia, syngamus laryngeus, syphilis, systemic lupus erythematosus, systemic vasculitis in sarcoidosis, Takayasu's arteritis, tendinitis (tendonitis), thromboangiitis obliterans (Buerger's Disease), tick-borne encephalitis in dogs, toxic epidermal necrolysis (TEN), toxocariasis, toxoplasmosis, trauma, traumatic laryngitis, trichinosis, trypanosomiasis, tuberculosis, tularemia, ulcerative colitis, urticaria (hives), vasculitis, vasculitis and malignancy, vasculitis and rheumatoid arthritis, vasculitis in systemic lupus erythematosus, vasculitis in the idiopathic inflammatory myopathies, vasculitis of the central nervous system, vasculitis secondary to bacterial, fungal, and parasitic infection, viral disorders, viral laryngitis, vitiligo, vocal abuse, vocal-cord hemorrhage, Vogt Koyanagi Harada syndrome, Wegener's granulomatosis, Whipple's disease, osteoarthritis, septic arthritis, bone resorption, postmenopausal osteoporosis, sepsis, gram negative sepsis, septic shock, endotoxin shock, systemic inflammatory response syndrome, irritable bowel syndrome, Jarisch Heryheimer reactions, adult respiratory distress syndrome, acute pulmonary fibrotic diseases, pulmonary sarcoidosis, allergic respiratory diseases, COPD (chronic obstructive pulmonary disease), silicosis, coal worker's pneumoconiosis, alveolar injury, hepatic failure, liver disease during acute inflammation, immunodeficiency and fibrotic diseases, dermatosis, including psoriasis, atopic dermatitis, and ultraviolet radiation (UV)-induced skin damage.

36. A method Use according to claim 22, wherein the transplant rejection is selected from heart transplant rejection, heart-lung transplant rejection, lung transplant rejection, liver transplant rejection, kidney transplant rejection, pancreas transplant rejection, spleen transplant rejection, skin transplant rejection, tissue transplant rejection, bone marrow transplant rejection, spinal marrow transplant rejection, hormone producing glands transplant rejection, gonads and gonadal gland transplant rejection, graft-versus-host-diseases and host-versus-graft-diseases, systemic lupus erythematosis, ischemia reperfusion injury, allograft rejection, chronic lung, kidney and heart allograft rejection, complications due to total hip replacement, and alkylosing spondylitis.
4-[5-(3,4-Dimethoxy-benzylamino)-pyridin-3-yl]-phenol,
3-[5-(4-Chloro-benzylamino)-pyridin-3-yl]-benzamide,
3-[5-(3,4-Difluoro-benzylamino)-pyridin-3-yl]-benzamide,
4-[5-(3,4-Difluoro-benzylamino)-pyridin-3-yl]-phenol,
3-{[5-(4-Hydroxy-phenyl)-pyridin-3-ylamino]-methyl}-phenol,
3-{[5-(3-Hydroxymethyl-phenyl)-pyridin-3-ylamino]-methyl}-phenol,
3-[5-(3-Hydroxy-benzylamino)-pyridin-3-yl]-N-(2-hydroxy-ethyl)-benzamide,
3-{5-[(3-Hydroxybenzyl)amino]pyridin-3-yl}phenol,
3-{[5-(4-Hydroxymethyl-phenyl)-pyridin-3-ylamino]-methyl}-phenol,
3-[5-(3-Hydroxy-benzylamino)-pyridin-3-yl]-benzamide,
3-{[5-(3-Amino-phenyl)-pyridin-3-ylamino]-methyl}-phenol, or
3-[(5-Phenyl-pyridin-3-ylamino)-methyl]-phenol.

37. A method according to claim 22, wherein the neurodegenerative disease is selected from: Alzheimer disease, Parkinson disease, Huntington disease, amyotrophic lateral sclerosis, AIDS-related dementia, retinitis pigmentosa, spinal muscular atrophy and cerebellar degeneration, fragile X-associated tremor/ataxia syndrome (FXTAS), progressive supranuclear palsy (PSP), and striatonigral degeneration (SND) which is included with olivopontocerebellar degeneration (OPCD), Shy Drager syndrome (SDS) in a syndrome known as multiple system atrophy (MSA), acute encephalitis, brain injury, amyotrophic lateral sclerosis and inflammatory pain, regenerative (recovery) treatment of CNS disorders such as spinal cord injury, acute neuronal injury (stroke, traumatic brain injury) progressive supranuclear palsy, subacute sclerosing panencephalitic parkinsonism, postencephalitic parkinsonism, pugilistic encephalitis, guam parkinsonism-dementia complex, corticobasal degeneration, frontotemporal dementia, AIDS associated dementia, and mood disorders.

38. A pharmaceutical composition comprising at least one compound according to claim 1 as an active ingredient together with at least one pharmaceutically acceptable carrier, excipient or diluent.

39. A pharmaceutical composition according to claim 38, wherein the pharmaceutical composition is formulated as a pill, tablet, tab, film tablet, coated tablet, dragee, multi-layer tablet, capsule, powder, granulate, deposit, sustained release formulation, controlled release formulation, mini- and micro-formulation, nano-formulation, liposomal formulation, dispersion, suspension, liquid formulation, drop, injection, spray, ointment, cream, paste, syrup, lotion, and/or gel.

40. A compound according to claim 1, with the proviso that the compound is not:
4-[5-(4-Chloro-benzylamino)-pyridin-3-yl]-phenol,
4-[5-(3,4-Dichloro-benzylamino)-pyridin-3-yl]-phenol,
